(19) United States
(12) Patent Application Publication
(10) Pub. No.: US 2025/0339531 A1
(43) Pub. Date: Nov. 6, 2025

(54) DUX4 POLYPEPTIDES AND NUCLEIC ACIDS FOR TREATING INFLAMMATORY AND AUTOIMMUNE CONDITIONS

(71) Applicant: Fred Hutchinson Cancer Center, Seattle, WA (US)

(72) Inventors: Stephen J. Tapscott, Seattle, WA (US); Amy Elizabeth Spens, Seattle, WA (US); Danielle Christine Hamm, Seattle, WA (US); Robert K. Bradley, Seattle, WA (US)

(73) Assignee: Fred Hutchinson Cancer Center, Seattle, WA (US)

(21) Appl. No.: 18/708,819

(22) PCT Filed: Nov. 14, 2022
(86) PCT No.: PCT/US2022/079809

§ 371 Date: May 9, 2024

Related U.S. Application Data
(60) Provisional application No. 63/278,814, filed on Nov. 12, 2021.

(51) Int. Cl. A61K 40/30; A61P 35/00

(52) U.S. Cl. CPC A61K 40/30; A61P 35/00

(57) ABSTRACT

The current disclosure provides novel methods and compositions to suppress immunity and are useful for treating autoimmune and inflammatory conditions, and in some circumstances, cancers. Accordingly, aspects of the disclosure relate to a method for treating an inflammatory, autoimmune, autoinflammatory or cancer disease or condition in a subject comprising administering a DUX4 polypeptide or nucleic acid encoding a DUX4 polypeptide to the subject. Further aspects relate to a method for treating a tissue transplant subject, the method comprising administering a DUX4 polypeptide or nucleic acid encoding a DUX4 polypeptide to the subject. The present inventions also relate to universal donor stem cells that overcome immune rejection in cell-based transplantation therapies.

### CROSS-REFERENCES TO RELATED APPLICATIONS

This application is a National Stage of International Application No. PCT/US2022/079809, filed Nov. 14, 2022, which claims the benefit of U.S. Patent Application No. 63/278,814, filed Nov. 12, 2021, the disclosures of which are incorporated herein by reference in their entirety.

### STATEMENT OF GOVERNMENT LICENSE RIGHTS

This invention was made with Government support under AR045203, CA015704, and CA254805 awarded by National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 1896-P46US.PNP_Seq_List_20241015. The XML file is 124,625 bytes; was created on Oct. 15, 2024; and is being submitted electronically via Patent Center with the filing of the specification.

### FIELD OF THE INVENTION

The present invention relates generally to the field of medicine. More particularly, it concerns pharmaceutical compositions and methods for treating autoimmunity and for treating subjects with transplanted cells. The present invention also relates to methods of generating a universal donor stem cell to overcome the immune rejection in cell-based transplantation therapies.

### BACKGROUND

Autoimmune and inflammatory diseases arise from an abnormal immune response of the body against substances and tissues normally present in the body. This may be restricted to certain organs (e.g., in autoimmune thyroiditis) or involve a particular tissue in different places (e.g., Goodpasture's disease which may affect the basement membrane in both the lung and the kidney). Autoimmune and auto-inflammatory diseases affect up to 50 million people in America alone. The treatment of these diseases typically involves immunosuppressants—medications that decrease or dampen the immune response. Conventional immunotherapies using immunosuppressants, such as cyclosporine, tacroliums, methotrexate or anti-TNFα/IL-6, non-specifically suppress the function of T cells, including non-pathogenic T cells in the host. Therefore, treatment with these immunosuppressants often results in the development of severe infections and sometimes leads to lethal consequences.

The promise of universal donor stem cells has been underrealized due to immune rejection by the graft versus the host. There is a need in the art for additional therapeutic strategies for immunosuppression and methods for the generation and production of universal donor stem cells.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: shows DUX4 inhibits signaling through the IFIH1, RIGI, and CGAS pathways. MB135iDUX4 myoblasts were transfected with long double stranded RNAs (poly(I:C)), short dsRNA with a 5-prime triphosphate, or treated with 2′-5′-cGAMP to stimulate the IFIH1, RIGI, or STING (the downstream signaling factor in the cGAS pathway). Treatment with doxycycline (+) was used to induce the expression of DUX4 and RNA was measured for two ISG genes, ISG20 and IFIH1. Each gene had very low basal expression and was induced by the different treatments (−), whereas the presence of DUX4 (+) substantially blunted the ISG induction to all treatments. Numbers indicate the percentage of (+) induction relative to (−) induction.

FIG. 2. shows DUX4 blocks pathways induced through IRF and STAT signaling pathways. Similar to the design in FIG. 1, MB135iDUX4 cells were treated with inducers of the IFIH1, RIGI, or cGAS pathways (poly(I:C), short dsRNA, or cGAMP, respectively) that are predominantly regulated by IRF factors and also with IFNB1 or IFNG, Type I and Type II interferons, respectively, that predominantly regulated genes through STAT1 and STAT2 factors. RT-PCR was used to measure gene expression (IFIH1 and ISG20 in the top and bottom panels, respectively) in untreated or each treated condition. The first whisker plot in each pair represents the average level of the induced ISG without DUX4 (set to 1 and converted to a log 2 scale) and the second whisker plot represents the log 2-fold change of the induction in the presence of DUX4. These data show that pathways that rely on IRFs and STATs are both blocked by the induction of DUX4.

FIG. 3. shows alignment of the carboxyterminal regions of the DUX4 gene from humans (residues 371-424 of SEQ ID NO:3), Mouse (residues 626-674 of SEQ ID NO: 15) and the orthologous genes from additional mammals (SEQ ID NOS: 39-44) shows the conservation of the (L)LxxL(L) motifs.

FIG. 4. shows mutations of the two LLxxL motifs in the carboxyterminal region of DUX4 renders it inactive as a transcription factor. MB135 cells transduced with a doxycycline inducible DUX4 (iDUX4-CA) (SEQ ID NO:2 encoding the polypeptide of SEQ ID NO: 3) or a similar construct with the first LLxxL motif mutated and the second LLxxL motif deleted (iDUX4-mL1dL2) (SEQ ID NO: 4 encoding the polypeptide of SEQ ID NO:5). The level of expression in each panel, the left whisker plot indicates the expression of the DUX4-target gene ZSCAN4 in the absence of doxycycline induction of the DUX4 and the right whisker plot indicates the expression in the presence of doxycycline induction of DUX4, showing that DUX4 is a robust activator of its target gene ZSCAN4 whereas the mutant is no longer active as a transcription factor.

FIG. 5. shows mutations of the two (L)LxxL(L) motifs in the carboxyterminal region of DUX4 renders it inactive as a repressor of ISG induction by IFNG. MB135 myoblasts containing a doxycycline DUX4 (iDUX4-CA) (SEQ ID NO:2), encoding the polypeptide of SEQ ID NO:3 or DUX4 with the first LLxxL motif mutated and the second LxxLL motif deleted (iDUX4-mL1dL2) (SEQ ID NO:4), encoding the polypeptide of SEQ ID NO:5. RT-PCR determination of the abundance of the ISG gene mRNA indicated. The level of expression following stimulation by IFNG in the absence of doxycycline induction of the DUX4 wild-type or mutant (left whisker plot in each pair) is set to 1 and plotted on a log 2-scale. The log 2-fold change comparing stimulation by IFNG in the absence or presence of doxycycline induction of DUX4 or the DUX4 mutant is shown in the second whisker plot of each pair. DUX4 suppresses IFNG induction of all for ISGs, whereas the mutant does not suppress the IFNG induction of any of the four ISGs.

FIG. 6. shows specific regions of the DUX4 protein, but not its ability to induce gene transcription, are necessary to inhibit ISG induction. The top panels show the expression of the DUX4 regulated gene ZSCAN4 based on RT-PCR. Each box shows data for the MB 135 cell line transduced with doxycycline-inducible constructs that express DUX4 (iDUX4-CA) (SEQ ID NOS: 2-3), DUX4 with a mutation in the first homeodomain that prevents DNA binding (iDUX4-F67A) (SEQ ID NOS: 6-7), the terminal 271 amino acids of DUX4 that lack the two DNA binding homeodomains (longCTD) (SEQ ID NOS: 8-9), the terminal 86 amino acids of DUX4 (iNLS-CTD) (SEQ ID NOS: 12-13), or the longCTD region with disrupting mutations in the first (L)LxxL(L) motif and deletion of the second (L)LxxL(L) motif (longCTDmL1dL2) (SEQ ID NOS: 4-5). Top Panels: In each box the left whisker plot shows the expression level of the DUX4 regulated gene ZSCAN4 in the absence of doxycycline induction of the transgene, and the right whisker plot shows the expression in the presence of doxycycline induction of the transgene. Only the wild-type DUX4 shows transcriptional activation of its target gene. Bottom Panels: In each box a pair of whisker plots is shown for the level of mRNA expression for each gene assayed (IFIH1, ISG20, CXCL9, CD74): the left whisker plot of each pair shows the level of expression following IFNG induction with the average set to 1 and shown on a log 2 scale, and the right whisker plot in each pair shows the relative change in that level in the presence of both IFNG and of doxycycline induction of the transgene. The transcriptionally inactive protein DUX4-F67A fully suppresses the ISG response to IFNG compared to wild-type DUX4, the longCTD suppresses three of the four IFNG-induced genes, the iNLS CTD suppresses two of the four IFNG-induced, and the longCTD with the LLxxL(L) motifs disrupted does not suppress any of the IFNG-induced genes. This result demonstrates that specific regions of the DUX4 protein suppress distinct pathways of IFNG signaling to induce ISG genes.

FIG. 7. shows the transcriptional activation function of DUX4 is not necessary to suppress MHC class I protein expression while the DUX4 protein is present in the continuous protocol, but the transcription activation function of DUX4 is necessary to suppress MHC class I protein expression in the pulsed protocol at a time after the DUX4 protein has declined. Top panel: shows the timing for the continuous and the pulsed protocols with numbers representing hours. In the continuous protocol, doxycycline is added at time 0 hrs and left on continuously for 20 hrs with IFNG added for the last 16 hrs. In the pulsed protocol, the doxycycline is washed off after the first 4 hrs and IFNG is added later at 24 hrs for an additional 16 hrs. Lower panel: shows a western of MB135 cells with a doxycycline inducible DUX4 (MB135iDUX4ca#23) (SEQ ID NOS: 2-3) or DUX4 with the F67A mutation (SEQ ID NOS: 6-7) that disrupts DNA binding (MB135iDUX4(F67A) treated in the “continuous” or “pulsed” protocol as indicated. The transcriptionally active DUX4 protein suppresses MHC class I proteins and activates a DUX4-regulated gene H3.X/Y in both protocols, and even more effectively in the pulsed protocol, whereas the transcriptionally inactive DUX4-F67A protein inhibits MHC class I proteins in only the continuous protocol and does not induce expression of the DUX4-regulated H3.X/Y. These results indicate that there is a block to MHC class I induction in the presence of the DUX4 protein that does not require its transcriptional activity and is similar to the block in ISG induction, whereas there is also a longer duration block to MHC class I expression that requires the transcriptional activity of DUX4.

FIG. 8. shows the domain of DUX4 containing the LLxxL motifs is not necessary to suppress MHC class I proteins in the pulse protocol when this region is replaced with a generic activation domain. MB135 myoblasts were transduced with a doxycycline inducible vector expressing a DUX4-VP16 chimeric protein that has the first last 94 amino acids of DUX4 replaced with the generic activation domain of the VP16 protein. Protein lysates from cells uninduced (−/−), induced with IFNG (−/+) or induced with doxycycline and IFNG (+/24 hr) in the “pulsed” protocol. MHC class I proteins were suppressed by the DUX4-VP16 fusion that maintained transcriptional activity but lacked the LLxxL domains. E14-3 is an antibody to the amino-terminal region of DUX4 and GAPDH is used as a loading control.

FIGS. 9A-9E. show a brief pulse of DUX4 generates long-lasting suppression of IFN-γ-induced canonical MHC class I protein, but not the mRNAs or HLA-E. Schematic of experiment. MB135iDUX4 cells were untreated or subjected to continuous DUX4 (const.) or a brief pulse (pulse) of DUX4 expression prior to IFNG stimulation 4 or 28 hours later to induce MHC class I (FIG. 9A). Immunoblot for canonical (HLA-A/B/C; pan-MHC antibody) and non-canonical (HLA-E) MHC class I. A brief pulse of DUX4 suppressed HLA-A/B/C proteins even a day later (FIG. 9B). B2M and HLA-B mRNA levels, illustrating that HLA-A/B/C suppression occurs at the level of protein, not mRNA (FIGS. 9C and 9D, respectively). Similar results were obtained for HLA-A/C but for melanoma cells (Mel375iDUX4) (FIG. 9E). These data demonstrate that a brief pulse of DUX4 causes perdurant suppression of HLA-A/B/C proteins while maintaining HLA-E in multiple cell types. DUX4 similarly suppresses cell surface levels of canonical MHC class I (data not shown).

FIGS. 10A-10B. show DUX4-induced suppression of HLA-A/B/C persists for at least four days after a brief pulse of DUX4. MB135iDUX4 cells untreated or subjected to continuous DUX4 or a pulse of DUX4 expression, followed by IFNG stimulation to induce MHC class I 28, 52, or 76 hours later (FIG. 10A). Immunoblot for canonical (HLA-A/B/C; pan-MHC antibody) MHC class I (FIG. 10B). A brief pulse of DUX4 suppressed MHC class I even four days later, when DUX4 protein was absent.

FIG. 11. show either inhibition of the proteasome or expression of DUX4 will suppress MHC class I protein induction by IFNG but not induction of HLA-E. MB135iDUX4 myoblasts were treated as indicated in the continuous protocol (+/− doxycycline at 0 hrs, +/−IFNG or MG132 at 4 hrs, harvest at 20 hrs). Western shows IFNG induction of MHC class I and HLAE, and that MHC class I induction, but not HLA-E is blocked by either DUX4 or MG132, an inhibitor of the proteasome.

FIGS. 12A-12D. show the transcriptional activation function of DUX4 is necessary to fully suppress the IFNG induction of PSMB9 or PSMB10 protein expression while the DUX4 protein that lacks transcriptional activation function only partly suppresses these proteins in the continuous protocol and not at all in the pulse protocol. Schematic of continuous and pulse protocols (FIG. 12A). RT-PCR showing relative RNA abundance for the proteins shown in panel D following the continuous (FIG. 12B) or pulsed (FIG. 12C) protocol for DUX4, showing suppression some IFNG-induced RNAs in the continuous protocol but nearly full induction in the pulsed protocols. Same blot as in FIG. 7 showing suppression of MHC class I proteins by DUX4 with the added probing of the PSMB9 and PSMB10 proteins, two of the three components of the immunoproteasome induced by IFNG and other cytokines/interferons (FIG. 12D). The continuous or pulse DUX4 protocols completely block protein accumulation of PSMB9 and PSMB10, whereas the RNAs for these proteins are induced to nearly the same levels by IFNG in the pulse protocol, indicating a mainly post-transcriptional regulation of these proteins.

FIG. 13. shows a 3×FLAG-tagged version of the longCTD (SEQ ID NOS: 8-9) or the longCTDmL1dL2 (SEQ ID NOS: 10-11) can be used to identify interacting proteins. MB135 cells transduced with doxycycline inducible versions of these expression constructs were either not induced with doxycycline and treated with IFNG (+IFNG mix, where mix indicates that the samples from the longCTD and longCTDmL1dL2 were mixed together), or with IFNG and doxycycline (+IFNG+Dox). The cells were lysed in a lysis buffer and proteins purified that associated with magnetic beads coated with an anti-FLAG antibody. Proteins eluted from the beads were separated by gel electrophoresis and visualized with a silver stain. The location of the band representing the 3×FLAG tagged protein is indicated by the labeled arrow. Distinct protein bands are co-precipitated with each of the FLAG-tagged proteins, some of which are enhanced in the longCTD sample compared to the longCTDmL1dL2 sample (small arrows).

FIGS. 14A-14D. show DUX4 expression alters the distribution of STAT1, a key component of the IFNG signaling pathway. MB135iDUX4 cells were treated with IFNG in the absence of doxycycline induction of DUX4 (FIGS. 14A and 14B, DAPI and anti-STAT1, respectively) or in the presence of DUX4 induction by doxycycline (FIGS. 14C and 14D, DAPI and anti-STAT1, respectively). In the absence of DUX4, the IFNG treated cells show a homogeneous distribution of STAT1 in the nucleus and some in the cytoplasm, whereas in the presence of DUX4 the STAT1 is either absent or mislocalized into clumps in the nucleus (arrows indicating examples).

FIG. 15. shows DUX4 protein schematic. Dark grey bars represent homeodomains (HDs), black bars represent LLxxL motifs (L1, L2).

FIG. 16. shows DUX4 protein schematic. Dark grey bars represent homeodomains (HDs), white bars represent mutated (mL1) or deleted (mL2) LLxxL(L) motifs.

FIG. 17. shows DUX4 protein schematic. Dark grey bars represent homeodomains (HDs), black bars represent LLxxL(L) motifs (L1, L2). White bars represent mutations.

FIG. 18. shows DUX4 protein schematic. Black bars represent LLxxL(L) motifs (L1, L2).

FIG. 19. shows DUX4 protein schematic. White bars represent mutated or deleted LLxxL(L) motifs (mL1).

FIG. 20. shows DUX4 protein schematic. Black bars represent (L)LxxL(L) motifs (L1, L2).

FIG. 21 shows an alignment of the homeodomains from the DUX protein in different species, homeodomain 1 represented by residues 18-78 of SEQ ID NO:3 and SEQ ID NOS: 45-69; and homeodomain 2 represented by residues 91-157 of SEQ ID NO:3 and SEQ ID NOS: 70-94.

FIG. 22 shows that STAT1 is associated with the DUX4 longCTD and the longCTDmL1dL2 in IFNG treated MB135 cells. Cellular proteins were extracted from IFNG treated MB135 cells expressing the doxycycline inducible longCTD (SEQ ID NOS: 8-9) or the longCTDmL1dL2 (SEQ ID NOS: 10-11) (+IFNG+Dox) and a 50:50 mix of these cells in the presence of IFNG treatment but not doxycycline (+IFNG mix) and mixed with magnetic beads coated with an anti-flag antibody, and the beads and associated proteins purified. The flag-tagged longCTD and the flag-tagged long-CTDmL1dL2 were both purified using the anti-flag antibody (Anti-Flag(M2)(F1804). STAT1 also co-purified together with the DUX4 CTD proteins (Anti-STAT1[EPR4407], indicating a protein complex containing both the DUX4 CTD proteins and STAT1.

FIG. 23 shows that mouse DUX suppresses the IFNG induction of ISG genes in human MB135 cells. Human MB135 myoblasts with a doxycycline-inducible codon altered mouse DUX have a strong induction of IFIH1, ISG20, CXCL9, and CD74 in response to IFNG, but this induction is completely blocked by the doxycycline induction mouse DUX, indicating that the LLxxL(L) and other regions conserved between mouse DUX and human DUX4 have functional conservation in modulating the response to IFNG.

FIGS. 24A-24B. show DUX4 suppresses interferon-stimulated gene (ISG) induction. MB135 cells expressing doxycycline-inducible DUX4 (MB135-iDUX4), parental MB135 cells, or MB135 cells expressing doxycycline-inducible DUXB (MB135-iDUXB) were untreated, treated with IFNG, or treated with doxycycline and IFNG. RT-qPCR was used to evaluate expression of a DUX4 target gene, ZSCAN4, and interferon-stimulated genes IFIH1, ISG20, CXCL9, and CD74. Ct values were normalized to the housekeeping gene RPL27. Data represent the mean±SD of three biological replicates with three technical replicates each (FIG. 24A). (See FIG. 31A for data for biological replicates in independent cell lines). MB135-iDUX4 cells were untreated, treated with either IFNβ (Type-1 IFN pathway), poly(I:C) (IFIH1/MDA5 pathway), RIG-I ligand (DDX58/RIGI pathway), or cGAMP (cGAS/STING pathway), or treated with doxycycline and the same immune reagent. RT-qPCR was used to evaluate expression of IFIH1, ISG20, CXCL9, and CD74. Ct values were normalized to the housekeeping gene RPL27. Data represent the mean±SD of three biological replicates with three technical replicates each (FIG. 24B).

FIGS. 25A-25D. show DUX4 transcriptional activity is not necessary for ISG suppression, whereas the C-terminal domain (CTD) is both necessary and sufficient. MB135 cell lines with the indicated doxycycline inducible transgene±doxycycline, were evaluated for ZSCAN4 expression by RT-qPCR as a measure of the ability of the construct to activate a DUX4-target gene. Ct values were normalized to the housekeeping gene RPL27. Data represent the mean±SD of three biological replicates (FIG. 25A). MB135 cell lines with the indicated doxycycline inducible transgene were treated with IFNG±doxycycline. RT-qPCR was used to evaluate expression of IFIH1, ISG20, CXCL9, and CD74 and Ct values were normalized to the housekeeping gene RPL27, then normalized to the IFNG-only treatment to set the induced level to 100%. Data represent the mean±SD of three biological replicates with three technical replicates each (FIGS. 25B-25D). Light gray, homeodomains; medium gray, conserved region of CTD; black, (L)LxxL(L) motifs; * indicates sites of mutation for F67A in HD1 and mutation of first LLDELL to AADEAA. (See FIG. 31 for data related to additional cell lines).

FIG. 26. shows that the DUX4 protein interacts with STAT1 and additional immune response regulators. Left panel, representative candidate interactors identified by mass spectrometry of proteins that co-immunoprecipitated with the DUX4-CTD and their relative ranking in the candidate list (see Table 1 for full list). Right panel, validation western blot of proteins that co-immunoprecipitate with the DUX4-CTD in cell lysates from MB135 cells expressing doxycycline-inducible 3×FLAG-DUXB or 3×FLAG-DUX4-CTD, ±IFNG treatment.

FIGS. 27A-27B. show that the DUX4-CTD preferentially interacts with pSTAT1-Y701. Western blot showing input and immunoprecipitated proteins from either 3×FLAG-iDUXB (iDUXB) or 3×-FLAG-iDUX4-CTD cells (iDUX4) precipitated with anti-FLAG and probed with the indicated antibodies. Serial deletions of the iDUX4-CTD were assayed as indicated (FIG. 27A). Anti-FLAG immunoprecipitation from 3×-FLAG-iDUX4-CTD or 3×FLAG-iDUXB cells containing doxycycline inducible 3×MYC-iSTAT1, -iSTAT1-Y701A, or -iSTAT1-S727A with or without IFNG treatment and probed with the indicated antibodies (FIG. 27B).

FIGS. 28A-28E. show that the DUX4-CTD tethers phosphorylated STAT1 in the nucleus and moderately decreases STAT1 occupancy at ISG promoters. Immunofluorescence with anti-FLAG or anti-STAT1 of MB135-3×FLAG-iDUX4-CTD or MB135-3×FLAG-iDUXB cells treated with IFNG, doxycycline, or both (FIG. 28A). Chromatin immunoprecipitation using anti-STAT1 or IgG from MB135-iDUX4-CTD cells untreated, or IFNG and doxycycline. Ab1: 50:50 mix of STAT1 antibodies Abcam ab239360 and ab234400; Ab2: Abcam ab109320. ChIP-qPCR analysis relative to a standard curve constructed from purified Input DNA was used to determine the quantity of DNA per IP sample, which was then graphed as percent (%) of Input. Data represent the mean±SD of two biological replicates with 3 technical replicates each (FIG. 28B, left four panels). RT-qPCR of RNA from cells used for STAT1 ChIP showing induction of ISGs by IFNG and suppression by DUX4-CTD (FIG. 28B, right panel). Proximity-ligation assay (PLA) shows co-localization of endogenous STAT1 and pSTAT1 701 with the iDUX4-CTD compared to the interaction with the DUXB-CTD, in the nuclear compartment of IFNG- and doxycycline-treated MB135 cells. Mean fluorescent intensity (MFI) of the nuclei in the PLA channel was measured for 10 images per cell line and treatment and plotted (**** p<0.0001, unpaired t-test). An unpaired t-test was used because the samples are biologically independent (FIG. 28C).

FIGS. 28D-28E. show that the DUX4-CTD decreases STAT1 occupancy at ISG promoters and blocks Pol-II recruitment. Chromatin immunoprecipitation using anti-STAT1 or IgG from MB135-iDUX4-CTD cells untreated, IFNG-treated, or IFNG and doxycycline treated. Ab1: 50:50 mix of STAT1 antibodies Abcam ab239360 and ab234400; Ab2: Abcam ab109320. ChIP-qPCR analysis relative to a standard curve constructed from purified input DNA was used to determine the quantity of DNA per IP sample, which was then graphed as a percent (%) of input. Data represent the mean±SD of two biological replicates with 3 technical replicates each (**** p<0.0001, *** p<0.01, ** p<0.05, unpaired t-test). An unpaired t-test was used because the samples are biologically independent (FIG. 28D, left four panels). RT-qPCR of RNA from cells used for STAT1 ChIP showing induction of ISGs by IFNG and suppression by DUX4-CTD (FIG. 28D, right panel). CUT &Tag data showing the intensity of Pol-II signal across a 2000 bp window centered on the TSS of ISGs (left) or IFNG-unchanged genes (right) in untreated, IFNG-treated, or IFNG and doxycycline treated MB135-iDUX4-CTD cells (FIG. 28E).

FIGS. 29A-29G. show endogenous DUX4 expression in FSHD myotubes, a sarcoma cell line expressing a CIC-DUX4 fusion gene, and expression of mouse Dux in MB135 cells suppress ISGs. FSHD MB200 myoblasts were differentiated into myotubes, which results in the expression on endogenous DUX4 in a subset of myotubes. Cultures were treated±IFNG and DUX4 and IDO1 visualized by immunofluorescence. A representative image of DUX4+ and DUX4-myotubes shows IDO1 induction only in the DUX4-myotubes (FIG. 29A). RT-PCR of the indicated genes in MB135-iDux cells treated with IFNG, ±doxycycline. Ct values were normalized to the housekeeping gene RPL27, then normalized to the IFNG-only treatment to set the induced level to 100%. Data represent the mean±SD of three biological replicates with three technical replicates each (FIG. 29B). Western blot showing input and immunoprecipitated proteins from either 3×FLAG-iDux or 3×-FLAG-iDUXB cells+/−IFNG precipitated with anti-FLAG and probed with the indicated antibodies (FIG. 29C). RT-qPCR of the indicated genes in MB135 parental or Kitra-SRS that express a CIC DUX4-fusion gene containing the DUX4 CTD (FIG. 29D, left panel). Cells were transfected with control or CIC- and DUX4-targeting siRNAs. Ct values were normalized to the housekeeping gene RPL27. Data represent the mean±SD of three biological replicates with three technical replicates each (FIG. 29D, left panel). Western blot showing lysates from MB135 or Kitra-SRS cells treated with control or CIC- and DUX4-targeting siRNAs±IFNG and probed with the indicated antibodies (FIG. 29D, right panel). RT-qPCR of the indicated genes in MB135 with an inducible CIC (MB135-iCIC) or an inducible CIC-DUX4 fusion gene (MB135-iCIC-DUX4). Cells were untreated, IFNG-treated, or IFNG and doxycycline treated. Ct values were normalized to the housekeeping gene RPL27, then normalized to the IFNG-only treatment to set the induced level to 100%. Data represent the mean±SD of three biological replicates with three technical replicates each (FIG. 29E). RT-PCR of the indicated genes in MB135-iDux cells untreated or treated with IFNG±doxycycline. Ct values were normalized to the housekeeping gene RPL27, then normalized to the IFNG-only treatment to set the induced level to 100%. Data represent the mean±SD of three biological replicates with three technical replicates each (FIG. 29F, Left panel). Western blot showing input and immunoprecipitated proteins from either 3×FLAG-iDux or 3×-FLAG-iDUXB cells±IFNG precipitated with anti-FLAG and probed with the indicated antibodies (FIG. 29F, right panel).

FIG. 29G shows a model supported by the data showing how the DUX4-CTD might prevent STAT1 ISG induction. In the absence of the DUX4-CTD, pSTAT1 Y701 (black “P”) dimerizes, translocates to the nucleus, binds its GAS motif in the ISG promoter, acquires secondary phosphorylation at S727 (grey “P”), and recruits a stable transcription complex that includes Pol II to drive transcription of ISGs (FIG. 29G, Top). In the presence of the DUX4-CTD, STAT1 is phosphorylated, translocates to the nucleus, and binds its GAS motif as evidenced by the pSTAT1 S727 in complex with the CTD (FIG. 29G, Bottom). However, diminished steady-state occupancy of STAT1 at the ISG promoters and absence of Pol-II recruitment indicate that the STAT1-DUX4-CTD complex does not stably bind DNA and fails to recruit Pol-II and the pre-initiation complex. The (L)LxxL(L) motifs (black bars in DUX4-CTD) are necessary to interfere with transcription suppression and likely prevent STAT1 from interacting with a factor in the pre-initiation complex or recruit a co-repressor.

FIGS. 30A-30B. show transgene diagrams. Schematic depiction of transgenes used in this disclosure highlighting the N-terminal homeodomains (light grey in DUX4, no fill in DUXB, light grey in mouse Dux (mouse Dux constructs are shown below the DUXB and above endogenous CIC constructs)), DNA-binding HMG box (dark grey in CIC and CIC-DUX4), conserved C-terminal domain (medium grey in DUX4, CIC-DUX4, and mouse Dux), (L)LxxL(L) (black in DUX4, CIC-DUX4, and mouse Dux), mutations (* and black bar F67A, * replacement of (L)LxxL(L) with AADEAA), and 3×FLAG-NLS cassette regions (no fill) (FIG. 30A) (DUX 4 (residues 369-424 of SEQ ID NO:3); DUX4mL1 (SEQ ID NO: 99); DUX4dL2 (SEQ ID NO:100); DUX4mL1dL2 (residues 369-419 of SEQ ID NO: 5); Mouse Dux (residues 622-674 of SEQ ID NO:15); Inducible 3×FLAG-CIC (SEQ ID NO: 102); Inducible 3×FLAG-CICDUX4 (SEQ ID NO: 104); Inducible 3×FLAG-NLS-DUXB (SEQ ID NO:106). Nucleotide sequences for Inducible 3×FLAG-CIC (SEQ ID NO: 101); Inducible 3×FLAG-CICDUX4 (SEQ ID NO: 103); Inducible 3×FLAG-NLS-DUXB (SEQ ID NO:105). MUSCLE alignment of the terminal ˜50aa of the human DUX4, mutated human DUX4 (mL1, dL2, mL1dL2), and mouse Dux constructs used (FIG. 30B).

FIGS. 31A-31F. show data for biological replicates in independent cell lines for each DUX4 construct. Additional subcloned MB 135 cell lines of the iDUX4 (FIG. 31A), iDUX4-F67A (FIG. 31B), iDUX4-CTD (FIG. 31C), iDUX4aamL1DL2 (FIG. 31D), iDUX4-CTDmL1dL2 (FIG. 31E), iDUX4aa339-324 (FIG. 31F) treated with IFNG+doxycycline. RT-qPCR shows ISG expression graphed as a % of IFNG-only. Immunofluorescence panels show protein expression and nuclear localization using an antibody against the N-terminal (E14-3) or C-terminal (E5-5) residues of DUX4 as appropriate for the construct.

FIGS. 32A-32B. show in vitro translated DUX4-CTD does not co-immunoprecipitate with in vitro translated/phosphorylated STAT1. 3×MYC-STAT1, 3×MYC-STAT1-S727E, 3×FLAG-DUX4-CTD, and 3×FLAG-DUXB proteins were prepared using a rabbit reticulocyte lysate system. 3×MYC-STAT1 was incubated with 3×FLAG-DUX4-CTD, both either phosphorylated at Y701 with JAK1 kinase or untreated. Western blots show inputs and proteins immunoprecipitated with anti-FLAG and probed for STAT1, pSTAT1-Y701, MYC-tag, and FLAG-tag, as indicated (FIG. 32A). 3×MYC-STAT1-S727E was phosphorylated at Y701 with JAK1 kinase and incubated with 3×FLAG-DUX4-CTD or 3×FLAG-DUXB (FIG. 32B).

FIG. 33. shows expression of the DUX4-CTD elevates total levels of pSTAT1-Y701 and pSTAT1-S727. MB135-iDUXB or MB135-iDUX4-CTD cells were left untreated, treated with doxycycline, treated with IFNG, or treated with both doxycycline and IFNG. Total cellular protein was harvested and analyzed by SDS-PAGE and western blotting with antibodies against STAT1, pSTAT1-Y701, pSTAT1-S727, FLAG tag, and GAPDH as a loading control.

FIGS. 34A-34B. show Mouse Dux contains a triplication of the (L)LxxL(L)-containing region. Mouse Dux protein sequence with homeodomains in bold and (L)LxxL(L) motifs underlined (SEQ ID NO:15) (FIG. 34A). Alignment of a partial triplication of the mouse Dux protein with aa258-440 aligning with aa441-623 and aa624-650 aligning with aa258-284 (SEQ ID NO:15) (FIG. 34B).

FIGS. 35A-35E. show DUX4 suppresses antigen presentation factors through two mechanisms, transcriptionally and post-transcriptionally. Schematic of experimental time course (FIG. 35A). MB135iDUX4ca myoblasts were treated with doxycycline (DOX) to induce the DUX4 codon altered (ca) transgene, either for 20 hours continuously or a brief 4-hour pulse, followed by interferon-gamma (IFNG) treatment. Immunoblot analysis of DUX4, MHC Class I (MHC-I), immunoproteasome subunits (PSMB8, PSMB9, and PSMB10), constitutive proteasome subunit PSMB6, and GAPDH protein following treatment with doxycycline and/or IFNG (FIG. 35B). Cells expressing DUX4 continuously (left) were harvested 20 hours post-dox addition, and cells expressing a pulse of DUX4 (right) harvested at 44 hours. Normalized RNA-seq read counts of MHC-I mRNAs HLA-A, HLA-B, HLA-C, and immunoproteasome subunits PSMB8, PSMB9, and PSMB10 (FIG. 35C). Immunoblot analysis of DUX4, DUX4-targets H3.X/Y/Z, MHC-I, PSMB9, PSMB10, and GAPDH in MB135 myoblasts expressing dox-inducible DUX4 ((FIG. 35D, left) or DNA-binding mutant DUX4(F67A) ((FIG. 35D, right). DUX4 antibody (αE14-3) used to detect both wildtype and mutant DUX4 proteins. Immunoblot analysis of extended experimental time course outlined in (FIG. 35A) (FIG. 35E). Prolonged protein suppression of IFNG-stimulated MHC-I, PSMB8, PSMB9, and PSMB10 occurs in cells expressing DUX4 continuously and persists days after a DUX4 pulse.

FIGS. 36A-36F. show a pulse of DUX4 negatively regulates the status of multiple translational regulators. Immunoblot analysis of DUX4, target gene H3.X/Y, and translational regulators 0-7 days following a 4-hour pulse of doxycycline in MB135iDUX4ca myoblasts (FIG. 36A). Genotype of polyclonal PKR knockout in MB135iDUX4ca myoblasts (FIG. 36B). Immunoblot analysis of MB135iDUX4ca myoblasts with wildtype PKR or in PKR knockout background (FIG. 36C). Cells were treated with IFNG alone, with doxycycline for 20 hours continuously and stimulated with IFNG the terminal 16 hours, or with a brief 4-hour pulse of doxycycline followed by 16-hour IFNG treatment 48 hours later. Immunoblot analysis of MB135 parental myoblasts that were treated with mTOR inhibitors Everolimus or Torin2 for 24 hours, then treated again with inhibitor with and without IFNG for an additional 16 hours (FIG. 36D). Immunoblot analysis of MB135 parental myoblasts that were treated with 4EGI-I for 48 hours, then treated again with 4EGI-1 with and without IFNG for an additional 16 hours (FIG. 36E). FIG. 36F shows schematic of cap-dependent translation initiation complex (FIG. 36F, top) and immunoblot analysis of m7GTP pull-downs of eIF4E and associated eIF4G, eIF4A, and 4EBP1 (FIG. 36F, bottom) from MB135iDUX4 myoblasts treated with and without a pulse of DUX4 harvested at 68 hours.

FIGS. 37A-37E. show metabolic labeling of nascent protein synthesis following a pulse of DUX4 shows broad suppression. Schematic of experimental time course (FIG. 37A). Cells were metabolically labeled with [35S]-methionine/cysteine for 8 hours following a doxycycline-induced pulse of DUX4 and harvested 20, 44, 68, or 92h post-doxycycline treatment. Phosphorscreen autoradiography of samples pulsed with wildtype DUX4 (FIG. 37B top, left) or DUX4 containing DNA-binding mutant F67A (FIG. 37B top, right) in human myoblasts; Coomassie stain of total protein (FIG. 37B, bottom). Quantification of relative 35S signal normalized to paired 0-hour condition shows a reduction in labeled nascent proteins several days following a pulse of wildtype DUX4, and no significant change in cells expressing mutant DUX4(F67A) (FIG. 37C). Immunoblot analysis of samples harvested in parallel with 35S-labeled samples confirms transient doxycycline induction of DUX4 and DUX4(F67A) DNA-binding mutant, suppression of PSMB9, and hypophosphorylation of 4EBP1 (FIG. 37D). Immunofluorescence of HPG/Click-it labeled proteins in cells supplemented with or without HPG treatment, treated with translational inhibitor cycloheximide (CHX), or in cells labeled 20-96 hours following a pulse of DUX4 in MB135-iDUX4ca myoblasts (FIG. 37E).

FIGS. 38A-38E. show Ribosome footprinting revealing DUX4 effects on translation efficiency and a reduction in 5′ ribosome occupancy. Experimental schematic illustrating genome-wide quantification of mRNA counts and ribosome-protected mRNA fragments using RNA sequencing (RNA-seq) and ribosome profiling (Ribo-seq) in MB135iDUX4 myoblasts pulsed with and without DUX4 and treated with and without IFNG (FIG. 38A). Metagene analysis of ribosome-protected fragments (RPFs) along the length of cellular mRNAs in untreated MB135iDUX4 myoblasts, MB135iDUX4 myoblasts treated with doxycycline for 4 hours to induce a pulse of DUX4, IFNG treatment alone, and combined DUX4 pulse+IFNG (FIG. 38B). Schematic of annotated mRNA features 5′ untranslated region (UTR), Translation Start Site (TSS, −/+13 nucleotides around start codon), first coding exon, coding sequences (CDS), and 3′UTR (FIG. 38C). Box plots of translational changes occurring at the level of mRNA features (|log 2FC>1|, p-adj<0.05). Statistical comparisons indicated were conducted using one-way ANOVA, * p<0.001, ** p<0.0001, p<2.2e-16 (FIG. 38D). Gene Ontology analysis for transcripts with significantly decreased ribosome occupancy in 5′UTR, TSS, and first coding exon in MB135iDUX4 myoblasts treated with a DUX4 pulse+IFNG versus IFNG (FIG. 38E).

FIGS. 39A-39I. show Polysome profiling indicating DUX4 reprogramming of the translatome. Absorbance at 254 nm across a density gradient fractionation system. MB135iDUX4 myoblasts treated with IFNG alone, or 68 hours following a pulse of DUX4 with IFNG treatment (DUX4 pulse+IFNG); traces represent mean±SD of biological triplicates (FIG. 39A). RNA-sequencing analysis of mRNA levels in high polysome fractions relative to sub-polysome fractions (high/sub). Volcano plot showing log 2 fold-change differential abundance in cells treated with DUX4 pulse+IFNG versus IFNG (significance defined as base mean >50, |log 2FC>1|, p-adj<0.01) (FIG. 39B). Gene Ontology analysis for transcripts with significantly increased polysome abundance (high/sub) in MB135iDUX4 myoblasts treated with a DUX4 pulse+IFNG versus IFNG (FIG. 39C). Relative polysome abundance for 5′TOP mRNAs compared to all other mRNAs. Polysome abundance represents mRNA read counts in the high polysome fraction relative to the sub-polysome fraction. Log 2 fold-change represent differential read counts in MB135iDUX4 myoblasts treated with a DUX4 pulse+IFNG versus IFNG treatment alone (FIG. 39D). Log 2 fold-change in polysome abundance (high/sub) of MHC-I mRNAs, iProteasome subunits, select TOP mRNAs, and myogenic factors. Data represent mean±SD of biological triplicates (FIG. 39E). Immunoblot analysis of total protein lysate harvested for polysome profiling samples representing biological triplicates. MB135iDUX4 myoblasts treated with a DUX4 pulse+IFNG expressed DUX4 targets DUXA, H3.X, H3.Y, MBD3L2, and ZSCAN4. DUX4 pulsed cells showed enhanced phosphorylation of eEF2 at Thr56 and reduced phosphorylation of eIF4E at Ser209. MHC-I, iProteasome subunits PSMB8, PSMB9, and PSMB10, TOP mRNA-encoded ribosomal proteins RPL10A, RPL4, RPS6, RPS15A, and myogenic factors MYOD1 and DES were suppressed in cells following a DUX4 pulse+IFNG relative to cells treated with IFNG alone. GAPDH serves as loading control (FIG. 39F). RNA-sequencing analysis of high polysome fractions (high/high). Volcano plot showing log 2 fold-change of DUX4 pulse+IFNG relative to IFNG (significance defined as base mean >50, |log 2FC>1|, p-adj<0.01). Genes belonging to previously characterized DUX4 transcriptional program highlighted (n=84) (FIG. 39G). 5′UTR analysis of predicted minimum free energy (MFE) per 100 nucleotides, including all annotated mRNAs (n=78179), a subset of DUX4 target genes (n=84; transcription start site coordinates defined in data not shown), and mRNAs with significantly reduced translation efficiency (TE; significance defined as base mean >50, log 2FC<−1, p-adj<0.05) measured by Ribo-seq (RPFs mapping to 5′-region spanning the 5′UTR and first coding exon normalized to RNA-seq reads; n=1208) and polysome profiling (high/sub; n=2398). Statistical comparisons indicated were conducted using Mann-Whitney U test, * p<0.001, *** p<1.0e-12 (FIG. 39H). Model of polysome abundance resulting from DUX4-induced inhibition of translation initiation and elongation (FIG. 39I).

FIGS. 40A-40E. show expression of endogenous DUX4 correlates with translational suppression and reduced MHC-I surface antigens in cancer cells. Immunofluorescence of HPG Click-iT labeled nascent proteins in differentiated FSHD myotubes and SuSa cells co-stained for DUX4-target gene H3.X/Y (FIG. 40A). RT-qPCR analysis of unsynchronized (unsync) SuSa cells relative to a time course following release from synchronization with gapmer-mediated Control (CTRL) or DUX4 knockdown. Data represent mean±SD of technical triplicates (FIG. 40B). FACS analysis of MHC-I surface levels on SuSa cells 3 days after synchronization, treated with and without IFNG 16 hours prior to collection (FIG. 40C). Expression of housekeeping gene RPL27, DUX4 targets H3.X/Y, ZSCAN4, LEUTX (FIG. 40D) and MHC-I genes measured by RT-qPCR analysis of SuSa cells treated with and without IFNG, and expression levels in sorted IFNG-treated cells based on “high” or “low” MHC-I surface levels highlighted in (FIG. 40C). Data represent mean±SD of technical triplicates (FIG. 40E).

FIGS. 41A-41D. show expression of DUX4-CTD fragment suppresses interferon-induced MHC Class I expression and requires intact (L)LxxL(L) motifs. Human melanoma MEL375 cells were transduced with doxycycline inducible vectors expressing the 3×Flag-nls-tagged DUX4 CTD-154-424 (iDUX4-CTD) (SEQ ID NOS: 8 and 9) or the similar constructs with a mutation in the first (L)LxxL(L) motif and a deletion of the second (L)LxxL(L) motif (iDUX4-mL1dL2) (SEQ ID NOS: 10 and 11). In the absence of doxycycline induction of the transgenes, IFNG treatment results in increased MHC-class I proteins (FIG. 41A); whereas the DUX4-CTD suppresses IFNG induction of MHC proteins and the mutations of the (L)LxxL(L) almost completely abrogate this suppressive activity. As shown in FIGS. 41B and 41C, this correlates with decreased MHC surface protein expression as determined by FACs analyses. NYESO TCR-engineered T cells induce IFNG and IL-2 secretion in the presence of the NYESO expressing MEL375 cells. In contrast, a pulse-expression of DUX4 24 hrs prior to T-cell addition, completely blocks the T-cell activation by the NYESO expressing MEL375 cells (FIG. 41D).

### DETAILED DESCRIPTION

While illustrative embodiments have been illustrated and described, it will be appreciated that various changes can be made therein without departing from the spirit and scope of the invention.

DUX4 is aberrantly expressed in both FSHD muscle and cancers, but the physiological consequences of DUX4 expression in these two disease states are quite different. Sustained expression of DUX4 in skeletal muscle causes apoptosis, in contrast to DUX4's importance during early embryogenesis and apparent compatibility with many malignancies. Expression of DUX4 in several cell lines (plurior multipotent progenitor cells of early Xenopus or zebrafish embryos which lack D4Z4 repeats), or in cultured mammalian cells) demonstrate that DUX4 protein is highly toxic resulting in caspase-3-mediated apoptosis and can negatively affect myogenesis. WO 2021/022223 discloses hypoimmunogenic cells expressing DUX4 as well as related methods of their use and generation. However, the prior art overlooks the toxic effect of unregulated or ubiquitously overexpressed DUX4. The present inventors have elucidated mechanisms utilized by DUX4 and identified specific regions of DUX4 involved in suppressing adaptive and innate immune responses, to circumvent the toxicity associated with expression of DUX4. Thus, the present disclosure provides improved methods and compositions comprising DUX4 that are effective in overcoming the toxicity associated with the expression of DUX4 in a cell to provide for an effective evasion of both adaptive and innate immune responses crucial for the treatment of autoimmune and auto-inflammatory diseases.

The inventors have shown that DUX4 can inhibit the induction of some innate immune response genes that were normally induced by lentiviral transduction and that DUX4 can prevent MHC class I protein accumulation and presentation in response to interferon-gamma (IFNG) signaling. One mechanism for the DUX4-mediated inhibition of the innate immune response was shown to be the induction of the defensin family proteins encoded by the DUX4-induced genes DEFB103A and DEFB103B. However, subsequent studies showed that these defensins were not responsible for a large portion of the activity of DUX4 in suppressing the innate immune response pathways or in suppressing MHC class I presentation. The inventors have now identified two additional, and more dominant, mechanisms of innate and adaptive immune suppression by DUX4: (1) an inhibition of the interferon stimulated gene (ISG) network through the DUX4 protein that is independent of the transcriptional activity of DUX4 and the inventors have identified specific regions of the DUX4 protein that can suppress different components and pathways of the ISG network, including components of the adaptive immune response pathways; and (2) a DUX4-mediated long-term suppression of the MHC class I antigen expression and suppression of the immunoproteosome. The identification of these two distinct mechanisms of DUX4-mediated modulation of the innate and adaptive immune response provide the basis for developing new therapeutics that can manipulate specific components of the innate and adaptive immune responses and specific pathways in the ISG network, developing molecular mimics of these activities to enhance or impede specific immune signaling pathways, selectively inhibiting the immunoproteasome, and identifying a target gene or genes that are critical for regulating MHC Class I protein expression and expression of the components of the immunoproteasome. The ability to fine-tune the molecular activity of the DUX4-mediated modulation of the immune response enables the ability to minimize toxicity associated with treatment of wild-type DUX4 to cells given it has many different functions.

### I. DUX4 Polypeptides

The iDUX4-wt and iDUX4-CA sequences are shown in SEQ ID NOS: 1-3 below. LLxxL(L) motifs are in uppercase letters in the nucleotide and underlined in the amino acid sequences.

```
WT Nucleotide Sequence:
  (SEQ ID NO: 1)
ATGgccccccgacaccctcggacagcaccctccccgcggaagcccggggacgaggacggcgacggagactcgtt
tggaccccgagccaaagcgaggccctgcgagcctgctttgagcggaacccgtacccgggcatcgccaccagagaacggctggcc
caggccatcggcattccggagcccagggtccagatttggtttcagaatgagaggtcacgccagctgaggcagcaccggcgggaat
ctcggccctggcccgggagacgcggcccgccagaaggccggcgaaagcggaccgccgtcaccggatcccagaccgccctgctc
ctccgagcctttgagaaggatcgctttccaggcatcgccgcccgggaggagctggccagagagacgggcctcccggagtccagga
ttcagatctggtttcagaatcgaagggccaggcacccgggacagggtggcagggcgcccgcgcaggcaggcggcctgtgcagcg
cggcccccggcgggggtcaccctgctccctcgtgggtcgccttcgcccacaccggcgcgtggggaacggggcttcccgcacccc
acgtgccctgcgcgcctggggctctcccacagggggctttcgtgagccaggcagcgagggccgcccccgcgctgcagcccagcc
aggccgcgccggcagaggggatctcccaacctgccccggcgcgcggggatttcgcctacgccgccccggctcctccggacggg
gcgctctcccaccctcaggctcctcgctggcctccgcacccgggcaaaagccgggaggaccgggacccgcagcgcgacggcct
gccgggcccctgcgcggtggcacagcctgggcccgctcaagcggggccgcagggccaaggggtgcttgcgccacccacgtccc
aggggagtccgtggtggggctggggccggggtccccaggtcgccggggggcgtgggaaccccaagccggggcagctccacc
tccccagcccgcgcccccggacgcctccgcctccgcgcggcaggggcagatgcaaggcatcccggcgccctcccaggcgctcc
aggagccggcgccctggtctgcactcccctgcggcctgCTGCTGgatgagCTCCTGgcgagcccggagtttctgcag
caggcgcaacctctcctagaaacggaggccccgggggagctggaggcctcggaagaggccgcctcgctggaagcacccctcag
cgaggaagaataccgggctCTGCTGgaggagCTTtag.
Codon altered (CA) Nucleotide Sequence:
  (SEQ ID NO: 2)
ATGgcattgcctacaccttcagactctacgctgcctgcagaggctaggggaagaggtagacggcggcgattggtgtgg
actccatcacaatccgaagctcttcgcgcatgcttcgagcgcaatccctatccggggattgccacaagggagaggcttgcacaggct
atcggaatcccggaaccgagagtgcagatctggttccaaaatgaacgctctcggcagctcagacagcatcgcagggagtcccgccc
gtggccaggaagaaggggaccacctgaaggaagaagaaaacgcacagcggtgactggcagccaaacggctctgctgctccgcg
ctttcgagaaagatcggttccccggaattgccgcacgcgaagaactcgccagagaaactgggctcccagaatcacgaatacagattt
ggttccagaaccgcagagcaagacacccaggccaggggggacgggcacctgctcaggccggtggactctgctctgctgcccctg
ggggcggccatccagcaccttcctgggtggctttcgctcatactggcgcttggggtaccgggctgcctgctccgcatgttccctgtgct
ccaggggccctcccgcagggagcgtttgtttcccaggcagctagggctgcacctgccctgcaaccatcacaggcagcgccagctg
aaggcatcagccaacccgccccagcccgcggagattttgcttatgcagcgccagcacctccagacggtgccctgagccaccccca
agcccccagatggccccctcaccctggtaagtcccgggaagaccgcgatccccaacgagatggactgcccggtccttgcgctgtgg
cccagccaggacctgctcaagccggccctcaggggcaaggagtgctggccccacctacaagccagggatctccctggtggggttg
gggacgcggacctcaggttgctggagccgcttgggagcctcaggccggagctgcaccgccgccacaaccggcccctcccgacgc
gtcagcgtccgcccgacaaggccagatgcagggaatcccagcacctagccaagctcttcaagagcctgccccttggagcgcactg
ccgtgtgggctgCTCCTGgatgaaCTCCTGgctagcccagaatttctccagcaggcacagccactcctggaaacagaa
gctccgggagagctcgaagcctccgaagaagcagcaagcctggaggcacctctttccgaggaggagtatagagccCTTCTG
gaagaaCTTtga.
Protein:
  (SEQ ID NO: 3)
MALPTPSDSTLPAEARGRGRRRRLVWTPSQSEALRACFERNPYPGIATRERLA
QAIGIPEPRVQIWFQNERSRQLRQHRRESRPWPGRRGPPEGRRKRTAVTGSQTALLL
RAFEKDRFPGIAAREELARETGLPESRIQIWFQNRRARHPGQGGRAPAQAGGLCSAA
PGGGHPAPSWVAFAHTGAWGTGLPAPHVPCAPGALPQGAFVSQAARAAPALQPSQ
AAPAEGISQPAPARGDFAYAAPAPPDGALSHPQAPRWPPHPGKSREDRDPQRDGLP
GPCAVAQPGPAQAGPQGQGVLAPPTSQGSPWWGWGRGPQVAGAAWEPQAGAAPP
PQPAPPDASASARQGQMQGIPAPSQALQEPAPWSALPCGLLLDELLASPEFLQQAQP
LLETEAPGELEASEEAASLEAPLSEEEYRALLEEL*.
The iDUX4-mL1dL2 (a0012) are shown below as SEQ ID NOS: 4-5.
LLxxL(L) motifs are in uppercase letters in the nucleotide and underlined in the amino acid sequences.
Codon altered (CA) Nucleotide Sequence:
(SEQ ID NO: 4)
ATGgcattgcctacaccttcagactctacgctgcctgcagaggctaggggaagaggtagacggcggcgattggtgtgg
actccatcacaatccgaagctcttcgcgcatgcttcgagcgcaatccctatccggggattgccacaagggagaggcttgcacaggct
atcggaatcccggaaccgagagtgcagatctggttccaaaatgaacgctctcggcagctcagacagcatcgcagggagtcccgccc
gtggccaggaagaaggggaccacctgaaggaagaagaaaacgcacagcggtgactggcagccaaacggctctgctgctccgcg
ctttcgagaaagatcggttccccggaattgccgcacgcgaagaactcgccagagaaactgggctcccagaatcacgaatacagattt
ggttccagaaccgcagagcaagacacccaggccaggggggacgggcacctgctcaggccggtggactctgctctgctgcccctg
ggggcggccatccagcaccttcctgggtggctttcgctcatactggcgcttggggtaccgggctgcctgctccgcatgttccctgtgct
ccaggggccctcccgcagggagcgtttgtttcccaggcagctagggctgcacctgccctgcaaccatcacaggcagcgccagctg
aaggcatcagccaacccgccccagcccgcggagattttgcttatgcagcgccagcacctccagacggtgccctgagccaccccca
agcccccagatggccccctcaccctggtaagtcccgggaagaccgcgatccccaacgagatggactgcccggtccttgcgctgtgg
cccagccaggacctgctcaagccggccctcaggggcaaggagtgctggccccacctacaagccagggatctccctggtggggttg
gggacgcggacctcaggttgctggagccgcttgggagcctcaggccggagctgcaccgccgccacaaccggcccctcccgacgc
gtcagcgtccgcccgacaaggccagatgcagggaatcccagcacctagccaagctcttcaagagcctgccccttggagcgcactg
ccgtgtgggctgctcGCCgCtgaaGCCGCAgctagcccagaatttctccagcaggcacagccactcctggaaacagaag
ctccgggagagctcgaagcctccgaagaagcagcaagcctggaggcacctctttccgaggaggagtatagagcctga.
Protein:
  (SEQ ID NO: 5)
MALPTPSDSTLPAEARGRGRRRRLVWTPSQSEALRACFERNPYPGIATRERLA
QAIGIPEPRVQIWFQNERSRQLRQHRRESRPWPGRRGPPEGRRKRTAVTGSQTALLL
RAFEKDRFPGIAAREELARETGLPESRIQIWFQNRRARHPGQGGRAPAQAGGLCSAA
PGGGHPAPSWVAFAHTGAWGTGLPAPHVPCAPGALPQGAFVSQAARAAPALQPSQ
AAPAEGISQPAPARGDFAYAAPAPPDGALSHPQAPRWPPHPGKSREDRDPQRDGLP
GPCAVAQPGPAQAGPQGQGVLAPPTSQGSPWWGWGRGPQVAGAAWEPQAGAAPP
PQPAPPDASASARQGQMQGIPAPSQALQEPAPWSALPCGLLAAEAAASPEFLQQAQP
LLETEAPGELEASEEAASLEAPLSEEEYRA*.
iDUX4-F67A (a0021) is shown in SEQ ID NOS: 6-7.
LLxxL(L) motifs are in uppercase letters in the nucleotide and underlined in the amino acid sequences.
F67A mutation is in uppercase letters and underlined.
CA Nucleotide Sequence:
  (SEQ ID NO: 6)
ATGgcattgcctacaccttcagactctacgctgcctgcagaggctaggggaagaggtagacggcggcgattggtgtgg
actccatcacaatccgaagctcttcgcgcatgcttcgagcgcaatccctatccggggattgccacaagggagaggcttgcacaggct
atcggaatcccggaaccgagagtgcagatctggGCAcaaaatgaacgctctcggcagctcagacagcatcgcagggagtcccg
cccgtggccaggaagaaggggaccacctgaaggaagaagaaaacgcacagcggtgactggcagccaaacggctctgctgctcc
gcgctttcgagaaagatcggttccccggaattgccgcacgcgaagaactcgccagagaaactgggctcccagaatcacgaatacag
atttggttccagaaccgcagagcaagacacccaggccaggggggacgggcacctgctcaggccggtggactctgctctgctgccc
ctgggggcggccatccagcaccttcctgggtggctttcgctcatactggcgcttggggtaccgggctgcctgctccgcatgttccctgt
gctccaggggccctcccgcagggagcgtttgtttcccaggcagctagggctgcacctgccctgcaaccatcacaggcagcgccag
ctgaaggcatcagccaacccgccccagcccgcggagattttgcttatgcagcgccagcacctccagacggtgccctgagccacccc
caagcccccagatggccccctcaccctggtaagtcccgggaagaccgcgatccccaacgagatggactgcccggtccttgcgctgt
ggcccagccaggacctgctcaagccggccctcaggggcaaggagtgctggccccacctacaagccagggatctccctggtgggg
ttggggacgcggacctcaggttgctggagccgcttgggagcctcaggccggagctgcaccgccgccacaaccggcccctcccga
cgcgtcagcgtccgcccgacaaggccagatgcagggaatcccagcacctagccaagctcttcaagagcctgccccttggagcgca
ctgccgtgtgggctgCTCCTGgatgaaCTCCTGgctagcccagaatttctccagcaggcacagccactcctggaaacag
aagctccgggagagctcgaagcctccgaagaagcagcaagcctggaggcacctctttccgaggaggagtatagagccCTTCT
GgaagaaCTTtga.
Protein:
  (SEQ ID NO: 7)
MALPTPSDSTLPAEARGRGRRRRLVWTPSQSEALRACFERNPYPGIATRERLA
QAIGIPEPRVQIWAQNERSRQLRQHRRESRPWPGRRGPPEGRRKRTAVTGSQTALLL
RAFEKDRFPGIAAREELARETGLPESRIQIWFQNRRARHPGQGGRAPAQAGGLCSAA
PGGGHPAPSWVAFAHTGAWGTGLPAPHVPCAPGALPQGAFVSQAARAAPALQPSQ
AAPAEGISQPAPARGDFAYAAPAPPDGALSHPQAPRWPPHPGKSREDRDPQRDGLP
GPCAVAQPGPAQAGPQGQGVLAPPTSQGSPWWGWGRGPQVAGAAWEPQAGAAPP
PQPAPPDASASARQGQMQGIPAPSQALQEPAPWSALPCGLLLDELLASPEFLQQAQP
LLETEAPGELEASEEAASLEAPLSEEEYRALLEEL*.
i3XFLAG-NLS-CTDaa154-424 (longCTD)(a0033, made by SB) is shown in SEQ ID NO: 8-9.
3XFLAG is underlined in lowercase letters, NLS from SMCHD1 is italicized in uppercase letters,
NLS from SV40 is underlined in lowercase letters.
LLxxL(L) motifs are in uppercase letters in the nucleotide and lowercase in the amino acid sequences.
CA Nucleotide Sequence:
  (SEQ ID NO: 8)
ATGgactacaaagaccatgacggtgattataaagatcatgacatcgattacaaggatgacgatgacaagCCCCCC
AAGAGGATGAGGAGGGAGccaaaaaagaagagaaaggtaggacgggcacctgctcaggccggtggactctgctct
gctgcccctgggggcggccatccagcaccttcctgggtggctttcgctcatactggcgcttggggtaccgggctgcctgctccgcatg
ttccctgtgctccaggggccctcccgcagggagcgtttgtttcccaggcagctagggctgcacctgccctgcaaccatcacaggcag
cgccagctgaaggcatcagccaacccgccccagcccgcggagattttgcttatgcagcgccagcacctccagacggtgccctgag
ccacccccaagcccccagatggccccctcaccctggtaagtcccgggaagaccgcgatccccaacgagatggactgcccggtcct
tgcgctgtggcccagccaggacctgctcaagccggccctcaggggcaaggagtgctggccccacctacaagccagggatctccct
ggtggggttggggacgcggacctcaggttgctggagccgcttgggagcctcaggccggagctgcaccgccgccacaaccggccc
ctcccgacgcgtcagcgtccgcccgacaaggccagatgcagggaatcccagcacctagccaagctcttcaagagcctgccccttgg
agcgcactgccgtgtgggctgCTCCTGgatgaaCTCCTGgctagcccagaatttctccagcaggcacagccactcctgg
aaacagaagctccgggagagctcgaagcctccgaagaagcagcaagcctggaggcacctctttccgaggaggagtatagagccC
TTCTGgaagaaCTTtga.
Protein Sequence:
  (SEQ ID NO: 9)
DYKDHDGDYKDHDIDYKDDDDKPPKRMRREPKKKRKVGRAPAQAGGLCSA
APGGGHPAPSWVAFAHTGAWGTGLPAPHVPCAPGALPQGAFVSQAARAAPALQPS
QAAPAEGISQPAPARGDFAYAAPAPPDGALSHPQAPRWPPHPGKSREDRDPQRDGL
PGPCAVAQPGPAQAGPQGQGVLAPPTSQGSPWWGWGRGPQVAGAAWEPQAGAAP
PPQPAPPDASASARQGQMQGIPAPSQALQEPAPWSALPCGLlldellASPEFLQQAQPLL
ETEAPGELEASEEAASLEAPLSEEEYRAlleel*.
i3XFLAG-NLS-CTDaa154-419mL1dL2 (or longCTDmL1DL2) (a0034, made by SB) is shown in SEQ ID NO: 10-11.
3XFLAG is underlined in lowercase letters, NLS from SMCHD1 is italicized in uppercase letters,
NLS from SV40 is underlined in lowercase letters.
LLxxL(L) motif mutation is in uppercase letters in the nucleotide and lowercase in
the amino acid sequences.
CA Nucleotide Sequence:
  (SEQ ID NO: 10)
ATGgactacaaagaccatgacggtgattataaagatcatgacatcgattacaaggatgacgatgacaagCCCCCC
AAGAGGATGAGGAGGGAGccaaaaaagaagagaaaggtaggacgggcacctgctcaggccggtggactctgctct
gctgcccctgggggcggccatccagcaccttcctgggtggctttcgctcatactggcgcttggggtaccgggctgcctgctccgcatg
ttccctgtgctccaggggccctcccgcagggagcgtttgtttcccaggcagctagggctgcacctgccctgcaaccatcacaggcag
cgccagctgaaggcatcagccaacccgccccagcccgcggagattttgcttatgcagcgccagcacctccagacggtgccctgag
ccacccccaagcccccagatggccccctcaccctggtaagtcccgggaagaccgcgatccccaacgagatggactgcccggtcct
tgcgctgtggcccagccaggacctgctcaagccggccctcaggggcaaggagtgctggccccacctacaagccagggatctccct
ggtggggttggggacgcggacctcaggttgctggagccgcttgggagcctcaggccggagctgcaccgccgccacaaccggccc
ctcccgacgcgtcagcgtccgcccgacaaggccagatgcagggaatcccagcacctagccaagctcttcaagagcctgccccttgg
agcgcactgccgtgtgggctgctcGCCGCTgaaGCCGCAgctagcccagaatttctccagcaggcacagccactcctg
gaaacagaagctccgggagagctcgaagcctccgaagaagcagcaagcctggaggcacctctttccgaggaggagtatagagcct
ga.
Protein:
  (SEQ ID NO: 11)
MDYKDHDGDYKDHDIDYKDDDDKPPKRMRREPKKKRKVGRAPAQAGGLC
SAAPGGGHPAPSWVAFAHTGAWGTGLPAPHVPCAPGALPQGAFVSQAARAAPALQ
PSQAAPAEGISQPAPARGDFAYAAPAPPDGALSHPQAPRWPPHPGKSREDRDPQRDG
LPGPCAVAQPGPAQAGPQGQGVLAPPTSQGSPWWGWGRGPQVAGAAWEPQAGAA
PPPQPAPPDASASARQGQMQGIPAPSQALQEPAPWSALPCGLLaaeaaASPEFLQQAQP
LLETEAPGELEASEEAASLEAPLSEEEYRA*.
i3XFLAG-NLS-CTDaa339-424 (or iNLS-CTD) (a0020, 3XFLAG added by NS) is shown in SEQ ID NOS: 12-13.
3XFLAG is underlined in lowercase letters, NLS from SV40 is italicized in uppercase letters.
(L)LxxL(L) motifs are in uppercase letters for nucleotide and in lowercase in the amino acid sequences.
CA Nucleotide Sequence:
  (SEQ ID NO: 12)
ATGgactacaaagaccatgacggtgattataaagatcatgacatcgattacaaggatgacgatgacaagCCAAAAA
AGAAGAGAAAGGTAaacgcgtcagcgtccgcccgacaaggccagatgcagggaatcccagcacctagccaagctcttc
aagagcctgccccttggagcgcactgccgtgtgggctgCTCCTGgatgaaCTCCTGgctagcccagaatttctccagca
ggcacagccactcctggaaacagaagctccgggagagctcgaagcctccgaagaagcagcaagcctggaggcacctctttccga
ggaggagtatagagccCTTCTGgaagaaCTTtga.
Protein:
  (SEQ ID NO: 13)
MDYKDHDGDYKDHDIDYKDDDDKPKKKRKVNASASARQGQMQGIPAPSQA
LQEPAPWSALPCGLlldellASPEFLQQAQPLLETEAPGELEASEEAASLEAPLSEEEYRAlleel*.
Mouse Dux nucleotide sequence (NM_001081954) is shown in SEQ ID NO: 14.
  (SEQ ID NO: 14)
atggcagaagctggcagccctgttggtggcagtggtgtggcacgggaatcccggcggcgcaggaagacggtttggcaggcctgg
caagagcaggccctgctatcaactttcaagaagaagagatacctgagcttcaaggagaggaaggagctggccaagcgaatggggg
tctcagattgccgcatccgcgtgtggtttcagaaccgcaggaatcgcagtggagaggaggggcatgcctcaaagaggtccatcaga
ggctccaggcggctagcctcgccacagctccaggaagagcttggatccaggccacagggtagaggcatgcgctcatctggcagaa
ggcctcgcactcgactcacctcgctacagctcaggatcctagggcaagcctttgagaggaacccacgaccaggctttgctaccaggg
aggagctggcgcgtgacacagggttgcccgaggacacgatccacatatggtttcaaaaccgaagagctcggcggcgccacagga
ggggcaggcccacagctcaagatcaagacttgctggcgtcacaagggtcggatggggcccctgcaggtccggaaggcagagagc
gtgaaggtgcccaggagaacttgttgccacaggaagaagcaggaagtacgggcatggatacctcgagccctagcgacttgccctcc
ttctgcggagagtcccagcctttccaagtggcacagccccgtggagcaggccaacaagaggcccccactcgagcaggcaacgca
ggctctctggaacccctccttgatcagctgctggatgaagtccaagtagaagagcctgctccagcccctctgaatttggatggagacc
ctggtggcagggtgcatgaaggttcccaggagagcttttggccacaggaagaagcaggaagtacaggcatggatacttctagcccc
agcgactcaaactccttctgcagagagtcccagccttcccaagtggcacagccctgtggagcgggccaagaagatgcccgcactca
agcagacagcacaggccctctggaactcctcctccttgatcaactgctggacgaagtccaaaaggaagagcatgtgccagtcccact
ggattggggtagaaatcctggcagcagggagcatgaaggttcccaggacagcttactgcccctggaggaagcagtaaattcgggca
tggatacctcgatccctagcatctggccaaccttctgcagagaatcccagcctccccaagtggcacagccctctggaccaggccaag
cacaggcccccactcaaggtgggaacacggaccccctggagctcttcctctatcaactgttggatgaagtccaagtagaagagcatg
ctccagcccctctgaattgggatgtagatcctggtggcagggtgcatgaaggttcgtgggagagcttttggccacaggaagaagcag
gaagtacaggcctggatacttcaagccccagcgactcaaactccttcttcagagagtccaagccttcccaagtggcacagcgccgtg
gagcgggccaagaagatgcccgcactcaagcagacagcacaggccctctggaactcctcctctttgatcaactgctggacgaagtc
caaaaggaagagcatgtgccagccccactggattggggtagaaatcctggcagcatggagcatgaaggttcccaggacagcttact
gcccctggaggaagcagcaaattcgggcagggatacctcgatccctagcatctggccagccttctgcagaaaatcccagcctcccc
aagtggcacagccctctggaccaggccaagcacaggcccccattcaaggtgggaacacggaccccctggagctcttccttgatcaa
ctgctgaccgaagtccaacttgaggagcaggggcctgcccctgtgaatgtggaggaaacatgggagcaaatggacacaacacctg
atctgcctctcacttcagaagaatatcagactcttctagatatgctctga.
Mouse Dux protein sequence with homeodomains in lower case and underlined
are shown in SEQ ID NO: 15.
  (SEQ ID NO: 15)
MAEAGSPVGGSGVARESrrrrktvwqawqeqallstfkkkrylsfkerkelakrmgvsdcrirvwfqnR
RNRSGEEGHASKRSIRGSRRLASPQLQEELGSRPQGRGMRSSgrrprtrltslqlrilggafernprp
gfatreelardtglpedtihiwfqnrrarRRHRRGRPTAQDQDLLASQGSDGAPAGPEGREREGAQE
NLLPQEEAGSTGMDTSSPSDLPSFCGESQPFQVAQPRGAGQQEAPTRAGNAGSLEPL
LDQLLDEVQVEEPAPAPLNLDGDPGGRVHEGSQESFWPQEEAGSTGMDTSSPSDSN
SFCRESQPSQVAQPCGAGQEDARTQADSTGPLELLLLDQLLDEVQKEEHVPVPLDW
GRNPGSREHEGSQDSLLPLEEAVNSGMDTSIPSIWPTFCRESQPPQVAQPSGPGQAQA
PTQGGNTDPLELFLYQLLDEVQVEEHAPAPLNWDVDPGGRVHEGSWESFWPQEEA
GSTGLDTSSPSDSNSFFRESKPSQVAQRRGAGQEDARTQADSTGPLELLLFDQLLDE
VQKEEHVPAPLDWGRNPGSMEHEGSQDSLLPLEEAANSGRDTSIPSIWPAFCRKSQP
PQVAQPSGPGQAQAPIQGGNTDPLELFLDQLLTEVQLEEQGPAPVNVEETWEQMDT
TPDLPLTSEEYQTLLDML.
SEQ ID NO: 16 shows the Codon altered mouse Dux (iDuxCA):
  (SEQ ID NO: 16)
ATGGCAGAAGCAGGGTCACCCGTTGGTGGGAGCGGCGTAGCTAGAGAGA
GTAGACGCAGGAGGAAAACCGTTTGGCAAGCCTGGCAGGAACAAGCCTTGTTGT
CAACATTCAAGAAGAAGCGCTATTTGTCCTTTAAGGAGCGGAAAGAGTTGGCAA
AAAGGATGGGAGTAAGTGATTGTCGCATAAGGGTATGGTTTCAGAACCGGAGGA
ATAGGAGCGGGGAAGAGGGCCATGCCAGTAAGCGCAGCATCAGGGGGAGCCGG
CGCTTGGCAAGTCCCCAGCTCCAGGAAGAACTCGGGTCACGACCACAGGGCAGG
GGTATGCGCTCCTCCGGCCGGAGACCAAGAACTAGATTGACATCACTTCAACTT
CGGATCCTTGGTCAGGCTTTTGAACGGAATCCAAGGCCAGGGTTTGCCACACGC
GAAGAACTCGCAAGAGACACAGGCCTCCCCGAAGATACTATACACATATGGTTT
CAGAACAGAAGAGCTCGCCGCCGACACAGGAGGGGCAGACCCACTGCCCAGGA
CCAAGATCTCCTCGCCAGCCAGGGAAGTGATGGTGCACCAGCAGGTCCTGAAGG
GCGCGAAAGAGAAGGCGCCCAGGAGAATCTCCTCCCTCAAGAAGAAGCCGGCA
GCACAGGCATGGATACTAGCTCACCTAGCGATCTCCCCAGCTTCTGTGGAGAGT
CCCAGCCTTTCCAGGTGGCTCAGCCAAGAGGGGCTGGACAACAAGAAGCACCCA
CAAGAGCAGGTAACGCAGGCAGCTTGGAACCTCTGCTGGACCAACTGTTGGACG
AGGTTCAGGTAGAGGAACCAGCACCTGCTCCATTGAATCTGGACGGGGACCCTG
GGGGACGAGTGCACGAGGGGAGTCAAGAATCATTCTGGCCTCAGGAAGAGGCA
GGTAGTACTGGCATGGACACAAGTTCCCCAAGCGACAGTAACTCATTTTGTAGG
GAGTCACAGCCTTCCCAGGTAGCCCAACCATGTGGGGCTGGCCAAGAGGATGCA
AGGACACAGGCAGATTCTACTGGGCCATTGGAGTTGCTCCTCCTCGATCAGCTCT
TGGATGAGGTTCAGAAAGAGGAGCACGTTCCCGTGCCACTGGATTGGGGCAGAA
ATCCTGGCTCACGGGAACATGAGGGCTCCCAAGACTCCTTGTTGCCTCTTGAGGA
GGCAGTCAATAGTGGAATGGACACCAGTATACCAAGCATCTGGCCTACTTTCTG
CCGCGAGTCACAACCACCCCAAGTAGCACAGCCATCCGGACCTGGACAAGCTCA
AGCTCCCACTCAAGGAGGCAACACAGACCCCCTGGAGCTGTTTTTGTACCAGCT
GCTCGATGAAGTGCAGGTCGAGGAACACGCTCCCGCTCCCCTCAATTGGGACGT
GGACCCTGGGGGGCGCGTGCACGAGGGGAGTTGGGAGAGTTTCTGGCCCCAAG
AGGAAGCTGGAAGTACCGGGCTTGATACCTCAAGTCCATCTGACAGCAACTCAT
TCTTCCGGGAGTCTAAACCATCTCAAGTCGCCCAAAGACGGGGCGCTGGCCAAG
AGGACGCTAGAACCCAAGCCGACAGCACAGGTCCTCTTGAGTTGCTCCTGTTCG
ACCAGCTCCTCGATGAGGTCCAGAAAGAGGAACACGTTCCTGCTCCCCTGGACT
GGGGTAGAAATCCTGGAAGTATGGAACATGAGGGCAGCCAAGACTCACTCCTTC
CCTTGGAGGAAGCAGCCAACAGCGGACGCGATACAAGTATTCCATCCATCTGGC
CTGCTTTCTGCCGCAAATCCCAACCCCCCCAAGTGGCCCAACCATCTGGCCCCGG
TCAAGCCCAGGCCCCTATACAGGGCGGCAATACCGACCCATTGGAGCTTTTTCTT
GACCAGTTGCTCACTGAGGTACAGCTCGAAGAGCAAGGCCCCGCTCCCGTCAAT
GTAGAGGAAACCTGGGAGCAGATGGATACCACCCCCGATTTGCCACTTACATCC
GAGGAGTACCAAACACTGTTGGATATGCTCTAA.
```

### Polypeptide Embodiments

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein can be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” can be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) can comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides can be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-16.

In some embodiments, the protein or polypeptide can comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOS: 1-16.

In some embodiments, the protein, polypeptide, or nucleic acid can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOS: 1-16.

In some embodiments, the polypeptide, protein, or nucleic acid can comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS: 1-16 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-16.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-16 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-16.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes can be amplified and/or expressed using the techniques disclosed herein or as would be known to those of ordinary skill in the art.

### A. Variant Polypeptides

The following is a discussion of changing the amino acid subunits of a protein to create an equivalent, or even improved, second-generation variant polypeptide or peptide. For example, certain amino acids can be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes can be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure can affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wildtype protein. Individual residues can be deleted, or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This can include the insertion of one or more amino acid residues. Terminal additions can also be generated and can include fusion proteins which are multimers or concatemers of one or more peptides or polypeptides described or referenced herein.

Substitutional variants typically contain the exchange of one amino acid for another at one or more sites within the protein or polypeptide, and can be designed to modulate one or more properties of the polypeptide, with or without the loss of other functions or properties. Substitutions can be conservative, that is, one amino acid is replaced with one of similar chemical properties. “Conservative amino acid substitutions” can involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions can encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions can be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions can involve the exchange of a member of one of the amino acid classes for a member from another class.

### B. Considerations for Substitutions

One skilled in the art can determine suitable variants of polypeptides as set forth herein using well-known techniques. One skilled in the art can identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure can be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids can be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one can also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art can predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art can generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein-structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) can be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### Nucleic Acid Embodiments

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides can be single-stranded (coding or antisense) or double-stranded, and may be RNA, DNA (genomic, cDNA or synthetic), analogs thereof, or a combination thereof. Additional coding or non-coding sequences can, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide can be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, can be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length can be employed, with the total length preferably being limited by the ease of preparation and use in the intended recombinant nucleic acid protocol. In some cases, a nucleic acid sequence can encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide can be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more specific amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe can be used in diagnostic methods or a nucleic acid molecule PCR primer can be used to amplify regions of DNA that could be used, inter alia, to isolate nucleic acid sequences for use in producing variable domains of antibodies. See, e.g., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### Administration of Therapeutic Compositions

The therapy provided herein can comprise administration of a combination of therapeutic agents, such as, for example, a first DUX4 polypeptide therapy and a second anti-inflammatory therapy. The therapies can be administered in any suitable manner known in the art. For example, the first and second treatment can be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies can be administered in one composition or in more than one composition, such as 2 compositions, 3 compositions, or 4 compositions. Various combinations of the agents can be employed.

The therapeutic agents of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the second anti-inflammatory therapy is administered intravenously, intramuscularly, subcutaneously, topically, orally, transdermally, intraperitoneally, intraorbitally, by implantation, by inhalation, intrathecally, intraventricularly, or intranasally. The appropriate dosage can be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but can comprise continuous infusion over a set period of time. In some embodiments, a unit dose comprises a single administrable dose. Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### Therapeutic Applications

Methods include treatment of inflammatory and autoimmune diseases or disorders. Methods can be employed with respect to individuals who has tested positive for such diseases or disorders or who are deemed to be at risk for developing such a condition or related condition.

The methods provided herein can be used to induce or modify an immune response in a person having, suspected of having, or at risk of developing an inflammatory condition or complication relating to transplanted tissues from a non-self donor, or heterologous transplanted tissues used in cellular therapies. Methods may be employed with respect to individuals who have tested positive for autoreactivity or allo-reactivity or who are deemed to be at risk for developing such a condition or related condition.

The methods described herein can also be useful in treating or preventing disorders for which antigenic determinants are poorly characterized. Such disorders include, for example, rheumatoid arthritis, allergy, asthma, systemic onset juvenile arthritis, inflammatory bowel disease, and Crohn's disease. The methods and compositions described herein are also particularly useful for disorders such as GVHD and graft rejection.

Embodiments can be used to treat or ameliorate a number of immune-mediated, inflammatory, or autoimmune-inflammatory diseases, e.g., allergies, asthma, diabetes (e.g. type 1 diabetes), graft rejection, etc. Examples of such diseases or disorders also include, but are not limited to arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopeniarpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Celiac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

In some embodiments, the compositions and methods described herein are used to treat an inflammatory component of a disorder listed herein and/or known in the art. Accordingly, the methods and compositions described herein can be used to treat a subject suffering from inflammation. In some embodiments, the inflammation is acute. In other embodiments, the inflammation is chronic. In further embodiments, the compositions and methods described herein are used to treat or prevent a cancer by treating or preventing an inflammatory component associated with the cancer. In some embodiments, the methods exclude the treatment of cancer. In some embodiments, the subject is one that does not have cancer and/or has not been diagnosed with cancer.

In some embodiments, the administration of the DUX4 polypeptide or nucleic acid encoding the DUX4 polypeptide, as described herein, suppresses innate immunity in a cell or cells, in vitro or in vivo in a subject. In some embodiments, the DUX4 polypeptide or nucleic acid encoding the DUX4 polypeptide, as described herein, suppresses or inhibits the interferon stimulated gene network to suppress the innate immunity in a cell or cells, in vitro or in vivo in a subject. In some embodiments, the administration of the DUX4 polypeptide or nucleic acid encoding the DUX4 polypeptide, as described herein, suppresses or inhibits antigen presentation in a cell or cells, in vitro or in vivo in the subject, in an extended but transient manner. For example, the DUX4 peptide, contacted to a cell or expressed within a cell (in vitro or in vivo) can inhibit or reduce expression levels of one or more of canonical MHC-I subunits HLA-A, HLA-B, and HLA-C, and/or one or more of immunoproteasome subunits PSMB8, PSMB9, and PSMB10. The inhibition or reduction can occur for at least 12, 24, 36, 48, 60, 72, 84, or 96 hours after contact or expression of the Dux peptide.

In some embodiments, the compositions and methods described herein are used to treat a condition or disorder through the utilization of a universal donor stem cells described herein (or the progeny thereof).

In some embodiments, the present disclosure relates to a hypoimmunogenic cell comprising an exogenous DUX4 polypeptide or a nucleic acid encoding a DUX4 polypeptide for use in a method of treating a disease or a condition. In some embodiments the exogenous DUX4 polypeptide or the nucleic acid encoding a DUX4 polypeptide comprises a transcriptionally inactive DUX4 protein. In some embodiments the exogenous DUX4 polypeptide comprises a DNA-binding deficient DUX4 polypeptide. In some embodiments, the exogenous DUX4 polypeptide comprises an amino acid mutation in a homeodomain region of DUX4 or comprises a DUX4 polypeptide fragment lacking at least one homeodomain of the DUX4 protein. In some embodiments, the exogenous DUX4 polypeptide comprises a DUX4 protein or fragment thereof with at least 80% identity to a polypeptide comprising at least 80 contiguous amino acids of the DUX4 carboxy terminal region corresponding to amino acids 154-424 of the amino acid sequence as set forth in SEQ ID NO: 3. In some embodiments, the exogenous DUX4 polypeptide lacks the amino terminus of the DUX4 polypeptide or a portion thereof, wherein the amino terminus corresponds to amino acids 1-153 of the amino acid sequence as set forth in SEQ ID NO:3. In some embodiments, the exogenous DUX4 polypeptide comprises at least one (L)LxxL(L) motif, optionally at least one LxxL, LLxxL, LxxLL, and/or LLxxLL motif. In some embodiments, the exogenous DUX4 polypeptide comprises at least two (L)LxxL(L) motifs, optionally at least two LxxL, LLxxL, LxxLL, and/or LLxxLL motifs, alone or in any combination. In some embodiments, the exogenous DUX4 polypeptide corresponds to a human DUX4 polypeptide. In some embodiments, the nucleic acid encoding the exogenous DUX4 polypeptide is a codon altered sequence comprising one or more base substitutions to reduce the total number of CpG sites while preserving the DUX4 protein sequence. In some embodiments, the codon altered sequence comprises a nucleotide sequence encoding a Dux 4 protein or a fragment thereof. In some embodiments, the nucleic acid encodes for an inducible expression of DUX4 comprising a pulsed expression of DUX4 protein. In some embodiments, the nucleic acid encodes for a constitutive expression of DUX4 comprising a continuous expression of DUX4 protein.

In some embodiments, the present disclosure pertains to a hypoimmunogenic cell comprising an exogenous DUX4 polypeptide or a nucleic acid encoding a DUX4 polypeptide comprising a transcriptionally active DUX4 protein. In some embodiments, the nucleic acid encodes for an inducible expression of DUX4 comprising a pulsed expression of DUX4 protein.

In some embodiments, the hypoimmunogenic cells of the present disclosure further comprise a reduced expression of MHC-I and MHC-II human leukocyte antigens (HLA) relative to the wild-type cell of the same cell type. In some embodiments, the hypoimmunogenic cells further comprise a modification to increase expression of one or more tolerogenic factors selected from CD47, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9.

The present disclosure also contemplates pharmaceutical compositions comprising the hypoimmunogenic cells as disclosed herein.

In some embodiments, the present disclosure relates to a method of modulating immune response in a cell comprising contacting the cell with or expressing a DUX4 polypeptide or nucleic acid encoding a DUX4 polypeptide in the cell. In some embodiments the method inhibits expression levels of interferon stimulated genes, one or more of canonical MHC-I subunits HLA-A, HLA-B, and HLA-C, and/or one or more of immunoproteasome subunits PSMB8, PSMB9, and PSMB10.

In some embodiments, there is disclosed a method of inhibiting antigen presentation by a cell, comprising contacting the cell with or expressing a DUX4 polypeptide or nucleic acid encoding a DUX4 polypeptide in the cell.

Transfer of Genetic Material into Cells

Methods and compositions of the disclosure relate to cells comprising nucleic acids expressing DUX4 polypeptides. In certain embodiments, engineered nucleases can be used to introduce nucleic acid sequences for genetic modification of any cells used herein, particularly the starting cells, such as somatic cells or differentiated cells as described herein.

Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome, and harness the cell's endogenous mechanisms to repair the induced break by natural processes of homologous recombination (HR) and nonhomologous end-joining (NHEJ).

Non-limiting engineered nucleases include: Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in certain aspects of the methods and compositions.

It is commonly practiced in genetic analysis that in order to understand the function of a gene or a protein function one interferes with it in a sequence-specific way and monitors its effects on the organism. However, in some organisms it is difficult or impossible to perform site-specific mutagenesis, and therefore more indirect methods have to be used, such as silencing the gene of interest by short RNA interference (siRNA). Yet gene disruption by siRNA can be variable and incomplete. Genome editing with nucleases such as ZEN is different from siRNA in that the engineered nuclease is able to modify DNA-binding specificity and therefore can in principle cut any targeted position in the genome, and introduce modification of the endogenous sequences for genes that are impossible to specifically target by conventional RNAi. Furthermore, the specificity of ZFNs and TALENs are enhanced as two ZFNs are required in the recognition of their portion of the target and subsequently direct to the neighboring sequences.

Meganucleases, found commonly in microbial species, have the unique property of having very long recognition sequences (>14 bp) thus making them naturally very specific. This can be exploited to make site-specific DSB in genome editing; however, the challenge is that not enough meganucleases are known, or may ever be known, to cover all possible target sequences. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting amino acids of the meganuclease to design sequence specific meganuclease in a method named rationally designed meganuclease (U.S. Pat. No. 8,021,867 B2, incorporated herein by reference).

Meganuclease have the benefit of causing less toxicity in cells compared to methods such as ZENs likely because of more stringent DNA sequence recognition; however, the construction of sequence specific enzymes for all possible sequences is costly and time consuming as one is not benefiting from combinatorial possibilities that methods such as ZFNs and TALENs utilize. So, there are both advantages and disadvantages.

As opposed to meganucleases, the concept behind ZFNs and TALENs is more based on a non-specific DNA cutting enzyme which would then be linked to specific DNA sequence recognizing peptides such as zinc fingers and transcription activator-like effectors (TALEs). One way was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity. An example of a restriction enzyme with such properties is FokI. Additionally, FokI has the advantage of requiring dimerization to have nuclease activity and this means the specificity increases dramatically as each nuclease partner would recognize a unique DNA sequence. To enhance this effect, FokI nucleases have been engineered that can only function as heterodimers and have increased catalytic activity. The heterodimer functioning nucleases would avoid the possibility of unwanted homodimer activity and thus increase specificity of the DSB.

Although the nuclease portion of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases. The CRISPR nuclease system is a prokaryotic immune system that confers resistance to foreign genetic elements such as plasmids and phages and provides a form of acquired immunity. CRISPRs are DNA loci containing short repetitions of base sequences. In the context of a prokaryotic immune system, each repetition is followed by short segments of spacer DNA belonging to foreign genetic elements that the prokaryote was exposed to. This CRISPR array of repeats interspersed with spacers can be transcribed into RNA. The RNA can be processed to a mature form and associate with a cas (CRISPR-associated) nuclease. A CRISPR-Cas system including an RNA having a sequence that can hybridize to the foreign genetic elements and Cas nuclease can then recognize and cut these exogenous genetic elements in the genome. A CRISPR-Cas system does not require the generation of customized proteins to target specific sequences, but rather a single Cas enzyme can be programmed by a short guide RNA molecule (crRNA) to recognize a specific DNA target. The CRISPR-Cas systems of bacterial and archaeal adaptive immunity show extreme diversity of protein composition and genomic loci architecture. The CRISPR-Cas system loci have more than 50 gene families and there are no strictly universal genes, indicating fast evolution and extreme diversity of loci architecture. So far, adopting a multi-pronged approach, there is comprehensive cas gene identification of 395 profiles for 93 Cas proteins. Classification includes signature gene profiles plus signatures of locus architecture. A new classification of CRISPR-Cas systems is proposed in which these systems are broadly divided into two classes, Class 1 with multi-subunit effector complexes and Class 2 with single-subunit effector modules exemplified by the Cas9 protein. Efficient gene editing in human CD34⁺ cells using electroporation of CRISPR/Cas9 mRNA and single-stranded oligodeoxyribonucleotide (ssODN) as a donor template for HDR has been demonstrated. De Ravin et al., Sci Transl Med. 9 (372): eaah3480 (2017). Novel effector proteins associated with Class 2 CRISPR-Cas systems may be developed as powerful genome engineering tools and the prediction of putative novel effector proteins and their engineering and optimization is important. In addition to the Class 1 and Class 2 CRISPR-Cas systems, more recently a putative Class 2, Type V CRISPR-Cas class exemplified by Cpf1 has been identified Zetsche et al., Cell 163 (3): 759-771 (2015). Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto. The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence, thus the enzyme can repeatedly cut a specified locus after indel (insertion and deletion) formation, increasing the efficiency of HDR. Moreover, staggered DSBs with sticky ends permit orientation-specific donor template insertion, which is advantageous in non-dividing cells.

### Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits can comprise components, which may be individually packaged or placed in a container, such as a tube, bottle, vial, syringe, or other suitable container means.

Individual components can also be provided in a kit in concentrated amounts; in some embodiments, a component is provided individually in the same concentration as it would be in a solution with other components. Concentrations of components can be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any polypeptide described or identified herein, which includes nucleic acid primers/primer sets and probes that are identical to or complementary to all or part of a polypeptide described herein.

In certain aspects, negative and/or positive control nucleic acids, probes, and inhibitors are included in some kit embodiments. In addition, a kit may include a sample that is a negative or positive control for methylation of one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: DUX4 Polypeptides for Inhibition of the ISG Network

A. Different Regions of the DUX4 Protein, Independent of their Ability to Transcriptionally Activate a DUX4-Target Gene, Suppress Different Components of the ISG Network.
DUX4 Suppresses the Activation of the ISG Network Through the IFIH1, DDX68, and cGAS Pathways.

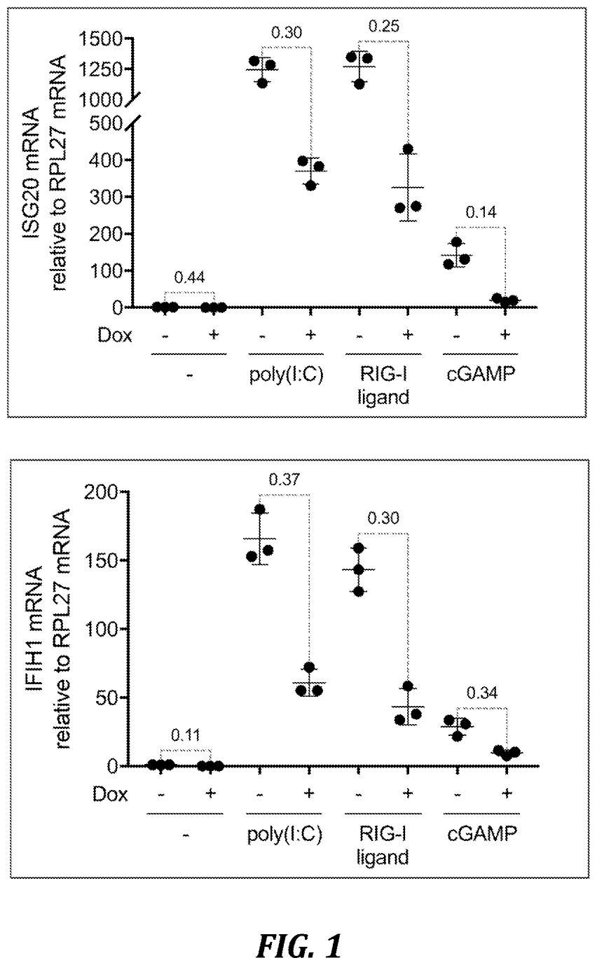

It was previously shown that DUX4 suppresses ISG induction following retroviral infection. To determine which of the several pathways might be blocked by DUX4, the inventors treated cells with long double-stranded RNAs (dsRNAs) as activators of IFIH1, a short dsRNA with a 5-prime triphosphate as an activator for DDX68, or dsDNAs as activators of cGAS (FIG. 1). Each treatment induced ISG genes ISG20 and IFIH1 (FIG. 1). In all cases, the doxycycline induction of DUX4 expression blunted the induction of these genes by each of the stimulated pathways. These pathways are thought to converge on specific common transcription factors such as IRF1 and/or IRF3, indicating that DUX4 is preventing a factor from functioning that is shared by each of these different pathways or is blocking the activity of multiple factors that together are necessary for these three pathways.

### DUX4 Blocks the Type II Interferon (Interferon-Gamma (IFNG)) and Part of the Type I (Interferon-Beta (IFNB1)) Stimulated ISG Network.

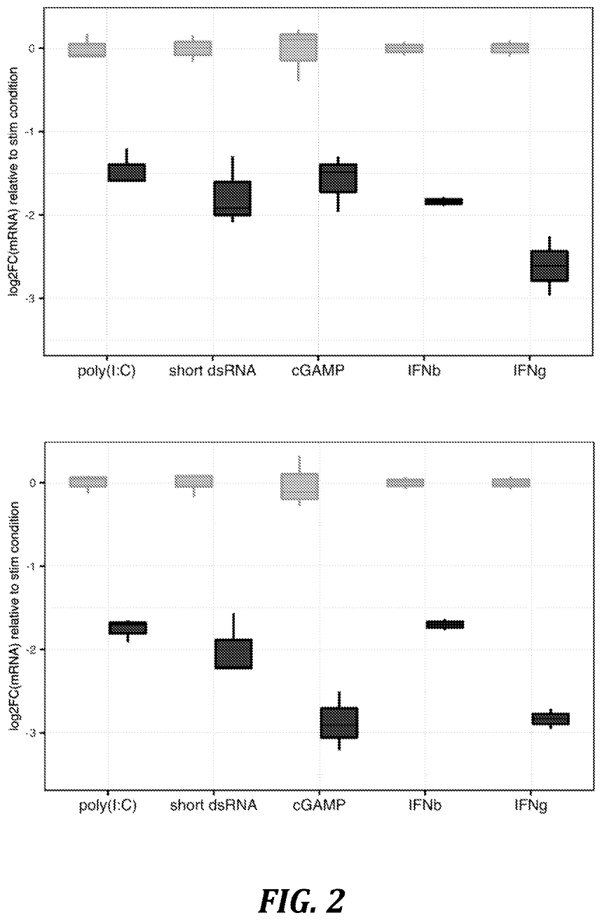

Stimulation by IFNG stimulates the ISG response through the JAK-STAT signaling pathway that for some genes also requires IRF9. IFNG induces expression of many ISG genes through a pathway largely mediated by STAT1. IFNB1 acts through STAT1, STAT2, and IRF9. Similar to the inhibition of ISG genes by the IFIH1, DDX68, and cGAS pathways, DUX4 blocks induction of ISGs IFIH1 and ISG20 by IFNG and IFNB1 (FIG. 2).

Together, these results indicate a broad inhibition of multiple immune signaling pathways by DUX4. These findings go significantly beyond the earlier observation that DUX4 inhibits ISG response to lentiviral transduction.

### the C-Terminal Region of DUX4 is Required to Block ISG Induction.

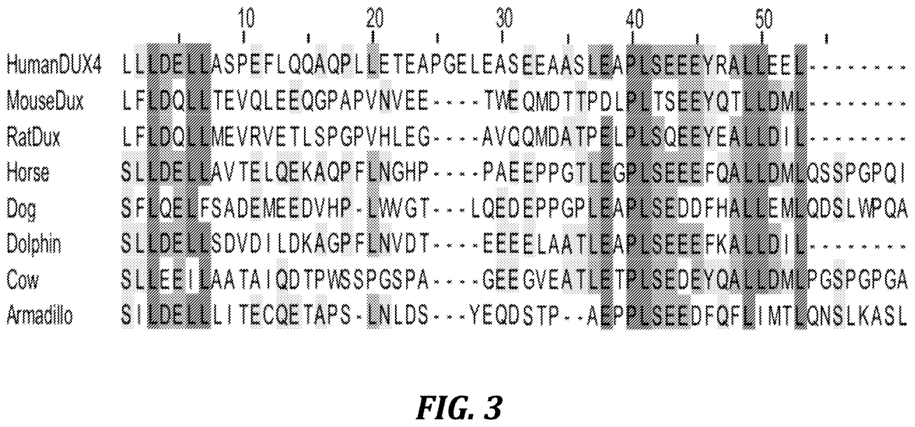

There are three relatively highly conserved regions of the DUX4/DUXC gene family: the two homeodomain regions and the distal carboxyterminal region with conserved LxxL, LxxLL, LLxxLL and LLxxL motifs (hereinafter (L)LxxL(L)) (FIG. 3). This C-terminal region has also been shown to be necessary for DUX4 to function as a transcriptional activator because deletion of this region results in no transcriptional activity of DUX4.

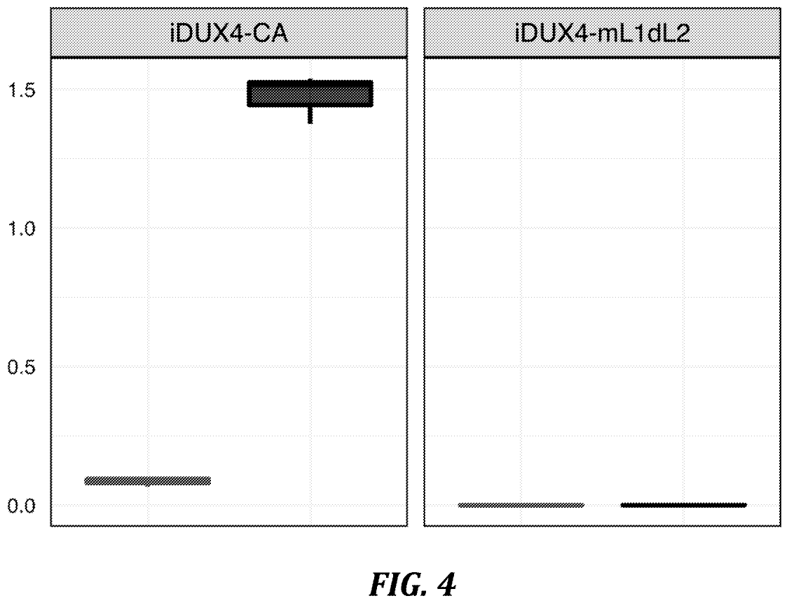

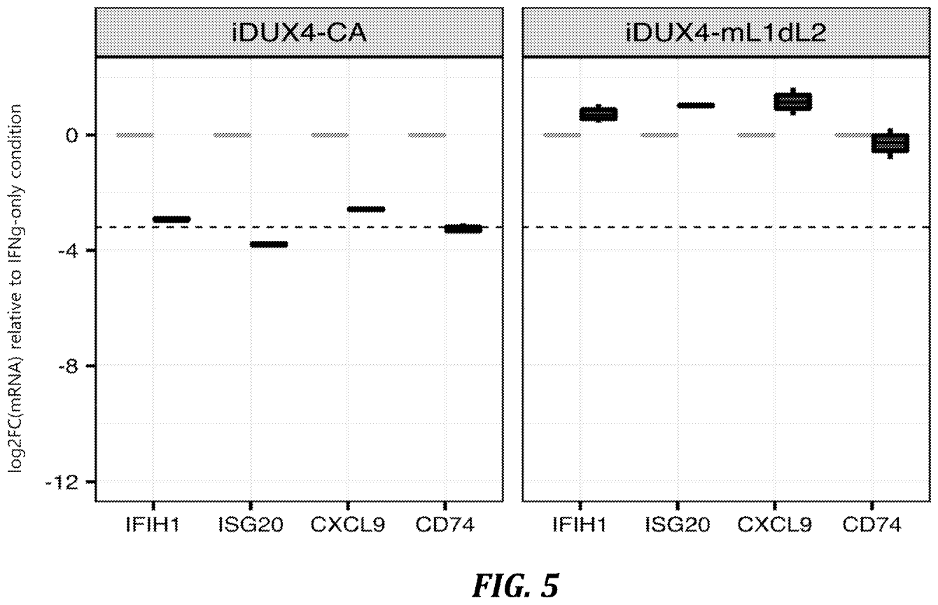

Mutations that disrupt the first (L)LxxL(L) motif and delete the second (L)LxxL(L) motif (SEQ ID NOS: 4-5), iDUX4-mL1dL2) completely prevent DUX4 from activating gene transcription (FIG. 4) and prevent the inhibition of ISG induction by INFG (FIG. 5).

The sequence of the wildtype DUX4 open reading frame (ORF) and the codon altered version of the DUX4 ORF is shown as SEQ ID NO:1 and SEQ ID NO:2, respectively, with the corresponding protein sequence and (L)LxxL(L) motifs identified (SEQ ID NO:3). The codon altered DUX4 sequence (SEQ ID NO:2) was used for all studies to avoid silencing of the transgene.

Inhibition of ISGs Requires Specific Regions of the DUX4 Protein but does not Require Transcriptional Activation of Another Gene by DUX4.

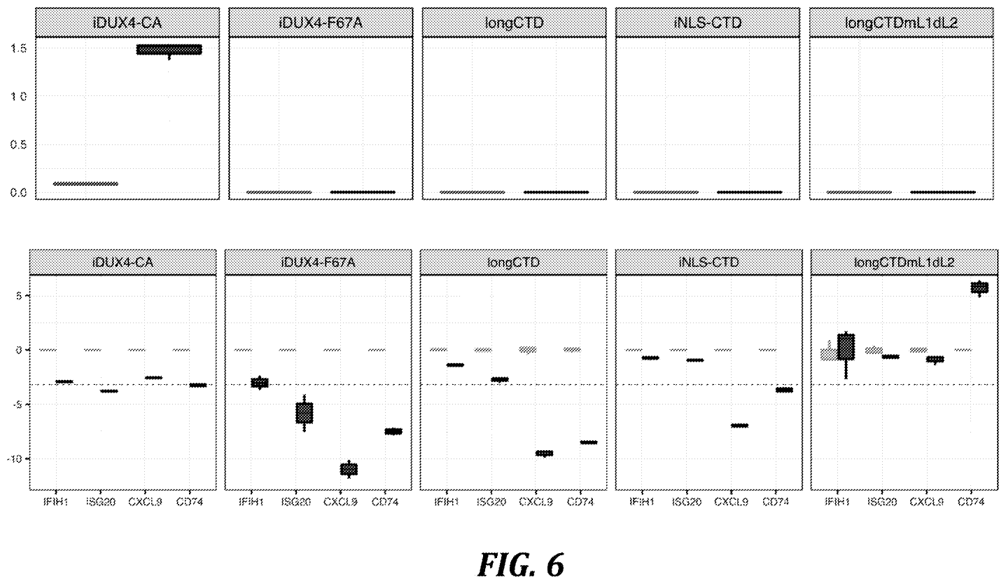

A mutation of a single amino acid in the first homeodomain of DUX4 from a phenylalanine to an alanine (DUX4-F67A) prevents DNA binding of DUX4 and prevents the transcriptional activation of DUX4 regulated genes (FIG. 6, top panel iDUX4-F67A). The sequence of iDUX4-F67A is shown as SEQ ID NOS: 6-7. The expression of this transcriptionally inactive protein is sufficient to block IFNG-induced ISG expression (FIG. 6, lower panel iDUX4-F67A). An expression construct encoding an amino-terminal 3×FLAG tag and nuclear localization signals (nls) in frame with the carboxyterminal 271 amino acids of DUX4 (aa154-424) that does not include the two DUX4 homeodomains (SEQ ID NO:8-9: i3×FLAG-NLS-CTDaa154-424 or longCTD) is also sufficient to block most or all ISG induction by IFNG (FIG. 6, lower panel, longCTD). A similar 3×FLAG tag nls construct encoding only the terminal 86 amino acids of DUX4 (aa 339-424) (i3×FLAG-NLS-CTDaa339-424 or iNLS-CTD) (SEQ ID NOS: 12 and 13) suppresses a portion of the IFNG-induced ISG genes, CXCL9 and CD74 (SEQ ID NOS: 12-13, lower panel iNLS-CTD). A construct identical to longCTD with the first (L)LxxL(L) motif disrupted by mutations and the second (L)LxxL(L) motif deleted (i3×FLAG-NLS-CTDaa154-419mL1dL2 or longCTDmL1dL2) (SEQ ID NOS: 10 and 11) does not suppress any of the IFNG-induced ISG genes (FIG. 6, lower panel, longCTDmL1dL2).

Together these data demonstrate that portions of DUX4 that do not contain transcriptional activity can suppress different pathways induced by IFNG that regulate ISG expression. These identify the conserved (L)LxxL(L) motifs as necessary for inhibiting ISG induction and sufficient for inhibiting the induction of a subset of ISG genes, and a second region between the (L)LxxL(L) domains and the homeodomains as necessary together with the (L)LxxL(L) containing region to inhibit the full set of ISG genes.

B. DUX4 has Two Distinct Mechanisms of Blocking MHC Class I Presentation: a Non-Transcriptional Mechanism Similar to the Inhibition of the ISG Genes Mediated by Regions of the DUX4 Protein and a Second, Long-Term Suppression that Requires DUX4 Transcriptional Activity.
Transcriptionally Active DUX4 has a Long-Term Suppression of MHC Class I Proteins that Indicates the Induction of a Gene or RNA that Mediates the Long-Term Suppression.

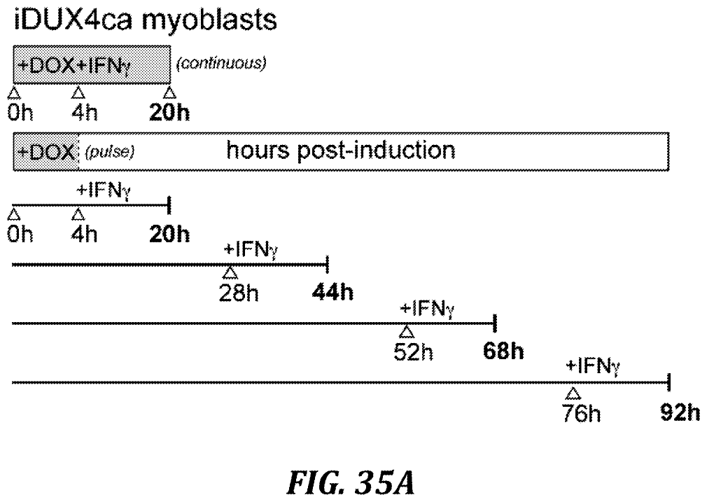

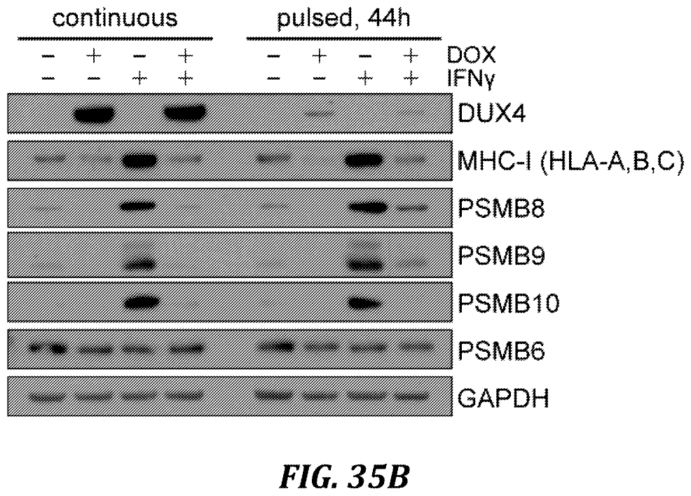

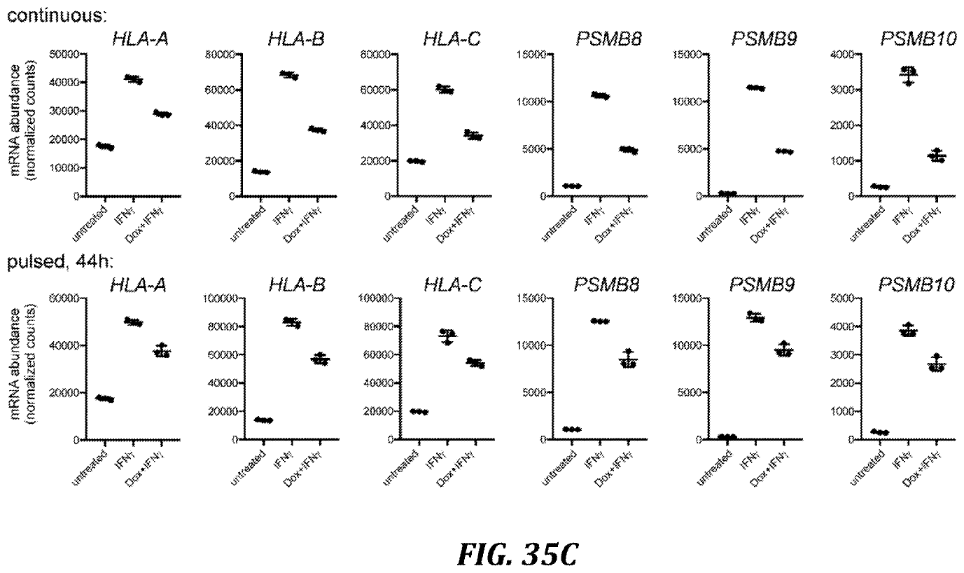

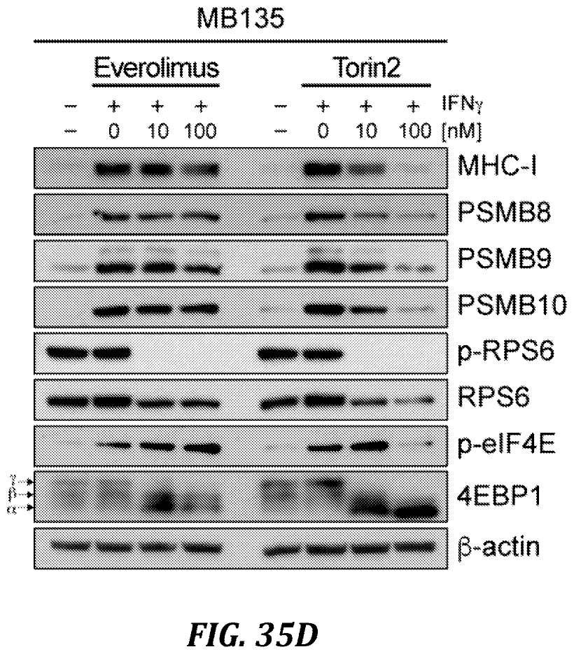

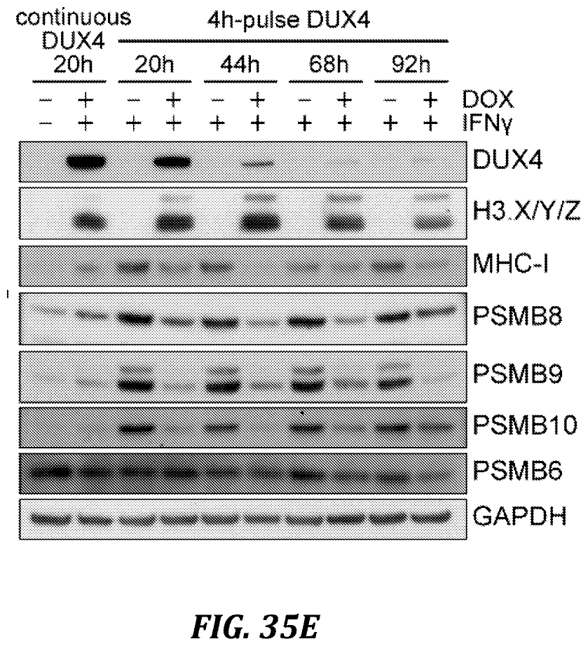

To determine the direct effect of the DUX4 protein on MHC class I expression compared to secondary mechanisms that might be mediated through genes regulated by DUX4, the ability of DUX4 to suppress MHC class I proteins was compared in either a “continuous” or “pulsed” protocol of DUX4 expression (FIG. 35A).

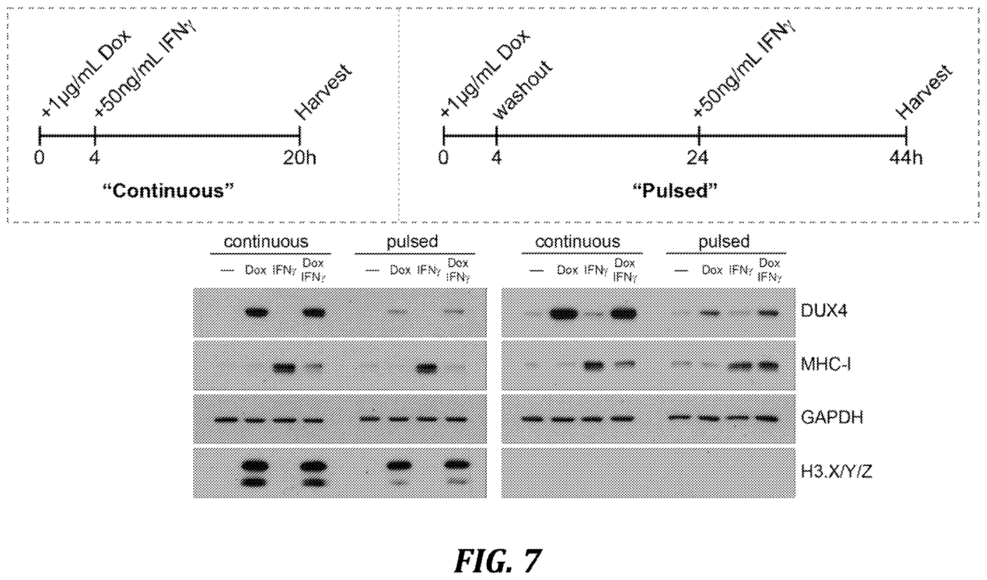

In the “continuous” protocol (similar to the experiments shown in the prior FIGS. 1, 2, 4, 5, and 6), doxycycline is added to induce DUX4 expression at time 0 and four hours later IFNG is added for an additional 16 hours, resulting in 20 hours of continuous DUX4 induction with the last 16 hrs exposed to IFNG. Under this continuous protocol, both the full-length DUX4 (SEQ ID NOS: 2-3) and the DUX4-F67A (SEQ ID NOS: 6-7) (a mutant in the first homeodomain that does not activate gene transcription, see FIG. 6 top panel) suppress the IFNG induction of MHC class I expression (FIG. 7, lower panel “continuous”). The DUX4 target genes H3.X and H3.Y are induced by the full-length DUX4 but not the F67A mutant as shown by the accumulation of H3.X/Y proteins, consistent with the F67A mutation disrupting DNA binding by DUX4.

In the “pulsed” protocol, doxycycline is added at time 0 to induce DUX4 expression, but after four hours the media is changed to remove the doxycycline, resulting in a pulse of DUX4 mRNA and protein sufficient to induce DUX4 target gene expression (see Resnick et al., Cell Rep. 2019 Nov. 12; 29(7):1812-1820.e5). IFNG is added at 28 hours for a period of 16 hours prior to harvesting the cells. The full-length DUX4 suppresses MHC class I expression as well, or slightly better, in the pulsed protocol than in the continuous protocol; whereas the F67A mutant does not suppress MHC class I in the pulsed protocol (FIG. 7, lower panel, “pulsed”). This difference is not likely to be due to different amounts of the DUX4 and mutant DUX4 proteins because at the time of harvest both DUX4 and the F67A mutant DUX4 protein levels have declined to similarly low amounts relative to the continuous protocol, but could be due to the activation of DUX4 target genes by the full-length DUX4 since the H3.X/Y proteins show persistent expression in the pulsed protocol.

### The Suppression of MHC Class I in the Pulse Protocol is Likely Due to a Gene or RNA Transcriptionally Induced by DUX4.

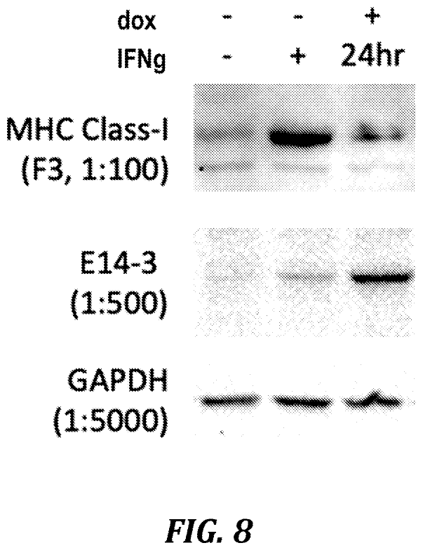

As shown in FIG. 7, the DUX4-F67A mutation that is transcriptionally inactive does not suppress MHC class I in the pulse protocol. In contrast, a DUX4 chimera that removes the carboxyterminal 94 amino acids and replaces them with the heterologous activation domain from the VP16 protein, a protein fragment typically used as a classical generic transcriptional activation domain, maintains the ability to suppress MHC class I protein in the pulse protocol (FIG. 8) despite lacking the DUX4 region with the two (L)LxxL(L) motifs that are required for suppression of the ISG pathways (see FIG. 6). Together these data demonstrate that it is the transcriptional activation function of DUX4 that provides longer-term suppression of MHC class I proteins in the pulse protocol.

### A Brief Pulse of DUX4 Suppresses the Canonical MHC Class I Proteins Through a Post-Transcriptional Mechanism but not the Expression of the Noncanonical MHC Protein HLA-E.

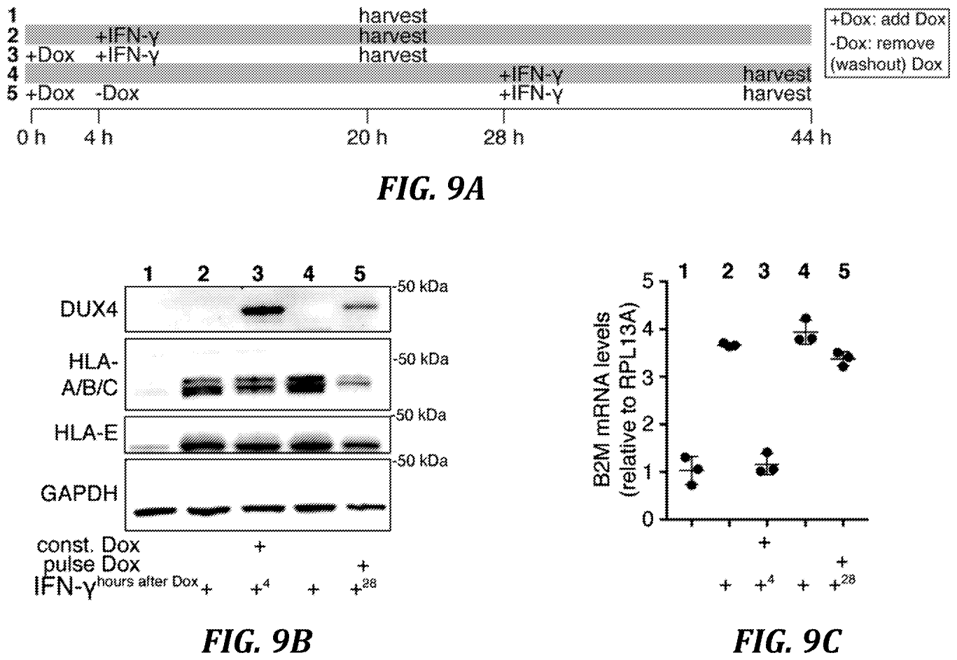

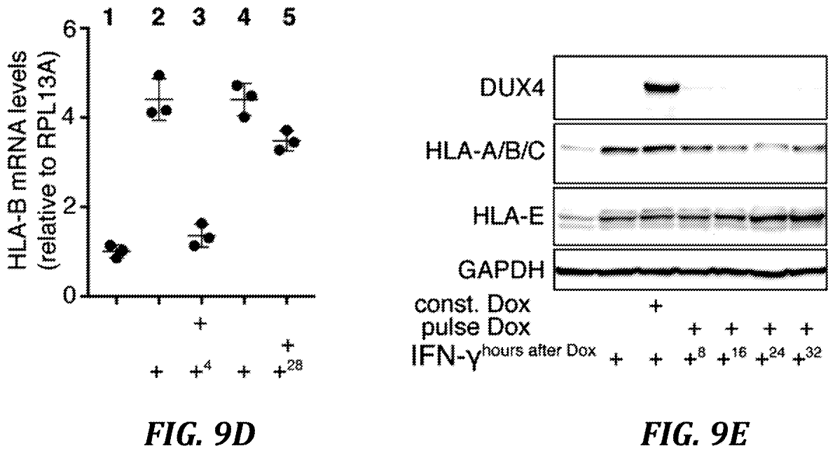

The suppression of MHC class I proteins in the continuous protocol in the MB135iDUX4 cells correlates with the suppression of their respective mRNAs, consistent with the DUX4 protein-mediated inhibition of the ISG response described in section 3 above, whereas the suppression of MHC class I proteins in the pulse protocol occurs without significant decrease in their respective mRNAs, indicating a post-transcriptional mechanism of suppressing the MHC class I proteins in the pulse protocol (FIG. 9). The continuous protocol results in a modest suppression of MHC class I protein (FIG. 9B, lane 3) and a profound inhibition of the IFNG-induction of the mRNA for MHC class I genes (FIG. 9C and FIG. 9D, lanes 3); whereas the pulse protocol produces a more profound suppression of the MHC class I proteins and little suppression of their mRNAs. The pulse protocol also produces a prolonged suppression of MHC class I proteins in the MEL375 melanoma cell line, similar to that seen in the MB135iDUX4 line (FIG. 9E). Of note, while the pulse protocol suppresses the canonical MHC class I proteins in multiple cell types, it does not suppress the noncanonical MHC class E (HLA-E) that is necessary to protect from natural killer cell activity.

DUX4 Suppression of MHC Class I Proteins Persists for at Least Four Days after a Brief Pulse of DUX4, Long after the Loss of the DUX4 Protein.

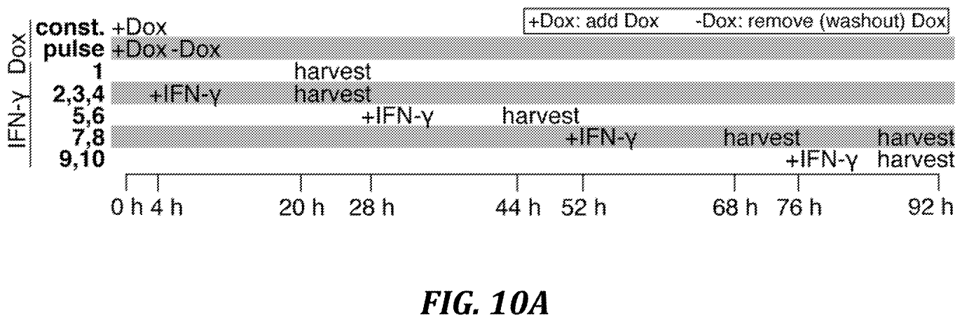

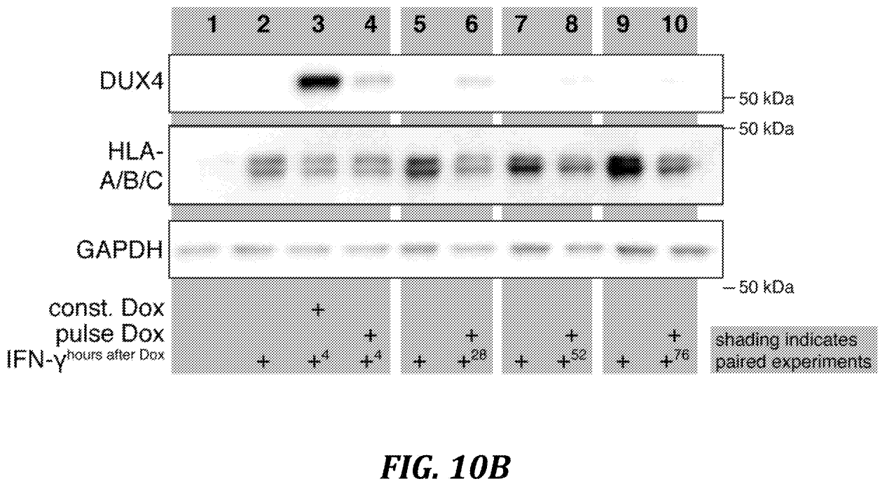

While the suppression of the ISG response occurs in the presence of the DUX4 protein containing the (L)LxxL(L) motifs (see section A above), the suppression of the MHC class I proteins becomes more pronounced between 24 and 72 hours after a pulse of DUX4 (FIG. 10), consistent with the induction of a separate gene or RNA or protein by the pulse of DUX4 protein as the cause of MHC class I suppression.

### Inhibiting the Proteasome Suppresses Canonical MHC Class I Proteins but not Non-Canonical HLA-E, Similar to the DUX4 Pulse Protocol.

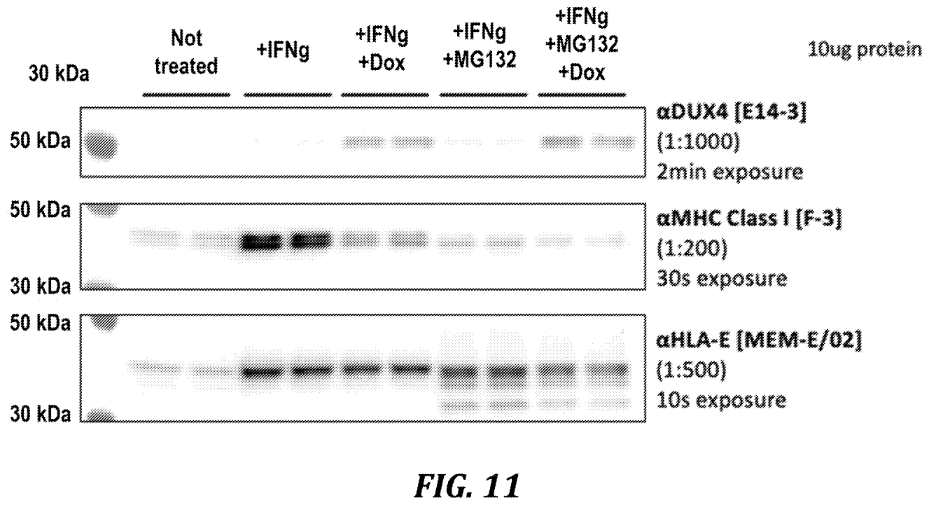

Interferon induces the expression of components of the proteasome, referred to as the immunoproteasome, that result in the generation of high affinity peptides that stabilize the canonical MHC class I proteins, whereas HLA-E stabilization does not require peptide production by the proteasome. Preventing peptide production by treating cells with the proteasome inhibitor MG132 shows a similar suppression of MHC class I proteins and stable expression of HLA-E protein as the pulse protocol for DUX4 (FIG. 11), indicating that one mechanism through which DUX4 suppresses MHC class I expression might be through preventing the formation or function of the IFNG-induced components of the immunoproteasome.

### DUX4 Suppresses the Ifng Induction of the Immunoproteasome by Two Mechanisms: The Inhibition of the ISG Gene Response by the DUX4 Protein and the Post-Translational Inhibition of Immunoproteasome Protein Accumulation.

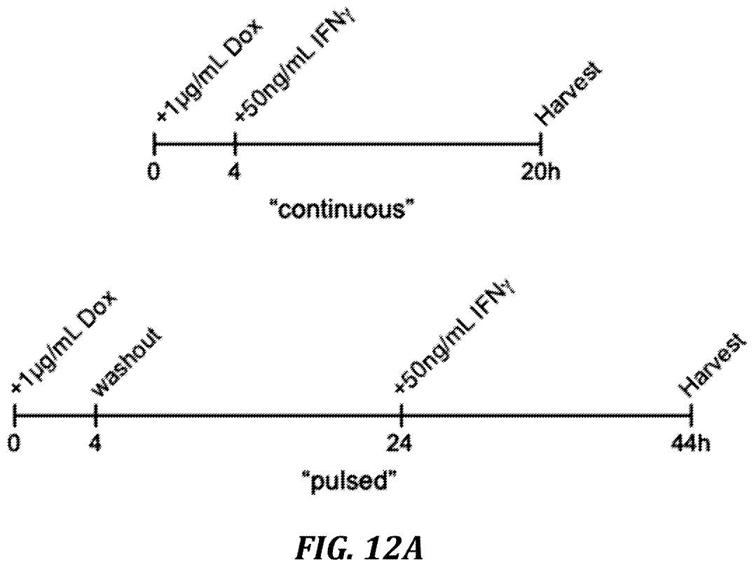

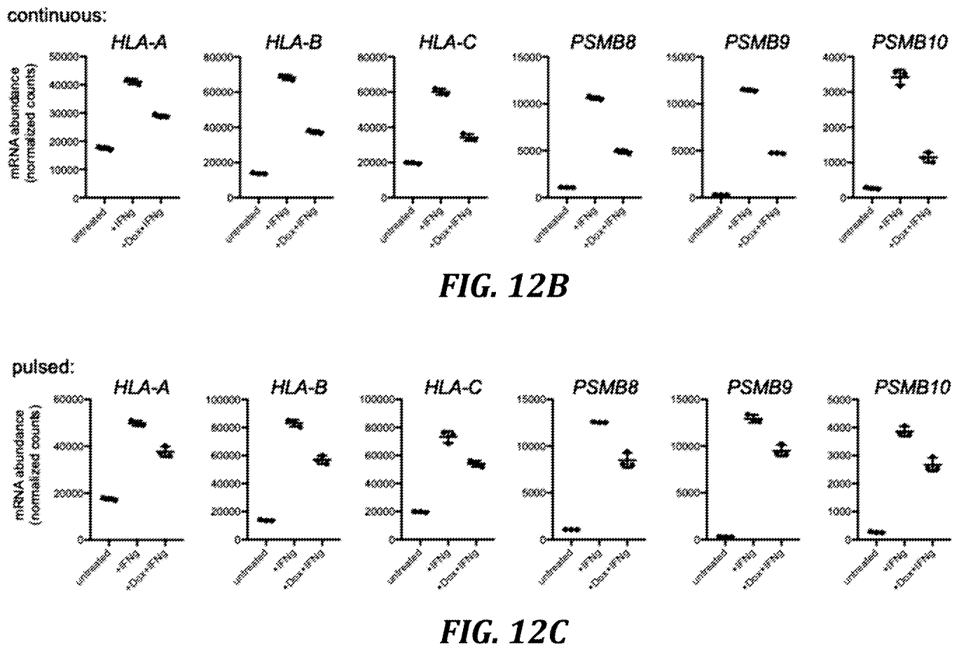

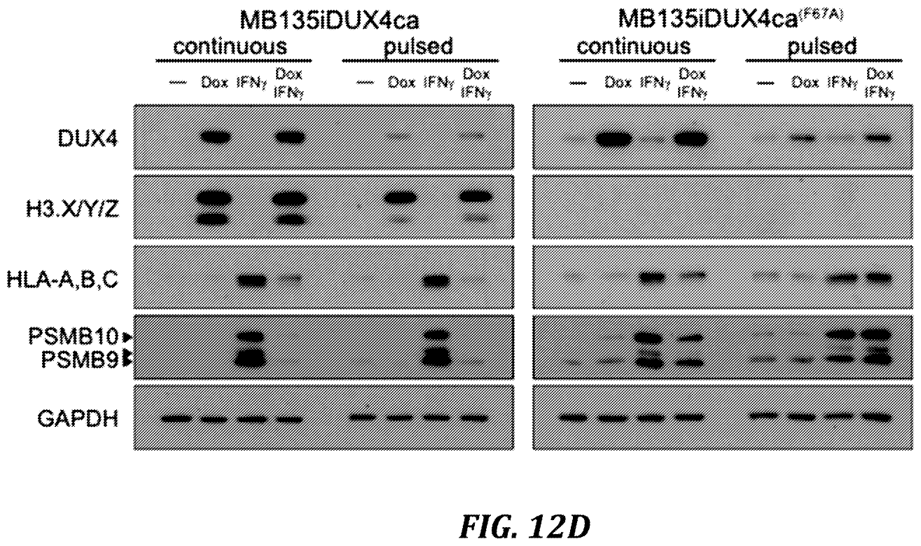

In the continuous protocol DUX4 partially suppresses the IFNG induction of the mRNAs for PSMB8, 9, and 10, whereas there is nearly full induction of these mRNAs in the pulsed protocol (FIGS. 12A-12C). Despite partial or nearly full induction of these mRNAs by IFNG, the expression of the PSMB9 and PSMB10 proteins is completely blocked by the continuous or pulsed expression of DUX4 (FIG. 12D, MB135iDUX4ca). In contrast to the transcriptionally active DUX4, the transcriptionally inactive DUX4 F67A (SEQ ID NOS: 6-7) mutant only partially inhibits the IFNG induction of PSMB9 and PSMB10 in the continuous protocol, and does not inhibit at all in the pulsed protocol (FIG. 12D, MB135iDUX4ca (F67A)), similar to its inhibition of the MHC class I proteins.

These data indicate that DUX4 prevents the formation of the immunoproteasome in response to IFNG through two mechanisms: a direct inhibition of ISG gene induction by the DUX4 protein and a longer-term and more robust post-translational inhibition of the accumulation of the immunoproteasome protein(s) that depends on the transcriptional activity of DUX4, most likely through a gene or transcript induced by DUX4.

### Identification of Pathways Mediating DUX4 Activity.

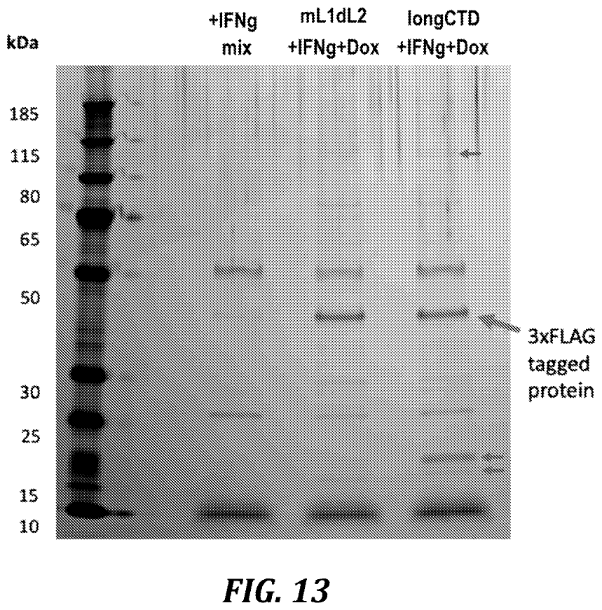

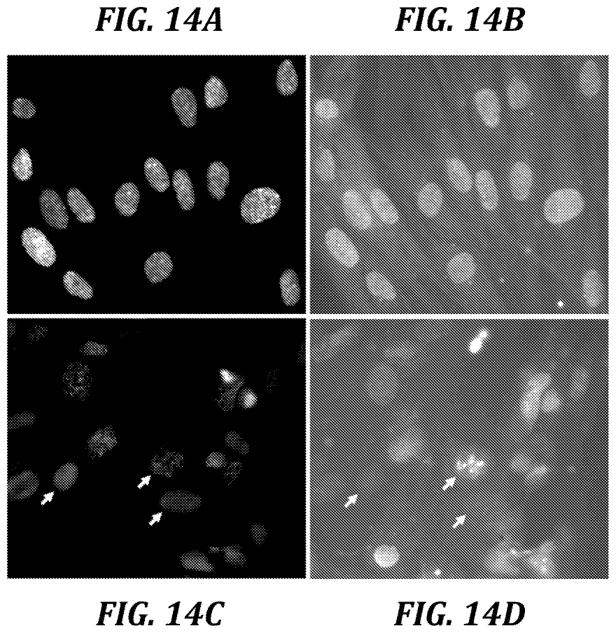

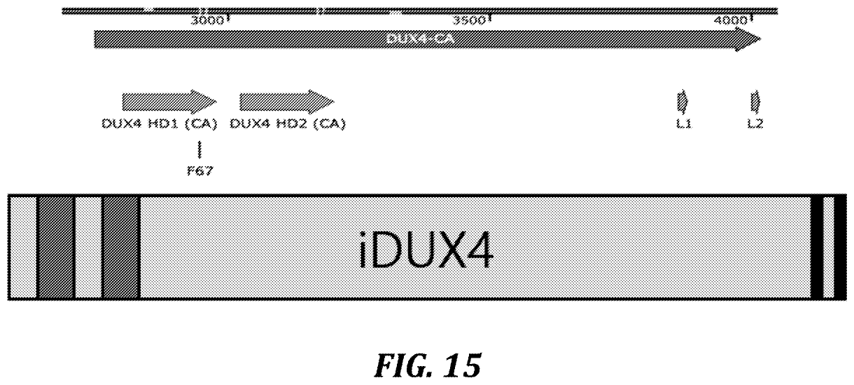

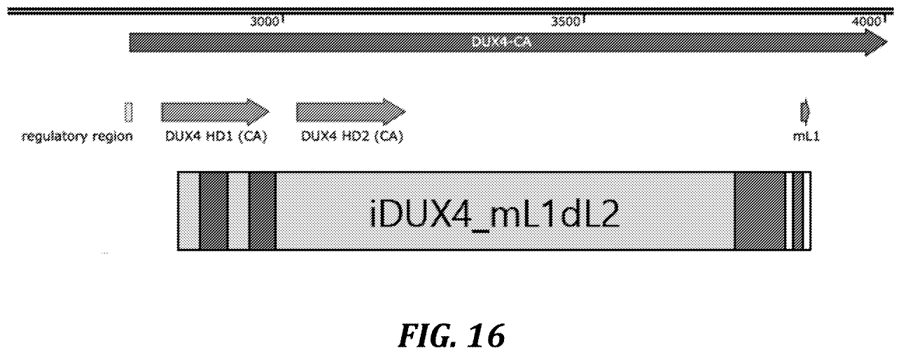

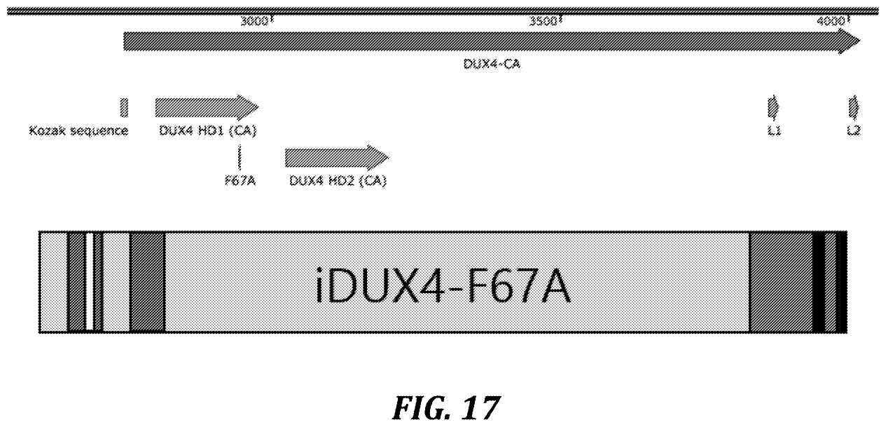

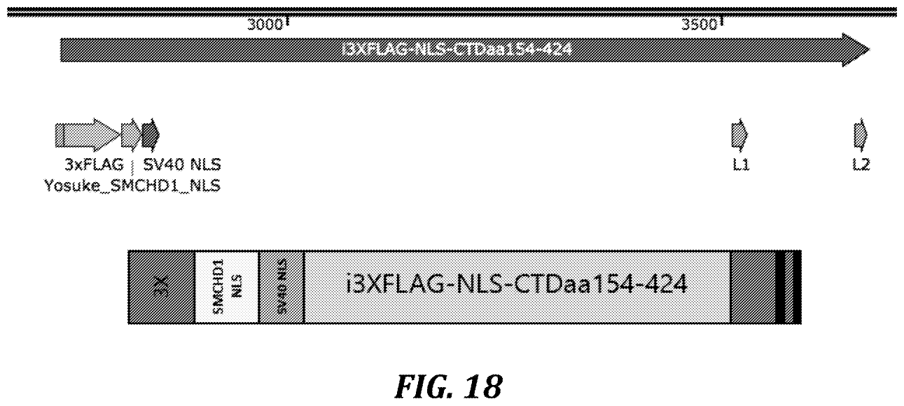

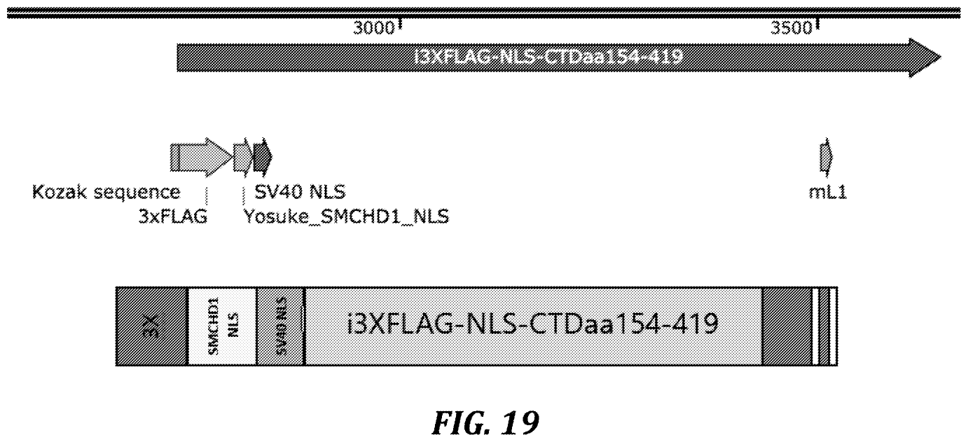

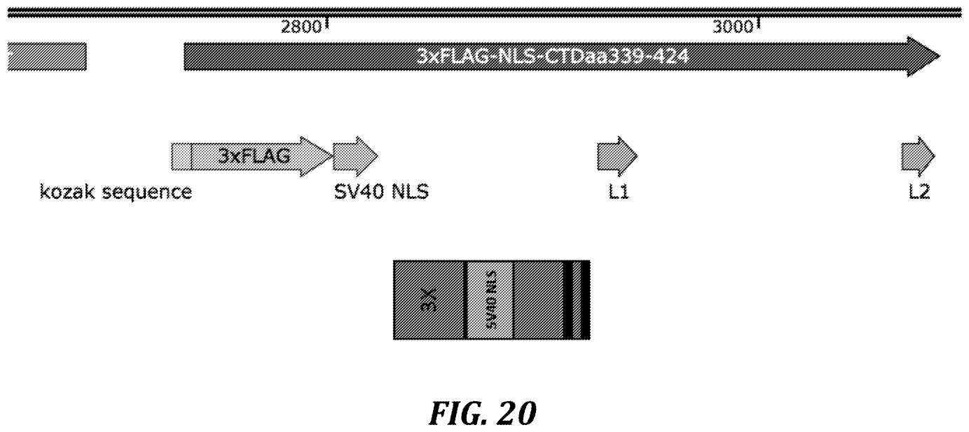

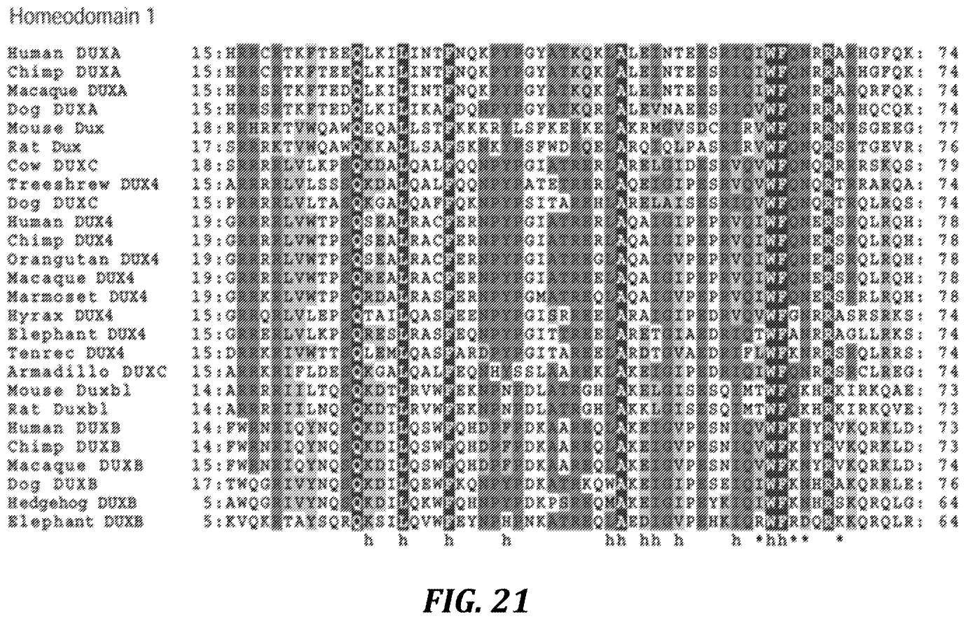

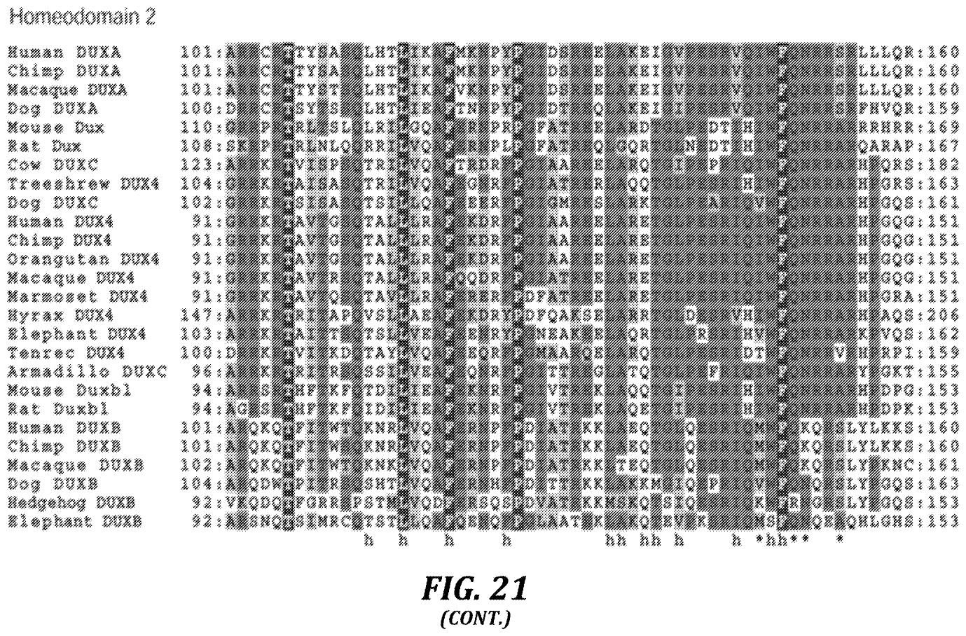

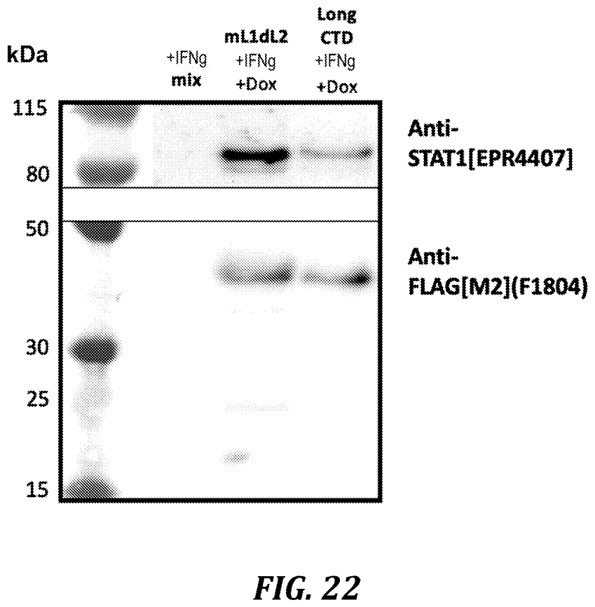

Knowing that the longCTD fragment of DUX4 inhibits ISG induction by IFNG, this 3×FLAG tagged protein can be used to identify proteins that interact with it. FIG. 13 shows that the 3×FLAG longCTD (SEQ ID NOS: 8-9) can be used to isolate proteins that interact it but not with the same region that has mutations in the first (L)LxxL(L) motif and deletion of the second (L)LxxL(L) motif (longCTDmL1dL2) (three example bands are indicated by the blue arrows). The proteins from isolated from MB135 myoblasts that co-purify with an expressed 3×FLAG longCTD were identified using mass spectrometry and included the STAT1 protein, a necessary regulator of the signaling by IFNG. FIG. 14 shows immunofluorescence with an antibody to STAT1. STAT1 has an homogeneous nuclear distribution in MB135iDUX4 cells treated with IFNG without doxycycline induction of DUX4, whereas when DUX4 is induced by doxycline, the STAT1 protein is no longer evenly distributed in the nucleus and shows overall decreased abundance, indicating that the DUX4 interaction with STAT1 prevents its normal distribution and accumulation in response to IFNG. FIG. 22 shows that STAT1 is co-immunoprecipitated with flag-tagged DUX4 CTD proteins. A flag-tagged DUX4 longCTD containing the two (L)LxxL(L) motifs (SEQ ID NOS: 8-9) or a similar longCTD with the first (L)LxxL(L) motif mutated and the second deleted (SEQ ID NOS: 10-11) both show binding to STAT1, as indicated by the STAT1 co-immunoprecipitation with the flag-tagged CTD proteins. Therefore expressing portions of DUX4 and isolating associated proteins can identify STAT1 as a DUX4 interacting protein that regulates the IFNG ISG response and these studies provide a roadmap for identifying other factors that interact with DUX4 to regulate innate immune signaling.

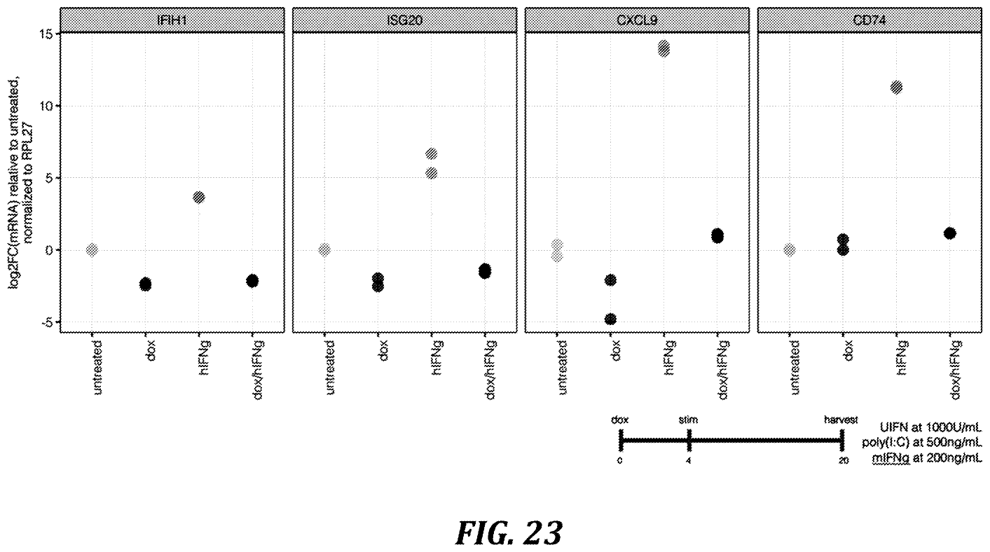

Mouse DUX suppresses the IFNG stimulated ISG response in human MB135 cells indicating functionally conserved regions of human DUX4 and mouse DUX that can be used to design peptides that modulate the ISG response. Mouse DUX is the ortholog of human DUX4. Mouse DUX (SEQ ID NO:14 (nucleotide) and SEQ ID NO:15 (protein), and it has two homeodomains (indicated in SEQ ID NO:15) and a conserved pair of carboxyterminal (L)LxxL(L) motifs (see FIG. 3). In addition, mouse DUX has duplicated regions containing the (L)Lxx(L) domains and triplicated a portion of this region, suggesting that it might have strong activity in suppressing the IFNG induction of the ISG response in human cells. MB135 human myoblasts engineered to have a doxycycline inducible codon altered mouse DUX (iDuxCA, SEQ ID NO:16) showed normal IFNG induction of the ISG genes IFIH1, ISG20, CXCL9, and CD74, whereas the doxycycline induction of the mouse Dux in the “continuous protocol” (see FIG. 7, top panel), completely blocked the IFNG induction of these ISG genes (FIG. 23). These results demonstrate that the inhibition of the ISG response is conserved between mouse DUX and human DUX4 and regions of protein conservation can be used to refine the interaction surfaces between DUX4/Dux and modulators of the ISG response.

Together these studies identify multiple distinct mechanisms of action through which DUX4 expression inhibits distinct pathways in the innate and adaptive immune responses. Section 3 shows that the DUX4 protein, independent of its activity as a transcription factor, has at least two domains necessary and sufficient for inhibition of the different ISG inducing pathways: RIGI, IFIH1, cGAS, and the interferon-gamma and interferon-beta stimulated pathways. These pathways are mediated by factors in the IRF, STAT, and NFKb pathways, indicating that these DUX4 domains together can broadly inhibit multiple arms of the innate-immune response signaling pathways, and that isolated domains can target individual components of a select set of pathways. And that this knowledge can be used to identify the relevant interacting molecules, as was shown for STAT1.

These data also show that DUX4 suppresses MHC class I expression through two distinct mechanisms: blocking the ISG pathways through the mechanisms described above, and a second mechanism that requires the transcriptional activity of DUX4, indicating that this second pathway requires the DUX4-mediated induction of an RNA, either coding or non-coding, that has a long-term suppression on MHC class I expression, but not on the expression of the non-canonical HLA-E protein necessary to protect against natural killer cells.

These data also show that DUX4 profoundly suppresses the IFNG stimulated components of the immunoproteasome. Part of this suppression is consistent with the inhibition of the ISG response that is mediated by the DUX4 protein and a major part of this suppression requires the transcriptional function of DUX4, indicating that DUX4 induces transcription of a factor that prevents the translation of the immunoproteasome mRNAs (e.g., by induction of a regulatory RNA or RNA binding protein) or results in the degradation of the immunoproteasome proteins (e.g., by induction of a ubiquitin ligase targeting these proteins or a protein that prevents their incorporation into the proteasome).

Furthermore, the data identify STAT1 as a protein that interacts with the DUX4 CTD and provides a roadmap for identification of additional proteins. The data also show that mouse Dux robustly inhibits the IFNG ISG in human MB135 cells, providing a method to refine interacting motifs and design functional peptides.

### Example 2: Human DUX4 and Mouse Dux Interact with STAT1 and Broadly Inhibit Interferon-Stimulated Gene Induction

This Example discloses an expanded study of the inhibitory effects of DUX4 as disclosed in Example 1

DUX4 activates the first wave of zygotic gene expression in the early embryo. Mis-expression of DUX4 in skeletal muscle causes facioscapulohumeral dystrophy (FSHD), whereas expression in cancers suppresses IFNG-induction of MHC Class I and contributes to immune evasion. It is shown that the DUX4 protein broadly suppresses immune signaling pathways, including IFNG, IFNβ, DDX58, IFIH1 and cGAS mediated pathways. A conserved region containing (L)LxxL(L) motifs in the DUX4 carboxyterminal domain (CTD) was necessary to suppress interferon stimulated genes (ISGs). Co-immunoprecipitation identified DUX4-CTD interaction with multiple immune signaling factors, including STAT1. The DUX4-CTD (L)LxxL(L) region interacts with phosphorylated STAT1, sequesters it in the nucleus, modestly reduces its DNA binding, and prevents STAT1 from inducing ISG transcription. Mouse Dux similarly interacted with STAT1 and suppressed IFNG induction of ISGs. These findings identify an evolved role of the DUXC family in modulating immune signaling pathways with implications for development, cancers, and FSHD.

Double homeobox (DUX) genes encode a family of transcription factors that originated in placental mammals, consisting of DUXA, DUXB and DUXC subfamilies that all have similar paired homeodomains. The DUXC family is characterized by a small, conserved region at the carboxy-terminus of the protein that includes two (L)LxxL(L) motifs and surrounding conserved amino acids. Members of this family, including mouse Dux and human DUX4, are expressed in a brief burst at early stages of development and regulate an initial wave of zygotic gene activation. While DUX4 expression has also been reported in testes and thymus, it is silenced in most somatic tissues.

Mis-expression of DUX4 in skeletal muscle is the causative factor of facioscapulohumeral muscular dystrophy (FSHD), the third most prevalent human muscular dystrophy. DUX4 expression in skeletal muscle activates the early embryonic totipotent program, suppresses the skeletal muscle program, and ultimately results in muscle cell loss. Many of the genes induced by DUX4 in skeletal muscle encode proteins that are normally restricted to immune-privileged tissues and their expression in skeletal muscle could induce an immune response. In this context it is interesting that FSHD muscle pathology is characterized by focal immune cell infiltrates. However, the inventors' prior studies have also suggested that DUX4 might suppress antigen presentation and aspects of an immune response. Expression of DUX4 in cultured muscle cells blocked lentiviral induction of innate immune response genes such as IFIH1. More recently it was reported that expression of DUX4 in primary cancers and engineered cancer cell lines blocks the interferon-gamma (IFNG) mediated induction of MHC Class I antigen presentation and promotes resistance to immune checkpoint blockade treatments, such as anti-CTLA-4 and anti-PD-1 therapies. The scope and mechanism(s) of how DUX4 suppresses immune signaling remains unknown.

DUX4 contains one LxxLL and one LLxxL motif at its C-terminal end that are among the most highly conserved regions of DUXC-family. LxxLL motifs are alpha-helical protein-interaction domains that were first identified in nuclear-receptor signaling pathways. Proteins containing LxxLL motifs, such as the Protein Inhibitor of Activated STAT or PIAS family, have been shown to modulate immune signaling of STATs, IRFs, NF-kB, and other transcription factors. PIAS proteins block the function of these transcription factors in four ways: preventing DNA binding, recruiting co-repressors, stimulating SUMOylation, or sequestering them within nuclear or sub-nuclear structures.

In this example it is shown that the DUX4 protein has a repressive effect on multiple immune-related signaling pathways, including the IFNG, cGAS, IFIH1 and DDX58 pathways. Notably, a transcriptionally inactive C-terminal fragment of DUX4 is sufficient to block signaling through these pathways and this requires the (L)LxxL(L) domains. Immunoprecipitation and mass spectrometry identified the IFNG-signaling effector STAT1 and several other proteins involved in immune signaling as proteins that interact with the DUX4 C-terminal domain. It is shown here that the (L)LxxL(L) region interacts with STAT1 phosphorylated at Y701, sequesters it in the nucleus, and interferes with its DNA binding and transcriptional activation of interferon stimulated genes (ISGs). Mouse Dux similarly interacts with STAT1 and blocks IFNG stimulation of ISGs. These findings suggest an evolved role of the DUXC family in modulating immune signaling pathways and have implications for the role of DUX4 in development, cancers, and FSHD.

### DUX4 Broadly Suppresses Interferon-Stimulated Gene (ISG) Induction

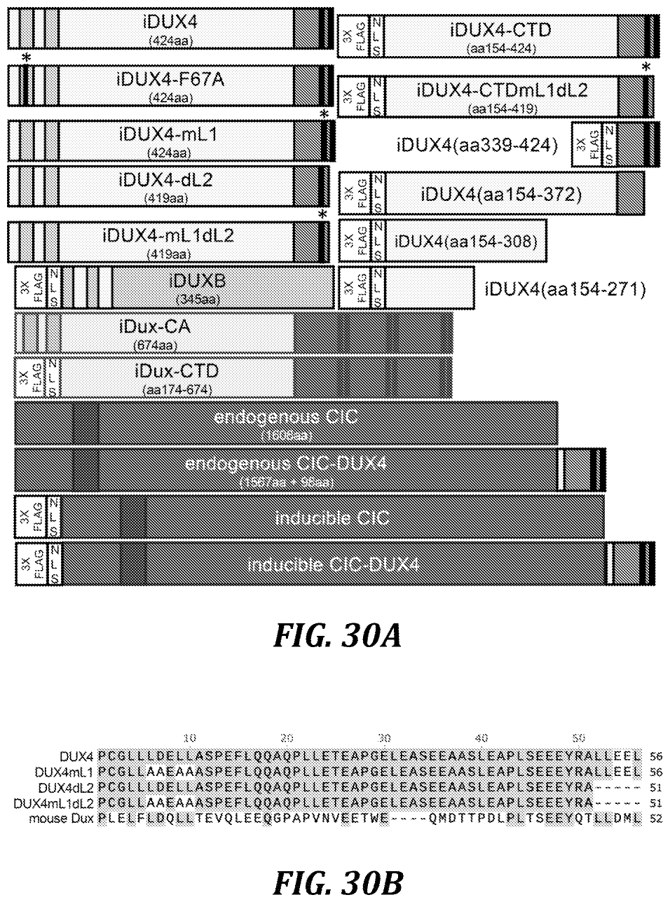

The inventors' prior studies showed that DUX4 inhibited ISG induction in response to lentiviral infection and suppressed induction of MHC Class I proteins in response to interferon-gamma (IFNG, Type-II interferon). To determine whether DUX4 broadly inhibited ISG induction by IFNG, the MB135iDUX4 cell line, a human skeletal muscle cell line with an integrated doxycycline inducible DUX4 (iDUX4) transgene, was used (Jagannathan et al., 2016, Model systems of DUX4 expression recapitulate the transcriptional profile of FSHD cells. Hum Mol Genet 25, 4419-4431.). (See FIG. 30 for schematics of the transgenes used in this example). Doxycycline induction of DUX4 expression in the MB135-iDUX4 cell line has been validated as an accurate cell model of the transcriptional consequences of DUX4 expression in FSHD muscle cells (Jagannathan et al., 2016, supra) and in the early embryo (Hendrickson et al., 2017, Conserved roles of mouse DUX and human DUX4 in activating cleavage-stage genes and MERVL/HERVL retrotransposons. Nat Genet 49, 925-934; Whiddon et al., 2017, Conservation and innovation in the DUX4-family gene network. Nat Genet 49, 935-940). Using a stringent 8-fold induction cut-off (log 2 fold-change >3), RNA-seq showed that IFNG treatment induced 113 genes, whereas the expression of DUX4 suppressed ISG induction by IFNG more than 4-fold for 76 (67%) of these genes and more than 2-fold for 102 genes (90%) (data not shown).

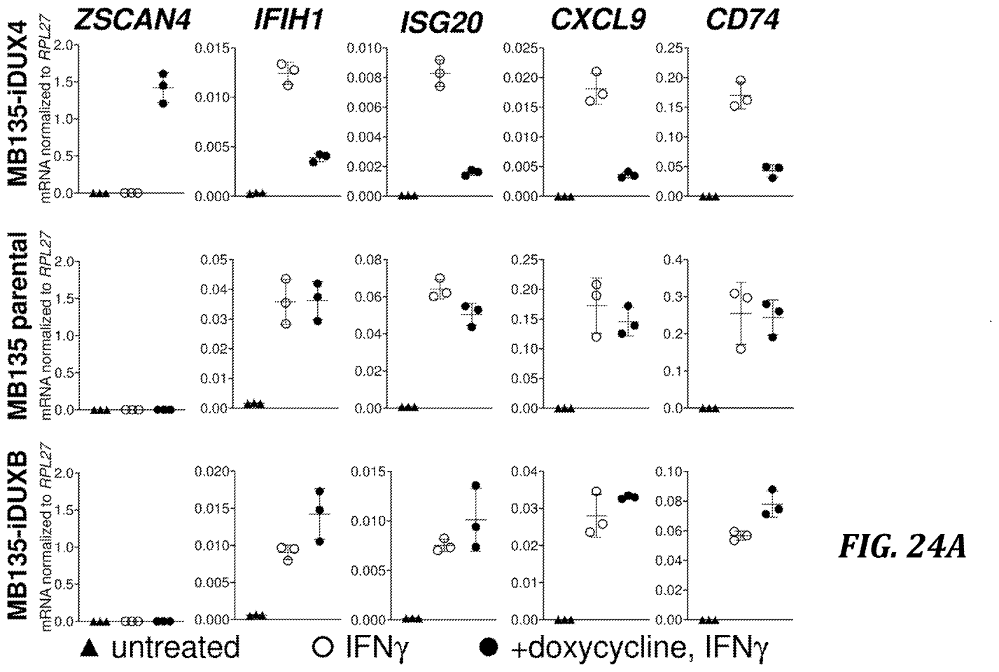

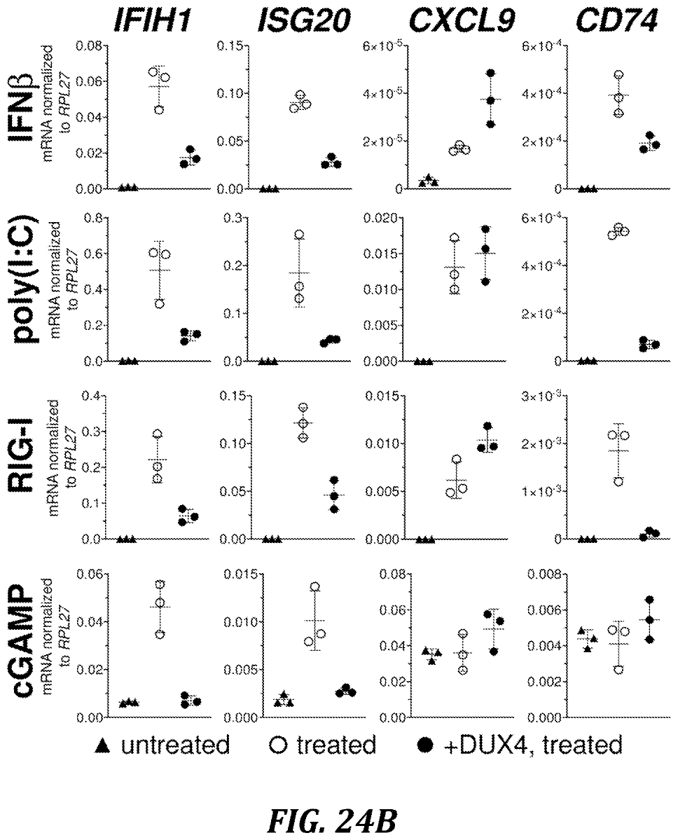

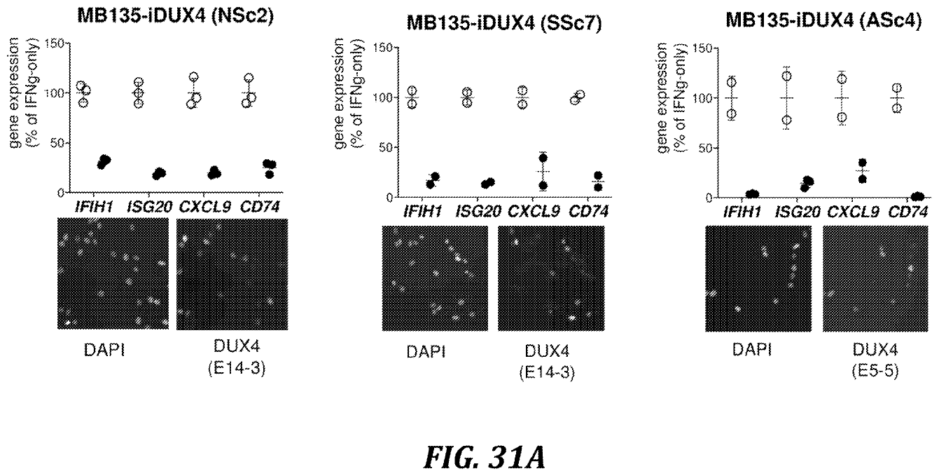

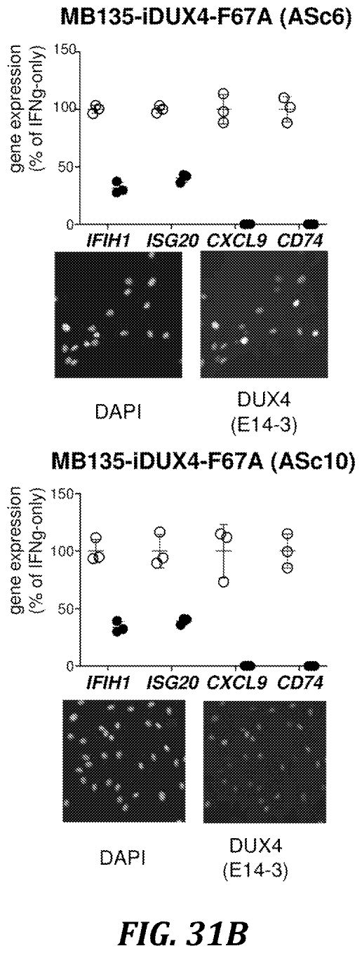

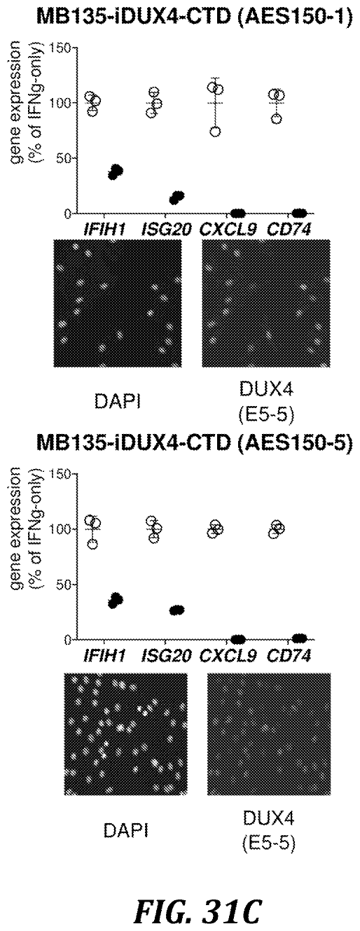

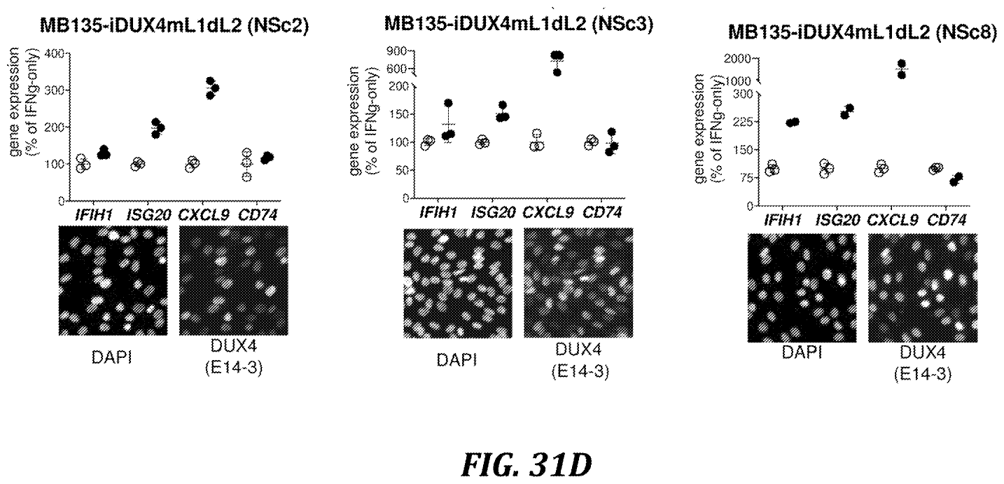

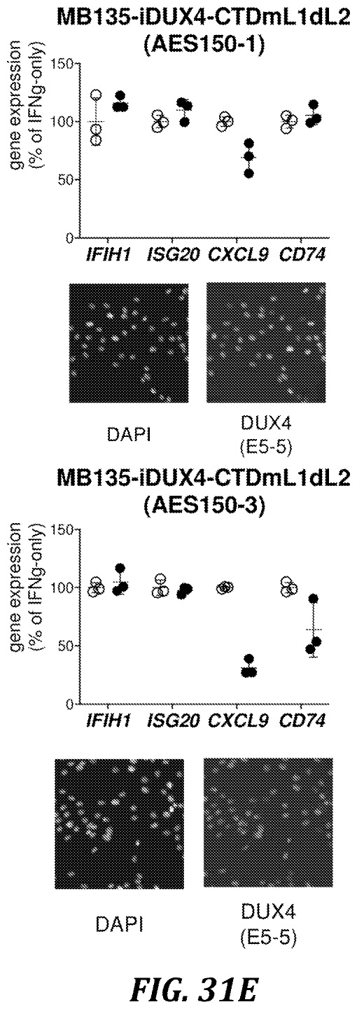

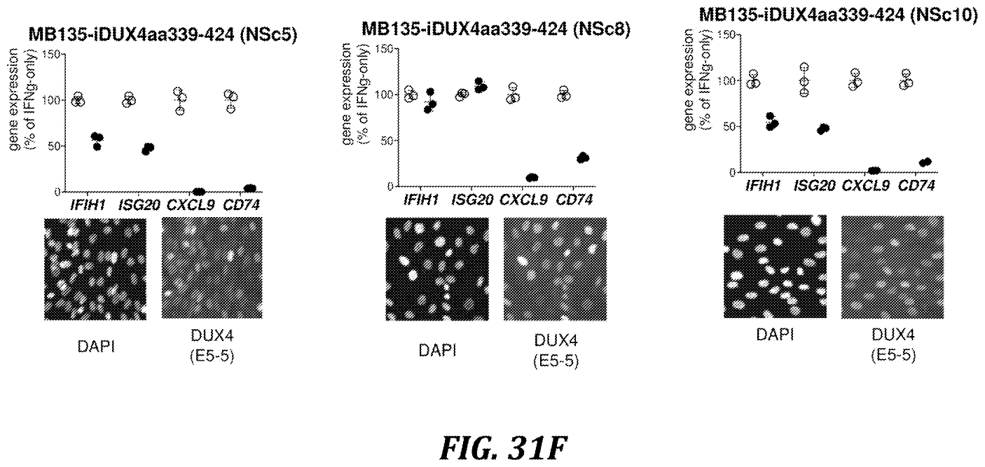

Informed by the RNA-seq results, RT-qPCR was used to measure the response of four ISGs that represent different components of the response to immune signaling: the RNA helicase IFIH1; the interferon-stimulated exonuclease ISG20; the chemoattractant CXCL9; and the major histocompatibility complex class II (MHC-II) chaperone CD74. IFNG-induction of all four genes was robustly blocked by DUX4 expression while a DUX4-target gene ZSCAN4 was strongly induced, indicating that the ISG suppression did not represent a universal block to gene induction (FIG. 24A, MB135-iDUX4 and FIG. 31A); whereas doxycycline treatment in the absence of iDUX4 did not suppress ISG induction (FIG. 24A, MB135 parental). (For this and subsequent constructs, FIGS. 31A-31E show RT-qPCR data from additional independent cell lines together with protein expression and nuclear localization.) In contrast to DUX4, DUXB, a related factor in the DUX family, did not suppress ISG induction by IFNG (FIG. 24A, MB135-iDUXB).

IFNG induces ISGs through the activation of the JAK-STAT signaling pathway. ISGs can also be induced by activation of the innate immune response sensors for double-stranded RNAs and DNAs (dsRNA and dsDNA). These dsRNA/dsDNA pathways, mediated by IFIH1, DDX58, and cGAS, signal primarily through transcription factors IRF3 and NF-kB. To determine whether DUX4 also blocks ISG induction through these signaling pathways, the MB135-iDUX4 cells were transfected with three different innate immune stimuli: poly(I:C), a long dsRNA mimic to stimulate IFIH1; RIG-I ligand, a short dsRNA with a 5′-ppp to stimulate DDX58; or cGAMP, a signaling component of the cGAS dsDNA sensing pathway. Additionally, the cells were stimulated with interferon-beta (IFNβ, Type-I interferon), which primarily signals through JAK-STAT pathways. For all signaling pathways, DUX4 suppressed the induction of a subset of the panel of ISG genes induced by each ligand (FIG. 24B) One exception, CXCL9 was induced by IFNβ, poly(I:C), and the RIG-I ligand but not suppressed by DUX4 (FIG. 24B). cGAMP did not induce CXCL9 or CD74, precluding evaluation of the role of DUX4 in regulating these ISGs. Because these signaling pathways rely on distinct transcription factors, these data suggest that DUX4 might interfere with multiple signaling factors. Thereafter, further efforts focused on identifying the mechanism behind the suppression of IFNG-mediated transcription, as this pathway was most broadly suppressed by DUX4.

### DUX4 Transcriptional Activity is not Necessary for ISG Suppression

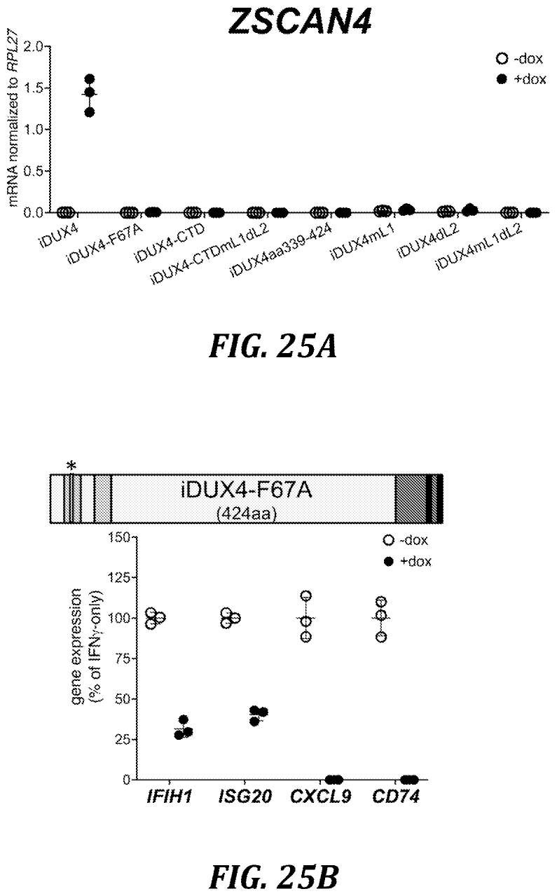

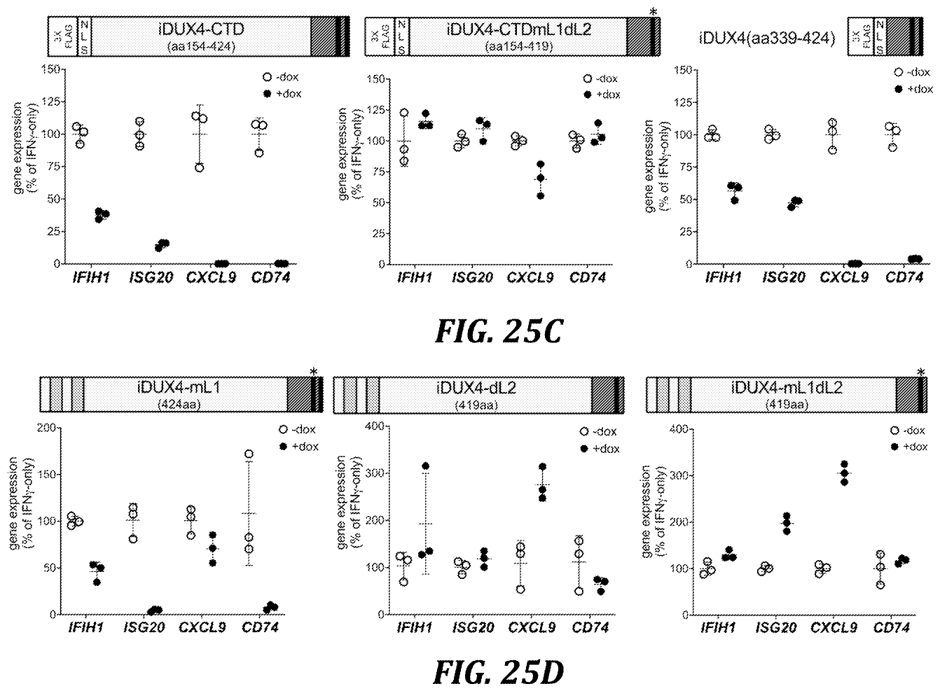

There are two conserved regions of the DUX4 protein, the N-terminal homeodomains (aa19-78, aa94-153) and an ˜50 amino acid region at the end of the C-terminal domain (CTD) that is required for transcriptional activation by DUX4 (aa371-424). A mutation in the first homeodomain, F67A (SEQ ID NOS: 6-7), prevents DUX4 DNA binding and target gene activation. When expressed in MB135 cells, iDUX4-F67A did not activate ZSCAN4 yet still suppressed ISG induction by IFNG (FIGS. 25A-25B, and FIG. 31B). A second construct, iDUX4aa154-424, which has the N-terminal homeodomain region replaced by a 3×FLAG tag and nuclear localization signals (3×FLAG-NLS) cassette (hereafter called iDUX4-CTD) (SEQ ID NOS: 8-9), was also transcriptionally silent yet equally suppressed activation of ISGs (FIGS. 25A-25C and FIG. 31C). RNA sequencing analysis using the same criteria to characterize ISG suppression by the full-length DUX4 demonstrated that the F67A mutant suppressed 70% of induced genes by more than 2-fold, or 41% of induced genes by more than 4-fold; whereas the iDUX4-CTD showed 90% or 52% suppression, respectively (data not shown). Together, these data indicate that DUX4 transcriptional activity is not necessary to suppress IFNG-mediated gene induction.

### the C-Terminal Domain (CTD) is Necessary and Sufficient to Suppress ISGs

The DUX4-CTD contains a pair of (L)LxxL(L) motifs, LLDELL and LLEEL, that are conserved in the DUXC/DUX4 family. DUX4 transgenes mutating the first motif, deleting the second motif, or both (iDUX4mL1, iDUX4dL2, iDUX4mL1dL2) failed to activate the DUX4 target ZSCAN4 (FIG. 25A). iDUX4mL1dL2 and iDUX4dL2 both lost the ability to suppress the panel of ISGs, whereas iDUX4mL1 showed partial activity, suppressing 3 of the 4 ISGs (FIG. 25D and FIG. 31D), indicating that these (L)LxxL(L) motifs are necessary for both ISG suppression and for transcriptional activation by DUX4.

To test sufficiency, two additional C-terminal fragments of DUX4 were generated (FIG. 25C). The first, iDUX4-CTDmL1dL2, contains the CTD of iDUX4mL1dL2 with its N-terminal HDs replaced with the 3×FLAG-NLS cassette (SEQ ID NOS: 10-11). Similar to iDUX4mL1dL2, iDUX4-CTDmL1dL2 did not block the panel of ISGs (FIG. 25C and FIG. 31E). The second construct, iDUX4aa339-424, contains only the C-terminal 85 aa residues including both (L)LxxL(L) motifs (SEQ ID NOS: 12-13), and maintained ISG suppression, though not as strongly on the IFIH1 and ISG20 genes (FIG. 25C and FIG. 31F). In summary, these data support a model in which the DUX4-CTD is both necessary and sufficient to suppress a major portion of the ISG response to IFNG.

the DUX4 Protein Interacts with STAT1 and Additional Immune Response Regulators

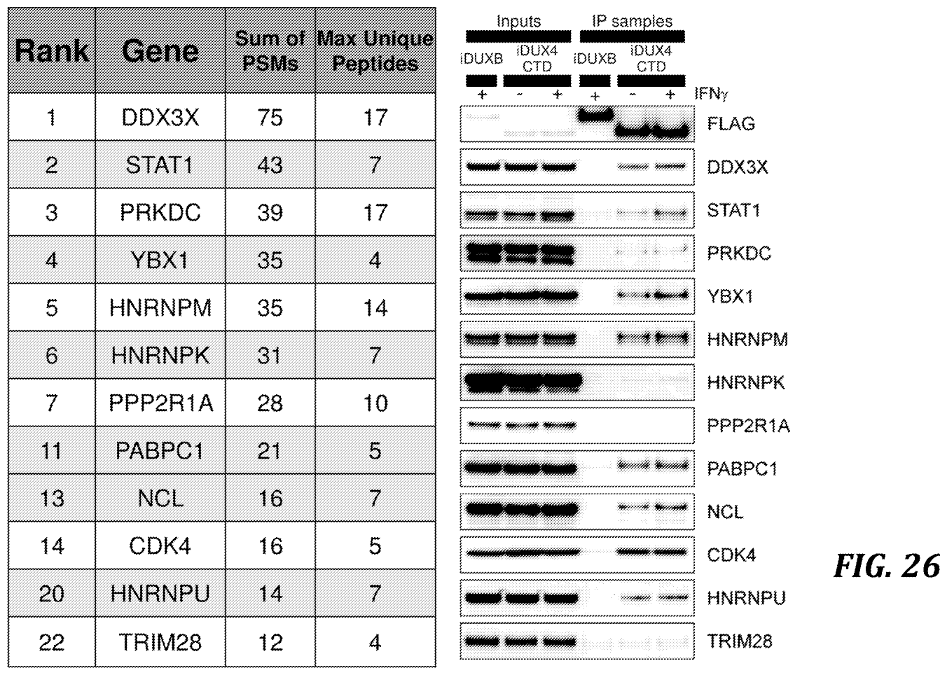

As an unbiased method to identify proteins that interact with the C-terminal region of DUX4, liquid chromatography mass spectroscopy (LC-MS) was used to identify proteins that co-immunoprecipitated with DUX4-CTD constructs expressed in MB135 myoblasts. In the first experiment, MB135iDUX4-CTD cells were used, either untreated, treated with doxycycline alone, or with both doxycycline and IFNG. In the second experiment, MB135iDUX4-CTD and MB135iDUX4mL1dL2 cells were used, both treated with doxycycline and IFNG, compared to these two cell lines untreated and combined as a control. Proteins with a minimum of 2 peptide spectrum matches (PSMs) in at least one sample that were identified in both experiments were assigned to one of ten categories (see Methods) to separate candidate interactors from other categories that might be co-purified because of obligate interactions (e.g., proteasome or ribosome) or might be less likely to be physiologically relevant (e.g., cytoskeletal proteins). Candidate interactors were then ranked based on the total PSMs for that protein across all samples. (It is important to note that the “bait” constructs were expressed at low levels in the samples not treated with doxycycline and that the immunoprecipitation concentrated this background, which might account for some of the candidate proteins appearing in the untreated samples.) STAT1 and DDX3X, two key regulators of innate immune signaling, ranked at the top of the list of candidate DUX4 interactors, together with several other proteins implicated in modulating innate immune signaling (FIG. 26, left panel and Table 1). Western blot analysis using independent biological samples from a co-IP experiment with MB135-iDUX4-CTD and MB135-iDUXB (as a control) validated the DUX4-CTD interactions with DDX3X, STAT1, PRKDC, YBX1, HNRNPM, PABPC1, NCL, CDK4, and HNRPU (FIG. 26, right panel).

The DUX4-CTD Preferentially Interacts with Stat1 Phosphorylated at Y701

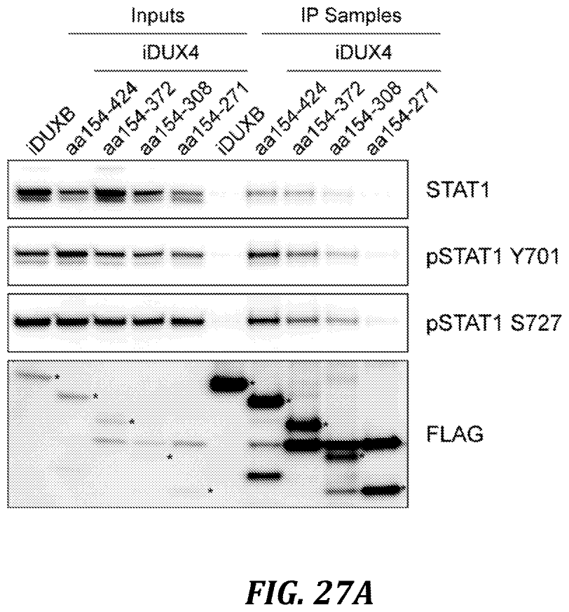

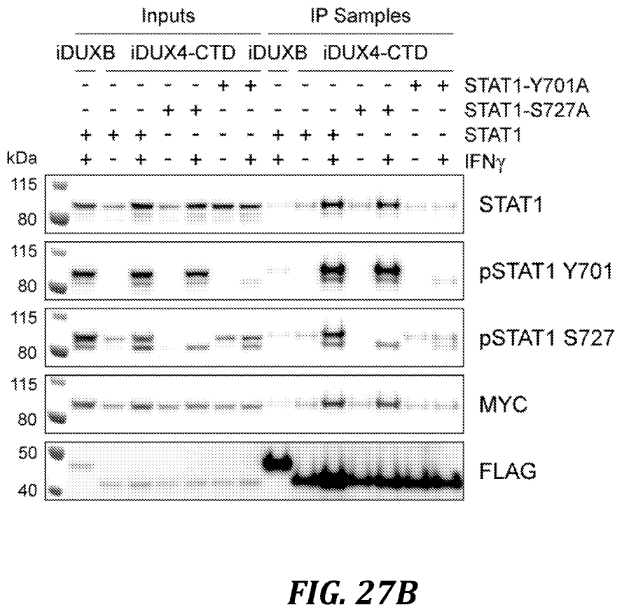

Because of its central role in IFNG signaling, the interaction of STAT1 with DUX4 was studied. To map the region(s) of the DUX4-CTD necessary to interact with STAT1, a truncation series was expressed in MB135 cells (all with an N-terminal 3×FLAG tag and NLS): iDUX4-CTD (aa154-424) (of SEQ ID NO:3), iDUX4aa154-372 (of SEQ ID NO:3), iDUX4aa154-308 (of SEQ ID NO:3), and iDUX4aa154-271 (of SEQ ID NO:3). The region of DUX4 between amino acids 271 and 372 was necessary for co-IP of STAT1, whereas the region between 372 and 424 containing the (L)LxxL(L) motifs appeared necessary for enhanced binding to the phosphorylated forms of STAT1, pSTAT1 Y701 and S727 (FIG. 27A). Together with the validation Western that showed enhanced binding of STAT1 in IFNG treated samples (see FIG. 26), this suggested that the DUX4 CTD showed stronger interaction with phosphorylated STAT1.

To determine which phosphorylation site(s) of STAT1 enhanced interaction with DUX4, the FLAG-tagged iDUX4-CTD was co-expressed with a MYC-tagged iSTAT1 or STAT1 mutants Y701A or S727A, wherein doxycycline would induce expression of both the DUX4 and STAT1 transgenes. The wild-type STAT1 and STAT1-S727A showed enhanced binding to the CTD with IFNG treatment, whereas IFNG did not enhance the binding of STAT1-Y701A (FIG. 27B), indicating that phosphorylation at Y701 is necessary for enhanced interaction with the DUX4-CTD.

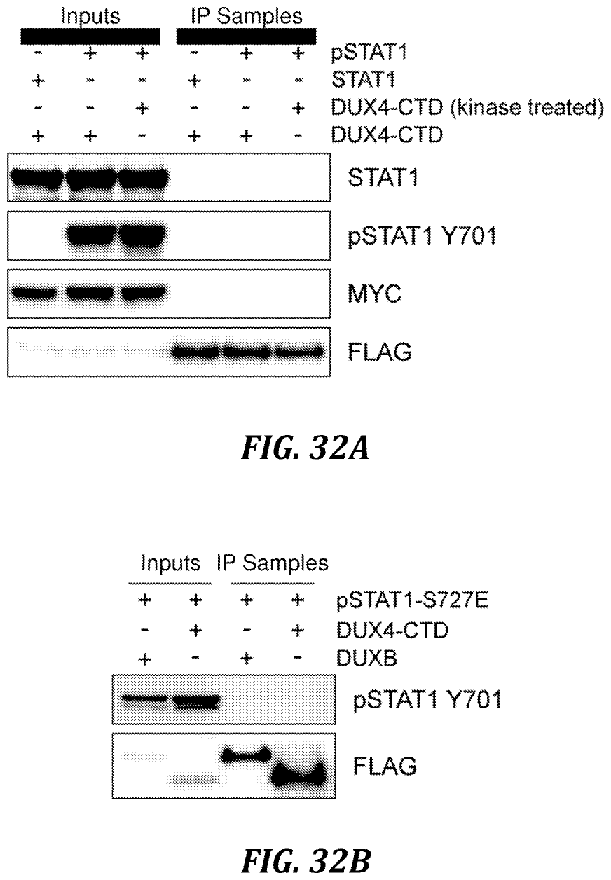

In vitro translated FLAG-tagged/NLS-DUX4-CTD did not interact with in vitro translated STAT1, nor with the phospho-mimic STAT1-S727E with or without treatment with the JAK1 kinase to phosphorylate Y701A (FIG. 32). Therefore, it is likely that the interaction of STAT1 with DUX4 is indirect and requires an unknown scaffold protein, or that DUX4 itself requires a modification to interact with STAT1.

### The DUX4-CTD Tethers Phosphorylated-STAT1 in the Nucleus Following Ifng Treatment

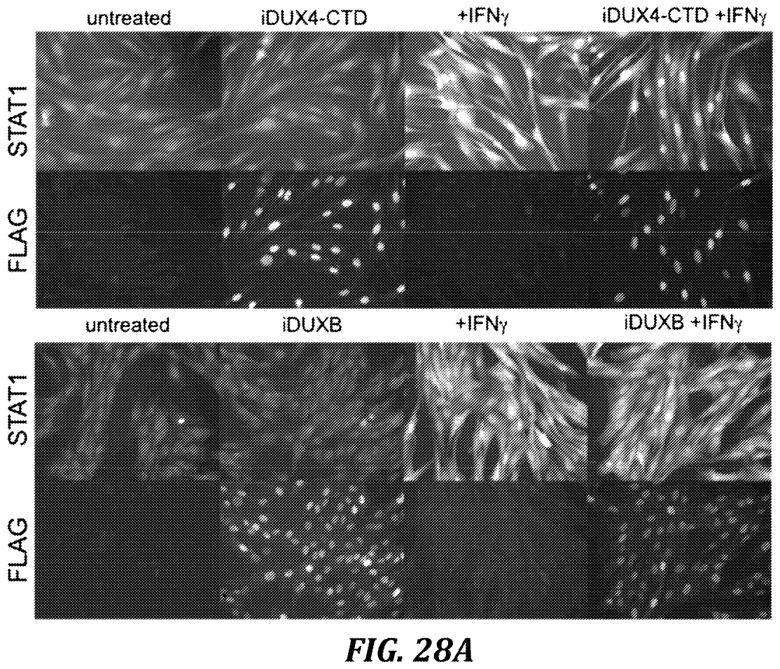

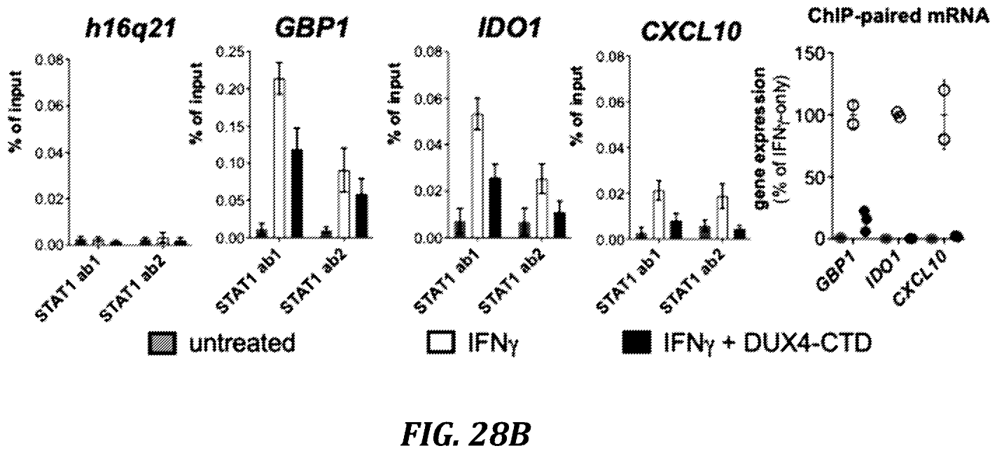

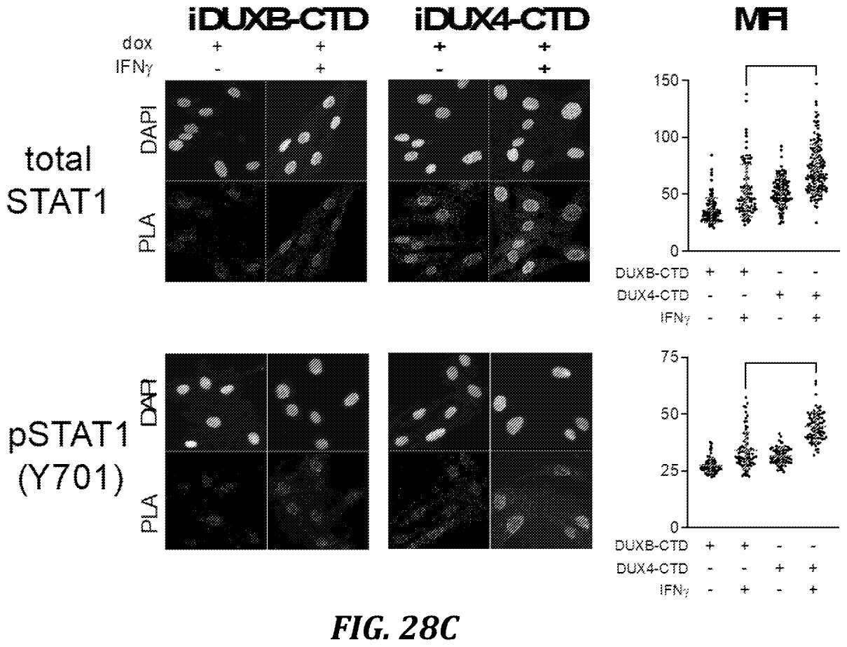

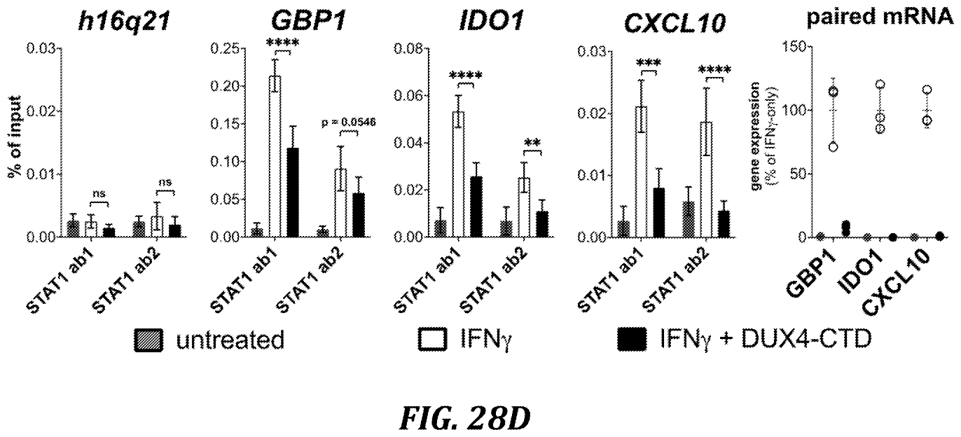

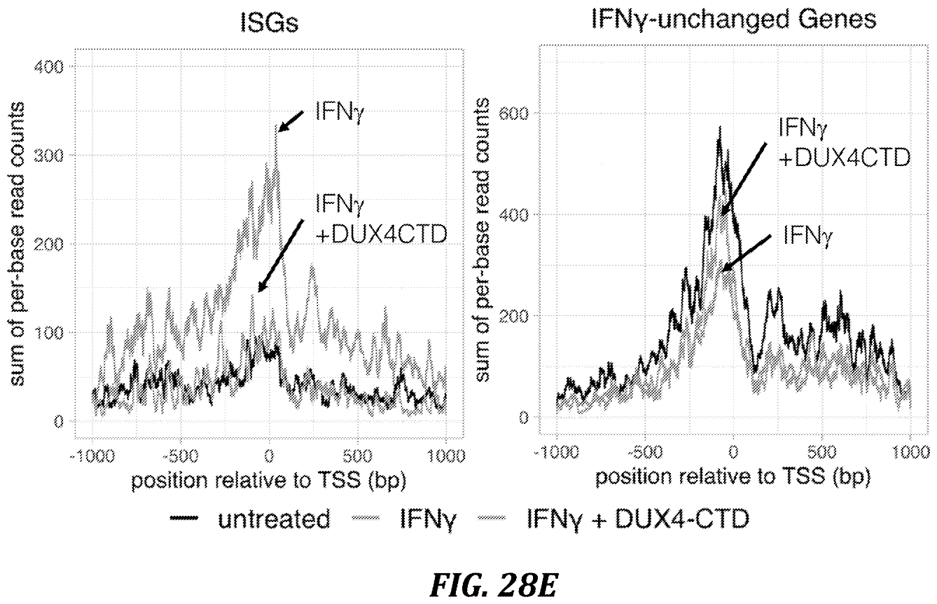

Immunofluorescence showed that while IFNG-treatment increased the STAT1 signal in both the cytoplasm and nucleus, the presence of DUX4-CTD increased the proportion of nuclear STAT1, whereas the presence of DUXB did not (FIG. 28A). This suggests that the interaction between the DUX4-CTD and STAT1 is interfering with the ability of STAT1 to leave the nucleus. This could be due to physical blocking of protein export preventing STAT1 dephosphorylation, which is necessary before STAT1 can return to the cytoplasm to reinitiate the signaling cascade.

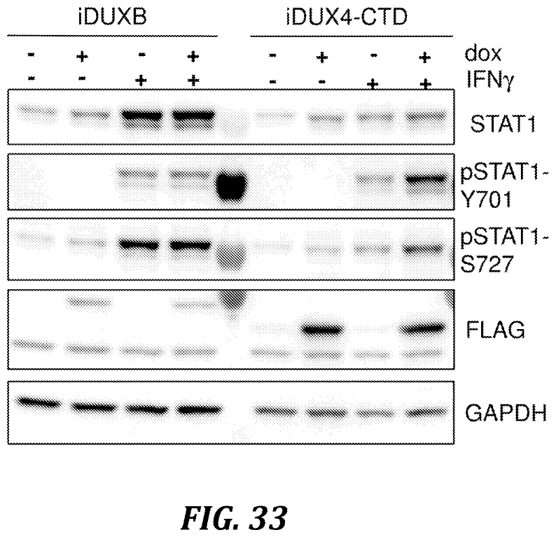

Western analysis of whole-cell extracts showed that expressing DUX4-CTD increased the abundance of pSTAT1 (both pY701 and pS727), but not total STAT1 protein (FIG. 33), whereas iDUXB did not increase either form of phosphorylated STAT1. Furthermore, proximity Ligation Assay (PLA) indicated close interaction between the iDUX4-CTD and endogenous pSTAT1-Y701 in the nucleus of MB135 cells treated with doxycycline and IFNG (FIG. 28C). Therefore, the interaction between DUX4-CTD and STAT1 is enhanced by phosphorylation of STAT1-Y701 and this interaction happens within the nuclei of DUX4-CTD expressing cells.

Together these data demonstrate that the DUX4-CTD does not prevent the accumulation of phosphorylated STAT1 in the nucleus and higher overall levels of phosphorylated STAT1.

### the DUX4-CTD Decreases STAT1 Occupancy at ISG Promoters

The association of DUX4-CTD with pSTAT1-S727 suggested that DUX4 might interact with DNA-bound STAT1 or with STAT1 post-DNA binding and prior to dephosphorylation since pSTAT1-S727 occurs following STAT1 binding DNA. Chromatin immunoprecipitation (ChIP) was performed on MB135-iDUX4-CTD cells to assess STAT1 binding to ISG promoters (FIG. 28B). Compared to a gene-desert region where there should not be STAT1 binding (h16q21), there was a robust induction of STAT1 binding following IFNG treatment at the promoters of several ISGs (GBP1, IDO1, CXCL10) with previously characterized STAT1 binding sites (Rosowski et al., 2014) (FIGS. 28B and 28D, left four panels). Treatment with IFNG following induction of DUX4-CTD diminished STAT1 occupancy at all three ISGs, and paired RT-qPCR confirmed that the DUX4-CTD robustly suppressed the RNA induction by IFNG (FIG. 28D, right panel). CUT&Tag (Cleavage Under Target & Tagmentation) (Kaya-Okur et al., 2019) was used to assess Pol-II occupancy genome wide and found that DUX4-CTD blocked Pol-II recruitment to ISGs without affecting occupancy at other genes (FIG. 28E). Together with the prior data showing that the DUX4-CTD results in increased nuclear phospho-STAT1, these results indicate that the DUX4-CTD interferes with the transcriptional activity of DNA bound STAT1.

Endogenous DUX4 Expression in FSHD Myotubes is Associated with Suppressed ISGS

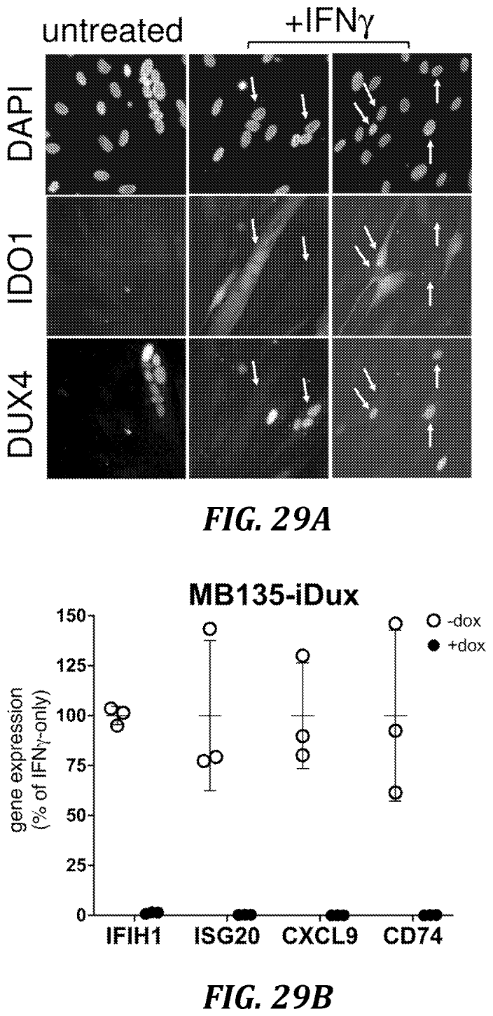

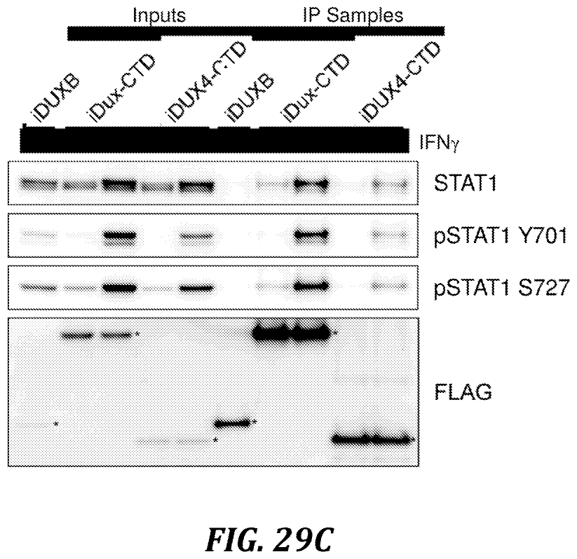

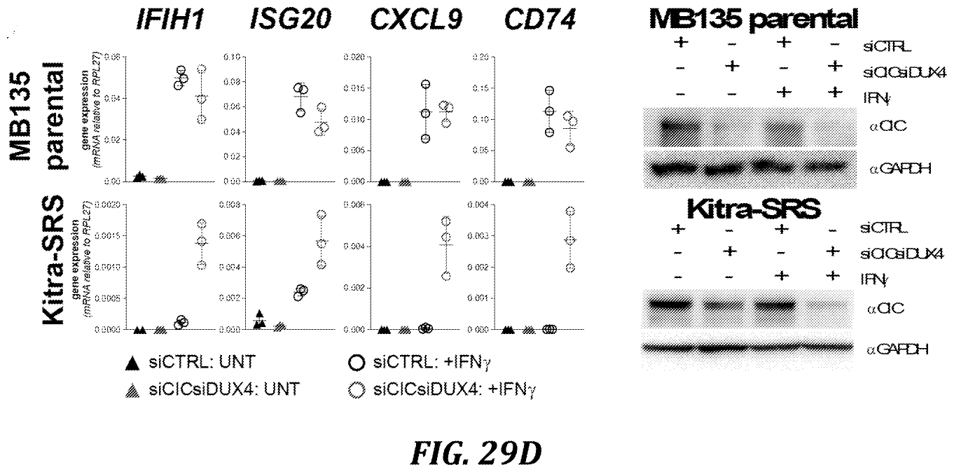

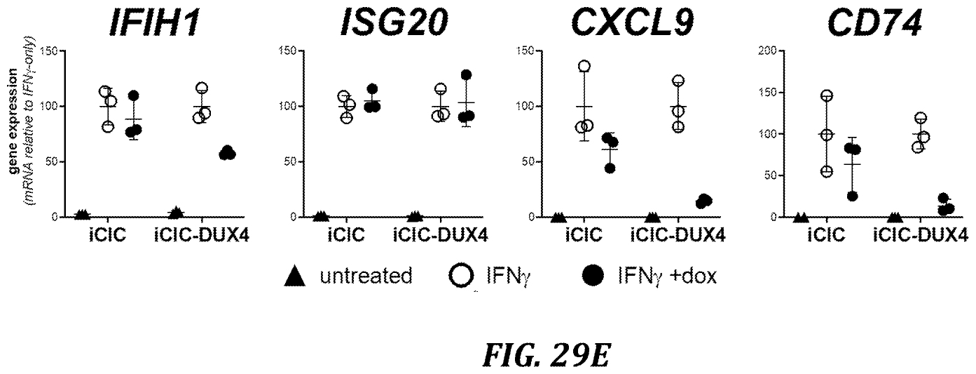

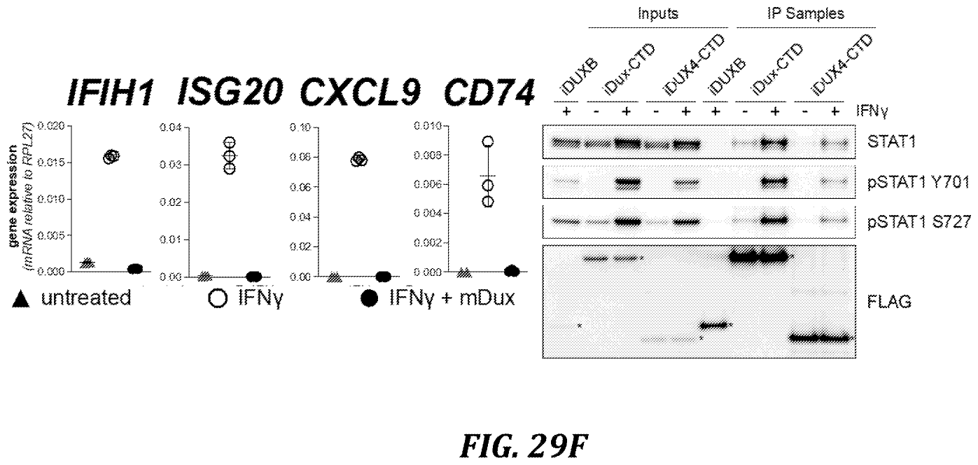

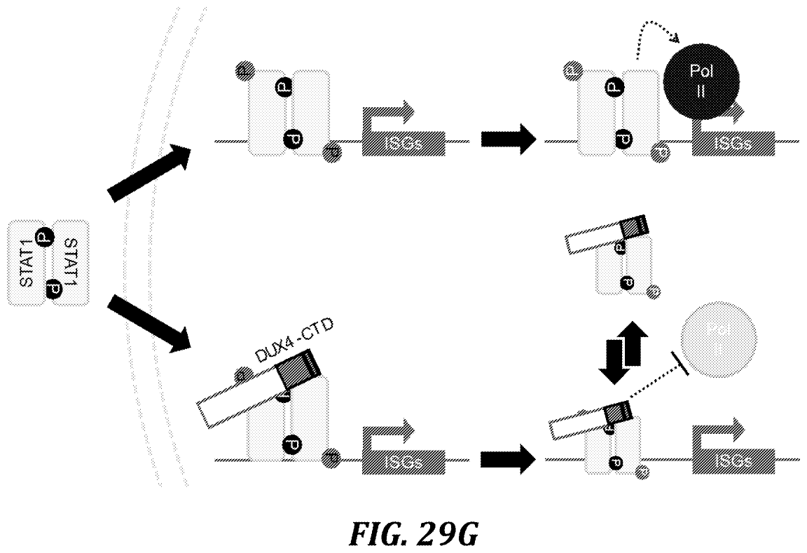

To determine whether endogenous DUX4 suppresses IFNG signaling, IFNG induction of IDO1 in FSHD myotubes was assessed. Differentiation of FSHD myoblasts into multinucleated myotubes results in distinct populations of DUX4-expressing and DUX4-negative myotubes in the same culture, allowing for side-by-side evaluation of DUX4-positive and DUX4-negative muscle cells in the same culture. The IFNG induction of IDO1 was determined as a representative ISG based on its low basal expression in skeletal muscle and the demonstration that it is suppressed in the MB135-iDUX4 cells (FIG. 29B). Treatment with IFNG produced a reliable IDO1 signal within the nucleus and cytoplasm of individual myotubes that did not express DUX4, whereas DUX4-positive myotubes did not show IDO1 expression in response to IFNG (FIG. 29A). Therefore, similar to the inventors' MB135-iDUX4 studies, endogenous DUX4 expressed at a physiological level is sufficient to prevent ISG induction by IFNG.

### Endogenous CIC-DUX4 Fusion Gene Suppresses ISG Induction in a Sarcoma Cell Line

The majority of EWSR1 fusion-negative small blue round cell sarcomas have a genetic re-arrangement between CIC and DUX4 that creates a fusion protein containing the carboxyterminal (L)LxxL(L) motif region of DUX4 (Graham et al., 2012; Kawamura-Saito et al., 2006). The Kitra-SRS sarcoma cell line was confirmed to express a CIC-DUX4 fusion mRNA containing the terminal 98 amino acids of DUX4 as previously described (Nakai et al., 2019). Compared to MB135 myoblasts, Kitra-SRS cells showed absent-to-low induction of ISGs when treated with IFNG and control siRNAs. In contrast, siRNA knockdown of the CIC-DUX4 fusion in the KitraSRS cells resulted in a substantially increased IFNG-induction of ISGs, whereas knockdown of CIC in the MB135 cells did not alter ISG induction (FIG. 29D). To confirm that the CIC-DUX4 fusion was suppressing ISG induction, a doxycycline inducible CIC (SEQ ID NOS: 101-102) or the Kitra-SRS CIC-DUX4 (SEQ ID NOS: 104-105) fusion protein (FIG. 29H) was expressed in MB135 cells and showed that the CIC-DUX4 fusion, but not CIC, suppressed IFNγ-induction of ISGs IFIH1, CXCL9, and CD74, although not ISG20 (FIG. 29E).

### Conservation of ISG Repression and STAT1 Interaction in Mouse Dux

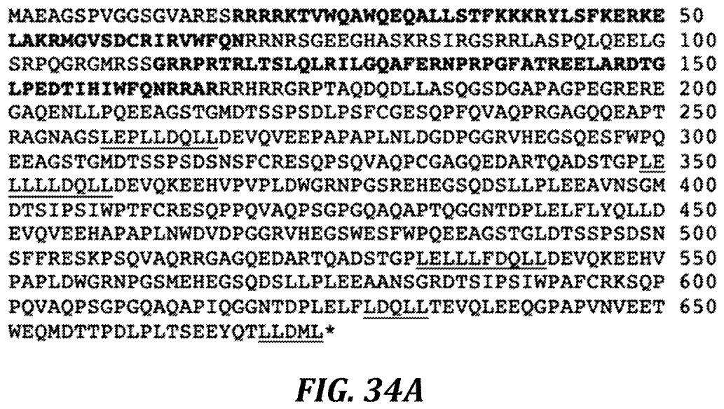

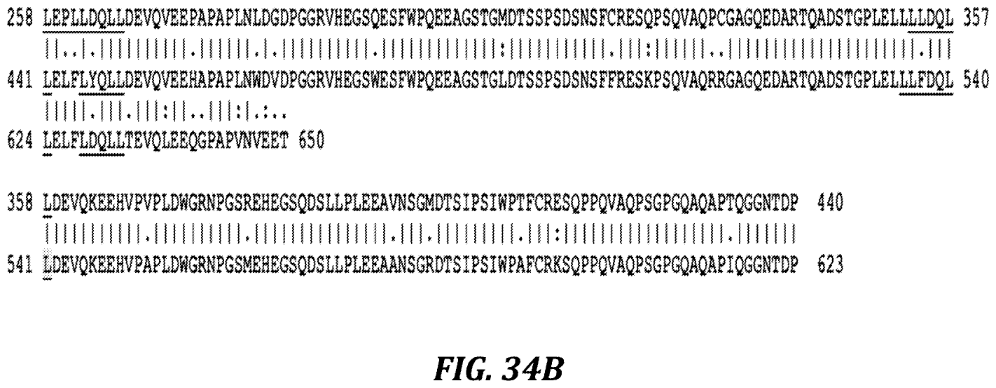

Dux, the mouse ortholog of human DUX4, is expressed at the equivalent developmental stage to human DUX4, activates a parallel transcriptional program, and contains the (L)LxxL(L) motifs that has been shown to be necessary for ISG repression by human DUX4. In fact, the mouse Dux sequence contains a 60aa triplication of the (L)LxxL(L)-containing region (FIG. 34). Accordingly, a doxycycline-inducible mouse Dux transgene was introduced into human MB135 cells (MB135iDux) and the full-length Dux protein was found to repress the panel of ISGs even more robustly than the full-length or CTD portion of human DUX4 (FIG. 29F, left panel). Similar to human DUX4, Western analysis confirmed the co-immunoprecipitation of STAT1 and both phosphorylated pSTAT1-Y701 and pSTAT1-S727 with mouse Dux (FIG. 29F, right panel). These data demonstrate that the suppression of ISG induction and interaction with phosphorylated STAT1 is conserved in the DUXC family.

Together these data demonstrate that the c-terminal domain of DUX4 is necessary and sufficient to broadly suppress ISG induction by IFNG, as well as partially inhibit induction through other pathways mediated by IFNβ, cGAS, IFIH1, and DDX58. Factors that co-immunoprecipitate with the DUX4-CTD were identified, including STAT1, that have roles in regulating immune signaling pathways. The DUX4-CTD colocalizes with STAT1 in the nucleus, diminishes steady-state STAT1 occupancy at ISG promoters, and prevents Pol-II recruitment and transcriptional activation of ISGs by IFNG. Whereas the conserved DUX4 (L)LxxL(L) motifs are necessary to suppress transcriptional activation by STAT1, they are not necessary for the interaction of DUX4 and STAT1. The suppression of IFNG signaling by endogenous DUX4 in FSHD muscle cells and the CIC-DUX4 fusion protein in sarcomas provides support for the biological relevance of these findings.

These data support a simple model of how DUX4 inhibits STAT1 activity (FIG. 29G). IFNG binding to its receptor, IFNGR, leads to the phosphorylation of STAT1 at Y701, subsequently STAT1 forms a homodimer, translocates to the nucleus, and binds the gamma-activated sequence (GAS) in the promoters of ISGs. DNA-bound STAT1 is additionally phosphorylated at S727 and recruits Pol-II to the ISG promoters (Sadzak et al., 2008; Wen et al., 1995). These data also demonstrate that DUX4-CTD interacts with STAT1 phospho-Y701 in the absence of phospho-S727 (i.e., binds the S727A STAT1 mutant), yet also efficiently co-immunoprecipitates with STAT1 phospho-S727 from cell lysates. This indicates that despite DUX4 interacting with STAT1 phospho-Y701, DNA binding of this complex is not fully impaired because of the association with STAT1 phospho-727. The ChIP and CUT&Tag studies show decreased STAT1 steady-state occupancy of ISG promoters and failure to recruit Pol-II. Together, these data support a model of DUX4 interaction with pSTAT1-Y701 that prevents the formation of a stable DNA-bound complex and recruitment of Pol-II, but likely not the initial binding of STAT1 to DNA because of the abundance of phospho-S727 associated with DUX4. The (L)LxxL(L) motifs are necessary to prevent transcriptional activation, presumably by blocking Pol-II recruitment, but not necessary for the interaction of DUX4 with STAT1. This could be due to recruitment of a repressor, or by simply blocking the interaction of STAT1 with an intermediate factor necessary to recruit Pol-II.

The (L)LxxL(L)-dependent inhibition of STAT1 by DUX4 in the current disclosure bears a striking similarity to the inhibitory mechanisms displayed by LxxLL-containing members of the PIAS family. LxxLL motifs were first identified in nuclear-receptor (NR) signaling pathways where they were found to facilitate protein-protein interactions between unbound NRs and co-repressors such as RIP140 and HDACs, or agonist-bound NRs and co-activators such as CBP/p300. LxxLL motifs have since been characterized in multiple protein families, including the PIAS family, and specifically implicated in modulating immune transcriptional networks via interaction with and inhibition of STATs, IRFs, and NF-kB. While the (L)LxxL(L) region of DUX4 is required for suppression of IFNG-mediated ISG induction and its enhanced interaction with pSTAT1-Y701, it is not required for its apparently weaker interaction with unphosphorylated STAT1. In a similar manner, the LxxLL motif of PIASγ is not required for initial binding to STAT1, but is required to suppress ISG induction mediated by STAT1 in response to both IFNβ and IFNG. The same motif is required for the trans-repression of androgen receptor (AR) signaling by PIASγ and of Erythroid Krüppel-like factor (EKLF or KLF1) by PIAS3, though again it is not required for the initial interaction of either pair. Studies hypothesize that this trans-repression relies on the recruitment of co-repressors, although the specific interactors were not determined. Additionally, just as DUX4 appears modestly reduce IFNG-mediated binding of STAT1 to DNA, PIAS proteins can suppress transcriptional networks by blocking DNA binding, as with PIAS3 and STAT3 or PIAS1 and NF-kB p65. Finally, the LxxLL-containing PIASγ was shown to robustly repress the Wnt-responsive transcription factor LEF1 and sequester it within sub-nuclear punctae, reminiscent of the ability of the DUX4-CTD to sequester STAT1 within the nucleus. Studies describe mechanisms of transcriptional suppression by LxxLL motifs in PIAS and other proteins that have strong parallels to the (L)LxxL(L) motifs in human DUX4 and mouse Dux. It is important to emphasize that the xx amino acids in the DUXC family are acidic and there is conservation of some flanking amino acids as well, suggesting that the DUXC family likely evolved target specificity through these larger areas of conservation.

In addition to STAT1, the mass spectrometry identified several proteins that interact with the DUX4-CTD that might also have a role in modulating immune signaling. DDX3X and PRKDC are the top ranked candidates, together with STAT1. DDX3X has been shown to regulate RNA processing, translation, and innate immune signaling. It was also shown to be a pathway specific regulator of IRF3 and IRF7 in part by acting as a scaffolding factor necessary for IKK-ε and TBK1 phosphorylation of IRFs. DDX3X was also shown to be a sensor of dsRNA and viral stem-loop RNA with a role in the initial induction of ISGs, including IFIH1 and DDX5 that then serve to amplify the signaling mechanisms. PRKDC is known mostly for its major roles in DNA repair but also has been implicated in regulating the response to cytoplasmic DNA through the cGAS and IRF3 pathway. Many of the other validated interactors with the DUX4-CTD (see FIG. 26) have independently been identified as interacting with STAT proteins, DDX3X or with other members of the validated group, suggesting that the DUX4-CTD might interact with a multiprotein complex composed of several members of the validated interactors that will require future studies to further define.

The current findings also provide a molecular mechanism for the suppression of IFNG stimulated genes in DUX4-expressing cancers. It was previously reported that the full-length DUX4 is expressed in a diverse set of solid cancers (Chew et al., 2019, DUX4 Suppresses MHC Class I to Promote Cancer Immune Evasion and Resistance to Checkpoint Blockade. Dev Cell 50, 658-671 e657). Cancers expressing DUX4 had diminished IFNG-induced MHC Class I expression, reduced anti-tumor immune cell infiltration, and showed resistance to immune checkpoint blockade. The current study suggests that targeting DUX4 or its interaction with STAT1 can improve immune-based therapies for DUX4-expressing cancers.

In addition, because the transcriptional program of DUX4 is not necessary to inhibit ISG expression, these findings raise the possibility that the portions of the DUX4 protein retained in cancers characterized by DUX4 translocations might retain partial immune modulatory activity. CIC-DUX4 sarcomas generate a fusion protein consisting of the N-terminal region of CIC that contains its DNA binding domain and the CTD of DUX4 that contains its activation domain. The translocation of the DUX4 activation domain to the DNA binding domain of CIC is thought to convert the CIC repressive factor into a transcriptional activator. Because this CTD region of DUX4 also contains the (L)LxxL(L) motifs, there is a reasonable anticipation that these fusion proteins might also modulate IFNG and other signaling pathways. This raises the possibility that targeting this fusion protein might enhance immune surveillance or immunotherapies. In contrast, a subset of adolescent B-cell leukemias are caused by a translocation of a portion of DUX4 into the IGH locus, producing a transcript that has the DUX4 C-terminal region but deletes the (L)LxxL(L) motif containing region of the DUX4-CTD. Although these DUX4-IGH fusions would not be anticipated to interfere with IFNG signaling in the same manner described in the current study, it is notable that the CTD interacting factors identified here maintain interaction with DUX4-CTD deletions that remove the (L)LxxL(L) motifs (see FIG. 27A and data not shown): this raises the possibility that these DUX4-IGH fusions might alter some aspects of immune signaling yet to be identified.

The conservation of the (L)LxxL(L) motifs in mouse Dux and its similar interaction with STAT1 and inhibition of IFNG signaling indicates that this is a conserved function of the DUXC family. DUX4, Dux, and the canine DUXC all induce expression of endogenous retroelements, as well as pericentromeric satellite repeats that form dsRNAs that, at least in the case of DUX4, induce a dsRNA response that results in activation of PKR and phosphorylation of EIF2α. Therefore, it is possible that the interaction with STAT1 and other immune signaling modulators might prevent the activation of the ISG pathway while permitting the PKR response, although the biological consequences remain to be further explored. It is also interesting that DUX4, Dux and possibly other members of the DUXC family are expressed in immune privileged tissues—i.e., cleavage embryo, testis and thymus—and this study suggests that their expression might contribute to this immune privileged state. In this regard, it is compelling that the DUX family arose at the origin of placental mammals and it is intriguing to speculate that the immune modulation beginning to be uncovered has a role in establishing maternal-fetal tolerance, although expression of DUX4 at later stages of trophectoderm and placental development require further study.

TABLE 1
proteins that are candidates for interaction with DUX4.

| | | | | | 4. |
|---|---|---|---|---|---|
| | | | | 4. + | mL1dL2 + |
| | | | | IFNG | Dox + |
| Accession | Description | Cat | Gene_name | mix | IFNG |
| A0A0D9SF53 | ATP-dependent RNA helicase DDX3X | 1 | DDX3X | 1 | 2 |
| | OS = Homo sapiens OX = 9606 | | | | |
| | GN = DDX3X PE = 1 SV = 1 | | | | |
| P42224 | Signal transducer and activator of | 1 | STAT1 | 3 | 18 |
| | transcription 1-alpha/beta OS = Homo | | | | |
| | sapiens OX = 9606 GN = STAT1 PE = | | | | |
| | 1 SV = 2 | | | | |
| P78527 | DNA-dependent protein kinase catalytic | 1 | PRKDC | 11 | 5 |
| | subunit OS = Homo sapiens OX = 9606 | | | | |
| | GN = PRKDC PE = 1 SV = 3 | | | | |
| H0Y449 | Nuclease-sensitive element-binding protein | 1 | YBX1 | 66 | 10 |
| | 1 (Fragment) OS = Homo sapiens OX = | | | | |
| | 9606 GN = YBX1 PE = 1 SV = 1 | | | | |
| A0A087X0X3 | Heterogeneous nuclear ribonucleoprotein M | 1 | HNRNPM | | 3 |
| | OS = Homo sapiens OX = 9606 | | | | |
| | GN = HNRNPM PE = 1 SV = 1 | | | | |
| P61978 | Heterogeneous nuclear ribonucleoprotein K | 1 | HNRNPK | 3 | 12 |
| | OS = Homo sapiens OX = 9606 | | | | |
| | GN = HNRNPK PE = 1 SV = 1 | | | | |
| P30153 | Serine/threonine-protein phosphatase 2A 65 | 1 | PPP2R1A | | 5 |
| | kDa regulatory subunit A alpha isoform | | | | |
| | OS = Homo sapiens OX = 9606 | | | | |
| | GN = PPP2R1A PE = 1 SV = 4 | | | | |
| Q16576 | Histone-binding protein RBBP7 OS = Homo | 1 | RBBP7 | | 16 |
| | sapiens OX = 9606 GN = RBBP7 | | | | |
| | PE = 1 SV = 1 | | | | |
| Q9NQC3 | Reticulon-4 OS = Homo sapiens OX = | 1 | RTN4 | 5 | 5 |
| | 9606 GN = RTN4 PE = 1 SV = 2 | | | | |
| O14929 | Histone acetyltransferase type B catalytic | 1 | HAT1 | 1 | 10 |
| | subunit OS = Homo sapiens OX = 9606 | | | | |
| | GN = HAT1 PE = 1 SV = 1 | | | | |
| P11940 | Polyadenylate-binding protein 1 OS = Homo | 1 | PABPC1 | | 5 |
| | sapiens OX = 9606 GN = PABPC1 | | | | |
| | PE = 1 SV = 2 | | | | |
| G8JLB6 | Heterogeneous nuclear ribonucleoprotein H | 1 | HNRNPH1 | | 7 |
| | OS = Homo sapiens OX = 9606 | | | | |
| | GN = HNRNPH1 PE = 1 SV = 1 | | | | |
| P19338 | Nucleolin OS = Homo sapiens OX = 9606 | 1 | NCL | 2 | 9 |
| | GN = NCL PE = 1 SV = 3 | | | | |
| P11802 | Cyclin-dependent kinase 4 OS = Homo | 1 | CDK4 | | 3 |
| | sapiens OX = 9606 GN = CDK4 | | | | |
| | PE = 1 SV = 2 | | | | |
| P06748 | Nucleophosmin OS = Homo sapiens | 1 | NPM1 | 2 | 4 |
| | OX = 9606 GN = NPM1 PE = 1 SV = 2 | | | | |
| Q15393 | Splicing factor 3B subunit 3 OS = Homo | 1 | SF3B3 | | 10 |
| | sapiens OX = 9606 GN = SF3B3 | | | | |
| | PE = 1 SV = 4 | | | | |
| Q93009 | Ubiquitin carboxyl-terminal hydrolase 7 | 1 | USP7 | | 9 |
| | OS = Homo sapiens OX = 9606 | | | | |
| | GN = USP7 PE = 1 SV = 2 | | | | |
| Q93008 | Probable ubiquitin carboxyl-terminal | 1 | USP9X | | 7 |
| | hydrolase FAF-X OS = Homo sapiens | | | | |
| | OX = 9606 GN = USP9X PE = 1 SV = 3 | | | | |
| P13489 | Ribonuclease inhibitor OS = Homo sapiens | 1 | RNH1 | | 4 |
| | OX = 9606 GN = RNH1 PE = 1 SV = 2 | | | | |
| Q00839 | Heterogeneous nuclear ribonucleoprotein U | 1 | HNRNPU | | 1 |
| | OS = Homo sapiens OX = 9606 | | | | |
| | GN = HNRNPU PE = 1 SV = 6 | | | | |
| Q09666 | Neuroblast differentiation-associated | 1 | AHNAK | 1 | |
| | protein AHNAK OS = Homo sapiens | | | | |
| | OX = 9606 GN = AHNAK PE = 1 SV = 2 | | | | |
| Q13263 | Transcription intermediary factor 1-beta | 1 | TRIM28 | 1 | 8 |
| | OS = Homo sapiens OX = 9606 | | | | |
| | GN = TRIM28 PE = 1 SV = 5 | | | | |
| P16989 | Y-box-binding protein 3 OS = Homo sapiens | 1 | YBX3 | | 7 |
| | OX = 9606 GN = YBX3 PE = 1 SV = 4 | | | | |
| Q14444 | Caprin-1 OS = Homo sapiens OX = 9606 | 1 | CAPRIN1 | | 1 |
| | GN = CAPRIN1 PE = 1 SV = 2 | | | | |
| Q15208 | Serine/threonine-protein kinase 38 | 1 | STK38 | 4 | |
| | OS = Homo sapiens OX = 9606 | | | | |
| | GN = STK38 PE = 1 SV = 1 | | | | |
| Q63ZY3 | KN motif and ankyrin repeat domain- | 1 | KANK2 | | 1 |
| | containing protein 2 OS = Homo sapiens | | | | |
| | OX = 9606 GN = KANK2 PE = 1 SV = 1 | | | | |
| P55209 | Nucleosome assembly protein 1-like 1 | 1 | NAP1L1 | | 2 |
| | OS = Homo sapiens OX = 9606 | | | | |
| | GN = NAP1L1 PE = 1 SV = 1 | | | | |
| Q15233 | Non-POU domain-containing octamer- | 1 | NONO | 1 | |
| | binding protein OS = Homo sapiens | | | | |
| | OX = 9606 GN = NONO PE = 1 SV = 4 | | | | |
| P47929 | Galectin-7 OS = Homo sapiens OX = | 1 | LGALS7 | | |
| | 9606 GN = LGALS7 PE = 1 SV = 2 | | | | |
| Q13283 | Ras GTPase-activating protein-binding | 1 | G3BP1 | 1 | 1 |
| | protein 1 OS = Homo sapiens OX = | | | | |
| | 9606 GN = G3BP1 PE = 1 SV = 1 | | | | |
| Q8NC51 | Plasminogen activator inhibitor 1 RNA- | 1 | SERBP1 | | |
| | binding protein OS = Homo sapiens | | | | |
| | OX = 9606 GN = SERBP1 PE = 1 SV = 2 | | | | |
| Q9UN86 | Ras GTPase-activating protein-binding | 1 | G3BP2 | | 1 |
| | protein 2 OS = Homo sapiens OX = | | | | |
| | 9606 GN = G3BP2 PE = 1 SV = 2 | | | | |
| P26196 | Probable ATP-dependent RNA helicase | 1 | DDX6 | | |
| | DDX6 OS = Homo sapiens OX = 9606 | | | | |
| | GN = DDX6 PE = 1 SV = 2 | | | | |
| P13010 | X-ray repair cross-complementing protein 5 | 1 | XRCC5 | | 1 |
| | OS = Homo sapiens OX = 9606 | | | | |
| | GN = XRCC5 PE = 1 SV = 3 | | | | |
| P52597 | Heterogeneous nuclear ribonucleoprotein F | 1 | HNRNPF | | |
| | OS = Homo sapiens OX = 9606 | | | | |
| | GN = HNRNPF PE = 1 SV = 3 | | | | |
| E7ENX8 | Uncharacterized protein (Fragment) | 1 | NA | | 2 |
| | OS = Homo sapiens OX = 9606 | | (Homology | | |
| | PE = 3 SV = 1 | | to TAP2) | | |
| O94919 | Endonuclease domain-containing 1 protein | 1 | ENDOD1 | 1 | 1 |
| | OS = Homo sapiens OX = 9606 | | | | |
| | GN = ENDOD1 PE = 1 SV = 2 | | | | |
| P40763 | Signal transducer and activator of | 1 | STAT3 | | 1 |
| | transcription 3 OS = Homo sapiens | | | | |
| | OX = 9606 GN = STAT3 PE = 1 SV = 2 | | | | |
| F8W930 | Insulin-like growth factor 2 mRNA-binding | 1 | IGF2BP2 | | 1 |
| | protein 2 OS = Homo sapiens OX = 9606 | | | | |
| | GN = IGF2BP2 PE = 1 SV = 1 | | | | |
| P29508 | Serpin B3 OS = Homo sapiens OX = 9606 | 1 | SERPINB3 | 2 | |
| | GN = SERPINB3 PE = 1 SV = 2 | | | | |
| Q86YD1 | Prostate tumor-overexpressed gene 1 | 1 | PTOV1 | | |
| | protein OS = Homo sapiens OX = 9606 | | | | |
| | GN = PTOV1 PE = 1 SV = 1 | | | | |
| P17655 | Calpain-2 catalytic subunit OS = Homo | 1 | CAPN2 | | |
| | sapiens OX = 9606 GN = CAPN2 | | | | |
| | PE = 1 SV = 6 | | | | |
| P51784 | Ubiquitin carboxyl-terminal hydrolase 11 | 1 | USP11 | | 1 |
| | OS = Homo sapiens OX = 9606 | | | | |
| | GN = USP11 PE = 1 SV = 3 | | | | |
| A0A0U1RRM4 | Polypyrimidine tract-binding protein 1 | 1 | PTBP1 | | 1 |
| | OS = Homo sapiens OX = 9606 | | | | |
| | GN = PTBP1 PE = 1 SV = 1 | | | | |
| P35579 | Myosin-9 OS = Homo sapiens OX = 9606 | 2 | MYH9 | 10 | 1 |
| | GN = MYH9 PE = 1 SV = 4 | | | | |
| Q07065 | Cytoskeleton-associated protein 4 | 2 | CKAP4 | 24 | 19 |
| | OS = Homo sapiens OX = 9606 GN = | | | | |
| | CKAP4 PE = 1 SV = 2 | | | | |
| P12814 | Alpha-actinin-1 OS = Homo sapiens | 2 | ACTN1 | 9 | 22 |
| | OX = 9606 GN = ACTN1 PE = 1 SV = 2 | | | | |
| Q16643 | Drebrin OS = Homo sapiens OX = 9606 | 2 | DBN1 | | 2 |
| | GN = DBN1 PE = 1 SV = 4 | | | | |
| A0A087WVQ6 | Clathrin heavy chain OS = Homo sapiens | 2 | CLTC | | 8 |
| | OX = 9606 GN = CLTC PE = 1 SV = 1 | | | | |
| Q6NZI2 | Caveolae-associated protein 1 OS = Homo | 2 | CAVIN1 | 7 | 16 |
| | sapiens OX = 9606 GN = CAVIN1 | | | | |
| | PE = 1 SV = 1 | | | | |
| P21333 | Filamin-A OS = Homo sapiens OX = 9606 | 2 | FLNA | 3 | 4 |
| | GN = FLNA PE = 1 SV = 4 | | | | |
| Q14315 | Filamin-C OS = Homo sapiens OX = 9606 | 2 | FLNC | 1 | 2 |
| | GN = FLNC PE = 1 SV = 3 | | | | |
| P02545 | Prelamin-A/C OS = Homo sapiens OX = | 2 | LMNA | 4 | 8 |
| | 9606 GN = LMNA PE = 1 SV = 1 | | | | |
| P61158 | Actin-related protein 3 OS = Homo sapiens | 2 | ACTR3 | | |
| | OX = 9606 GN = ACTR3 PE = 1 SV = 3 | | | | |
| E9PK25 | Cofilin-1 OS = Homo sapiens OX = 9606 | 2 | CFL1 | 1 | 4 |
| | GN = CFL1 PE = 1 SV = 1 | | | | |
| B1AK88 | Capping protein (Actin filament) muscle Z- | 2 | CAPZB | 1 | 6 |
| | line, beta, isoform CRA_d OS = Homo | | | | |
| | sapiens OX = 9606 GN = CAPZB | | | | |
| | PE = 1 SV = 1 | | | | |
| P48681 | Nestin OS = Homo sapiens OX = 9606 | 2 | NES | 6 | 1 |
| | GN = NES PE = 1 SV = 2 | | | | |
| O75083 | WD repeat-containing protein 1 OS = Homo | 2 | WDR1 | | 2 |
| | sapiens OX = 9606 GN = WDR1 | | | | |
| | PE = 1 SV = 4 | | | | |
| B7Z6Z4 | cDNA FLJ56329, highly similar to Myosin | 2 | MYL6 | | |
| | light polypeptide 6 OS = Homo sapiens | | | | |
| | OX = 9606 GN = MYL6 PE = 1 SV = 1 | | | | |
| Q14247 | Src substrate cortactin OS = Homo sapiens | 2 | CTTN | | |
| | OX = 9606 GN = CTTN PE = 1 SV = 2 | | | | |
| Q9Y490 | Talin-1 OS = Homo sapiens OX = 9606 | 2 | TLN1 | | |
| | GN = TLN1 PE = 1 SV = 3 | | | | |
| Q13409 | Cytoplasmic dynein 1 intermediate chain 2 | 2 | DYNC1I2 | | |
| | OS = Homo sapiens OX = 9606 | | | | |
| | GN = DYNC1I2 PE = 1 SV = 3 | | | | |
| Q14203 | Dynactin subunit 1 OS = Homo sapiens | 2 | DCTN1 | | 1 |
| | OX = 9606 GN = DCTN1 PE = 1 SV = 3 | | | | |
| P60709 | Actin, cytoplasmic 1 OS = Homo sapiens | 3 | ACTB | 52 | 104 |
| | OX = 9606 GN = ACTB PE = 1 SV = 1 | | | | |
| P07437 | Tubulin beta chain OS = Homo sapiens | 3 | TUBB | 19 | 56 |
| | OX = 9606 GN = TUBB PE = 1 SV = 2 | | | | |
| P08670 | Vimentin OS = Homo sapiens OX = 9606 | 3 | VIM | 66 | 43 |
| | GN = VIM PE = 1 SV = 4 | | | | |
| P68371 | Tubulin beta-4B chain OS = Homo sapiens | 3 | TUBB4B | 13 | 47 |
| | OX = 9606 GN = TUBB4B PE = 1 SV = 1 | | | | |
| P62736 | Actin, aortic smooth muscle OS = Homo | 3 | ACTA2 | 23 | 68 |
| | sapiens OX = 9606 GN = ACTA2 PE = 1 | | | | |
| | SV = 1 | | | | |
| Q13885 | Tubulin beta-2A chain OS = Homo sapiens | 3 | TUBB2A | | |
| | OX = 9606 GN = TUBB2A PE = 1 SV = 1 | | | | |
| Q9BUF5 | Tubulin beta-6 chain OS = Homo sapiens | 3 | TUBB6 | | 23 |
| | OX = 9606 GN = TUBB6 PE = 1 SV = 1 | | | | |
| J3QRS3 | Myosin regulatory light chain 12A | 3 | MYL12A | 9 | 30 |
| | OS = Homo sapiens OX = 9606 | | | | |
| | GN = MYL12A PE = 1 SV = 1 | | | | |
| O43707 | Alpha-actinin-4 OS = Homo sapiens | 3 | ACTN4 | 9 | 24 |
| | OX = 9606 GN = ACTN4 PE = 1 SV = 2 | | | | |
| P17661 | Desmin OS = Homo sapiens OX = 9606 | 3 | DES | 24 | 10 |
| | GN = DES PE = 1 SV = 3 | | | | |
| F5H5D3 | Tubulin alpha chain OS = Homo sapiens | 3 | TUBA1C | | |
| | OX = 9606 GN = TUBA1C PE = 1 SV = 1 | | | | |
| P68366 | Tubulin alpha-4A chain OS = Homo sapiens | 3 | TUBA4A | | |
| | OX = 9606 GN = TUBA4A PE = 1 SV = 1 | | | | |
| P68363 | Tubulin alpha-1B chain OS = Homo sapiens | 3 | TUBA1B | 9 | 24 |
| | OX = 9606 GN = TUBA1B PE = 1 SV = 1 | | | | |
| Q15149 | Plectin OS = Homo sapiens OX = 9606 | 3 | PLEC | 3 | 16 |
| | GN = PLEC PE = 1 SV = 3 | | | | |
| Q3SY84 | Keratin, type II cytoskeletal 71 OS = Homo | 3 | KRT71 | | 21 |
| | sapiens OX = 9606 GN = KRT71 PE = | | | | |
| | 1 SV = 3 | | | | |
| A0A087WUZ3 | Spectrin beta chain OS = Homo sapiens | 3 | SPTBN1 | | |
| | OX = 9606 GN = SPTBN1 PE = 1 SV = 1 | | | | |
| A0A0D9SGF6 | Spectrin alpha chain, non-erythrocytic 1 | 3 | SPTAN1 | | |
| | OS = Homo sapiens OX = 9606 GN = | | | | |
| | SPTAN1 PE = 1 SV = 1 | | | | |
| Q9P2E9 | Ribosome-binding protein 1 OS = Homo | 4 | RRBP1 | 12 | 4 |
| | sapiens OX = 9606 GN = RRBP1 | | | | |
| | PE = 1 SV = 5 | | | | |
| P05388 | 60S acidic ribosomal protein P0 OS = Homo | 4 | RPLP0 | 7 | 40 |
| | sapiens OX = 9606 GN = RPLP0 | | | | |
| | PE = 1 SV = 1 | | | | |
| P46777 | 60S ribosomal protein L5 OS = Homo | 4 | RPL5 | | 67 |
| | sapiens OX = 9606 GN = RPL5 | | | | |
| | PE = 1 SV = 3 | | | | |
| P68104 | Elongation factor 1-alpha 1 OS = Homo | 4 | EEF1A1 | 4 | 3 |
| | sapiens OX = 9606 GN = EEF1A1 PE = 1 | | | | |
| | SV = 1 | | | | |
| P36578 | 60S ribosomal protein L4 OS = Homo | 4 | RPL4 | 1 | |
| | sapiens OX = 9606 GN = RPL4 PE = | | | | |
| | 1 SV = 5 | | | | |
| P23396 | 40S ribosomal protein S3 OS = Homo | 4 | RPS3 | 3 | 14 |
| | sapiens OX = 9606 GN = RPS3 PE = | | | | |
| | 1 SV = 2 | | | | |
| P26373 | 60S ribosomal protein L13 OS = Homo | 4 | RPL13 | 4 | 5 |
| | sapiens OX = 9606 GN = RPL13 PE = | | | | |
| | 1 SV = 4 | | | | |
| E9PRY8 | Elongation factor 1-delta OS = Homo sapiens | 4 | EEF1D | | 7 |
| | OX = 9606 GN = EEF1D PE = 1 SV = 1 | | | | |
| P61247 | 40S ribosomal protein S3a OS = Homo | 4 | RPS3A | 4 | 5 |
| | sapiens OX = 9606 GN = RPS3A PE = | | | | |
| | 1 SV = 2 | | | | |
| P62424 | 60S ribosomal protein L7a OS = Homo | 4 | RPL7A | 2 | 4 |
| | sapiens OX = 9606 GN = RPL7A PE = | | | | |
| | 1 SV = 2 | | | | |
| P39023 | 60S ribosomal protein L3 OS = Homo | 4 | RPL3 | | 1 |
| | sapiens OX = 9606 GN = RPL3 PE = | | | | |
| | 1 SV = 2 | | | | |
| P62917 | 60S ribosomal protein L8 OS = Homo | 4 | RPL8 | 2 | 4 |
| | sapiens OX = 9606 GN = RPL8 PE = | | | | |
| | 1 SV = 2 | | | | |
| P62241 | 40S ribosomal protein S8 OS = Homo | 4 | RPS8 | | 3 |
| | sapiens OX = 9606 GN = RPS8 PE = | | | | |
| | 1 SV = 2 | | | | |
| P62913 | 60S ribosomal protein L11 OS = Homo | 4 | RPL11 | 3 | 4 |
| | sapiens OX = 9606 GN = RPL11 PE = | | | | |
| | 1 SV = 2 | | | | |
| P61313 | 60S ribosomal protein L15 OS = Homo | 4 | RPL15 | 1 | 2 |
| | sapiens OX = 9606 GN = RPL15 PE = | | | | |
| | 1 SV = 2 | | | | |
| J3QQ67 | 60S ribosomal protein L18 (Fragment) | 4 | RPL18 | 1 | 4 |
| | OS = Homo sapiens OX = 9606 GN = | | | | |
| | RPL18 PE = 1 SV = 1 | | | | |
| P62753 | 40S ribosomal protein S6 OS = Homo | 4 | RPS6 | 3 | 2 |
| | sapiens OX = 9606 GN = RPS6 PE = 1 | | | | |
| | SV = 1 | | | | |
| P26641 | Elongation factor 1-gamma OS = Homo | 4 | EEF1G | | 1 |
| | sapiens OX = 9606 GN = EEF1G PE = 1 | | | | |
| | SV = 3 | | | | |
| P18124 | 60S ribosomal protein L7 OS = Homo | 4 | RPL7 | 2 | 5 |
| | sapiens OX = 9606 GN = RPL7 PE = 1 | | | | |
| | SV = 1 | | | | |
| P62263 | 40S ribosomal protein S14 OS = Homo | 4 | RPS14 | | 3 |
| | sapiens OX = 9606 GN = RPS14 | | | | |
| | PE = 1 SV = 3 | | | | |
| P62701 | 40S ribosomal protein S4, X isoform | 4 | RPS4X | | 1 |
| | OS = Homo sapiens OX = 9606 GN = | | | | |
| | RPS4X PE = 1 SV = 2 | | | | |
| Q14240 | Eukaryotic initiation factor 4A-II | 4 | EIF4A2 | | |
| | OS = Homo sapiens OX = 9606 GN = | | | | |
| | EIF4A2 PE = 1 SV = 2 | | | | |
| P84098 | 60S ribosomal protein L19 OS = Homo | 4 | RPL19 | 1 | 2 |
| | sapiens OX = 9606 GN = RPL19 PE = 1 | | | | |
| | SV = 1 | | | | |
| Q02543 | 60S ribosomal protein L18a OS = Homo | 4 | RPL18A | | 3 |
| | sapiens OX = 9606 GN = RPL18A PE = 1 | | | | |
| | SV = 2 | | | | |
| Q14152 | Eukaryotic translation initiation factor 3 | 4 | EIF3A | | 2 |
| | subunit A OS = Homo sapiens OX = 9606 | | | | |
| | GN = EIF3A PE = 1 SV = 1 | | | | |
| P47914 | 60S ribosomal protein L29 OS = Homo | 4 | RPL29 | 1 | 1 |
| | sapiens OX = 9606 GN = RPL29 PE = | | | | |
| | 1 SV = 2 | | | | |
| P50914 | 60S ribosomal protein L14 OS = Homo | 4 | RPL14 | | 2 |
| | sapiens OX = 9606 GN = RPL14 PE = | | | | |
| | 1 SV = 4 | | | | |
| Q13200 | 26S proteasome non-ATPase regulatory | 5 | PSMD2 | 1 | 3 |
| | subunit 2 OS = Homo sapiens OX = 9606 | | | | |
| | GN = PSMD2 PE = 1 SV = 3 | | | | |
| G3V5Z7 | Proteasome subunit alpha type OS = Homo | 5 | PSMA6 | | 4 |
| | sapiens OX = 9606 GN = PSMA6 PE = | | | | |
| | 1 SV = 1 | | | | |
| A0A087X1Z3 | Proteasome activator complex subunit 2 | 5 | PSME2 | | 4 |
| | OS = Homo sapiens OX = 9606 GN = | | | | |
| | PSME2 PE = 1 SV = 1 | | | | |
| P02751 | Fibronectin OS = Homo sapiens OX = 9606 | 6 | FN1 | 3 | |
| | GN = FN1 PE = 1 SV = 4 | | | | |
| P20020 | Plasma membrane calcium-transporting | 6 | ATP2B1 | 10 | 11 |
| | ATPase 1 OS = Homo sapiens OX = 9606 | | | | |
| | GN = ATP2B1 PE = 1 SV = 3 | | | | |
| P04899 | Guanine nucleotide-binding protein G(i) | 6 | GNAI2 | 7 | 10 |
| | subunit alpha-2 OS = Homo sapiens | | | | |
| | OX = 9606 GN = GNAI2 PE = 1 SV = 3 | | | | |
| E9PFW3 | AP-2 complex subunit mu OS = Homo | 6 | AP2M1 | | 1 |
| | sapiens OX = 9606 GN = AP2M1 PE = | | | | |
| | 1 SV = 1 | | | | |
| O00469 | Procollagen-lysine,2-oxoglutarate 5- | 6 | PLOD2 | 7 | 8 |
| | dioxygenase 2 OS = Homo sapiens OX = 9606 | | | | |
| | GN = PLOD2 PE = 1 SV = 2 | | | | |
| Q9ULV4 | Coronin-1C OS = Homo sapiens OX = 9606 | 6 | CORO1C | | |
| | GN = CORO1C PE = 1 SV = 1 | | | | |
| A0A0A0MTJ9 | Neutral cholesterol ester hydrolase 1 | 6 | NCEH1 | | 1 |
| | OS = Homo sapiens OX = 9606 GN = | | | | |
| | NCEH1 PE = 1 SV = 1 | | | | |
| P05023 | Sodium/potassium-transporting ATPase | 6 | ATP1A1 | 5 | 7 |
| | subunit alpha-1 OS = Homo sapiens | | | | |
| | OX = 9606 GN = ATP1A1 PE = 1 SV = 1 | | | | |
| O43865 | S-adenosylhomocysteine hydrolase-like | 6 | AHCYL1 | | 1 |
| | protein 1 OS = Homo sapiens OX = 9606 | | | | |
| | GN = AHCYL1 PE = 1 SV = 2 | | | | |
| Q96MG2 | Junctional sarcoplasmic reticulum protein 1 | 6 | JSRP1 | | 1 |
| | OS = Homo sapiens OX = 9606 GN = | | | | |
| | JSRP1 PE = 1 SV = 1 | | | | |
| P25705 | ATP synthase subunit alpha, mitochondrial | 6 | ATP5F1A | 6 | 4 |
| | OS = Homo sapiens OX = 9606 | | | | |
| | GN = ATP5F1A PE = 1 SV = 1 | | | | |
| A0A087WW87 | Immunoglobulin kappa variable 2-40 | 6 | IGKV2-40 | 5 | 1 |
| | OS = Homo sapiens OX = 9606 GN = | | | | |
| | IGKV2-40 PE = 3 SV = 2 | | | | |
| A0A2R8Y6G6 | Alpha-enolase OS = Homo sapiens OX = | 6 | ENO1 | 6 | 3 |
| | 9606 GN = ENO1 PE = 1 SV = 1 | | | | |
| Q02809 | Procollagen-lysine, 2-oxoglutarate 5- | 6 | PLOD1 | | 2 |
| | dioxygenase 1 OS = Homo sapiens OX = | | | | |
| | 9606 GN = PLOD1 PE = 1 SV = 2 | | | | |
| J3KPF3 | 4F2 cell-surface antigen heavy chain | 6 | SLC3A2 | 2 | 4 |
| | OS = Homo sapiens OX = 9606 | | | | |
| | GN = SLC3A2 PE = 1 SV = 1 | | | | |
| P05141 | ADP/ATP translocase 2 OS = Homo sapiens | 6 | SLC25A5 | | |
| | OX = 9606 GN = SLC25A5 PE = 1 SV = 7 | | | | |
| P05121 | Plasminogen activator inhibitor 1 | 6 | SERPINE1 | 1 | |
| | OS = Homo sapiens OX = 9606 | | | | |
| | GN = SERPINE1 PE = 1 SV = 1 | | | | |
| Q00325 | Phosphate carrier protein, mitochondrial | 6 | SLC25A3 | 3 | 2 |
| | OS = Homo sapiens OX = 9606 | | | | |
| | GN = SLC25A3 PE = 1 SV = 2 | | | | |
| P42704 | Leucine-rich PPR motif-containing protein, | 6 | LRPPRC | 1 | |
| | mitochondrial OS = Homo sapiens OX = | | | | |
| | 9606 GN = LRPPRC PE = 1 SV = 3 | | | | |
| O43175 | D-3-phosphoglycerate dehydrogenase | 6 | PHGDH | | 1 |
| | OS = Homo sapiens OX = 9606 GN = | | | | |
| | PHGDH PE = 1 SV = 4 | | | | |
| A6XMV9 | Protease serine 2 preproprotein OS = Homo | 6 | PRSS2 | | 1 |
| | sapiens OX = 9606 GN = PRSS2 PE = | | | | |
| | 1 SV = 1 | | | | |
| P06703 | Protein S100-A6 OS = Homo sapiens | 6 | S100A6 | 1 | |
| | OX = 9606 GN = S100A6 PE = 1 SV = 1 | | | | |
| P08123 | Collagen alpha-2(I) chain OS = Homo | 6 | COL1A2 | 2 | |
| | sapiens OX = 9606 GN = COL1A2 PE = 1 | | | | |
| | SV = 7 | | | | |
| P06702 | Protein S100-A9 OS = Homo sapiens | 6 | S100A9 | | 2 |
| | OX = 9606 GN = S100A9 PE = 1 SV = 1 | | | | |
| O60568 | Procollagen-lysine, 2-oxoglutarate 5- | 6 | PLOD3 | | |
| | dioxygenase 3 OS = Homo sapiens OX = | | | | |
| | 9606 GN = PLOD3 PE = 1 SV = 1 | | | | |
| P07237 | Protein disulfide-isomerase OS = Homo | 7 | P4HB | 9 | 20 |
| | sapiens OX = 9606 GN = P4HB PE = | | | | |
| | 1 SV = 3 | | | | |
| O95782 | AP-2 complex subunit alpha-1 OS = Homo | 7 | AP2A1 | | 5 |
| | sapiens OX = 9606 GN = AP2A1 PE = | | | | |
| | 1 SV = 3 | | | | |
| P13674 | Prolyl 4-hydroxylase subunit alpha-1 | 7 | P4HA1 | 1 | 27 |
| | OS = Homo sapiens OX = 9606 GN = | | | | |
| | P4HA1 PE = 1 SV = 2 | | | | |
| P63010 | AP-2 complex subunit beta OS = Homo | 7 | AP2B1 | | 1 |
| | sapiens OX = 9606 GN = AP2B1 PE = | | | | |
| | 1 SV = 1 | | | | |
| P04843 | Dolichyl-diphosphooligosaccharide--protein | 7 | RPN1 | 2 | 7 |
| | glycosyltransferase subunit 1 OS = Homo | | | | |
| | sapiens OX = 9606 GN = RPN1 | | | | |
| | PE = 1 SV = 1 | | | | |
| H7BZJ3 | Protein disulfide-isomerase A3 (Fragment) | 7 | PDIA3 | 9 | 3 |
| | OS = Homo sapiens OX = 9606 GN = | | | | |
| | PDIA3 PE = 1 SV = 1 | | | | |
| P50454 | Serpin H1 OS = Homo sapiens OX = 9606 | 7 | SERPINH1 | 5 | 5 |
| | GN = SERPINH1 PE = 1 SV = 2 | | | | |
| Q12797 | Aspartyl/asparaginyl beta-hydroxylase | 7 | ASPH | | 1 |
| | OS = Homo sapiens OX = 9606 GN = | | | | |
| | ASPH PE = 1 SV = 3 | | | | |
| A0A140T9T7 | Antigen peptide transporter 1 OS = Homo | 7 | TAP1 | 2 | 5 |
| | sapiens OX = 9606 GN = TAP1 PE = | | | | |
| | 1 SV = 1 | | | | |
| B4DR61 | cDNA FLJ59739, highly similar to Protein | 7 | SEC61A1 | 5 | 2 |
| | transport protein Sec61 subunit alpha | | | | |
| | isoform 1 OS = Homo sapiens OX = 9606 | | | | |
| | GN = SEC61A1 PE = 1 SV = 1 | | | | |
| P04844 | Dolichyl-diphosphooligosaccharide--protein | 7 | RPN2 | | 3 |
| | glycosyltransferase subunit 2 OS = Homo | | | | |
| | sapiens OX = 9606 GN = RPN2 | | | | |
| | PE = 1 SV = 3 | | | | |
| P11142 | Heat shock cognate 71 kDa protein | 9 | HSPA8 | 21 | 90 |
| | OS = Homo sapiens OX = 9606 GN = | | | | |
| | HSPA8 PE = 1 SV = 1 | | | | |
| P11021 | Endoplasmic reticulum chaperone BiP | 9 | HSPA5 | 31 | 49 |
| | OS = Homo sapiens OX = 9606 GN = | | | | |
| | HSPA5 PE = 1 SV = 2 | | | | |
| A0A0G2JIW1 | Heat shock 70 kDa protein 1B OS = Homo | 9 | HSPA1B | | 23 |
| | sapiens OX = 9606 GN = HSPA1B PE = 1 | | | | |
| | SV = 1 | | | | |
| P08238 | Heat shock protein HSP 90-beta OS = Homo | 9 | HSP90AB1 | | 6 |
| | sapiens OX = 9606 GN = HSP90AB1 PE = 1 | | | | |
| | SV = 4 | | | | |
| P38646 | Stress-70 protein, mitochondrial OS = Homo | 9 | HSPA9 | 1 | 11 |
| | sapiens OX = 9606 GN = HSPA9 | | | | |
| | PE = 1 SV = 2 | | | | |
| P07900 | Heat shock protein HSP 90-alpha | 9 | HSP90AA1 | 6 | 5 |
| | OS = Homo sapiens OX = 9606 | | | | |
| | GN = HSP90AA1 PE = 1 SV = 5 | | | | |
| P10809 | 60 kDa heat shock protein, mitochondrial | 9 | HSPD1 | 5 | 4 |
| | OS = Homo sapiens OX = 9606 GN = | | | | |
| | HSPD1 PE = 1 SV = 2 | | | | |
| P31947 | 14-3-3 protein sigma OS = Homo sapiens | 9 | SFN | 1 | |
| | OX = 9606 GN = SFN PE = 1 SV = 1 | | | | |
| P49368 | T-complex protein 1 subunit gamma | 9 | CCT3 | | 4 |
| | OS = Homo sapiens OX = 9606 GN = | | | | |
| | CCT3 PE = 1 SV = 4 | | | | |
| P78371 | T-complex protein 1 subunit beta | 9 | CCT2 | | 2 |
| | OS = Homo sapiens OX = 9606 GN = | | | | |
| | CCT2 PE = 1 SV = 4 | | | | |
| P50990 | T-complex protein 1 subunit theta | 9 | CCT8 | | 1 |
| | OS = Homo sapiens OX = 9606 GN = | | | | |
| | CCT8 PE = 1 SV = 4 | | | | |
| P50991 | T-complex protein 1 subunit delta | 9 | CCT4 | | 3 |
| | OS = Homo sapiens OX = 9606 GN = | | | | |
| | CCT4 PE = 1 SV = 4 | | | | |
| O95373 | Importin-7 OS = Homo sapiens OX = | 10 | IPO7 | | 3 |
| | 9606 GN = IPO7 PE = 1 SV = 1 | | | | |
| O60684 | Importin subunit alpha-7 OS = Homo sapiens | 10 | KPNA6 | | 6 |
| | OX = 9606 GN = KPNA6 PE = 1 SV = 1 | | | | |
| P52294 | Importin subunit alpha-5 OS = Homo sapiens | 10 | KPNA1 | | 1 |
| | OX = 9606 GN = KPNA1 PE = 1 SV = 3 | | | | |
| Q92973 | Transportin-1 OS = Homo sapiens OX = | 10 | TNPO1 | | 6 |
| | 9606 GN = TNPO1 PE = 1 SV = 2 | | | | |
| Q14974 | Importin subunit beta-1 OS = Homo sapiens | 10 | KPNB1 | 1 | 4 |
| | OX = 9606 GN = KPNB1 PE = 1 SV = 2 | | | | |
| P10644 | cAMP-dependent protein kinase type I- | 10 | PRKAR1A | | 4 |
| | alpha regulatory subunit OS = Homo sapiens | | | | |
| | OX = 9606 GN = PRKAR1A PE = 1 SV = 1 | | | | |
| P52292 | Importin subunit alpha-1 OS = Homo sapiens | 10 | KPNA2 | | 3 |
| | OX = 9606 GN = KPNA2 PE = 1 SV = 1 | | | | |
| H0Y8C6 | Importin-5 (Fragment) OS = Homo sapiens | 10 | IPO5 | | 1 |
| | OX = 9606 GN = IPO5 PE = 1 SV = 1 | | | | |

| | 4. | | | 3. + | | |
|---|---|---|---|---|---|---|
| | longCTD + | 3. + | 3. + | IFNg + | SUM | Max |
| | Dox + | IFNG | Dox | Dox | of | Unique |
| Accession | IFNG | longCTD | longCTD | longCTD | PSMS | Peptides |
| A0A0D9SF53 | | 6 | 29 | 37 | 775 | 17 |
| P42224 | 19 | | | 3 | 43 | 7 |
| P78527 | 2 | | 3 | 18 | 39 | 17 |
| H0Y449 | 7 | | 5 | 7 | 35 | 4 |
| A0A087X0X3 | 2 | | 9 | 21 | 35 | 14 |
| P61978 | 6 | | 2 | 8 | 31 | 7 |
| P30153 | 2 | 2 | 5 | 14 | 28 | 10 |
| Q16576 | 5 | | | 4 | 25 | 6 |
| Q9NQC3 | 6 | | | 7 | 23 | 3 |
| O14929 | 9 | | | 3 | 23 | 4 |
| P11940 | 3 | | | 13 | 21 | 5 |
| G8JLB6 | 3 | | 1 | 7 | 18 | 5 |
| P19338 | 1 | | | 4 | 16 | 7 |
| P11802 | 11 | | 1 | 1 | 16 | 5 |
| P06748 | | | 2 | 8 | 16 | 4 |
| Q15393 | 1 | | 1 | 3 | 15 | 5 |
| Q93009 | | | | 6 | 15 | 6 |
| Q93008 | 3 | | | 5 | 15 | 5 |
| P13489 | 4 | 1 | 3 | 3 | 15 | 3 |
| Q00839 | | | 4 | 9 | 14 | 7 |
| Q09666 | 1 | | 8 | 4 | 14 | 3 |
| Q13263 | 2 | | | 1 | 12 | 4 |
| P16989 | | | 1 | 3 | 11 | 3 |
| Q14444 | | | 5 | 5 | 11 | 5 |
| Q15208 | 6 | 1 | | | 11 | 5 |
| Q63ZY3 | 4 | | 3 | 3 | 11 | 3 |
| P55209 | 3 | | | 5 | 10 | 3 |
| Q15233 | | | 1 | 8 | 10 | 5 |
| P47929 | 6 | | 1 | 1 | 8 | 2 |
| Q13283 | 2 | | 1 | 2 | 7 | 2 |
| Q8NC51 | 1 | | 2 | 3 | 6 | 3 |
| Q9UN86 | 1 | | 1 | 3 | 6 | 2 |
| P26196 | 1 | | 2 | 3 | 6 | 2 |
| P13010 | | | | 5 | 6 | 4 |
| P52597 | 1 | | 1 | 4 | 6 | 3 |
| E7ENX8 | 1 | 1 | | 1 | 5 | 1 |
| O94919 | 2 | | | 1 | 5 | 2 |
| P40763 | | | 1 | 3 | 5 | 3 |
| F8W930 | 2 | | 1 | | 4 | 3 |
| P29508 | | | | 2 | 4 | 2 |
| Q86YD1 | 2 | | 2 | | 4 | 2 |
| P17655 | 3 | | | 1 | 4 | 2 |
| P51784 | 2 | | | 1 | 4 | 2 |
| A0A0U1RRM4 | | | | 2 | 3 | 2 |
| P35579 | 4 | 8 | 122 | 242 | 387 | 104 |
| Q07065 | 13 | 4 | 30 | 62 | 152 | 29 |
| P12814 | 34 | | 4 | 13 | 82 | 7 |
| Q16643 | 33 | | 3 | 13 | 51 | 14 |
| A0A087WVQ6 | 28 | | | 11 | 47 | 14 |
| Q6NZI2 | 16 | | | 3 | 42 | 5 |
| P21333 | 10 | | 1 | 12 | 30 | 22 |
| Q14315 | 12 | | 2 | 7 | 24 | 8 |
| P02545 | 6 | | | 3 | 21 | 4 |
| P61158 | 3 | | 5 | 13 | 21 | 10 |
| E9PK25 | 13 | | | 1 | 19 | 3 |
| B1AK88 | 11 | | | 1 | 19 | 4 |
| P48681 | 2 | 1 | 4 | 4 | 18 | 5 |
| O75083 | 11 | | 1 | 2 | 16 | 4 |
| B7Z6Z4 | 5 | | | 4 | 9 | 4 |
| Q14247 | 5 | | | 1 | 6 | 3 |
| Q9Y490 | 3 | | 1 | 1 | 5 | 2 |
| Q13409 | 4 | | | 1 | 5 | 2 |
| Q14203 | 2 | | | 1 | 4 | 1 |
| P60709 | 160 | 4 | 23 | 27 | 370 | 7 |
| P07437 | 110 | 14 | 41 | 75 | 315 | 4 |
| P08670 | 55 | 39 | 42 | 45 | 290 | 22 |
| P68371 | 100 | 11 | 42 | 70 | 283 | 3 |
| P62736 | 91 | | 13 | 15 | 210 | 4 |
| Q13885 | 99 | 13 | 35 | 61 | 208 | 1 |
| Q9BUF5 | 46 | | 20 | 39 | 128 | 5 |
| J3QRS3 | 62 | | | 3 | 104 | 4 |
| O43707 | 46 | | 2 | 11 | 92 | 10 |
| P17661 | 12 | 14 | 9 | 15 | 84 | 7 |
| F5H5D3 | 25 | 7 | | 47 | 79 | 6 |
| P68366 | 16 | | 14 | 39 | 69 | 5 |
| P68363 | | | 22 | | 55 | 9 |
| Q15149 | 7 | | 5 | 21 | 52 | 20 |
| Q3SY84 | | | | 9 | 30 | 4 |
| A0A087WUZ3 | 3 | | 1 | 9 | 13 | 9 |
| A0A0D9SGF6 | 1 | | 1 | 10 | 12 | 9 |
| Q9P2E9 | 4 | | 24 | 68 | 112 | 36 |
| P05388 | 36 | | | 5 | 88 | 13 |
| P46777 | 19 | | | 1 | 87 | 17 |
| P68104 | 9 | | 9 | 21 | 46 | 10 |
| P36578 | | 1 | 10 | 34 | 46 | 16 |
| P23396 | 11 | | 1 | 4 | 33 | 9 |
| P26373 | 2 | | 2 | 7 | 20 | 4 |
| E9PRY8 | 11 | | | 1 | 19 | 4 |
| P61247 | 2 | | 3 | 5 | 19 | 5 |
| P62424 | | 1 | 4 | 5 | 16 | 4 |
| P39023 | | | 3 | 11 | 15 | 7 |
| P62917 | 2 | 1 | 3 | 2 | 14 | 2 |
| P62241 | 2 | | 2 | 6 | 13 | 3 |
| P62913 | 3 | | 1 | 2 | 13 | 2 |
| P61313 | 1 | | 1 | 8 | 13 | 6 |
| J3QQ67 | | 1 | 1 | 5 | 12 | 3 |
| P62753 | 2 | 1 | 1 | 3 | 12 | 3 |
| P26641 | 1 | | 3 | 6 | 11 | 5 |
| P18124 | | | 1 | 1 | 9 | 2 |
| P62263 | | | 2 | 3 | 8 | 2 |
| P62701 | 3 | | 1 | 2 | 7 | 2 |
| Q14240 | 6 | | 1 | | 7 | 3 |
| P84098 | 1 | | | 2 | 6 | 2 |
| Q02543 | | | 1 | 2 | 6 | 2 |
| Q14152 | | | 1 | 2 | 5 | 2 |
| P47914 | | | 1 | 2 | 5 | 1 |
| P50914 | | | | 1 | 3 | 1 |
| Q13200 | 4 | | | 3 | 11 | 3 |
| G3V5Z7 | | | 1 | 1 | 6 | 3 |
| A0A087X1Z3 | | | | 1 | 5 | 3 |
| P02751 | | | 17 | 48 | 68 | 29 |
| P20020 | 13 | | 1 | 1 | 36 | 9 |
| P04899 | 11 | | | 1 | 29 | 4 |
| E9PFW3 | 3 | | 8 | 16 | 28 | 12 |
| O00469 | 7 | | | 3 | 25 | 4 |
| Q9ULV4 | 7 | | 4 | 13 | 24 | 9 |
| A0A0A0MTJ9 | 4 | 1 | 3 | 11 | 20 | 7 |
| P05023 | 4 | | | 3 | 19 | 4 |
| O43865 | | | 7 | 11 | 19 | 8 |
| Q96MG2 | 3 | | 4 | 10 | 18 | 5 |
| P25705 | 4 | | 1 | 2 | 17 | 3 |
| A0A087WW87 | 5 | | 2 | 2 | 15 | 1 |
| A0A2R8Y6G6 | 2 | | 1 | | 12 | 7 |
| Q02809 | 6 | | | 4 | 12 | 4 |
| J3KPF3 | 2 | | | 2 | 10 | 2 |
| P05141 | 6 | | | 3 | 9 | 3 |
| P05121 | 3 | | 1 | 3 | 8 | 3 |
| Q00325 | 2 | | | 1 | 8 | 1 |
| P42704 | | | 1 | 4 | 6 | 4 |
| O43175 | 1 | | 1 | 3 | 6 | 3 |
| A6XMV9 | 2 | | | 2 | 5 | 1 |
| P06703 | 2 | | | 1 | 4 | 2 |
| P08123 | | 1 | 1 | | 4 | 1 |
| P06702 | | | 1 | 1 | 4 | 1 |
| O60568 | 1 | | 1 | 2 | 4 | 1 |
| P07237 | 14 | | 3 | 5 | 51 | 7 |
| O95782 | 2 | | 10 | 31 | 48 | 16 |
| P13674 | 9 | | 3 | 5 | 45 | 8 |
| P63010 | | | 8 | 26 | 35 | 19 |
| P04843 | 2 | 1 | 4 | 14 | 30 | 10 |
| H7BZJ3 | | | | 2 | 14 | 2 |
| P50454 | | | | 1 | 11 | 4 |
| Q12797 | | | 1 | 9 | 11 | 6 |
| A0A140T9T7 | | | | 3 | 10 | 3 |
| B4DR61 | | | | 1 | 8 | 2 |
| P04844 | | | | 4 | 7 | 3 |
| P11142 | 105 | 2 | 27 | 54 | 299 | 19 |
| P11021 | 44 | 1 | 22 | 62 | 209 | 24 |
| A0A0G2JIW1 | 29 | | | 10 | 62 | 6 |
| P08238 | 10 | | 2 | 14 | 32 | 6 |
| P38646 | 7 | | 1 | 10 | 30 | 8 |
| P07900 | 10 | | | 9 | 30 | 3 |
| P10809 | 5 | | 1 | 2 | 17 | 4 |
| P31947 | 6 | | | 1 | 8 | 1 |
| P49368 | 2 | | | 1 | 7 | 2 |
| P78371 | 4 | | | 1 | 7 | 2 |
| P50990 | 3 | | | 2 | 6 | 2 |
| P50991 | 1 | | | 1 | 5 | 1 |
| O95373 | 1 | | 4 | 8 | 16 | 7 |
| O60684 | 1 | | 4 | 4 | 15 | 3 |
| P52294 | 2 | | 4 | 8 | 15 | 3 |
| Q92973 | 3 | | 1 | 4 | 14 | 3 |
| Q14974 | 3 | | | 5 | 13 | 4 |
| P10644 | 3 | | | 4 | 11 | 3 |
| P52292 | 1 | | | 1 | 5 | 3 |
| H0Y8C6 | | | | 3 | 4 | 3 |

### Example 3: DUX4 Induced Translational Control has Prolonged Effects on Antigen Presentation

This Example discloses additional characterization of the prolonged effects of DUX4 on antigen presentation. Briefly, it is shown that: immediate inhibition of immune signaling pathways does not require DUX4 transcriptional activity and is mediated by DUX4 transcriptional suppression of interferon stimulated gene expression; a pulse of DUX4 induces prolonged suppression of antigen presentation proteins, including canonical MHC-I molecules HLA-A, HLA-B, HLA-C, and immunoproteasome subunits PSMB8, PSMB9, and PMSB10; prolonged suppression of antigen presentation factors persists several days longer than the DUX4 protein, requires DUX4 transcriptional activity, and occurs post-transcriptionally; a pulse of DUX4 alters the status of multiple factors involved in translation initiation and elongation, including phosphorylation of eIF2-alpha, dephosphorylation of 4EBP1 and eIF4E, and phosphorylation of eEF2; inhibition of mTOR signaling is sufficient to block protein expression of MHC-I and the immunoproteasome subunits PSMB8, PSMB9, and PSMB10; a pulse of DUX4 broadly suppresses nascent protein synthesis, and genes sensitive to decreased translation efficiency induced by DUX4 are enriched in pathways involving RNA processing, translation, metabolism, and immune regulation; and metabolic labeling in cells endogenously expressing the DUX4 transcriptional program, including FSHD myotubes and cancer cells, shows a correlated reduction in nascent protein synthesis.

### DUX4 Activity Induces Prolonged Immune Suppression of Antigen Presentation Factors

In recent work, it was found that DUX4 blocks interferon-gamma (IFNG) induction of Major Histocompatibility Complex (MHC) Class I and surface antigen presentation. Hence, this investigation was to further elucidate the mechanisms of DUX4 immune suppression. To study the consequences of human DUX4 expression, a well characterized cellular model system of human myoblasts with a doxycycline inducible DUX4 transgene was used (MB135iDUX4; Jagannathan et al, 2016, Model systems of DUX4 expression recapitulate the transcriptional profile of FSHD cells. Hum Mol Genet; 25 (20): 4419-4431). Prolonged mis-expression of DUX4 induces cell death in nearly every cell type tested; however, endogenous DUX4 expression occurs in transient bursts of expression and with considerable heterogeneity in embryonic stem cells, FSHD, and cancer. It has been demonstrated that a short “pulse” of DUX4 in MB135iDUX4 cells induced a transcriptional program representative of FSHD muscle cells and the early cleavage-stage embryo without cytotoxic effects. Pulsed DUX4 expression in this cell culture system enabled reproducible and synchronized DUX4 induction with cell survival, permitting the investigation of mechanisms downstream of DUX4 which may have been otherwise masked by heterogeneous populations of DUX4-expressing cells.

To assay the impact of DUX4 on cellular immunogenicity, expression levels of canonical MHC-I subunits (HLA-A, HLA-B, HLA-C) and immunoproteasome subunits (PSMB8, PSMB9, PSMB10) that generate unique immunogenic antigens with high affinity for MHC-I were measured. Using the MB135iDUX4 cell culture system, both the immediate effect of continuous DUX4 expression on antigen presentation factors, as well as the prolonged consequences following a transient pulse of DUX4, were tested (FIG. 35A). Cells stimulated with IFNG triggered an immune response and showed elevated levels of MHC-I and immunoproteasome subunits. It was found that IFNG-induction of MHC-I and the immunoproteasome was suppressed in myoblasts continually expressing DUX4 (FIG. 35B, left). Remarkably, a brief pulse of DUX4 elicited the same degree of suppression when assayed forty-four hours later, even with diminished DUX4 protein levels (FIG. 35B, right). However, a subunit of the constitutively expressed proteasome complex—PSMB6—was not suppressed by DUX4, highlighting that DUX4-induced immune suppression is specific to newly synthesized immunoproteasome complex subunits and canonical MHC-I proteins (FIG. 35B).

As detailed in Example 2, it was discovered that DUX4 suppresses interferon stimulated gene (ISG) expression transcriptionally by sequestering STAT1 through protein-protein interaction. mRNA levels of MHC-I and immunoproteasome genes were measured under both continuous and pulsed DUX4 treatment conditions. Continuous DUX4 partly blocked IFNG stimulation of HLA-A, HLA-B, HLA-C, PSMB8, PMSB9, and PSMB10 mRNA levels, while a pulse of DUX4 still exhibited IFNG-induced mRNA levels significantly above untreated samples (FIG. 35C). Based on these observations, it was hypothesized that DUX4 mediates immune suppression via two distinct mechanisms: transcriptional and post-transcriptional regulation. DUX4 transcriptional suppression of ISGs does not require DUX4 transcriptional activity and is mediated by protein-protein interactions within the conserved C-terminal domain of DUX4 (Example 2). To determine if DUX4 transcriptional activity was necessary for post-transcriptional protein suppression, a mutation was introduced within the DNA binding domain of the DUX4 transgene (F67A), abrogating the DUX4 transcriptional program. Continuous expression of both wildtype DUX4 and mutant DUX4(F67A) suppressed IFNG-induced MHC-I and the immunoproteasome expression. However, while a pulse of wildtype DUX4 suppressed MHC-I and immunoproteasome subunits, protein levels were completely rescued following a pulse of mutant DUX4(F67A) (FIG. 35D). Thus, a novel post-transcriptional regulatory mechanism is identified that requires the transcriptional program of DUX4. The pulse time course was extended to assay the longevity of protein suppression induced by DUX4, treating cells with IFNG multiple days after a pulse of DUX4. Remarkably, a brief pulse of DUX4 suppressed IFNG-induced MHC-I and immunoproteasome protein levels for nearly four days (FIG. 35E). These data support the idea that transient DUX4 expression activates a unique transcriptional program that has long-term cellular consequences, including protein suppression of factors involved in antigen presentation critical for immune detection.

### DUX4 Disrupts Multiple Signaling Pathways Involved in Translational Regulation

The finding that prolonged suppression of antigen presentation factors requires DUX4 transcriptional activity suggested that DUX4 initiates post-transcriptional mechanisms impacting protein expression, for instance, changes in mRNA translation or protein degradation. Subcellular fractionation of nuclear and cytoplasmic mRNAs and proteins showed that a pulse of DUX4 did not disrupt HLA-A, HLA-B, HLA-C, PSMB8, PSMB9, or PSMB10 mRNA nuclear export or protein localization to the cytoplasm following IFNG simulation (data not shown). Additionally, treatment with proteasome inhibitor MG132 or autophagy inhibitor Bafilomycin did not rescue post-transcriptional suppression of IFNG-stimulated MHC-I, PSMB8, PSMB9, or PSMB10 following a pulse of DUX4 (data not shown), eliminating protein degradation as a causal mechanism acting downstream of DUX4 to suppress MHC-I and iProteasome subunits. Together these data suggested that suppression of antigen presentation factors following a pulse of DUX4 was likely due to translational inhibition.

iProteasome production of immunogenic antigens is linked to MHC-I stability; however, siRNA-mediated knockdown of PSMB8 and PSMB9 in parental MB135 myoblasts did not impact IFNG-induced MHC-I levels (data not shown). Furthermore, treatment with ONX-0914, a selective inhibitor of the iProteasome, did not reduce IFNG-induced MHC-I levels; conversely, treatment with the antineoplastic drug Carfilzomib, an inhibitor that blocks peptide production by both the constitutive proteasome and iProteasome, abrogated the accumulation of MHC-I following IFNG treatment (Data not shown). Thus, MHC-I stability does not require iProteasome-dependent proteolysis when the constitutive proteasome is active. These data indicate that DUX4 translationally suppresses MHC-I and the iProteasome subunits independently.

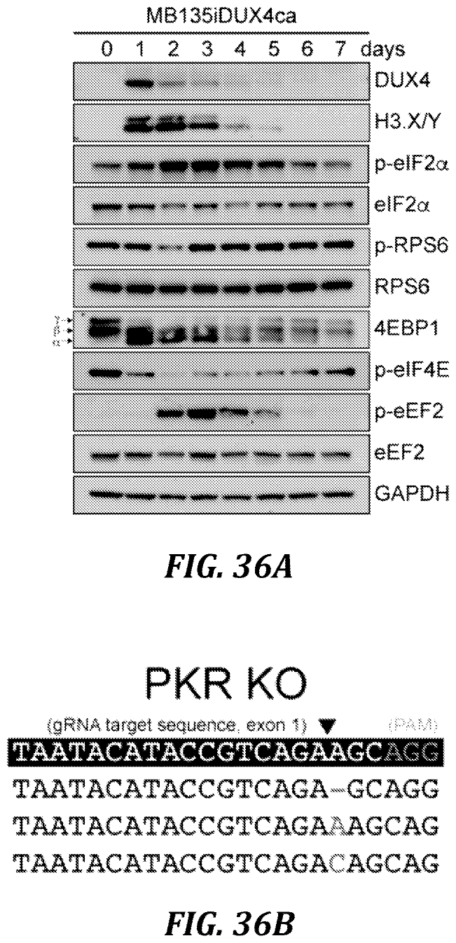

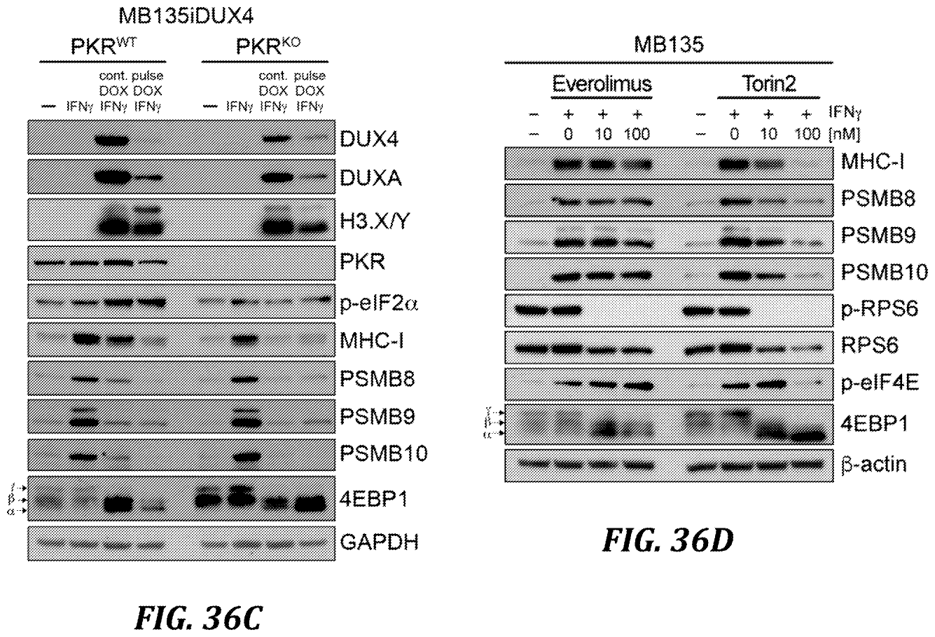

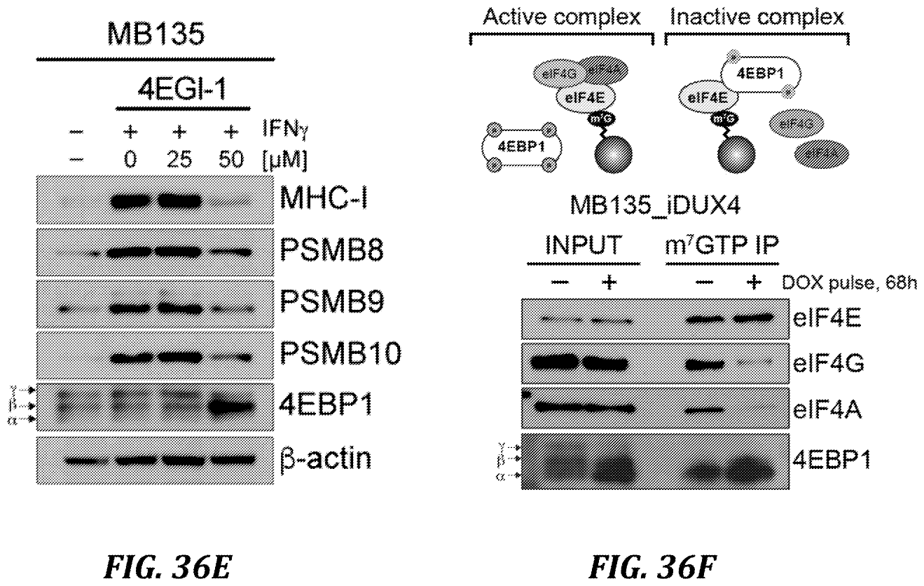

It has been shown that DUX4 induces nuclear double-stranded RNA (dsRNA) accumulation resulting in phosphorylation of PKR and eIF2-alpha; and determined that a pulse of DUX4 was sufficient to induce phosphorylation of eIF2-alpha as well (FIG. 36A). As a primary innate immune response to viral infection, dsRNA-activated host protein PKR plays a key role in blocking general translation through the phosphorylation of eIF2-alpha, disrupting initiator Met-tRNA loading onto the 40S ribosome, suppressing both viral and host translation. Furthermore, PKR has previously been implicated in suppression of IFNG-induced immunoproteasome components at the protein level following infection with dsRNA virus hepatitis C. It was therefore tested if the suppressive effects of DUX4 on immunoproteasome induction were mediated by PKR activation. The CRISPR/Cas9 system was used to generate a polyclonal PKR knockout (PKR-KO) in the MB135iDUX4 cell line (FIG. 36B). DUX4 suppression of immunoproteasome subunit PSMB9 was evident in both wild-type (WT) and PKR-KO cells with either continuous or pulsed DUX4 induction (FIG. 36C), indicating that PKR alone does not mediate prolonged DUX4 protein suppression.

Mechanistic Target of Rapamycin Complex 1 (mTORC1) signaling has also been reported to promote immunoproteasome formation. mTOR signaling is regulated by an array of intracellular and environmental cues, including nutrient and energy deprivation, cell stress, hypoxia, and DNA damage. Considering key pathogenic roles of DUX4 in FSHD include induction of cellular hypoxia response pathways, disrupted mitochondrial function, oxidative stress, and DNA damage, it was hypothesized that mTOR signaling may be disrupted by DUX4. Common themes also naturally occur during early embryonic development and in embryonic stem cells, including hypoxic environmental conditions, dynamic changes in metabolism, and unrepaired DNA damage. mTOR is best known for its function in promoting translation by directly phosphorylating ribosomal protein S6 kinase (S6K) and eIF4E-binding proteins (4EBPs). A pulse of DUX4 in MB135iDUX4 cells caused a brief reduction in phosphorylation of ribosomal protein S6 (RPS6), a major target of S6K, as well as prolonged dephosphorylation of 4EBP1 and a correlated loss of phosphorylated eukaryotic initiation factor 4E (eIF4E) (FIG. 36A). eIF4E is not a direct substrate of mTOR, however, hypophosphorylated forms of 4EBP antagonize eIF4E by sequestering it from protein complex formation with known activator kinase MNK and other translation initiation complex subunits eIF4G and eIF4A, effectively inhibiting translation initiation. It was also found that a pulse of DUX4 increased phosphorylation of eukaryotic elongation factor 2 (eEF2) on threonine 56 (FIG. 36A), a functional modification known to inhibit translation elongation that is regulated downstream of mTOR and other kinase signaling pathways. Collectively, these data suggest that DUX4 has adverse effects on translation mediated by mTOR inactivation or through parallel pathways that target mTOR-sensitive translational regulators.

To test if mTOR inactivation was sufficient to block MHC-I and immunoproteasome protein levels in the absence of DUX4, parental MB135 myoblasts were treated with mTOR inhibitors everolimus or Torin2. Everolimus is a second-generation rapamycin analog with binding specificity for mTORC1. As expected, treatment of MB135 cells with increasing concentrations of everolimus blocked S6K phosphorylation of RPS6; however, rapamycin-resistant 4EBP1 phosphorylation and eIF4E phosphorylation remained largely unaltered. Surprisingly, treatment with everolimus followed by IFNG did not block MHC-I or immunoproteasome protein expression (FIG. 36D, left), indicating that the S6K branch of mTORC1 signaling is not sufficient on its own to block protein synthesis of MHC-I and the immunoproteasome subunits. Conversely, it was found that treatment with Torin2, an ATP-competitive inhibitor of mTOR, both blocked phosphorylation of RPS6 as well as gave rise to complete hypophosphorylation of 4EBP1 and reduced eIF4E phosphorylation; this was sufficient to post-transcriptionally repress IFNG-induced MHC-I, PSMB8, PSMB9, and PSMB10 protein expression (FIG. 36D, right). To further validate the role of 4EBP1 in blocking MHC-I and immunoproteasome protein expression, MB135 cells were treated with 4EGI-1, a small molecule that pharmacologically mimics 4EBP function and inhibits eIF4E. Treatment with 50 uM 4EGI-1 suppressed IFNG-induced MHC-I and immunoproteasome subunits at the protein level (FIG. 36E). Thus, 4EBP1 appears to be a major translational regulator of several factors involved in antigen presentation in response to immune stimulus. Nonetheless, DUX4 alters the status of multiple factors involved in translational initiation and elongation that likely constitute functionally redundant mechanisms to reprogram cellular translation and modulate immune signaling.

To further validate the role of 4EBP1 in blocking MHC-I and iProteasome protein expression, we treated MB135 cells with 4EGI-1, a small molecule that pharmacologically mimics 4EBP function and inhibits eIF4E (Moerke et al., 2007). Treatment with 50 uM 4EGI-1 suppressed IFN-induced MHC-I and iProteasome subunits at the protein level (FIG. 36F) and had no effect on mRNA levels (data not shown). Thus, 4EBP1 appears to be a major translational regulator of several factors involved in antigen presentation in response to immune stimulus. It was discovered that DUX4 alters the status of 4EBP1 as well as multiple other factors involved in translational initiation and elongation, which likely constitute functionally redundant mechanisms to alter cellular translation.

### Transient DUX4 Activity Broadly Suppresses Nascent Protein Synthesis

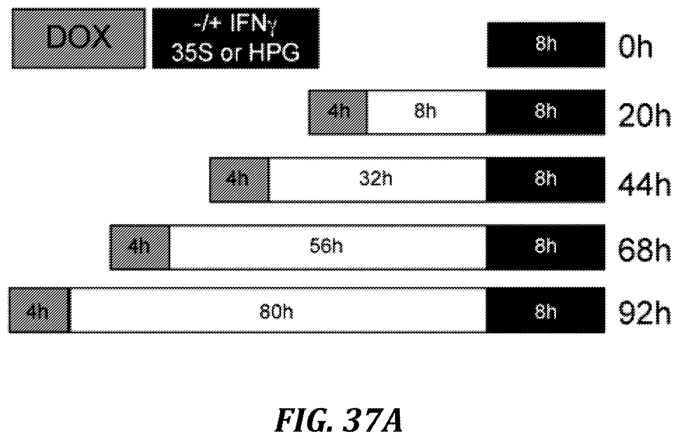

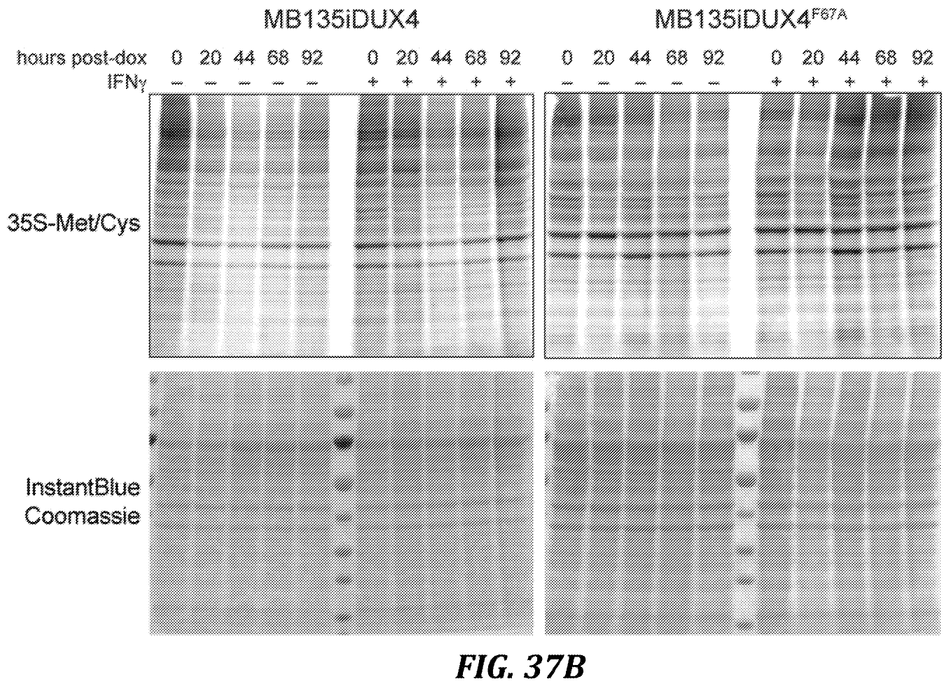

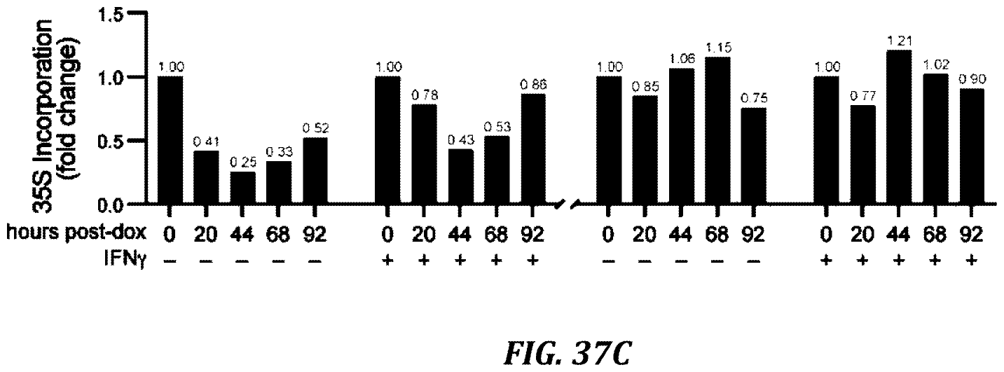

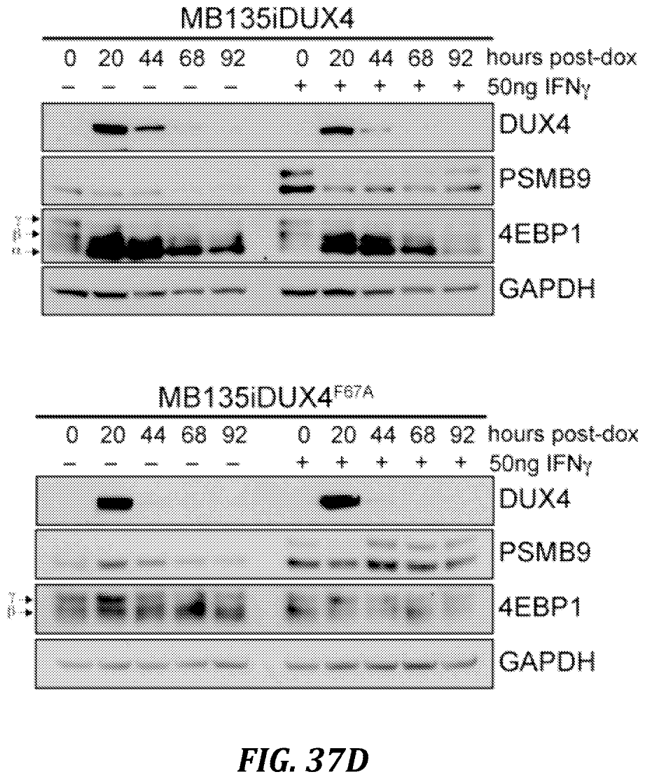

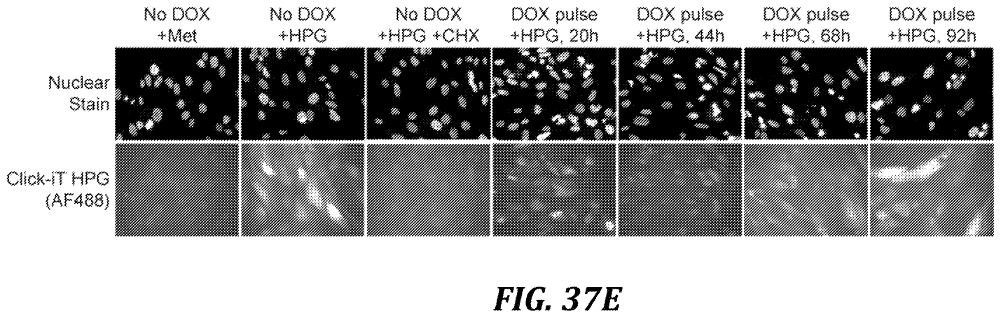

A careful balance of protein synthesis is critical for processes such as embryonic development and cellular adaptation to stress, and a wide range of signaling mechanisms can lead to the dampening of overall protein synthesis. It was found that collectively DUX4 disrupts the function of central regulators involved in both global and mRNA-specific translation. It was hypothesized that these changes induced by DUX4 could be representative of broader translational control. Indeed, metabolic labeling with 35S-methionine in MB135iDUX4 myoblasts showed that global de novo protein synthesis was transiently reduced following a pulse of DUX4 with and without IFNG (FIG. 37A-C). Induction of transcriptionally inactive DUX4(F67A) did not suppress protein translation, further demonstrating that the DUX4 transcriptional program was required for the broad protein suppression in these cells (FIG. 37A-C). Furthermore, reduced 35S-methionine signal correlated with PSMB9 suppression and increased levels of hypo-phosphorylated 4EBP1 throughout the time course following a pulse of DUX4 (FIG. 37D, top). However, neither suppression of PSMB9 or altered 4EBP1 phosphorylation was observed following a pulse of DUX4(F67A) (FIG. 37D, bottom). It was confirmed DUX4 inhibition of active protein synthesis by labeling cells with methionine ortholog L-homopropargylglycine (HPG), followed by fixation and Click-iT chemistry, wherein fluorescence microscopy showed a dramatic reduction in HPG-labeled peptides after a pulse of DUX4. As seen with 35S-methionine labeling, peak suppression occurred forty-four hours after a pulse of DUX4, comparable to the degree of inhibition induced by cycloheximide treatment alone, and HPG signal largely recovered by ninety-two hours (FIG. 37E). These findings establish a long-lived yet transitory role of DUX4-mediated suppression in governing broad translational reprogramming.

Ribosome Footprinting and Polysome Profiling Identify Specific mRNAs Translationally Altered by DUX4

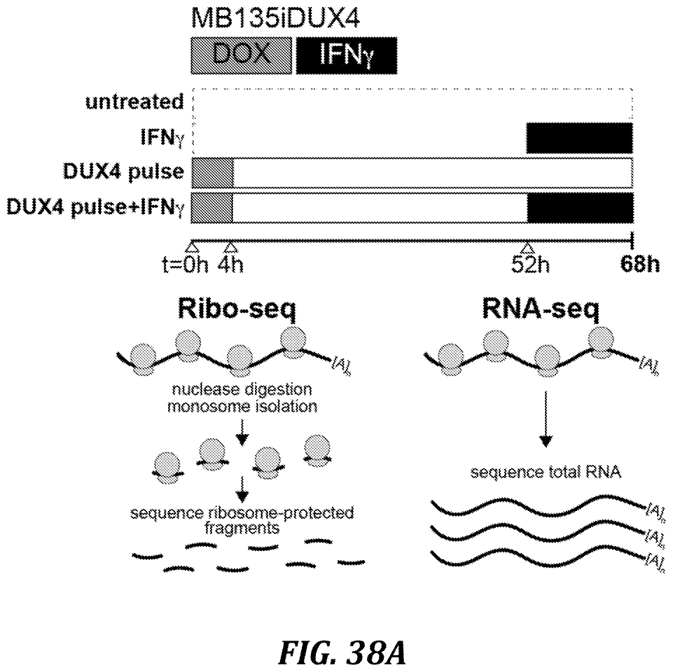

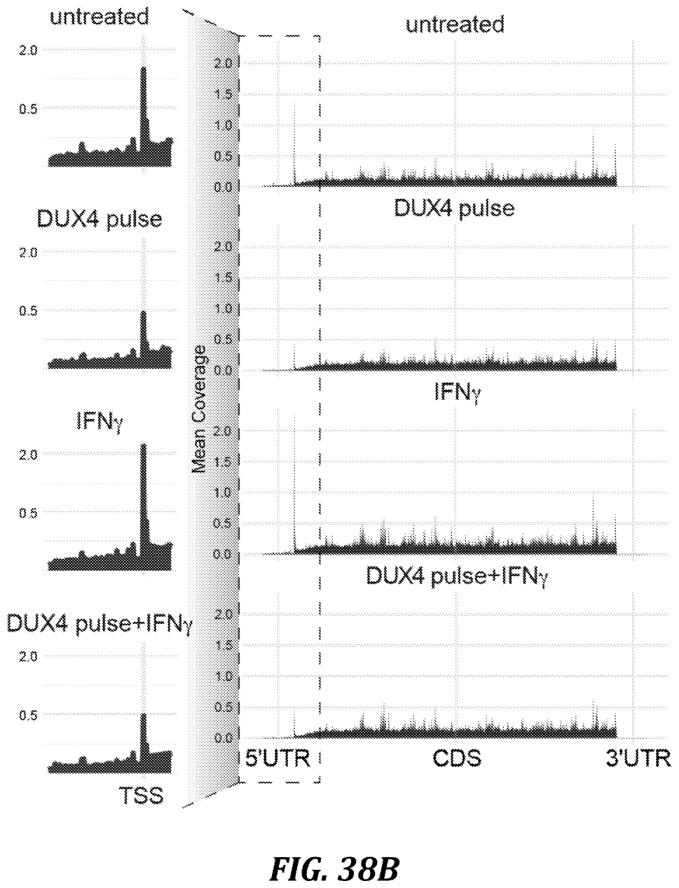

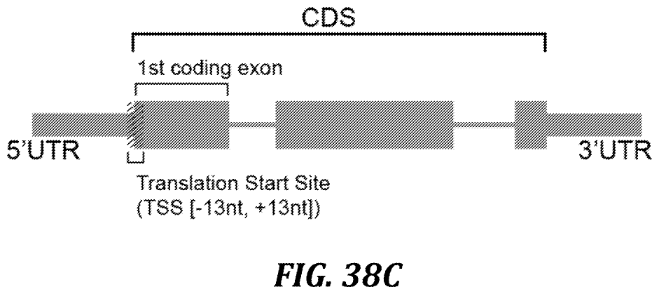

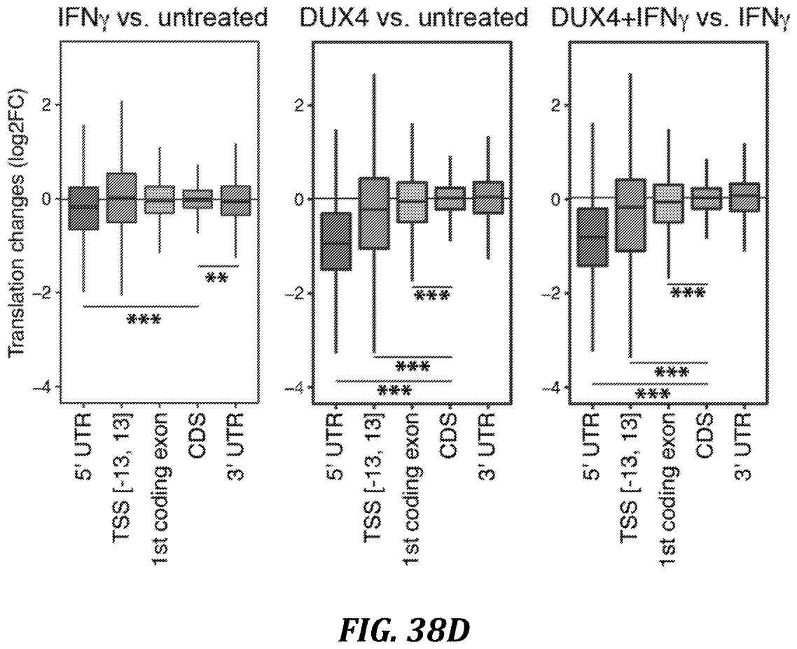

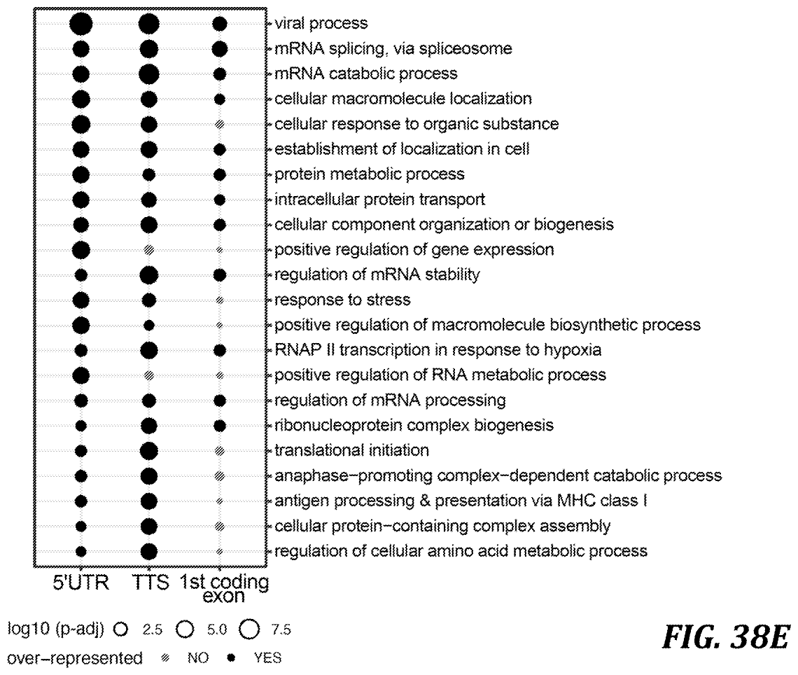

Ribosome footprinting reveals a DUX4-induced loss of 5′ ribosome occupancy To determine the mechanisms of translational suppression, ribosome footprinting (Ribo-seq) paired with RNA sequencing (RNA-seq) was performed on MB135iDUX4 myoblasts with four treatment conditions: untreated, IFNG alone, DUX4 pulse, and DUX4 pulse+IFNG harvested at 68 hours (FIG. 38A). Sequencing reads representing ribosome protected fragments (RPFs) displayed three-nucleotide periodicity and exhibited lengths of 26-29 nucleotides (nt) (data not shown)). Metagene analysis showed the majority of RPFs mapped to the coding region (CDS) and also revealed a depletion of Ribo-seq reads mapping to the mRNA region surrounding the translation start site (TSS) in samples pulsed with DUX4 (FIG. 38B). Differential translational efficiency (TE) was calculated by measuring Ribo-seq reads relative to RNA-seq reads and excluded DUX4-transcriptionally induced mRNAs because relative TE cannot be reliably calculated when transcripts are not abundantly expressed in both samples. Ribosome occupancy was largely reduced within the 5′UTR, at the TSS, and across the first coding exon in samples pulsed with DUX4 relative to untreated or IFNG treated cells, whereas fewer differential changes occurred across the CDS or 3′UTR (FIGS. 38C and 38D). In cells treated with a DUX4 pulse+IFNG versus IFNG, 26.7% of transcripts showed significantly reduced TE at the 5′UTR, 13.9% at the TSS, and 4.3% within the first exon (data not shown). These transcripts shared a large degree of overlap in enriched gene ontology (GO) terms, including processes involved in mRNA regulation, metabolism, translation, and antigen processing and MHC-I presentation (FIG. 38E). Thus, Ribo-seq analysis suggests that a subset of RNAs and biological processes, such as antigen presentation pathways, are particularly sensitive to DUX4 translational suppression.

### Genome-Wide Polysome Profiling Identifies Defects in Translation Initiation and Elongation.

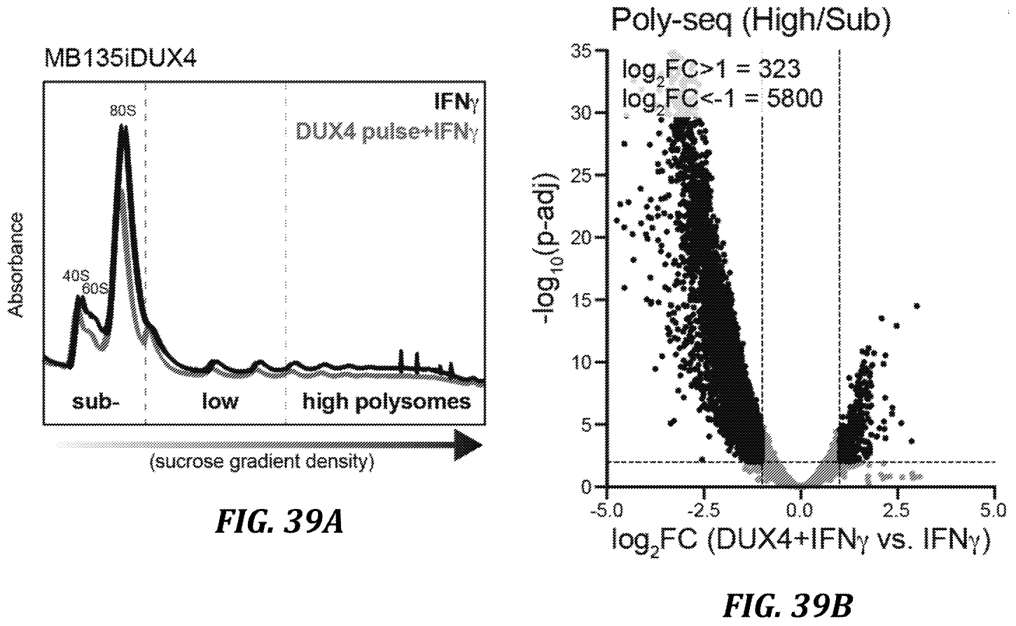

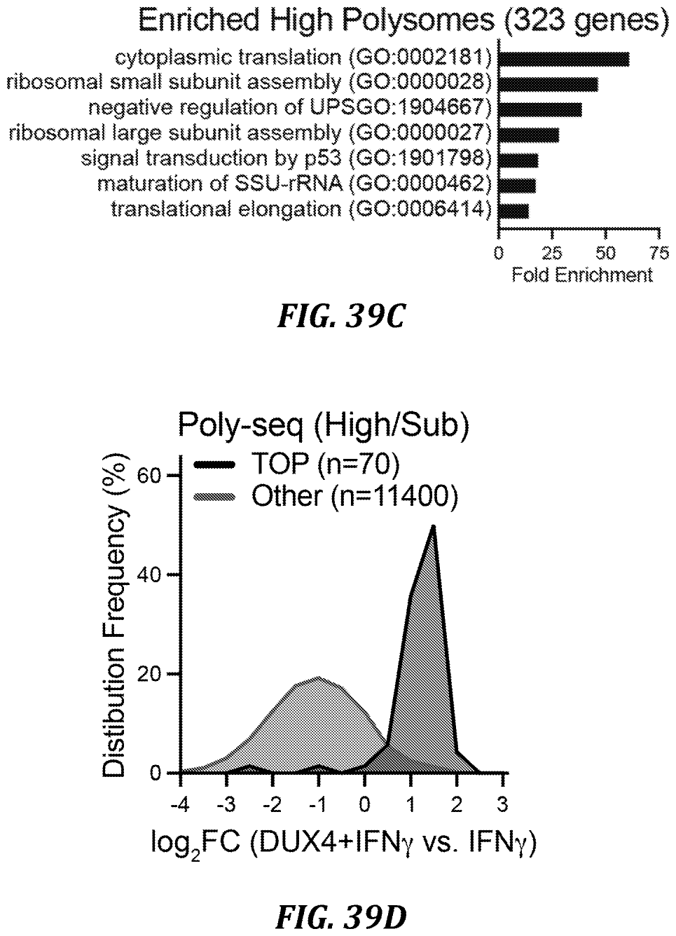

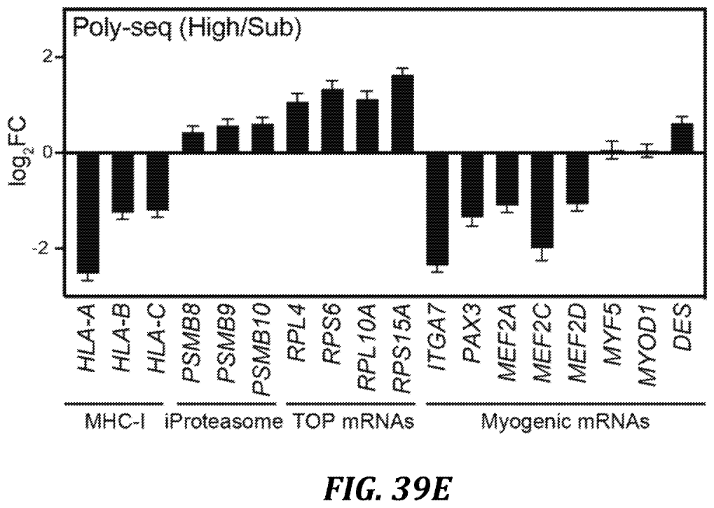

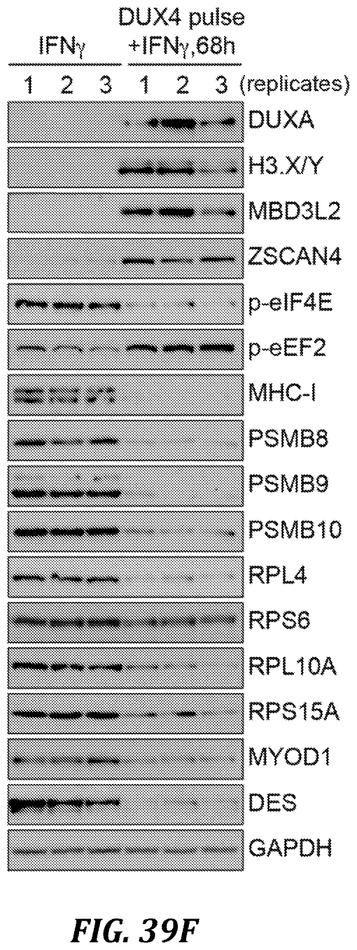

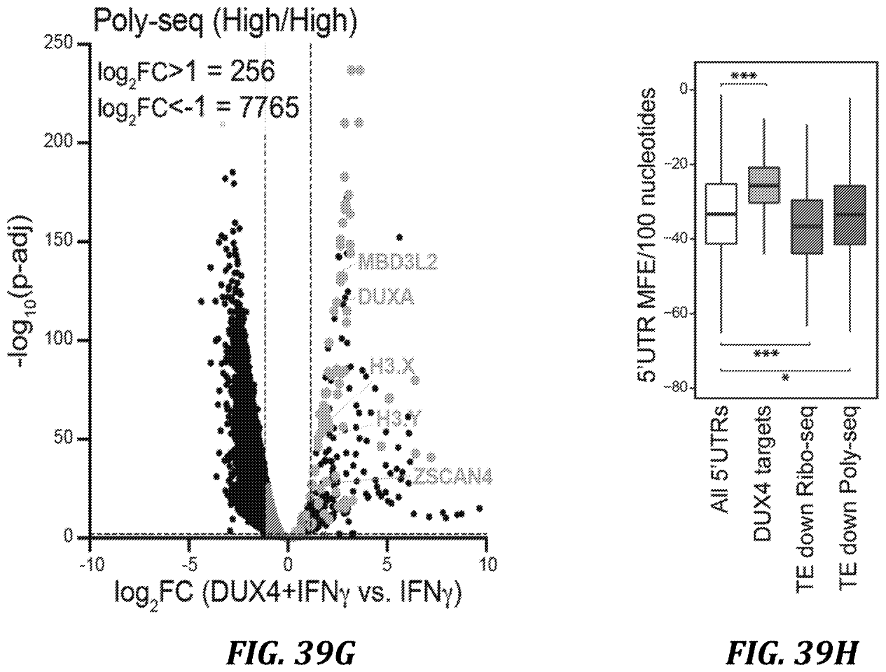

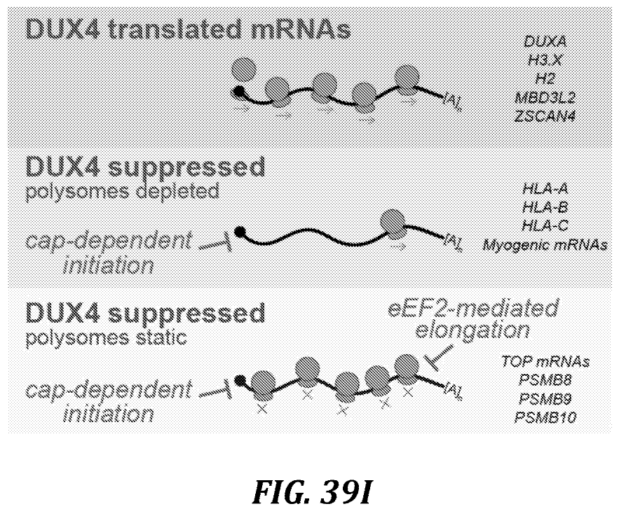

To directly measure ribosome density per mRNA, polysome profiling was performed on MB135iDUX4 myoblasts treated with identical experimental conditions as previously outlined: IFNG versus DUX4 pulse+IFNG harvested at 68 hours (FIG. 39A). RNA fractions representing sub-polysome (40S-60S-80S), low polysome (1-3 ribosomes), and high polysome (>3 ribosomes) populations were pooled and RNA-seq analysis performed. To measure changes in polysome occupancy, mRNAs that were significantly induced by DUX4 at the transcriptional level were filtered out, and mRNA abundance in each polysome fraction was determined relative to total input mRNA reads (data not shown). Notably, DUX4 results in a decrease in the high polysome fraction and an enrichment in the sub-polysome fraction. Similarly, the high-to-sub polysome (high/sub) ratio, which in theory decreases with translation initiation defects and increases with elongation defects, identified 5800 genes that had decreased polysome association and 323 genes with increased association (|log 2FC>1|; p-adj<0.01) (FIG. 39B), consistent with a broad suppression of translation initiation.

GO analysis of the 323 genes enriched in the high polysome fraction showed an abundance of mRNAs encoding ribosomal proteins and translation factors (FIG. 39C). Five-prime terminal oligopyrimidine (TOP) motifs are enriched in mRNAs that encode factors essential for protein synthesis (Cockman et al., 2020), and are particularly sensitive to mTORC1 regulation of initiation factors (Hsieh et al., 2012; Thoreen et al., 2012) and eEF2K-eEF2 control of translation elongation (Gismondi et al., 2014). Analysis of a set of mRNAs with known TOP-motifs (Cockman et al., 2020) showed that TOP mRNAs remain associated with polysomes following a pulse of DUX4, while most other transcripts are depleted in the high polysome fraction (FIG. 39D). However, TOP mRNA-encoded ribosomal proteins RPL10A, RPL4, RPS6, and RPS15A are suppressed by DUX4 even though the mRNAs remain bound by polysomes (FIGS. 39E-39F), consistent with an inhibition of elongation.

To elucidate DUX4 post-transcriptional suppression of MHC-I and iProteasome subunits specifically, their mRNA high-to-sub polysome ratios was similarly assessed. HLA-A, HLA-B, and HLA-C mRNAs showed reduced polysome association indicative of impaired translation initiation; however, PSMB8, PSMB9, and PSMB10 mRNAs remained associated with polysomes, similar to TOP mRNAs (FIG. 39E). Together these data suggest that translation of MHC-I mRNAs is particularly sensitive to initiation defects, while a subset of mRNAs, including TOP and iProteasome mRNAs, appear suppressed by DUX4 possibly through combinatorial inhibition of translation initiation and stalled elongation.

Previous analysis of the DUX4-induced transcriptome in mouse muscle cells showed that DUX4 can both activate and inhibit genes to prevent myogenic differentiation (Bosnakovski et al., 2017; Knopp et al., 2016). This led us to question whether a pulse of DUX4 also had a prolonged suppressive effect on myogenesis. Interestingly, several early myogenic markers, including ITGA7, PAX3, MEF2A, MEF2C, and MEF2D mRNAs, showed a loss of polysome abundance indicative of reduced translation initiation (FIG. 39E). While the master regulator of skeletal myogenesis, MYOD1, showed no change in polysome abundance, and mRNAs encoding the muscle-specific gene Desmin had an increase in polysomes (FIG. 39E), both were suppressed at the protein level following a pulse of DUX4 (FIG. 39F). These data demonstrate that DUX4 shifts the translatome from one cell lineage to a more naïve state.

Translation of DUX4-Induced mRNAs

Comparing the high polysome fractions of DUX4 pulse+IFNG samples to IFNG-treated samples provides a measure of how DUX4 changes the overall translatome and showed that DUX4-induced mRNAs were indeed associated with polysomes (FIG. 39G). Thousands of mRNAs were reduced in the DUX4 pulse+IFNG high polysome fraction (7765 genes; log 2FC<−1, p-adj<0.01); whereas 256 polysome-bound mRNAs were significantly upregulated following a pulse of DUX4 (log 2FC>1, p-adj<0.01), many of which are well-characterized target genes induced by DUX4 (Yao et al. 2014). Immunoblot analysis confirmed that the DUX4-induced mRNAs in the high polysome fraction correlated with protein translation of these mRNAs (FIG. 39F).

The enrichment of DUX4-induced mRNAs in the high polysome fraction might reflect their increased abundance following DUX4 expression or a relative resistance to the DUX4-mediated translational inhibition, or both. Thermodynamic stability and RNA secondary structures within the 5′UTR of an mRNA can influence translation initiation rates (Kozak, 1989; Svitkin et al., 2001) with higher predicted minimum free energy (MFE) showing higher translation efficiency. It has been previously observed that some DUX4-bound repetitive elements are co-opted to form alternative promoters for many DUX4 target genes (Yao et al., 2014; Young et al., 2013). To account for altered 5′UTR sequences starting at noncanonical promoters, the functional transcription start sites of 84 DUX4 targets were annotated (Data not shown). The predicted MFE of the 5′UTRs of the DUX4-induced mRNAs are significantly higher relative to the 5′UTRs of transcripts showing decreased transcription efficiency following a pulse of DUX4 as measured by Ribo-seq or polysome profiling (FIG. 39H). Therefore, DUX4-induced mRNAs are predicted to be less susceptible to inhibition of translation initiation, which correlates with their observed increase in protein expression and polysome association (FIGS. 39F-39G). Together, these data demonstrate that DUX4 orchestrates broad inhibition of mRNA translation coincident with de novo translation of DUX4-induced mRNAs (FIG. 39I), effectively reprogramming the cellular translatome

### Translational Suppression by Endogenous DUX4

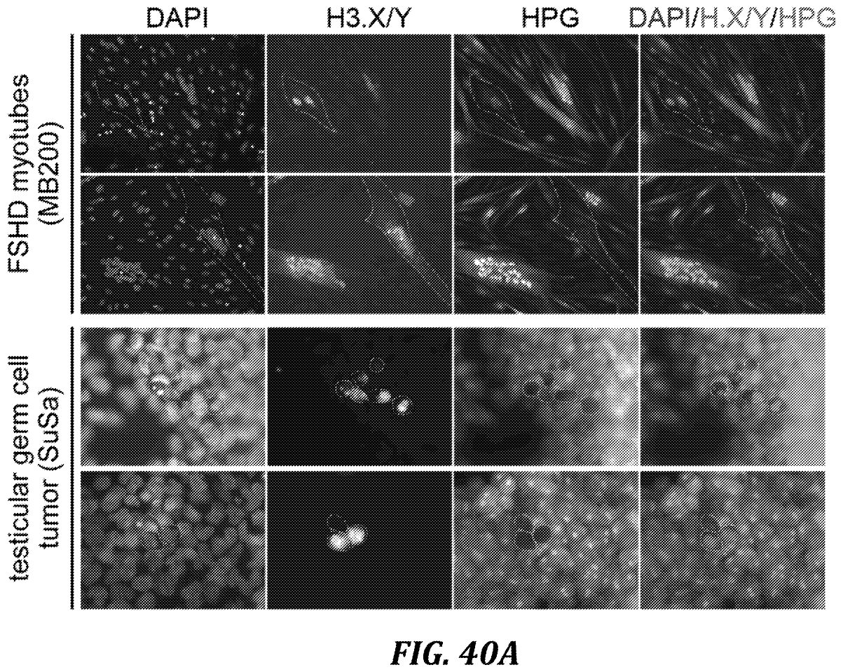

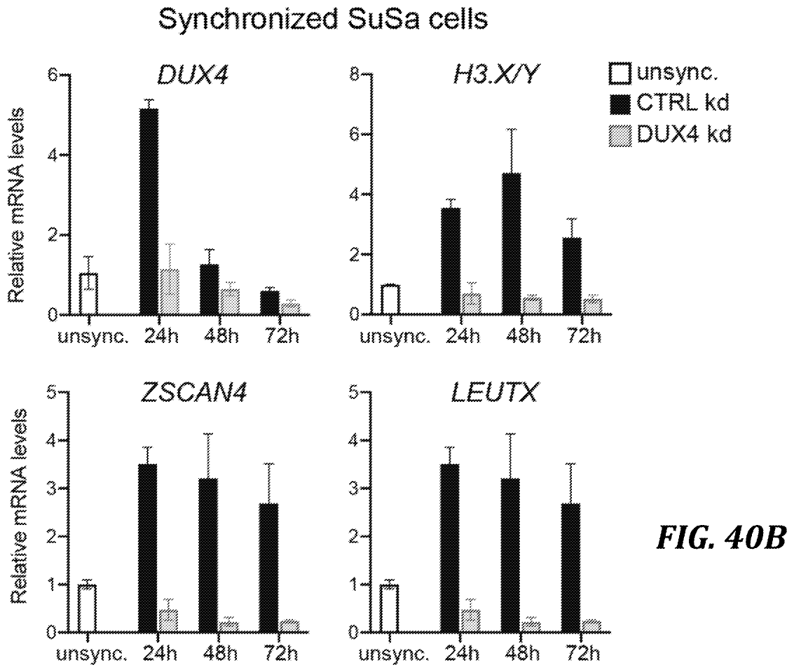

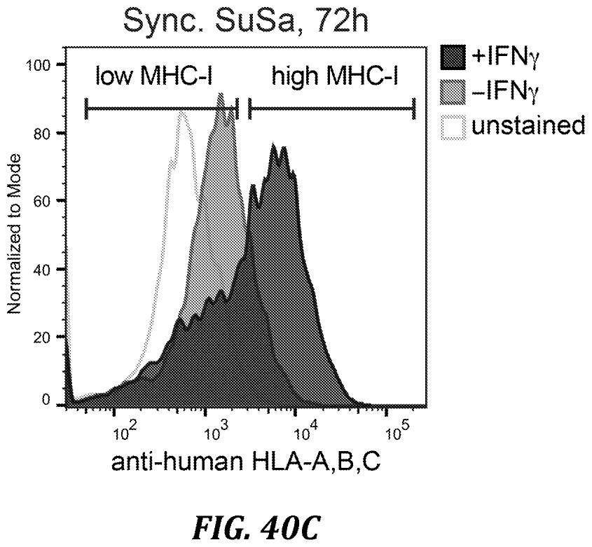

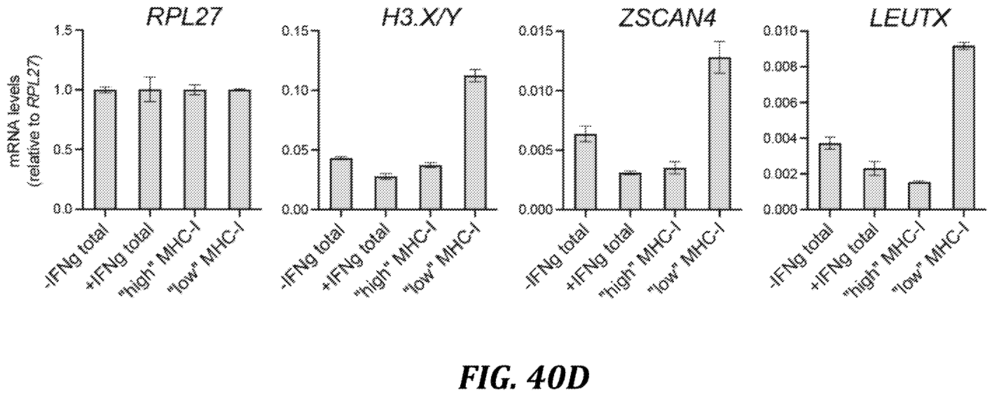

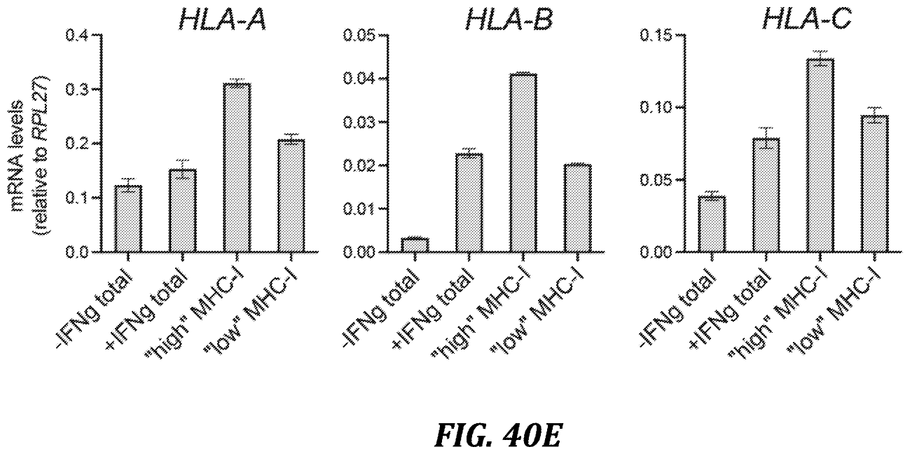

Intriguingly, broad translational inhibition has previously been reported in rare populations of mouse embryonic stem cells (mESCs) expressing ZSCAN4, a target gene of Dux, the mouse ortholog of DUX4; however, the precise molecular dynamics of this process remain unknown. It was tested whether a similar DUX4-induced block in translation occurs in rare populations of DUX4-expressing facioscapulohumeral muscular dystrophy (FSHD) muscle cells and cancer cells. To determine whether endogenous DUX4 expression alters protein translation similar to doxycycline-inducible DUX4 model system, patient-derived FSHD muscle cells and SuSa germinoma cells, two cell types previously shown to express endogenous DUX4 (Geng et al., 2012; Snider et al., 2010) were used. In both FSHD and SuSa cells, only a small percentage of cells express endogenous DUX4. Metabolic labeling of de novo protein synthesis with HPG in FSHD myotubes and SuSa cells showed a dramatic reduction in HPG Click-iT signal in cells expressing DUX4-induced H3.X and H3.Y (FIG. 40A), indicating translation of the DUX4 induced mRNA in cells with otherwise broadly suppressed protein translation. To study the secondary and long-lived consequences of endogenous DUX4 expression in SuSa cells, cells were using a release from confluence culture protocol that resulted in a synchronized burst of endogenous DUX4 expression and prolonged expression of DUX4 target genes H3.X/Y, ZSCAN4, and LEUTX (FIG. 40B), whereas gapmer-mediated knockdown of DUX4 abrogated target gene expression. IFNG-stimulated SuSa cells were flow-sorted into MHC-I “high” and “low” pools three days after release from confluence (approximately 48 hrs after the burst of DUX4 expression) (FIG. 40C). The cell population with low levels of MHC-I had elevated levels of DUX4 target gene expression relative to MHC-I “high” cells (FIG. 40D), whereas HLA-A, HLA-B, and HLA-C mRNA expression was only modestly reduced (FIG. 40E). Together, these data show that endogenous DUX4 expression in two independent cell types, FSHD muscle cells and SuSa cells, suppress protein translation and that this correlates with suppression of IFNG-induced MHC-I expression in SuSa cells.

This supports the model in which the loss of antigen presentation is in part due to translational inhibition driven by the DUX4 program, adding another layer of regulation that could allow DUX4-expressing cells to evade immune insults during development and in disease.

Together these data demonstrate that transient DUX4 expression results in the prolonged translational reprogramming of the cell. Critically, the data show that a relatively broad translational suppression profoundly suppresses the translation of some classes of mRNAs, including factors involved in antigen presentation, cellular translation, and myogenesis, and shifts the actively translated mRNAs to DUX4-induced transcripts. The mechanism appears multifactorial. Thus, transcriptional and translational regulatory mechanisms induced by DUX4 ultimately result in radical reprogramming of protein synthesis, underscoring the importance of understanding DUX4 biology beyond its role as a transcriptional activator.

Novel effects of DUX4 involving the disruption of mTORC1 signaling targets 4EBP1, eIF4E, and eEF2, key regulators of cellular translation initiation and elongation are disclosed herein. mTORC1 signaling is regulated by an array of intracellular and environmental cues, including nutrient and energy deprivation, cellular stress, hypoxia, and DNA damage. In line with the data presented herein, pathogenic roles of DUX4 in FSHD include induction of cellular hypoxia response pathways (Lek et al., 2020), disrupted mitochondrial function (Banerji et al., 2019; Heher et al., 2022; Turki et al., 2012), and oxidative stress and DNA damage (Dmitriev et al., 2016; Sasaki-Honda et al., 2018; Winokur et al., 2003). Similar processes are involved in early embryonic development and embryonic stem cell fate, including hypoxic environmental regulation (Houghton, 2021), dynamic changes in metabolism (Baumann et al., 2007; Leese, 2002; Rodriguez-Terrones et al., 2020), and DNA damage response pathways (Atashpaz et al., 2020; Grow et al., 2021; Srinivasan et al., 2020). Adequate reprogramming in response to adverse conditions integrates numerous transcriptional and post-transcriptional mechanisms that promote cell survival in developmental and disease contexts.

Together these data also demonstrate a broad suppressive effect on de novo protein synthesis lasting days following a pulse of DUX4 in muscle cells, requiring DUX4 transcriptional activity. Translational dynamics revealed by genome-wide profiling of ribosome protected fragments (RPFs) with Ribo-seq showed a marked loss of ribosome occupancy in the 5′ regulatory regions of mRNAs following a pulse of DUX4. These trends are consistent with a broad decline in translation initiation (Giess et al., 2020). To infer translational activity of all mRNAs following a pulse of DUX4, high throughput sequencing analysis of polysome fractions was performed. Indeed, many mRNAs were enriched in the sub-polysome fraction and significantly decreased in the high polysome fraction following a pulse of DUX4, indicative of decreased translational activity. Based on these data, MHC-I suppression may be characterized as a functional consequence underlying this broad translational suppression induced by DUX4. These data also suggest that DUX4 inhibition of elongation factor eEF2, 5′TOP mRNAs and iProteasome subunits are translationally suppressed while maintaining association with polysomes.

### Example 4: Continuous Expression of DUX4-CTD Suppresses MHC Class I Proteins in Human Melanoma Cells

### MHC Suppression by DUX4 CTD in MEL375 Cells

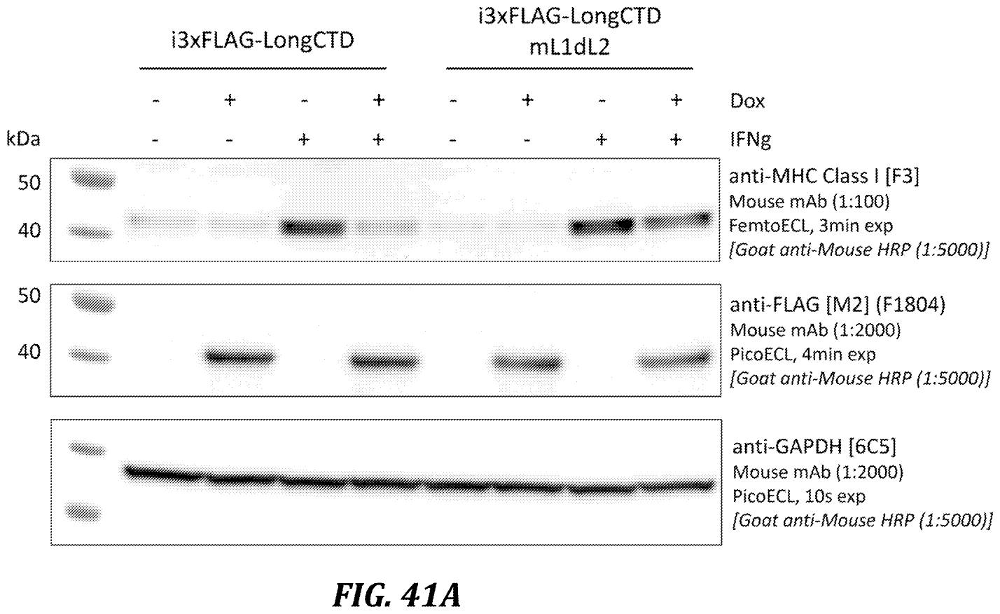

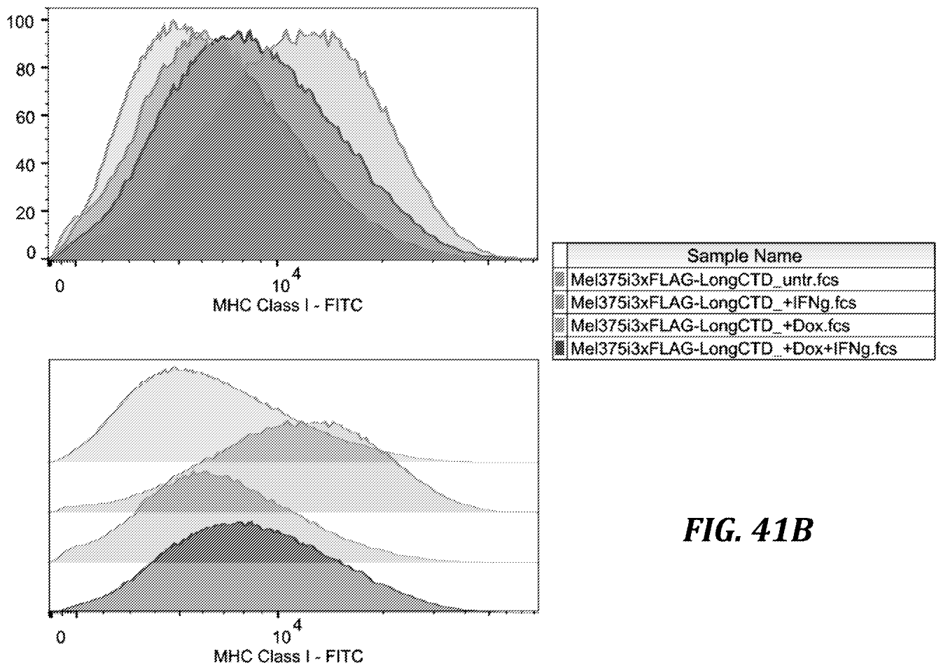

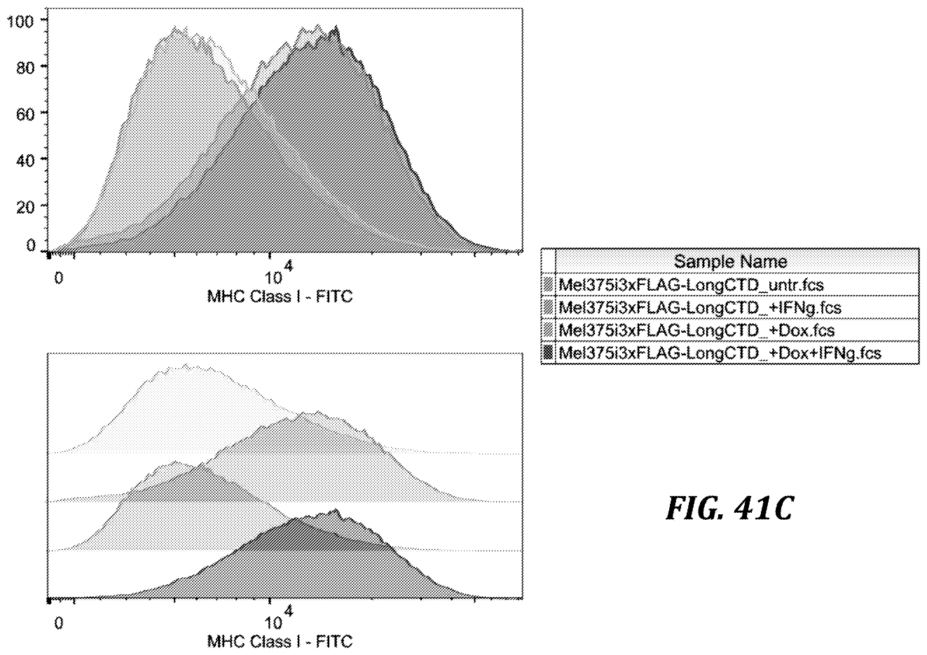

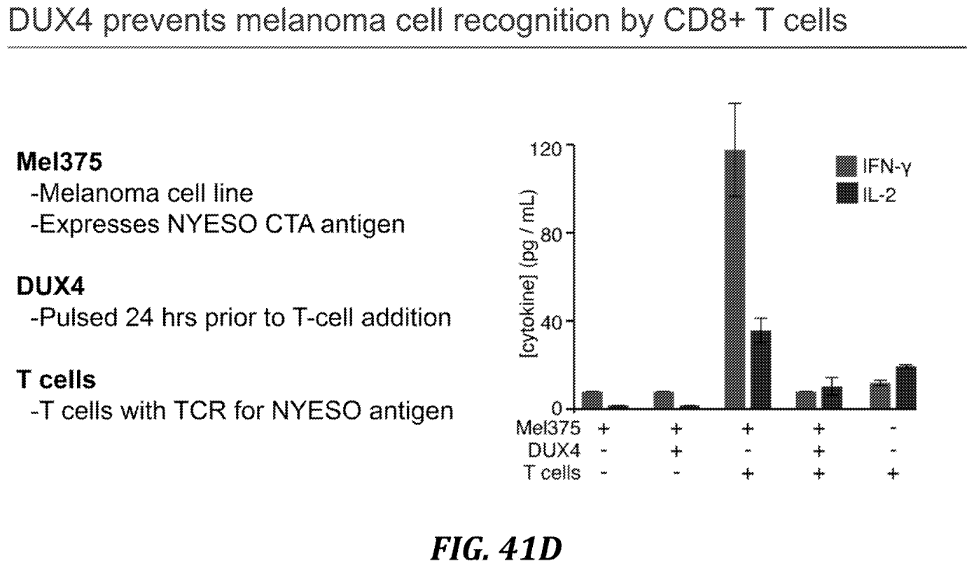

Expression of DUX4-CTD fragment suppresses interferon-induced MHC Class I expression and requires intact (L)LxxL(L) motifs. Human melanoma MEL375 cells were transduced with doxycycline inducible vectors expressing the 3×Flag-nls-tagged DUX4 CTD-154-424 (iDUX4-CTD) (SEQ ID NOS: 8-9) or the similar constructs with a mutation in the first (L)LxxL(L) motif and a deletion of the second (L)LxxL(L) motif (iDUX4-mL1dL2) (SEQ ID NOS: 10-11). Cells were treated with doxycycline (1 μg/ml) for four hours followed by the addition of interferon-gamma treatment for 16 hrs (50 ng/ml). As shown in FIG. 41A, in the absence of doxycycline induction of the transgenes, IFNG treatment results in increased MHC-class I proteins; whereas the DUX4-CTD suppresses IFNG induction of MHC proteins and the mutations of the (L)LxxL(L) almost completely abrogate this suppressive activity. As shown in FIGS. 41B and 41C, this correlates with decreased MHC surface protein expression as determined by FACs analyses.

### Suppression of T-Cell Activation by DUX4

To determine whether the combined short- and long-term mechanisms of MHC protein expression in response to cytokine signaling is sufficient to prevent T-cell activation, NYESO expressing MEL375 cells were used in an assay with T-cells engineered with the TCR (T Cell Receptor) for the NYESO antigen and measured T-cell activation by secretion of IFNG and IL-2. As shown in FIG. 41D, NYESO TCR-engineered T cells induce IFNG and IL-2 secretion in the presence of the NYESO expressing MEL375 cells. In contrast, a pulse-expression of DUX4 24 hrs prior to T-cell addition, completely blocks the T-cell activation by the NYESO expressing MEL375 cells.

These data also provide evidence of decreased de novo protein synthesis in FSHD and cancer cells endogenously expressing DUX4. These cell types, like human embryonic stem cells, exhibit cell-to-cell heterogeneity in culture, with only about 1-5% of cells spontaneously expressing the DUX4 transcriptional program (Rickard et al., 2015; Snider et al., 2010; Taubenschmid-Stowers et al., 2022). Similar expression profiles are found in mouse embryonic stem cells (ESCs), with a rare population of 2C-like cells driven by expression of mouse Dux, a functional ortholog of human DUX4 (Macfarlan et al., 2012; Zalzman et al., 2010). 2C-like cells have been associated with increased potency (Ishiuchi et al., 2015; Macfarlan et al., 2012) and a global reduction of translation (Hung et al., 2013), including suppression of pluripotency proteins like Dnmt1 and Oct4 (Eckersley-Maslin et al., 2016) and ribosomal proteins (Sun et al., 2021). Like the transient attenuation of protein synthesis following a pulse of DUX4 in myoblasts, translational suppression in mouse ESCs is lifted and protein synthesis resumes upon exit from the 2C-like state (Eckersley-Maslin et al., 2016; Sun et al., 2021), highlighting the dynamic nature of molecular events as cells transition between states. Like stem cells, tumors possess heterogeneity wherein some cancer cells undergo dedifferentiation and re-express developmental genes, giving rise to cells with expanded potential (Reya et al., 2001). Protein synthesis is frequently dysregulated in cancer, and paradoxically, instances of both enhanced or suppressed translation have been reported (Bhat et al., 2015; de la Parra et al., 2018; Jana et al., 2021; Lee et al., 2021; Liu et al., 2019; Xu & Ruggero, 2020).

Overall, these data suggest that transient DUX4 expression gives rise to a naïve cellular state through combined transcriptional and post-transcriptional mechanisms and that a pulse of DUX4 in muscle cells shifts the translatome to a characteristically totipotent signature while suppressing the myogenic program.

### Materials and Methods

### Cell Culture

All myoblast experiments were conducted in immortalized MB135 or MB200 myoblast cell lines (isolated from a control or FSHD2 subject, respectively cultured in Ham's F-10 Nutrient Mix (Gibco) supplemented with 15% fetal bovine serum (Hyclone), 100 U/100 μg/ml penicillin/streptomycin (Gibco), 1 μM dexamethasone (Sigma), and 10 ng/ml recombinant human basic fibroblast growth factor (PeproTech). To differentiate the myoblasts to myotubes, media was changed to DMEM supplemented with 10 μg/ml insulin (Sigma) and 10 μg/ml transferrin (Sigma). Cell lines containing doxycycline-inducible transgenes were additionally cultured with 2 μg/mL puromycin (Sigma). Transgenes were induced with 1 μg/mL of doxycycline (Sigma) for 4 hours prior to other treatments for a total of 20 hrs. The Kitra-SRS cells (RRID:CVCL_YI69) were provided by Dr. H. Otani and Osaka University (Nakai et al., 2019) and were cultured in DMEM supplemented with 10% fetal bovine serum (Hyclone) and 100 U/100 μg/ml penicillin/streptomycin (Gibco). Biological replicates consisted of independent but parallel experiments, such as simultaneously stimulating three cell culture plates with IFNγ. Technical replicates consisted of repeat measurements of the same biological sample, such as loading the same biological sample in triplicate for analysis by RT-qPCR.

### Cloning, Virus Production, and Monoclonal Cell Line Isolation

Human DUX4 and mouse Dux truncation constructs were created by cloning synthesized, codon-optimized gBlock fragments into the pCW57.1 vector (Addgene plasmid #41393) downstream of the doxycycline-inducible promoter. Lentiviral particles were created by transfecting 293T cells with the subcloned pCW57.1 expression vectors, psPAX2 (Addgene plasmid #12260), and pMD2.G (Addgene plasmid #12259) using Lipofectamine 2000 according to the manufacturer's instructions (Invitrogen). Myoblasts were transduced and selected using 2 μg/mL puromycin at low enough confluence to allow for isolation of clonal lines using cloning cylinders. Transgenic clonal lines were validated for protein size, expression level, and localization by western blot and immunofluorescence.

### Immune Stimulation and RT-qPCR

Myoblasts were transfected with either (final concentrations) 10 μM 2′,3′-cGAMP (Sigma Aldrich), 2 μg/mL poly(I:C) (Invitrogen), or 1 μg/mL 3′ppp-dsRNA RIG-I ligand (Dan Stetson Lab, UW) using Lipofectamine 2000 according to manufacturer's protocol, or were stimulated with 1000U IFNβ (PeproTech) or 200 ng/ml IFNγ (R+D Systems) by addition directly to cell culture medium. After 16 hours of immune stimulation, RNA was collected from cells using the NucleoSpin RNA Kit (Macherey-Nagel) according to manufacturer's instructions. RNA samples were quantified by nanodrop and 1 μg of RNA per sample was treated with DNase I Amplification Grade (Thermo Fisher), and then synthesized into cDNA using the Superscript IV First-Strand Synthesis System, including oligo dT primers (Invitrogen). qPCR was run in 384-well plates on an Applied Biosystems QuantStudio 6 Flex Real-Time PCR System (ABI) and analyzed in Microsoft Excel.

### RNA-Seq Library Preparation and Sequencing

RNA was extracted as described above from untreated, doxycycline-treated, IFNγ-treated, or doxycycline- and IFNγ-treated samples. RNA was submitted to the Fred Hutchinson Cancer Research Center Genomics Core for library preparation using the TruSeq3 Stranded mRNA kit (Illumina) followed by size and quality analysis by Tapestation (Agilent). Libraries were sequenced on a NextSeq P2-100 (Illumina).

### Immunofluorescence

Cells were fixed for 10 minutes with 2% paraformaldehyde (Thermo Scientific) for DUX4/STAT1 and 4% paraformaldehyde for DUX4/IDO1 then permeabilized for 10 minutes with 0.5% Triton X-100 (Sigma), both at room temperature with gentle shaking. Cells were then blocked for 2 hours with PBS/0.3M glycine/3% BSA at room temperature with gentle shaking. Primary antibodies were incubated at 4° C. overnight: mouse anti-FLAG M2 1:500 (Sigma #F1804), rabbit anti-STAT1 mAb 1:750 (Abcam #ab109320), rabbit anti-IDO1 (D5J4E) 1:100 (Cell Signaling Tech 86630S), and mouse anti-DUX4 (P2G4) 1:250 (Geng et al., 2011). Cells were washed three times with 1×PBS containing 3% BSA, then secondary antibodies were incubated for 1 hr at room temperature: FITC-conjugated donkey anti-rabbit (Jackson immunoResearch) or TRITC-conjugated donkey anti-mouse (Jackson ImmunoResearch). Cells were washed once with 1×PBS containing 3% BSA then stained with DAPI (Sigma) for 10′ at room temperature and then visualized.

### Fractionated Anti-FLAG Immunoprecipitation

Cells were lysed on the plate with digitonin lysis buffer pH 7.4 (37.5 μg/mL digitonin, 25 mM Tris-HCl pH 7.5, 125 mM NaCl, 1 mM EDTA, 5% glycerol) supplemented with Pierce Protease Inhibitors EDTA-free (PIA32955) and Pierce Phosphatase Inhibitors (PIA32957), transferred to a centrifuge tube and incubated for 10 minutes at 4° C. with rotation. Centrifugation at 2500 rcf at 4° C. for 5 min pelleted the nuclei, supernatant was discarded, and nuclei resuspended in 1 mL IP buffer pH 7.4 (25 mM Tris-HCl pH 7.5, 175 mM NaCl, 1 mM EDTA, 0.2% NP-40, 5% glycerol) and incubated for 1 hour at 4° C. with rotation then spun at 21000 rcf for 10 minutes at 4° C. to pellet insoluble debris. Protein concentration was determined using the Pierce BCA Protein Assay Kit (ThermoFisher, 23225). An equivalent amount of protein per sample was pre-cleared with Dynabeads Protein G (Invitrogen) bound to mouse IgG (Abcam #131368) for 1 hour at 4° C. with rotation. FLAG-tagged constructs were then immunoprecipitated with Dynabeads Protein G beads coupled to mouse anti-FLAG M2 mAb (Sigma F3165) for 3 hours at 4° C. with rotation. Beads were washed 3× with 1 mL IP buffer and eluted by adding 2× NuPage LDS Sample Buffer (Thermo Fisher, diluted from 4× with PBS) to the beads and heating for 10 minutes at 70° C.

### Liquid Chromatography Mass Spectroscopy (LC-MS)

For LC-MS, anti-FLAG immunoprecipitation was performed with beads cross-linked to the anti-FLAG antibody and the proteins competitively eluted with FLAG peptide. Eluted protein samples were electrophoresed into a NuPage 4-12% Bis-Tris gel, excised, and processed by the Fred Hutchinson Cancer Research Center Proteomics Core. Samples were reduced, alkylated, digested with trypsin, desalted, and run on the Orbitrap Eclipse Tribid Mass Spectrometer (Thermo Fisher). Proteomics data were analyzed using Proteome Discoverer 2.4 against a Uniprot human database that included common contaminants using Sequest HT and Percolator for scoring. Results were filtered to only include protein identifications from high confidence peptides with a 1% false discovery rate. Proteins that were identified in at least one sample from both independent experiments with at least 2 PSMs in one sample were assigned to one of ten categories: 1, candidates; 2, cytoskeletal associated; 3, cytoskeletal; 4, ribosome/translation associated; 5, proteasome associated; 6, membrane or extracellular; ER, golgi, or vesicle associated; 8, lipid metabolism; 9, chaperones; 10, nuclear import or nuclear membrane associated. The proteins in category 1 were further investigated for interactions with DUX4. It should be noted that this category assignment process de-prioritized groups of proteins based on assignment to a cellular compartment or function (e.g. ribosome/translation proteins might associate with DUX4 as part of a translation complex rather than having a role in immune signaling) and it is possible that some of the proteins assigned to the non-candidate categories might be functional interactors with DUX4 and have an important biological role.

### Chromatin Immunoprecipitation and Sequencing

Chromatin immunoprecipitation (ChIP) was performed as previously described (Nelson et al 2006) with the following modifications: Cells were plated and allowed to grow to 70-80% confluence. Pelleted nuclei were sonicated on a Diagenode Biruptor on “Low” for 10 min as 30 sec on/30 sec off, followed by 6 rounds of sonication on “High” for 10 min each as 30 sec on/30 sec off (70 minutes total sonication) in IP Buffer+0.5% SDS. For immunoprecipitation, 500 ng of chromatin was set aside per condition as an “Input” and 4 μg of antibody was added to 10 μg of chromatin in an equal volume of IP Buffer+0.5% SDS across samples. IP Buffer (150 mM NaCl, 50 mM Tris-HCl pH 7.4, 5 mM EDTA, 1% Triton X-100, 0.5% NP-40, +Roche complete mini protease inhibitor EDTA-free) was added to lower the percentage of SDS <0.1%, and tubes were incubated with rotation overnight at 4° C. During this time, protein-A agarose Fastflow beads (Millipore) were washed twice with IP Buffer and then blocked in IP Buffer containing 2% BSA by rotating overnight at 4° C. After clearing the chromatin as described, beads were aliquoted to fresh tubes and the top 90% of chromatin was transferred to the tubes containing the blocked bead slurry. Tubes were rotated for 1 hour at 4° C. Beads were washed (definition of a “wash” in the protocol) 5 times with cold IP Buffer containing 0.1% SDS, 2 times with cold IP Buffer containing 500 mM NaCl, and 2 times with cold PBS. DNA was isolated as described in the original protocol and used as a template in qPCR. Input DNA was used to create a standard curve. qPCR primers were previously published (Maston et al., 2012, Non-canonical TAF complexes regulate active promoters in human embryonic stem cells. Elife 1, e00068; Rosowski et al., 2014, Toxoplasma gondii Inhibits gamma interferon (IFN-gamma)- and IFN-beta-induced host cell STAT1 transcriptional activity by increasing the association of STAT1 with DNA. Infect Immun 82, 706-719) for ISGs and h16q21, respectively.

### Proximity Ligation Assay

Cells were fixed for 10 minutes with 4% paraformaldehyde (Thermo Scientific), permeabilized for 10 minutes with 0.5% Triton X-100 (Sigma), and then blocked for 2 hours at room temperature with PBS/0.3M glycine/3% BSA. Primary antibodies were diluted in PBS/3% BSA and incubated with samples overnight at 4° C.: anti-FLAG [M2] (F1804) (1:4000), anti-STAT1 [EPR4407] (1:1000), and anti-pSTAT1 Y701 [58D6] (1:1000). Samples were washed 3 times for 10 minutes with 1× Wash Buffer A (10 mM Tris, 150 mM NaCl, 0.05% Tween, adjusted pH to 7.4), and then incubated with Duolink In Situ PLA Probe Anti-Rabbit PLUS (Sigma, Cat #DUO92002) and Duolink In Situ PLA Probe Anti-Mouse MINUS (Sigma, Cat #DUO92004) diluted in PBS/3% BSA for 1 hour in a humidity chamber at 37° C. Samples were washed 3 times for 10 minutes with 1× Wash Buffer A, and then treated with ligase from the Duolink In Situ Detection Reagents Green kit (Sigma, Cat #DUO92014) for 30 minutes in a humidity chamber at 37° C. Samples were washed 3 times for 10 minutes with 1× Wash Buffer A, and then treated with polymerase from the Duolink In Situ Detection Reagents Green kit for 1 hour and 40 minutes in a humidity chamber at 37° C. Samples were washed 2 times for 10 minutes with 1× Wash Buffer B (200 mM Tris, 100 mM NaCl, adjusted pH to 7.5) and then once for 1 minute with 0.01× Wash Buffer B. Samples were mounted with Prolong Glass Antifade Mountant (ThermoFisher, Cat #P36983), and then visualized with a fluorescent microscope using FITC and DAPI filters.

### CUT&Tag

CUT&Tag was performed as previously described (Kaya-Okur et al., 2019) with the following modifications: MB135-iDUX4-CTD myoblasts were plated and allowed to grow to 70-80% confluence. Cells were left untreated, treated with 200 ng/ml IFNG for 16 hr, or pre-treated with 1 μg/mL doxycycline for 4 hr then had IFNG added directly to cell media for an additional 16 hr. Fresh cells were harvested and washed in PBS, crosslinked with 0.1% formaldehyde for 90 seconds, then counted and 1.25e6 cells were aliquoted per reaction tube. Drosophila S2 cells were spiked-in at a genomic ratio of 1:10. Nuclei were prepared from cells in Buffer NE1 (20 mM HEPES-KOH PH7.9, 10 mM KCl, 0.1% Triton X-100, 20% glycerol, 0.5 mM spermidine, Pierce Protease Inhibitors EDTA-free [PIA32955]) on ice for 10 min and then bound to concanavalin A-coated beads for 10 min. Primary antibody (dilution 1:50) was bound overnight at 4° C. in 25 μL per sample of Antibody Buffer (20 mM HEPES-KOH pH7.5, 150 mM NaCl, 0.5 mM spermidine, 0.01% digitonin, 2 mM EDTA, 1× Roche complete mini EDTA-free protease inhibitor). Secondary antibody (dilution 1:100) was bound in 25 μL per sample of Wash150 Buffer (20 mM HEPES-KOH PH7.5, 150 mM NaCl, 0.5 mM spermidine, 1× Roche complete mini EDTA-free protease inhibitor) for 30 min at room temperature. pAG-Tn5 pre-loaded adapter complexes (Epicypher) were added to the nuclei-bound beads for 1 hr at room temperature in 25 μL of Wash300 Buffer (20 mM HEPES-KOH PH7.5, 300 mM NaCl, 0.5 mM spermidine, 1× Roche complete mini EDTA-free protease inhibitor), then beads were washed and resuspended in Tagmentation Buffer (Wash300 Buffer+10 mM MgCl2) and incubated at 37° C. for 1 hr in a thermocycler with heated lid. Tagmentation was stopped by addition of EDTA, SDS, and proteinase K. DNA was extracted by Phenol-Chloroform and amplified by PCR using CUTANA High Fidelity 2×PCR Master Mix (EpiCypher) and cycling conditions: 5 min at 58° C.; 5 min at 72° C.; 45 sec at 98° C.; 14 cycles of 15 sec at 98° C., 10 sec at 60° C.; 1 min at 72° C. PCR products were cleaned up using SPRI beads (Agencourt) at a ratio of 1.3:1 according to manufacturer's instructions.

### CUT&Tag Analysis

CUT&Tag data were aligned to the GRCh38 patch 13 human genome e following the Benchtop CUT&Tag v3 protocol (Kaya-Okur et al., 2019). Subsequent to alignment we calculated 1× genome coverage normalization with read centering and read extension using deepTools' bamCoverage (Ramírez, 2016) then mapped the resulting coverage tracks to regions of interest using bedtools' map function (Aaron R. Quinlan, 2010). Coverage graphs were plotted using ggplot2 from the tidyverse package in R (Wickham H & E, 2019).

### RNA-Seq Analysis

Sequencing analysis was performed using R version 4.0.3 (R Core Team, 2020). Sequencing reads were trimmed using Trimmomatic (version 0.39) (Bolger et al., 2014, Trimmomatic: a flexible trimmer for Illumina sequence data. Bioinformatics 30, 2114-2120), and aligned to the Homo sapiens GRCh38 reference genome with the Rsubread aligner (Liao et al., 2019, The R package Rsubread is easier, faster, cheaper and better for alignment and quantification of RNA sequencing reads. Nucleic Acids Res 47, e47). Gene counts were analyzed using featureCounts (v2.0.1) (Liao et al., 2019, supra) and the Gencode v35 annotation file. Normalization and differential expression analysis were done with DESeq2 (v1.26.0) (Love et al., 2014, Moderated estimation of fold change and dispersion for RNA-seq data with DESeq2. Genome Biol 15, 550).

### In Vitro Transcription, Translation, and Immunoprecipitation

In vitro translated proteins were prepared using the TnT Quick Coupled Transcription Translation System (Promega, L2080) for the SP6 promoter on the pCS2-3×MYC-STAT1, pCS2-3×MYC-STAT1-S727E, pCS2-3×FLAG-DUX4aa154-424, and pCS2-3×FLAG-DUXB plasmids. To phosphorylate the 3×MYC-STAT1 and 3×MYC-STAT1-S727E proteins at Y701, 20 μL of the TnT reaction was mixed with 8 μL of 5× Kinase Buffer A (ThermoFisher, Cat #PV3189), 4 uL 10 mM ATP (ThermoFisher, Cat #PV3227), 0.4 ug recombinant JAK1 kinase (ThermoFisher, Cat #PV4774), and then nuclease-free ddH2O to a final volume of 40 μL and incubated for 1 hour at 30° C. The same procedure was used for treatment of 3×FLAG-DUX4-CTD with JAK1 kinase. For IPs, the in vitro translated proteins were mixed and incubated for 15 minutes in a 37° C. water bath. 0.3 mL of ice-cold in vitro IP buffer [20 mM Tris-HCl pH 7.5, 137 mM NaCl, 1 mM EDTA, 0.1% Tween20, 5% glycerol, adjusted to pH 7.4] was added to each sample and then 50 μL of Dynabeads Protein G bound to mouse IgG1 anti-FLAG [M2] (F3165) and incubated for 3 hours at 4° C. with gentle rotation. After immunoprecipitation of FLAG-tagged constructs, each sample was washed 3 times with 1 mL of ice-cold in vitro IP buffer in a 4° C. room. Then the immunoprecipitated proteins were eluted from the beads by incubation with 30 uL elution buffer (2×NuPage LDS Sample Buffer diluted with IP buffer) for 10 minutes at 70° C. at 1000 rpm.

### Antibodies

Antibodies against STAT1 [EPR4407] (ab109320), pSTAT1 Y701 [M135] (ab29045), pSTAT1 S727 [EPR3146] (ab109461), YBX1 [EP2708Y] (ab76149), PABPC1 (ab21060), hnRNPM [EPR13509 (B)] (ab177957), PRKDC [Y393] (ab32566), and HSP90AB1 [EPR16621] (ab203085) were purchased from Abcam. Antibodies against FLAG tag [M2] (F1804) and [M2] (F3146) were purchased from Sigma Aldrich. Antibodies against DDX3X [D19B4] (8192S), hnRNPK [R332] (4675S), TRIM28 [C42G12] (4124S), PPP2R1A [81G5] (2041S), NCL [D4C70] (14574S), and MYC tag [71D10] (2278S), and IDO1 [D5J4E] (86630S) were purchased from Cell Signaling Technology. Antibodies against hnRNPU (14599-1-AP), CDK4 (11026-1-AP), and HAT1 (11432-1-AP) were purchased from ProteinTech. The antibody against HSPA8 [SR39-04] (PIMA532002) was purchased from ThermoFisher. The antibody against DUX4 (P2G4) was described previously (Geng et al., 2011, Immunodetection of human double homeobox 4. Hybridoma (Larchmt) 30, 125-130). The antibodies Goat anti-Rabbit IgG HRP (ThermoFisher, A27036) and Rat anti-Mouse IgG HRP for IP (Abcam, ab131368) were used as secondary antibodies against rabbit and mouse primary antibodies for western blotting.

### CRISPR-Cas9 Knockout Generation

Generation of MB135iDUX4 PKR KO myoblasts was achieved using CRISPR/Cas9 technology (Doudna & Charpentier, 2014). A guide RNA (gRNA) sequence targeting EIF2AK2 (Li et al., 2017) was cloned into the BbsI site of the Cas9 (BB)-2A-GFP plasmid (Addgene) containing a U6 promoter and Cas9-GFP. MB135iDUX4 myoblasts were transfected with this construct using Lipofectamine 3000 Reagent (Invitrogen) according to the manufacturer protocol and incubated for 1.5 days. Fluorescence activated cell sorting (FACS) analysis was used to sort cells expressing GFP-tagged Cas9 using the BD FACS Aria II with BD FACS Diva software (BD Biosciences) to obtain individual clones. Individual PKR knockout clones were screened using immunoblot analysis, and mutant alleles were validated with Sanger sequencing.

### Starvation-Induced Cell Cycle Synchronization

SuSa cells were seeded at 90% confluence on a 0.1% gelatin-coated 10 cm plate and incubated for 8 days at 37° C. and 5% CO2. Cells were supplemented with fresh growth media and incubated for 1-3 hours to release from synchronization, lifted using trypsin, seeded onto gelatin-coated plates at 30% confluence, and harvested at terminal time points of 24, 48, or 72 hours. Where specified, synchronized cells were transfected with gapmers (described below) or supplemented with 50 ng/mL IFN□ for 16 hours prior to harvest. FACS analysis was used to sort cell populations with varying level of MHC class I surface molecules. Briefly, cells were harvested using trypsin and stained in Cell Staining Buffer (BioLegend) with a 1:50 dilution of Brilliant Violet 605-conjugated MHC class I antibody (BioLegend) for 30 minutes at 4° C. Cells were resuspended in FACS buffer (1×DPBS, 1% BSA, 5 mM EDTA) and flow sorted using FACSymphony S6 with BD FACSDiva software (BD Biosciences). Data were analyzed using FlowJo V10.5.3.

siRNA Transfections

ON-TARGETplus siRNAs were obtained from Horizon/Dharmacon. Transfections of siRNAs into myoblasts were carried out using Lipofectamine RNAiMAX (Invitrogen) according to the manufacturer's protocol. Briefly, cells were transfected with 3.25 μl/mL Lipofectamine RNAiMAX and 25 pmol/mL of either gene-specific siRNA(s) or a scrambled non-silencing control siRNA diluted in Opti-MEM Reduced Serum Medium (Gibco), and incubated for ˜20 hours. A double transfection protocol was followed with pulse experimental conditions to ensure prolonged depletion of proteins, where cells were transfected 20 hours before and 20 hours after a 4-hour pulse of doxycycline.

### Gapmer Transfections

Gapmers were obtained from Qiagen. Transfections of gapmers into SuSa cells were carried out using Lipofectamine RNAiMAX (Invitrogen) according to the manufacturer's protocol. Briefly, SuSa cells were synchronized and released as described above, and reverse transfected with 1 μl/mL Lipofectamine RNAiMAX and 25 pmol/mL of either a pool of two DUX4-targeting gapmers or a control gapmer targeting GFP diluted in Opti-MEM Reduced Serum Medium (Gibco). Cells were incubated with gapmers for 8 hours. To remove gapmer-supplemented media, cells in suspension were pelleted at 400×g for 5 minutes at 4° C., resuspended in fresh growth media, and re-seeded onto 0.1% gelatin-coated plates.

### Immunoblotting

Protein samples were harvested in RIPA buffer [150 mM NaCl, 1% NP-40, 0.5% Na-deocycholate, 1% SDS, 25 mM Tris-HCl pH7.4] supplemented with protease and phosphatase inhibitor tablets (Pierce), followed by sonication in Diagenode Bioruptor. Lysate was cleared by centrifugation at 16000×g and quantified using a Pierce BCA assay (Thermo Fisher Scientific). Samples were run on NuPAGE precast polyacrylamide gels (Invitrogen) and transferred to PVDF membrane. Membranes were blocked in PBS containing 0.1% Tween-20 and 5% non-fat dry milk before overnight incubation at 4° C. with primary antibodies (see resource table for a list of antibodies used in this study). Membranes were incubated with horseradish peroxidase-conjugated secondary antibodies for 1 hour at room temperature and SuperSignal chemiluminescent substrate (Thermo Scientific) was used for detection on film with a Mini-medical 90 processor. Membranes were stripped with Restore Western Blot Stripping Buffer (Pierce).

### Subcellular Fractionation

MB135iDUX4 myoblasts were pulsed with doxycycline for 4 hours, incubated for 24 hours, supplemented with 50 ng/mL IFNG for 16 hours, and harvested at a terminal time point of 44 hours. Samples divided for whole-cell lysate (WCL) and subcellular fractionation were suspended in 300 μL ice-cold Cyto-lysis buffer (10 mM Tris pH 7.4, 10 mM NaCl, 0.2% NP-40, 1 mM DTT in nuclease free water). Subcellular fractionation samples were centrifuged at 650 RCF to pellet nuclei while cytoplasmic lysate remained in the supernatant. WCL, cytoplasmic, nuclear RNA and protein were harvested for RT-qPCR and immunoblotting, respectively.

m7GTP Cap-Binding Assay

MB135iDUX4 myoblasts were treated with and without a 4-hour pulse of doxycycline and harvested at a terminal time point of 68 hours. Cells were lysed in cap binding buffer [10 mM Tris-HCl pH 7.5, 140 mM KCl, 4 mM MgCl2, 1 mM DTT, 1 mM EDTA, 1% NP-40] supplemented with protease and phosphatase inhibitor cocktails (Pierce)], incubated on ice for 30 minutes, and lysate cleared at 12000 rpm for 30 minutes at 4° C. Soluble lysate was quantified with Pierce BCA assay (Thermo Scientific) and diluted in cap binding buffer without NP-40 to bring final concentration to 0.5 mg/mL in 0.5% NP-40. 50 μl of pre-washed 7-methyl-GTP-Sepharose bead slurry (Jena Bioscience) was added to 400 μg protein and incubated at 4° C. for 1 hour. Samples were centrifuged at 5000 rpm for 5 minutes at 4° C., washed twice with cap binding buffer containing 0.5% NP-40 and twice with PBS. Beads were suspended in NuPAGE LDS Buffer (Invitrogen) and incubated at 95° C. for 10 minutes to elute associated proteins.

### [35S] Radiolabeling

Cells were treated with and without a 4-hour pulse of doxycycline. Eight hours prior to harvest, cells were incubated in DMEM depleted for methionine and cysteine (Gibco) supplemented with 90 microcurie 35S-methionine/cysteine (Perkin Elmer) and 50 ng/ml IFNI (R&D Systems). Protein samples were harvested in RIPA buffer [150 mM NaCl, 1% NP-40, 0.5% Na-deocycholate, 1% SDS, 25 mM Tris-HCl pH7.4] supplemented with protease and phosphatase inhibitor tablets (Pierce), followed by sonication in Diagenode Bioruptor. Lysate was cleared by centrifugation at 16000×g and quantified using a Pierce BCA assay (Thermo Scientific). Samples were run on NuPAGE precast polyacrylamide gels (Invitrogen). Gels were stained with InstantBlue Coomassie (Abcam) following manufacturer protocol, placed on whatman paper, wrapped in plastic wrap, and dried. Gels were exposed to phosphor screen, imaged on Typhoon Trio imager, and analyzed with ImageQuant.

### HPG Click-iT Labeling

Cells were incubated for 30 minutes in DMEM or RPMI media depleted for methionine and cysteine (Gibco), followed by a 1-hour incubation in methionine-depleted media supplemented with 200 μM HPG (Sigma). Cells were fixed with 4% paraformaldehyde (Electron Microscopy Sciences) diluted in PBS for 10 minutes at room temperature and stained with Click-iT HPG Alexa Fluor 488 Protein Synthesis Assay Kit (Invitrogen) according to manufacturer's protocol.

### Ribosome Footprinting

Ribo-seq was performed as described previously (Calviello et al., 2016) using two 70% confluent 15 cm plates of MB135iDUX4 myoblasts per treatment condition (n=3). MB135iDUX4 myoblasts were pulsed with or without doxycycline for 4 hours, incubated for 48 hours, supplemented with or without 50 ng/mL IFNG for 16 hours, and harvested at a terminal time point of 68 hours. To harvest, media was aspirated and each 15 cm plate of adherent cells was rinsed with 25 mL ice-cold PBS supplemented with 100 μg/mL cycloheximide (Sigma). After thorough removal of the PBS, plates were immersed in liquid nitrogen and placed on dry ice. Cells were lysed in mammalian polysome lysis buffer (20 mM Tris-HCl, pH 7.4, 150 mM NaCl, 5 mM MgCl2, 1 mM DTT, 100 μg/mL cycloheximide added fresh) supplemented with 1% Triton X-100 and 25 U/ml Turbo DNase (Ambion), transferred to wet ice, harvested by scraping and further lysed by trituration ten times through a 26-gauge needle. Lysates were cleared by centrifugation at 20,000×g for 10 min at 4° C., flash frozen in liquid nitrogen, and stored at −80° C. To isolate ribosome-protected RNA fragments, lysates were thawed, digested with 2.5 U/μL RNase I (Ambion) for 45 minutes at room temperature with gentle mixing, and treated with 0.65 U/μL SUPERaseIn RNase Inhibitor (Thermo Fisher Scientific) to stop nuclease activity. Ribosome complexes were isolated using MicroSpin S-400 HR Columns (GE Healthcare) and RNA extracted using the Direct-zol RNA Miniprep Kit (Zymo Research). The rRNA Removal Mix—Gold component of Illumina's TruSeq Stranded Total RNA Library Prep Gold kit was used to deplete rRNAs. Samples were precipitated using GlycoBlue Coprecipitant (Ambion) as carrier, 0.1 volume 3M sodium acetate and 3 volumes 100% Ethanol, incubated at −80° C. overnight, centrifuged for 45 minutes at 20,000×g at 4° C., and RNA pellet dissolved in nuclease-free ultrapure water. Ribosome-protected fragments were isolated by running samples on a 15% TBE-Urea gel with 10 bp DNA Ladder (Promega) and Marker-27nt and Marker-30nt (data not shown). Gels were stained with SYBR Gold (Invitrogen) and RNA fragments 27-30 nucleotides were excised. Gel slices were disrupted with a small pestle and RNA eluted using constant agitation in 0.3 M NaCl at 4° C. overnight. Samples were run through Costar Spin-X filter to remove acrylamide, and RNA was precipitated in 100% isopropanol with GlycoBlue incubated on dry ice for 1 hour, followed by centrifugation for 45 minutes at 20,000×g at 4° C. RNA pellet was dissolved in 10 mM Tris pH 8.0, treated with T4 polynucleotide kinase (Thermo Fisher Scientific), and precipitated with isopropanol and GlycoBlue. RNA samples were diluted to equal input concentrations, libraries were prepared using the NEXTflex Small RNA-Seq Kit v3 (PerkinElmer) following the manufacturer's instructions, and sequenced using 50 bp paired-end sequencing on the Illumina NextSeq platform by the Fred Hutchinson Cancer Center Genomics Core.

### Polysome Fractionation

Polysome profiling was performed using three 70% confluent 15 cm plates of MB135iDUX4 myoblasts per treatment condition (n=3). MB135iDUX4 myoblasts were pulsed with or without doxycycline for 4 hours, incubated for 48 hours, supplemented with 50 ng/mL IFNG for 16 hours, and harvested at a terminal time point of 68 hours. To harvest, culture medium was supplemented with 100 μg/mL cycloheximide (CHX; Sigma) and cells were incubated at 37° C. for 10 minutes. Media was aspirated and each 15 cm plate of adherent cells was rinsed with 25 mL ice-cold PBS supplemented with 100 μg/mL cycloheximide, lifted with 0.25% Trypsin-EDTA supplemented with CHX, and resuspended in growth media supplemented with CHX. Cells were pelleted at 300 RCF and washed twice with ice-cold PBS supplemented with CHX. Cell pellets were flash frozen in liquid nitrogen and stored at −80° C. Cells were lysed with Polysome Lysis Buffer (10 mM Tris pH 8, 140 mM NaCl, 7.5 mM MgCl2, 0.25% NP-40, 0.1% Triton X-100, 150 μg/mL CHX, 20 mM DTT, 640U/mL SUPERase-In RNase Inhibitor) and clarified before quantification with the Bio-Rad Protein Assay. 1.5 mg of clarified lysate was loaded onto a 10-50% sucrose gradient prepared in DEPC-treated water with 25 mM Tris pH 7.4, 25 mM NaCl, 5 mM MgCl2, 100 μg/mL heparin, and 2 mM DTT. Gradients were fractionated using a Biocomp Piston Gradient Fractionator. Samples were resuspended in Trizol and Drosophila S2 cells were added as an internal spike-in control. RNA was extracted using the Direct-zol RNA Miniprep Kit (Zymo Research). Relative mRNA abundance for each sample was normalized to Drosophila spike-in to account for differences in RNA extraction efficiency (Taruttis et al., 2017).

### RNA Library Preparation and Sequencing

Total RNA was isolated using the NucleoSpin RNA kit (Machery-Nagel) or Direct-zol RNA Mini-Prep Kit (Zymo Research) according to manufacturer's instructions. RNA-seq libraries were prepared using the Illumina TruSeq RNA Sample Prep v2 Kit and sequenced using 50 bp paired-end sequencing on the Illumina NextSeq platform by the Fred Hutchinson Cancer Center Genomics Core.

Primer Sequences: ChIP-qPCR:
h16q21 forward:
(SEQ ID NO: 17)
AAACAAGCATCAGGGTGGAC;
h16q21 reverse:
(SEQ ID NO: 18)
GATCCCACAAAGGAAAGGAAC;
GBP1 forward:
(SEQ ID NO: 19)
TGGACAAATTCGTAGAAAGACTCA;
GBP1 reverse:
(SEQ ID NO: 20)
GCACAAAAACTGTCCCCAAC;
IDO1 reverse:
(SEQ ID NO: 21)
CACAGTCATTGTATTCTCTTTGCTG;
IDO1 reverse:
(SEQ ID NO: 22)
GCATATGGCTTTCGTTACAGTC;
CXCL10 reverse:
(SEQ ID NO: 23)
AAAGGAACAGTCTGCCCTGA;
CXCL10 reverse:
(SEQ ID NO: 24)
GCCCTGCTCTCCCATACTTT.
RT-qPCR:
IFIH1 forward:
(SEQ ID NO: 25)
CTAGCCTGTTCTGGGGAAGA;
IFIH1 reverse:
(SEQ ID NO: 26)
AGTCGGCACACTTCTTTTGC
ISG20 forward:
(SEQ ID NO: 27)
GAGCGCCTCCTACACAAGAG;
ISG20 reverse:
(SEQ ID NO: 28)
CGGATTCTCTGGGAGATTTG;
CXCL9 forward:
(SEQ ID NO: 29)
TCTTTTCCTCTTGGGCATCA;
CXCL9 reverse:
(SEQ ID NO: 30)
TAGTCCCTTGGTTGGTGCTG;
CD74 forward:
(SEQ ID NO: 31)
CGCGACCTTATCTCCAACAA;
CD74 reverse:
(SEQ ID NO: 32)
CAGGATGGAAAAGCCTGTGT;
GBP1 forward:
(SEQ ID NO: 33)
TAGCAGACTTCTGTTCCTACATCT;
GBP1 reverse:
(SEQ ID NO: 34)
CCACTGCTGATGGCATTGACGT;
IDO1 forward:
(SEQ ID NO: 35)
GCCAGCTTCGAGAAAGAGTTG;
IDO1 reverse:
(SEQ ID NO: 36)
ATCCCAGAACTAGACGTGCAA;
CXCL10 forward:
(SEQ ID NO: 37)
GTGGCATTCAAGGAGTACCTC;
CXCL10 reverse:
(SEQ ID NO: 38)
TGATGGCCTTCGATTCTGGATT.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A hypoimmunogenic cell comprising an exogenous DUX4 polypeptide or an exogenous nucleic acid encoding a DUX4 polypeptide for use in a method of treating a disease or a condition, wherein the exogenous nucleic acid encodes for a constitutive or an inducible expression of DUX4.

2. (canceled)

3. The hypoimmunogenic cell according to claim 1, wherein the exogenous DUX4 polypeptide or the exogenous nucleic acid encoding a DUX4 polypeptide comprises a transcriptionally inactive DUX4 protein.

4. The hypoimmunogenic cell according to claim 1, wherein the exogenous DUX4 polypeptide comprises:
(i) a DNA-binding deficient DUX4 polypeptide;
(ii) an amino acid mutation in a homeodomain region of DUX4;
(iii) a DUX4 polypeptide fragment lacking at least one homeodomain of the DUX4 protein;
(iv) a DUX4 protein or fragment thereof with at least 80% identity to a polypeptide comprising at least 80 contiguous amino acids of the DUX4 carboxy terminal region corresponding to amino acids 154-424 of the amino acid sequence as set forth in SEQ ID NO:3;
(v) a DUX4 polypeptide lacking the amino terminus of the DUX4 polypeptide or a portion thereof, wherein the amino terminus corresponds to amino acids 1-153 of the amino acid sequence as set forth in SEQ ID NO:3;
(vi) at least one (L)LxxL(L) motif, optionally at least one LxxL, LLxxL, LxxLL, and/or LLxxLL motif; or
(vii) at least two (L)LxxL(L) motifs, optionally at least two LxxL, LLxxL, LxxLL, and/or LLxxLL motifs, alone or in any combination.

5-9. (canceled)

10. The hypoimmunogenic cell according to claim 1, wherein the exogenous DUX4 polypeptide corresponds to a mammalian DUX4 polypeptide, wherein the mammal is selected from human, sheep, bovines, pigs, horses, rabbits, guinea pigs, mice, hamsters, rats, and non-human primates.

11. The hypoimmunogenic cell according to claim 1, wherein the exogenous nucleic acid encoding the exogenous DUX4 polypeptide is a codon altered sequence comprising one or more base substitutions to reduce the total number of CpG sites while preserving the DUX4 protein sequence, and wherein the codon altered sequence comprises a nucleotide sequence encoding a DUX4 protein or a fragment thereof.

12. (canceled)

13. The hypoimmunogenic cell according to claim 1, wherein the exogenous nucleic acid sequence encoding a DUX4 comprises a DUX4 protein or fragment thereof with at least 80% identity to a polypeptide comprising at least 80 contiguous amino acids of the DUX4 carboxy terminal region corresponding to amino acids 154-424 of the amino acid sequence as set forth in SEQ ID NO:3.

14. The hypoimmunogenic cell according to claim 1, wherein the exogenous DUX4 polypeptide or the exogenous nucleic acid encoding a DUX4 polypeptide comprises a transcriptionally active DUX4 protein.

15. The hypoimmunogenic cell according to claim 1, wherein:
(i) the exogenous nucleic acid encodes for an inducible expression DUX4 comprising a pulsed or transient expression of the DUX4 polypeptide; or
(ii) the exogenous nucleic acid encodes for a constitutive expression of the DUX4 comprising a continuous expression of the DUX4 polypeptide.

16. (canceled)

17. The hypoimmunogenic cell according to claim 1, further comprising a reduced expression of MHC-I and MHC-II human leukocyte antigens (HLA) relative to the wild-type cell of the same cell type, wherein the MHC-II human leukocyte antigens are HLA-DP, HLA-DQ, and HLA-DR.

18. (canceled)

19. The hypoimmunogenic cell according to claim 1, wherein the cell comprises;
(i) a stem cell, an induced pluripotent cell (iPSC), or a progenitor cell;
(ii) an in vitro differentiated cell; or
(iii) a primary T cell, a chimeric antigen receptor cell, a somatic cell, a hematopoietic stem, a progenitor cell, an induced pluripotent stem cell, an embryonic stem cell, an adult stem cell, a cardiac cell, a skeletal muscle stem cell, a mesenchymal stem cell, a lymphocyte, or a pancreatic islet cell.

20-21. (canceled)

22. The hypoimmunogenic cell according to claim 1, wherein:
(i) the exogenous nucleic acid encoding the DUX4 polypeptide is integrated into the genome of the cell;
(ii) the exogenous nucleic acid encoding the DUX4 polypeptide is integrated into the genome of the cell by targeted integration; or
(iii) the exogenous nucleic acid encoding the DUX4 polypeptide is not integrated into the genome of the cell.

23.-24. (canceled)

25. The hypoimmunogenic cell according to claim 22, wherein the exogenous nucleic acid encodes for constitutive or inducible expression of DUX4.

26. The hypoimmunogenic cell according to claim 1, further comprising a modification to increase expression of one or more tolerogenic factors selected from CD47, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9.

27. The hypoimmunogenic cell according to claim 1, wherein the disease or the condition is selected from:
(i) cancer, diabetes, a bone disorder, a blood disease, an enzyme deficiency, or a hemoglobinopathy, or for mediating tissue repair; or
(ii) acute leukemia, myelodysplastic syndrome, chronic myeloid leukemia, severe aplastic anemia, indolent lymphoma, chronic lymphocytic leukemia, severe immunodeficiency syndromes, hemoglobinopathies, lymphoma, Hodgkin's disease, multiple myeloma, autoimmune disorders, immune deficiencies, organ repair, or sickle cell anemia.

28. (canceled)

29. The hypoimmunogenic cell according to claim 27, wherein:
(i) the hypoimmunogenic cell comprises an allogeneic cell or a non-autologous cell; and/or
(ii) the hypoimmunogenic cell is 0, 1, 2, 3, 4, 5, or 6 antigen mismatch with a subject.

30. (canceled)

31. The hypoimmunogenic cell according to claim 1, wherein the hypoimmunogenic cell is from a mammal selected from human, sheep, bovines, pigs, horses, rabbits, guinea pigs, mice, hamsters, rats, and non-human primates.

32. A pharmaceutical composition comprising the hypoimmunogenic cell according to claim 1.

33. A method of modulating immune response in a cell comprising contacting the cell with or expressing an exogenous DUX4 polypeptide or an exogenous nucleic acid encoding a DUX4 polypeptide in the cell.

34. The method according to claim 33, wherein:
(i) the DUX4 polypeptide is transcriptionally inactive;
(ii) the DUX4 polypeptide comprises a DNA-binding deficient DUX4 polypeptide;
(iii) the DUX4 polypeptide comprises an amino acid mutation in a homeodomain region of DUX4 or comprises a DUX4 polypeptide fragment lacking at least one homeodomain of the DUX4 protein;
(iv) the DUX4 polypeptide comprises a DUX4 protein or fragment thereof with at least 80% identity to a polypeptide comprising at least 80 contiguous amino acids of the DUX4 carboxy terminal region corresponding to amino acids 154-424 of the amino acid sequence as set forth in SEQ ID NO:3;
(v) the DUX4 polypeptide lacks the amino terminus of the DUX4 polypeptide or a portion thereof, wherein the amino terminus corresponds to amino acids 1-153 of the amino acid sequence as set forth in SEQ ID NO:3;
(vi) the DUX4 polypeptide comprises at least one (L)LxxL(L) motif, optionally at least one LxxL, LLxxL, LxxLL, and/or LLxxLL motif; or
(vii) the DUX4 polypeptide comprises at least two (L)LxxL(L) motifs, optionally at least two LxxL, LLxxL, LxxLL, and/or LLxxLL motifs, alone or in any combination.

35-40. (canceled)

41. The method according to claim 33, wherein the DUX4 polypeptide corresponds to a mammalian DUX4 polypeptide, wherein the mammal is selected from human, sheep, bovines, pigs, horses, rabbits, guinea pigs, mice, hamsters, rats, and non-human primates.

42. The method according to claim 33, the method further comprising reducing the expression of MHC-I and MHC-II human leukocyte antigens (HLA) in the cell relative to the wild-type cell of the same cell type, wherein the MHC-I human leukocyte antigens are HLA-A, HLA-B, and HLA-C, and wherein the MHC-II human leukocyte antigens are HLA-DP, HLA-DQ, and HLA-DR.

43. (canceled)

44. The method according to claim 33, wherein the cell comprises:
(i) a stem cell, an induced pluripotent cell (iPSC), or a progenitor cell;
(ii) an in vitro differentiated cell; or
(iii) a T cell, a hematopoietic stem or progenitor cell, a cardiac cell, a skeletal muscle stem cell, a mesenchymal stem cell, a lymphocyte, or a pancreatic islet cell.

45-46. (canceled)

47. The method according to claim 33, wherein DUX4 expression in the cell is:
(i) constitutive or inducible;
(ii) inducible and comprises contacting the cell with or expressing the DUX4 polypeptide or the nucleic acid encoding a DUX4 polypeptide in the cell transiently;
(iii) inducible and comprises contacting the cell with or expressing the DUX4 polypeptide or the exogenous nucleic acid encoding a DUX4 polypeptide in the cell transiently, wherein the DUX4 polypeptide is transcriptionally active;
(iv) constitutive and comprises contacting the cell with or expressing the DUX4 polypeptide or the nucleic acid encoding a DUX4 polypeptide in the cell continuously; or
(v) constitutive and comprises contacting the cell with or expressing the DUX4 polypeptide or the exogenous nucleic acid encoding a DUX4 polypeptide in the cell continuously, wherein the DUX4 polypeptide is transcriptionally inactive.

48-51. (canceled)

52. The method according to claim 33, further comprising a modification of the cell to increase expression of one or more tolerogenic factors selected from CD47, CD27, CD46, CD55, CD59, CD200, HLA-C, HLA-E, HLA-E heavy chain, HLA-G, PD-L1, IDO1, CTLA4-Ig, C1-Inhibitor, IL-10, IL-35, FASL, CCL21, Mfge8, and Serpinb9.

53. The method according to claim 33, wherein DUX4 contacted with or expressed in the cell inhibits expression levels of interferon stimulated genes, one or more of canonical MHC-I subunits HLA-A, HLA-B, and HLA-C, and/or one or more of immunoproteasome subunits PSMB8, PSMB9, and PSMB10.

54. The method according to claim 33, wherein the cell is from a mammal, and wherein the mammal is selected from human, sheep, bovines, pigs, horses, rabbits, guinea pigs, mice, hamsters, rats, and non-human primates.

55. The method according to claim 33, wherein the method is effective in inhibiting antigen presentation by a cell.

56-75. (canceled)

76. The method according to claim 55, wherein the DUX4 polypeptide corresponds to a human DUX4 polypeptide.

77. The method according to claim 55, wherein antigen presentation by the cell is reduced for at least 12, 24, 36, 48, 60, 72, 84, or 96 hours.

78. The method according to claim 55, wherein:
(i) the cell is in vitro;
(ii) the cell is in vivo in a subject and the method comprises administering an effective amount of the exogenous DUX4 polypeptide or the exogenous nucleic acid encoding a DUX4 polypeptide to the subject;
(iii) the cell is in vivo in a subject and the method comprises administering an effective amount of the exogenous DUX4 polypeptide or the exogenous nucleic acid encoding a DUX4 polypeptide to the subject, wherein the subject has cancer, an inflammatory and/or autoimmune disease or other disease that would benefit from inhibition of antigen presentation; or
(iv) the cell is from a mammal selected from human, sheep, bovines, pigs, horses, rabbits, guinea pigs, mice, hamsters, rats, and non-human primates.

79-98. (canceled)


SEQUENCE LISTING (106 sequences)

SEQ ID NO: 1 - DNA, 1275 bp, Homo sapiens
  source 1..1275: mol_type = other DNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttag

SEQ ID NO: 2 - DNA, 1275 bp, synthetic construct
  source 1..1275: mol_type = other DNA
atggcattgcctacaccttcagactctacgctgcctgcagaggctaggggaagaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctggttccaaaatgaacgctctcggcagctcagacagcatcgcagggagtcccgcccgtggccaggaagaaggggaccacctgaaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 3 - protein, 424 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 4 - DNA, 1260 bp, synthetic construct
  source 1..1260: mol_type = other DNA
atggcattgcctacaccttcagactctacgctgcctgcagaggctaggggaagaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctggttccaaaatgaacgctctcggcagctcagacagcatcgcagggagtcccgcccgtggccaggaagaaggggaccacctgaaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcgccgctgaagccgcagctagcccagaatttctccagcaggcacagccactcctggaaacagaagctccgggagagctcgaagcctccgaagaagcagcaagcctggaggcacctctttccgaggaggagtatagagcctga

SEQ ID NO: 5 - protein, 419 aa, synthetic construct
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLAAEAAASPEFLQQAQPLLETEAPGELEASEEAASLEAPLSEEEYRA

SEQ ID NO: 6 - DNA, 1275 bp, synthetic construct
  source 1..1275: mol_type = other DNA
atggcattgcctacaccttcagactctacgctgcctgcagaggctaggggaagaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctgggcacaaaatgaacgctctcggcagctcagacagcatcgcagggagtcccgcccgtggccaggaagaaggggaccacctgaaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 7 - protein, 424 aa, synthetic construct
MALPTPSDSTLPAEARGRGRRRRLVWTPSQSEALRACFERNPYPGIATRERLAQAIGIPEPRVQIWAQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 8 - DNA, 930 bp, synthetic construct
  source 1..930: mol_type = other DNA
atggactacaaagaccatgacggtgattataaagatcatgacatcgattacaaggatgacgatgacaagccccccaagaggatgaggagggagccaaaaaagaagagaaaggtaggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 9 - protein, 309 aa, synthetic construct
MDYKDHDGDYKDHDIDYKDDDDKPPKRMRREPKKKRKVGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 10 - DNA, 915 bp, synthetic construct
  source 1..915: mol_type = other DNA
atggactacaaagaccatgacggtgattataaagatcatgacatcgattacaaggatgacgatgacaagccccccaagaggatgaggagggagccaaaaaagaagagaaaggtaggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcgccgctgaagccgcagctagcccagaatttctccagcaggcacagccactcctggaaacagaagctccgggagagctcgaagcctccgaagaagcagcaagcctggaggcacctctttccgaggaggagtatagagcctga

SEQ ID NO: 11 - protein, 304 aa, synthetic construct
MDYKDHDGDYKDHDIDYKDDDDKPPKRMRREPKKKRKVGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLAAEAAASPEFLQQAQPLLETEAPGELEASEEAASLEAPLSEEEYRA

SEQ ID NO: 12 - DNA, 351 bp, synthetic construct
  source 1..351: mol_type = other DNA
atggactacaaagaccatgacggtgattataaagatcatgacatcgattacaaggatgacgatgacaagccaaaaaagaagagaaaggtaaacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 13 - protein, 116 aa, synthetic construct
MDYKDHDGDYKDHDIDYKDDDDKPKKKRKVNASASARQGQMQGIPAPSQALQEPAPWSALPCGLLLDELLASPEFLQQAQPLLETEAPGELEASEEAASLEAPLSEEEYRALLEEL

SEQ ID NO: 14 - DNA, 2025 bp, Mus musculus
  source 1..2025: mol_type = other DNA
atggcagaagctggcagccctgttggtggcagtggtgtggcacgggaatcccggcggcgcaggaagacggtttggcaggcctggcaagagcaggccctgctatcaactttcaagaagaagagatacctgagcttcaaggagaggaaggagctggccaagcgaatgggggtctcagattgccgcatccgcgtgtggtttcagaaccgcaggaatcgcagtggagaggaggggcatgcctcaaagaggtccatcagaggctccaggcggctagcctcgccacagctccaggaagagcttggatccaggccacagggtagaggcatgcgctcatctggcagaaggcctcgcactcgactcacctcgctacagctcaggatcctagggcaagcctttgagaggaacccacgaccaggctttgctaccagggaggagctggcgcgtgacacagggttgcccgaggacacgatccacatatggtttcaaaaccgaagagctcggcggcgccacaggaggggcaggcccacagctcaagatcaagacttgctggcgtcacaagggtcggatggggcccctgcaggtccggaaggcagagagcgtgaaggtgcccaggagaacttgttgccacaggaagaagcaggaagtacgggcatggatacctcgagccctagcgacttgccctccttctgcggagagtcccagcctttccaagtggcacagccccgtggagcaggccaacaagaggcccccactcgagcaggcaacgcaggctctctggaacccctccttgatcagctgctggatgaagtccaagtagaagagcctgctccagcccctctgaatttggatggagaccctggtggcagggtgcatgaaggttcccaggagagcttttggccacaggaagaagcaggaagtacaggcatggatacttctagccccagcgactcaaactccttctgcagagagtcccagccttcccaagtggcacagccctgtggagcgggccaagaagatgcccgcactcaagcagacagcacaggccctctggaactcctcctccttgatcaactgctggacgaagtccaaaaggaagagcatgtgccagtcccactggattggggtagaaatcctggcagcagggagcatgaaggttcccaggacagcttactgcccctggaggaagcagtaaattcgggcatggatacctcgatccctagcatctggccaaccttctgcagagaatcccagcctccccaagtggcacagccctctggaccaggccaagcacaggcccccactcaaggtgggaacacggaccccctggagctcttcctctatcaactgttggatgaagtccaagtagaagagcatgctccagcccctctgaattgggatgtagatcctggtggcagggtgcatgaaggttcgtgggagagcttttggccacaggaagaagcaggaagtacaggcctggatacttcaagccccagcgactcaaactccttcttcagagagtccaagccttcccaagtggcacagcgccgtggagcgggccaagaagatgcccgcactcaagcagacagcacaggccctctggaactcctcctctttgatcaactgctggacgaagtccaaaaggaagagcatgtgccagccccactggattggggtagaaatcctggcagcatggagcatgaaggttcccaggacagcttactgcccctggaggaagcagcaaattcgggcagggatacctcgatccctagcatctggccagccttctgcagaaaatcccagcctccccaagtggcacagccctctggaccaggccaagcacaggcccccattcaaggtgggaacacggaccccctggagctcttccttgatcaactgctgaccgaagtccaacttgaggagcaggggcctgcccctgtgaatgtggaggaaacatgggagcaaatggacacaacacctgatctgcctctcacttcagaagaatatcagactcttctagatatgctctga

SEQ ID NO: 15 - protein, 674 aa, Mus musculus
MAEAGSPVGGSGVARESRRRRKTVWQAWQEQALLSTFKKKRYLSFKERKELAKRMGVSDCRIRVWFQNRRNRSGEEGHASKRSIRGSRRLASPQLQEELGSRPQGRGMRSSGRRPRTRLTSLQLRILGQAFERNPRPGFATREELARDTGLPEDTIHIWFQNRRARRRHRRGRPTAQDQDLLASQGSDGAPAGPEGREREGAQENLLPQEEAGSTGMDTSSPSDLPSFCGESQPFQVAQPRGAGQQEAPTRAGNAGSLEPLLDQLLDEVQVEEPAPAPLNLDGDPGGRVHEGSQESFWPQEEAGSTGMDTSSPSDSNSFCRESQPSQVAQPCGAGQEDARTQADSTGPLELLLLDQLLDEVQKEEHVPVPLDWGRNPGSREHEGSQDSLLPLEEAVNSGMDTSIPSIWPTFCRESQPPQVAQPSGPGQAQAPTQGGNTDPLELFLYQLLDEVQVEEHAPAPLNWDVDPGGRVHEGSWESFWPQEEAGSTGLDTSSPSDSNSFFRESKPSQVAQRRGAGQEDARTQADSTGPLELLLFDQLLDEVQKEEHVPAPLDWGRNPGSMEHEGSQDSLLPLEEAANSGRDTSIPSIWPAFCRKSQPPQVAQPSGPGQAQAPIQGGNTDPLELFLDQLLTEVQLEEQGPAPVNVEETWEQMDTTPDLPLTSEEYQTLLDML

SEQ ID NO: 16 - DNA, 2025 bp, synthetic construct
  source 1..2025: mol_type = other DNA
atggcagaagcagggtcacccgttggtgggagcggcgtagctagagagagtagacgcaggaggaaaaccgtttggcaagcctggcaggaacaagccttgttgtcaacattcaagaagaagcgctatttgtcctttaaggagcggaaagagttggcaaaaaggatgggagtaagtgattgtcgcataagggtatggtttcagaaccggaggaataggagcggggaagagggccatgccagtaagcgcagcatcagggggagccggcgcttggcaagtccccagctccaggaagaactcgggtcacgaccacagggcaggggtatgcgctcctccggccggagaccaagaactagattgacatcacttcaacttcggatccttggtcaggcttttgaacggaatccaaggccagggtttgccacacgcgaagaactcgcaagagacacaggcctccccgaagatactatacacatatggtttcagaacagaagagctcgccgccgacacaggaggggcagacccactgcccaggaccaagatctcctcgccagccagggaagtgatggtgcaccagcaggtcctgaagggcgcgaaagagaaggcgcccaggagaatctcctccctcaagaagaagccggcagcacaggcatggatactagctcacctagcgatctccccagcttctgtggagagtcccagcctttccaggtggctcagccaagaggggctggacaacaagaagcacccacaagagcaggtaacgcaggcagcttggaacctctgctggaccaactgttggacgaggttcaggtagaggaaccagcacctgctccattgaatctggacggggaccctgggggacgagtgcacgaggggagtcaagaatcattctggcctcaggaagaggcaggtagtactggcatggacacaagttccccaagcgacagtaactcattttgtagggagtcacagccttcccaggtagcccaaccatgtggggctggccaagaggatgcaaggacacaggcagattctactgggccattggagttgctcctcctcgatcagctcttggatgaggttcagaaagaggagcacgttcccgtgccactggattggggcagaaatcctggctcacgggaacatgagggctcccaagactccttgttgcctcttgaggaggcagtcaatagtggaatggacaccagtataccaagcatctggcctactttctgccgcgagtcacaaccaccccaagtagcacagccatccggacctggacaagctcaagctcccactcaaggaggcaacacagaccccctggagctgtttttgtaccagctgctcgatgaagtgcaggtcgaggaacacgctcccgctcccctcaattgggacgtggaccctggggggcgcgtgcacgaggggagttgggagagtttctggccccaagaggaagctggaagtaccgggcttgatacctcaagtccatctgacagcaactcattcttccgggagtctaaaccatctcaagtcgcccaaagacggggcgctggccaagaggacgctagaacccaagccgacagcacaggtcctcttgagttgctcctgttcgaccagctcctcgatgaggtccagaaagaggaacacgttcctgctcccctggactggggtagaaatcctggaagtatggaacatgagggcagccaagactcactccttcccttggaggaagcagccaacagcggacgcgatacaagtattccatccatctggcctgctttctgccgcaaatcccaacccccccaagtggcccaaccatctggccccggtcaagcccaggcccctatacagggcggcaataccgacccattggagctttttcttgaccagttgctcactgaggtacagctcgaagagcaaggccccgctcccgtcaatgtagaggaaacctgggagcagatggataccacccccgatttgccacttacatccgaggagtaccaaacactgttggatatgctctaa

SEQ ID NO: 17 - DNA, 20 bp, synthetic construct
  source 1..20: mol_type = other DNA
aaacaagcatcagggtggac

SEQ ID NO: 18 - DNA, 21 bp, synthetic construct
  source 1..21: mol_type = other DNA
gatcccacaaaggaaaggaac

SEQ ID NO: 19 - DNA, 24 bp, synthetic construct
  source 1..24: mol_type = other DNA
tggacaaattcgtagaaagactca

SEQ ID NO: 20 - DNA, 20 bp, synthetic construct
  source 1..20: mol_type = other DNA
gcacaaaaactgtccccaac

SEQ ID NO: 21 - DNA, 25 bp, synthetic construct
  source 1..25: mol_type = other DNA
cacagtcattgtattctctttgctg

SEQ ID NO: 22 - DNA, 22 bp, synthetic construct
  source 1..22: mol_type = other DNA
gcatatggctttcgttacagtc

SEQ ID NO: 23 - DNA, 20 bp, synthetic construct
  source 1..20: mol_type = other DNA
aaaggaacagtctgccctga

SEQ ID NO: 24 - DNA, 20 bp, synthetic construct
  source 1..20: mol_type = other DNA
gccctgctctcccatacttt

SEQ ID NO: 25 - DNA, 20 bp, synthetic construct
  source 1..20: mol_type = other DNA
ctagcctgttctggggaaga

SEQ ID NO: 26 - DNA, 20 bp, synthetic construct
  source 1..20: mol_type = other DNA
agtcggcacacttcttttgc

SEQ ID NO: 27 - DNA, 20 bp, synthetic construct
  source 1..20: mol_type = other DNA
gagcgcctcctacacaagag

SEQ ID NO: 28 - DNA, 20 bp, synthetic construct
  source 1..20: mol_type = other DNA
cggattctctgggagatttg

SEQ ID NO: 29 - DNA, 20 bp, synthetic construct
  source 1..20: mol_type = other DNA
tcttttcctcttgggcatca

SEQ ID NO: 30 - DNA, 20 bp, synthetic construct
  source 1..20: mol_type = other DNA
tagtcccttggttggtgctg

SEQ ID NO: 31 - DNA, 20 bp, synthetic construct
  source 1..20: mol_type = other DNA
cgcgaccttatctccaacaa

SEQ ID NO: 32 - DNA, 20 bp, synthetic construct
  source 1..20: mol_type = other DNA
caggatggaaaagcctgtgt

SEQ ID NO: 33 - DNA, 24 bp, synthetic construct
  source 1..24: mol_type = other DNA
tagcagacttctgttcctacatct

SEQ ID NO: 34 - DNA, 22 bp, synthetic construct
  source 1..22: mol_type = other DNA
ccactgctgatggcattgacgt

SEQ ID NO: 35 - DNA, 21 bp, synthetic construct
  source 1..21: mol_type = other DNA
gccagcttcgagaaagagttg

SEQ ID NO: 36 - DNA, 21 bp, synthetic construct
  source 1..21: mol_type = other DNA
atcccagaactagacgtgcaa

SEQ ID NO: 37 - DNA, 21 bp, synthetic construct
  source 1..21: mol_type = other DNA
gtggcattcaaggagtacctc

SEQ ID NO: 38 - DNA, 22 bp, synthetic construct
  source 1..22: mol_type = other DNA
tgatggccttcgattctggatt

SEQ ID NO: 39 - protein, 49 aa, Rattus norvegicus
LFLDQLLMEVRVETLSPGPVHLEGAVQQMDATPELPLSQEEYEALLDIL

SEQ ID NO: 40 - protein, 57 aa, Equus sp.
SLLEDLLAVTELQEKAQPFLNGHPPAEEPPGTLEGPLSEEEFQALLDMLQSSPGPQI

SEQ ID NO: 41 - protein, 57 aa, Canis sp.
SFLQELFSADEMEEDVHPLWVGTPQEDEPPGPLEAPLSEDDFHALLEMLQDSLWPQA

SEQ ID NO: 42 - protein, 49 aa, Delphinus sp.
SLLDELLSDVDILDKAGPFLNVDTEEEELAATLEAPLSEEEFKALLDIL

SEQ ID NO: 43 - protein, 57 aa, Bos sp.
SLLEEILAATAIQDTPWSSPGSPAGEEGVEATLETPLSEDEYQALLDMLPGSPGPGA

SEQ ID NO: 44 - protein, 55 aa, Armadillidium sp.
SILDELLLITECQETAPSLNLDSYEQDSTPAEPPLSEEDFQFLIMTLQNSLKASL

SEQ ID NO: 45 - protein, 60 aa, Homo sapiens
HRRCRTKFTEEQLKILINTFNQKPYPGYATKQKLALEINTEESRIQIWFQNRRARHGFQK

SEQ ID NO: 46 - protein, 60 aa, Pan troglodytes; identical to SEQ ID NO: 45

SEQ ID NO: 47 - protein, 60 aa, Macaca fascicularis
HRRSRTKFTEDQLKILIKAFDQNPYPGYATKQRLALEVNAEESRIQVWFQNRRARHQCQK

SEQ ID NO: 48 - protein, 60 aa, Canis sp.; identical to SEQ ID NO: 47

SEQ ID NO: 49 - protein, 60 aa, Mus musculus
RRHRKTVWQAWQEQALLSTFKKKRYLSFKERKELAKRMGVSDCRIRVWFQNRRNRSGEEG

SEQ ID NO: 50 - protein, 60 aa, Rattus norvegicus
SRRRKTVWQAWQKKALLSAFSKNKYPSFWDRQELARQIQLPASRIRVWFQNQRSRTGEVR

SEQ ID NO: 51 - protein, 60 aa, Bos sp.
SRRRRLVLKPSQKDALQALFQQNPYPGIATRERLARELGIDESRVQVWFQNQRRRRSKQS

SEQ ID NO: 52 - protein, 60 aa, Tupaia belangeri
ARRRRLVLSSSQKDALQALFQQNPYPATETRERLAQEIGIPESRVQVWFQNQRTRRARQA

SEQ ID NO: 53 - protein, 60 aa, Canis sp.
PRRRRLVLTASQKGALQAFFQKNPYPSITAREHLARELAISESRIQVWFQNQRTRQLRQS

SEQ ID NO: 54 - protein, 60 aa, Pan troglodytes
GRRRRLVWTPSQSEALRACFERNPYPGIATRERLAQAIGIPEPRVQIWFQNERSRQLRQH

SEQ ID NO: 55 - protein, 60 aa, Pongo pygmaeus
GRRRRLVWTPSQSEALRACFERNPYPGIATREGLAQAIGVPEPRVQIWFQNERSRQLRQH

SEQ ID NO: 56 - protein, 60 aa, Macaca fascicularis
GRRRRLVWTPSQREALRACFERNPYPGIATREELAQAIGIPEPRVQIWFQNERSRQLRQH

SEQ ID NO: 57 - protein, 60 aa, Callithrix jacchus
GRRKRLVWTPSQRDALRASFERNPYPGMATREQLAQAIGVPEPRVQIWFQNERSRRLRQH

SEQ ID NO: 58 - protein, 60 aa, Procavia capensis
GRRQRLVLEPSQTAILQASFEENPYPGISRREELARAIGIPEDRVQVWFGNRRASRSRKS

SEQ ID NO: 59 - protein, 60 aa, Loxodonta africana
GRRERLVLKPSQRESLRASFEQNPYPGITTREELARETGIAEDRIQTWFANRRAGLLRKS

SEQ ID NO: 60 - protein, 60 aa, Echinops telfairi
DRRKRIVWTTSQLEMLQASFARDPYPGITAREELARDTGVAEDRIFLWFKNRRSRQLRRS

SEQ ID NO: 61 - protein, 60 aa, Armadillidium sp.
ARRKRIFLDESQKGALQALFEQNHYSSLAAREKLAKEIGIPEDRIQIWFQNRRSRCLREG

SEQ ID NO: 62 - protein, 60 aa, Mus musculus
ARRRRIILTQSQKDTLRVWFEKNPNPDLATRGHLAKELGISESQIMTWFQKHRKIRKQAE

SEQ ID NO: 63 - protein, 60 aa, Rattus norvegicus
ARRRRIILNQSQKDTLRVWFEKNPNPDLATRGHLAKKLGISESQIMTWFQKHRKIRKQVE

SEQ ID NO: 64 - protein, 60 aa, Homo sapiens
FWRNRIQYNQSQKDILQSWFQHDPFPDKAAREQLAKEIGVPESNIQVWFKNYRVKQRKLD

SEQ ID NO: 65 - protein, 60 aa, Pan troglodytes; identical to SEQ ID NO: 64

SEQ ID NO: 66 - protein, 60 aa, Macaca fascicularis; identical to SEQ ID NO: 64

SEQ ID NO: 67 - protein, 60 aa, Canis sp.
TWQGRIVYNQSQKDILQEWFKQNPYPDKATRKQWAKEIGIPESEIQIWFKNHRAKQRRLE

SEQ ID NO: 68 - protein, 60 aa, Erinaceus sp.
AWQGRIVYNQSQKDILQKWFQHNPYPDKPSREQMAKEIGIPEYKIQIWFKNHRSKQRQLG

SEQ ID NO: 69 - protein, 60 aa, Loxodonta africana
KVQKRTAYSQRAKSILQVWFEYNPHPNKATREQLAEDIGVPEHKIQRWFRDARKKQRQLR

SEQ ID NO: 70 - protein, 60 aa, Homo sapiens
ARRCRTTYSASQLHTLIKAFMKNPYPGIDSREELAKEIGVPESRVQIWFQNRRSRLLLQR

SEQ ID NO: 71 - protein, 60 aa, Pan troglodytes; identical to SEQ ID NO: 70

SEQ ID NO: 72 - protein, 60 aa, Macaca fascicularis
ARRCRTTYSTSQLHTLIKAFVKNPYPGIDSREELAKEIGVPESRVQIWFQNRRSRLLLQR

SEQ ID NO: 73 - protein, 60 aa, Canis sp.
DRRCRTSYTSSQLHTLEIAFTNNPYPGIDTREQLAKEIGIPESRVQIWFQNRRSRFHVQR

SEQ ID NO: 74 - protein, 60 aa, Mus musculus
GRRPRTRLTSLQLRILGQAFERNPRPGFATREELARDTGLPEDTIHIWFQNRRARRRHRR

SEQ ID NO: 75 - protein, 60 aa, Rattus norvegicus
SKRPRTRLNLQQRRILVQAFERNPLPGFATREQLGQRTGLNEDTIHIWFQNRRARQARAP

SEQ ID NO: 76 - protein, 60 aa, Bos sp.
ARRKRTVISPSQTRILVQAFTRDRFPGIAAREELARQTGIPEPRIQIWFQNRRARHPQRS

SEQ ID NO: 77 - protein, 60 aa, Tupaia belangeri
GRRKRTAISASQTRILVQAFEGNRFPGIATRERLAQQTGLPESRIHIWFQNRRARHPGRS

SEQ ID NO: 78 - protein, 60 aa, Canis sp.
GRRKRTSISASQTSILLQAFEEERFPGIGMRESLARKTGLPEARIQVWFQNRRARHPGQS

SEQ ID NO: 79 - protein, 60 aa, Pan troglodytes
GRRKRTAVTGSQTALLLRAFEKDRFPGIAAREELARETGLPESRIQIWFQNRRARHPGQG

SEQ ID NO: 80 - protein, 60 aa, Pongo pygmaeus; identical to SEQ ID NO: 79

SEQ ID NO: 81 - protein, 60 aa, Macaca fascicularis
GRRKRTAVTRSQTALLLRAFQQDRFPGIATREELARETGLPESRIQIWFQNRRARHPGQG

SEQ ID NO: 82 - protein, 60 aa, Callithrix jacchus
GRRKRTAVTQSQTAVLLRAFERERFPDFATREELARETGLPESRIQIWFQNRRARHPGRA

SEQ ID NO: 83 - protein, 60 aa, Procavia capensis
ARRKRTRITAPQVSLLAEAFEKDRYPDFQAKSELARRTGLDESRVHIWFQNRRARHPAQS

SEQ ID NO: 84 - protein, 60 aa, Loxodonta africana
ARRKRTAITTSQTSLLVEAFEENRYPGNEAJEELAQRTGLPRSRIHVWFQNRRARKPVQS

SEQ ID NO: 85 - protein, 60 aa, Echinops telfairi
DRRKRTVITKDQTAYLVQAFEEQRFPGMAARQELARQTGLPESRIDTWFQNRRVRHPRPI

SEQ ID NO: 86 - protein, 60 aa, Armadillidium sp.
ARRKRTRITRSQSSILVEAFEQNRFPGITTREGLATQTGLPEFRIQIWFQNRRARYPGKT

SEQ ID NO: 87 - protein, 60 aa, Mus musculus
ARRSRTHFTKFQTDILIEAFEKNRFPGIVTREKLAQQTGIPESRIHIWFQNRRARHPDPG

SEQ ID NO: 88 - protein, 60 aa, Rattus norvegicus
AGRSRTHFTKFQIDILIEAFEKNRFPGIVTREKLAQETGIPESRIHIWFQNRRARHPDPK

SEQ ID NO: 89 - protein, 60 aa, Homo sapiens
ARQKQTFITWTQKNRLVQAFERNPFPDIATRKKLAEQTGLQESRIQMWFQKQRSLYLKKS

SEQ ID NO: 90 - protein, 60 aa, Pan troglodytes
ARQKQTFITWSQKNRLVQAFERNPFPDIATRKKLAEQTGLQESRIQMWFQKQRSLYLKKS

SEQ ID NO: 91 - protein, 60 aa, Macaca fascicularis
ARQKQTFITWTQKNKLVQAFERNPFPDIATRKKLTEQTGLQESRIQMWFQKQRSLYPKNC

SEQ ID NO: 92 - protein, 60 aa, Canis sp.
ARQDWTPITRSQSHTLVQAFERNHFPDITTRKKLAKKMGIQEPRIQVWFQNQRSLYPGQS

SEQ ID NO: 93 - protein, 60 aa, Erinaceus sp.
VKQDQTFGRRSPSTMLVQDFERSQSPDVATRKKMSKQTSIQESRIQKWFRNGRSLYPGQS

SEQ ID NO: 94 - protein, 60 aa, Loxodonta africana
ARSNQTSIMRCQTSTLLQAFQENQFPGLAATEKLAKQTEVPKSRIQMSFQNQEAQHLGHS

SEQ ID NO: 95 - DNA, 23 bp, synthetic construct
  source 1..23: mol_type = other DNA
taatacataccgtcagaagcagg

SEQ ID NO: 96 - DNA, 22 bp, synthetic construct
  source 1..22: mol_type = other DNA
taatacataccgtcagagcagg

SEQ ID NO: 97 - DNA, 23 bp, synthetic construct
  source 1..23: mol_type = other DNA
taatacataccgtcagaaagcag

SEQ ID NO: 98 - DNA, 23 bp, synthetic construct
  source 1..23: mol_type = other DNA
taatacataccgtcagacagcag

SEQ ID NO: 99 - protein, 56 aa, synthetic construct
PCGLLAAEAAASPEFLQQAQPLLETEAPGELEASEEAASLEAPLSEEEYRALLEEL

SEQ ID NO: 100 - protein, 50 aa, synthetic construct
PCGLLLDELLASPEFLQQAQPLLETEAPGELEASEEAASLEAPLSEEYRA

SEQ ID NO: 101 - DNA, 4893 bp, synthetic construct
  source 1..4893: mol_type = other DNA
atggactacaaagaccatgacggtgattataaagatcatgacatcgattacaaggatgacgatgacaagtattcggcccacaggcccctgatgcccgcgtccagcgcggcctcccgtggcctcggcatgttcgtgtggacgaatgtggaacctcgctctgtggctgtgttcccttggcactccttagtccccttcctggcacccagccagcctgacccctccgtgcagccgagcgaggcccagcaacctgccagccacccagtggcctccaaccagagcaaagaacctgctgagtcggcagctgttgctcatgaacggccaccaggtgggacagggagtgctgaccctgagcggccccctggagccacatgccctgagagcccaggacccggacccccacaccctttgggggtggtggaatctggtaagggtccgcctcccaccacggaggaggaggcctccggccccccaggagagccccggctggacagtgagacagagagtgaccatgatgatgccttcctctccatcatgtctcctgagatccagttgcctctaccgcccggaaaacgtcggacccagtccctcagtgccctacccaaggaacgggactcatcttctgagaaggatggacgcagccccaacaagcgggagaaggaccacatccggcggcccatgaatgccttcatgatcttcagcaagcggcaccgggccctggtccaccagcgtcatcccaaccaggacaaccggaccgtcagcaagatcctgggcgagtggtggtatgccctggggcccaaggagaagcagaagtaccacgacctggccttccaggtgaaggaggcccacttcaaggcccacccagattggaagtggtgtaacaaggaccgaaagaagtccagctcagaggccaagcccacgagcctggggctggcaggagggcacaaggagacgcgggagcggagcatgtcggagacgggcactgctgctgcccctggggtgtcctctgagctcctgtccgttgcagcccagacactcctgagctcagacaccaaggctccggggagcagctcctgtggggcagaacggctacacacagttgggggacctggctcagcccggccccgagctttctcccacagcggggtacacagcctggacggcggagaagtagacagtcaggcgctacaggaactgacgcagatggtgtctggccctgcatcgtactctggcccaaagccttctacccagtatggagctccaggaccctttgcagcccctggtgagggaggtgccttggcggccactgggcggcccccgctgctgcccacccgagcttctcgttctcagcgtgcggccagtgaggacatgacgagtgatgaggagcgcatggtcatctgtgaggaggaaggggatgatgatgtcattgctgacgatggcttcggcaccactgacattgatctcaagtgcaaggagcgggtgaccgacagcgagagtggggacagctctggggaggacccagagggcaacaagggctttggtcggaaggtgttttcacctgtgatccgttcctcctttacccactgccgccccccactggaccctgagcccccagggcccccggatcctcctgtagcctttggcaaaggctatggttccgccccatcctcctctgcgtcctcgcctgcttcctcctcagcctcggcagccacctccttctcactgggctcaggaaccttcaaggcccaggagtctggtcagggcagcacagcgggccccctacggcccccaccccctggggctgggggtccagcgacaccttccaaggcaacccggttcctcccaatggatcctgccaccttccggcgcaagagacccgaaagtgtgggtggcctggagccaccaggcccctcagtcatcgcggcccctcccagcggaggaggaaacatcctgcagacactggtgctgcccccaaacaaggaggagcaagagggcggcggagccagagtgccctccgcccccgccccatcactggcctatggggccccagcagctcccctgtcccgtcctgccgccaccatggtcaccaatgtggtgcggcctgtcagcagcactcctgtgcccatcgcctctaagcccttccccacctctggccgggctgaggcgtctccaaatgacacagcaggtgccaggactgaaatgggcactgggtctcgggtgcctgggggctccccgctgggtgtcagcttagtgtattcggacaagaagtcggcagcagccacctcaccagccccacacttggtggctggacccctgctgggcactgtggggaaggcgcctgccactgtcactaacctactggtgggcaccccggggtatggggcccctgcgccccctgctgtccagttcattgcccagggggcccctggtggtgggaccactgcgggctcaggagcaggtgctgggagtggccccaatgggccagtacccctgggcatcctgcaaccaggtgccctgggcaaggctgggggaatcacccaggtacagtacatcctgcccacgctgccccagcagcttcaggtggcacctgccccagcaccagcccctgggaccaaggcagcggctcccagcggccctgcacccaccaccagcatccgtttcaccctcccaccgggcacttccaccaacggcaaagtcctggctgccactgcacccactcctggcatccccatcctgcagtctgtaccctccgccccaccccccaaagcccagtcagtttctcccgtgcaggccccgcccccgggtggctcagcccagctgctgcctgggaaggtcctagtgcctctggccgcccctagcatgtcagtgcggggtggaggggccggccagccactgccactggtgagcccgcccttctcagtacctgtgcagaatggtgcccagccccccagcaagatcatccagctgaccccggtgcctgtgagcacacccagcggcctggtgccgcccctgagcccagccacactccctggacccacctctcagcctcagaaggtcctgttgccctcctccaccagaatcacctatgtgcagtcagcgggcgggcacgcgctgcccctgggtaccagccctgcgtccagccaggctggaacagtcacctcgtacgggcccacgagctctgtagctctaggcttcacctcgctggggcccagcggccccgccttcgtgcagcccctgctctcagcaggccaagccccactgctggctcccggtcaggtgggcgtgtcacctgtgcccagtccccagctgccgcctgcctgtgcagcccccggaggtcctgtcataacagcattttactctggcagccctgcacccacctcctcagcacccctggcccagccatcccaggcccccccaagcctggtctacactgtggccaccagcacaaccccacctgcagccaccattctgcccaagggcccgccagcccctgccactgccaccccagccccgactagccctttccccagcgccacagcaggttccatgacctacagcttagtggcccccaaggcccagcggcccagcccgaaggccccccagaaagtgaaggcagccatcgccagcattcccgtggggtcctttgaggcaggtgcctctgggcggcctggccctgcaccccggcagcctctggagcctggcccagtccgagagccaactgccccagagtctgagcttgaggggcagcccacaccaccagcccctccacccctgccagagacctggactcccacggcccggagcagccccccactgcccccacctgctgaggagcggaccagcgccaagggccctgagaccatggccagcaaattccccagctcatcttcagactggcgcgtccctgggcagggcctggagaatcgtggggagcctcccactcctcccagcccggccccagctccagctgtagcccctggtggcagcagcgagagcagcagtgggcgggcagccggggacaccccggagcgcaaggaggcggctggtactggcaagaaggtgaaggtgcggcccccgcccctgaagaagacctttgactctgtggacaacagggtcctgtcagaagtggacttcgaagagcgctttgctgagttgcctgagtttcggcctgaggaggtgctgccctcccccaccctgcagtctctggccacctcaccccgggccatcctgggctcttaccgcaagaagaggaagaactccacggacctggattcagcacccgaggaccccacctcgcccaagcgcaagatgagaagacgctccagctgcagctcggagcccaacacccccaagagtgccaagtgcgagggggacatcttcacctttgaccgtacaggtacagaagccgaggacgtgcttggggagctagagtatgacaaggtgccatactcctccctgcggcgcaccctggaccagcgccgggccctggtcatgcagctctttcaggaccatggcttcttcccgtcagcccaggccacagccgccttccaggcccgctatgcagacatctttccctccaaggtttgtctgcagttgaagatccgtgaggtgcgccagaagatcatgcaggctgccactcccacggagcagccccctggagctgaggctcctctccctgtaccgccccccactggcaccgctgctgcccctgcccccactcccagccccgcagggggccctgaccccacctcacccagctcggactctggcacggcccaggctgccccgccactgcctccacccccagagtcggggcctggacagcctggctgggagggggctccccagccctcccccccacccccaggtccctccacagctgccacaggcaggtga

SEQ ID NO: 102 - protein, 1630 aa, synthetic construct
MDYKDHDGDYKDHDIDYKDDDDKYSAHRPLMPASSAASRGLGMFVWTNVEPRSVAVFPWHSLVPFLAPSQPDPSVQPSEAQQPASHPVASNQSKEPAESAAVAHERPPGGTGSADPERPPGATCPESPGPGPPHPLGVVESGKGPPPTTEEEASGPPGEPRLDSETESDHDDAFLSIMSPEIQLPLPPGKRRTQSLSALPKERDSSSEKDGRSPNKREKDHIRRPMNAFMIFSKRHRALVHQRHPNQDNRTVSKILGEWWYALGPKEKQKYHDLAFQVKEAHFKAHPDWKWCNKDRKKSSSEAKPTSLGLAGGHKETRERSMSETGTAAAPGVSSELLSVAAQTLLSSDTKAPGSSSCGAERLHTVGGPGSARPRAFSHSGVHSLDGGEVDSQALQELTQMVSGPASYSGPKPSTQYGAPGPFAAPGEGGALAATGRPPLLPTRASRSQRAASEDMTSDEERMVICEEEGDDDVIADDGFGTTDIDLKCKERVTDSESGDSSGEDPEGNKGFGRKVFSPVIRSSFTHCRPPLDPEPPGPPDPPVAFGKGYGSAPSSSASSPASSSASAATSFSLGSGTFKAQESGQGSTAGPLRPPPPGAGGPATPSKATRFLPMDPATFRRKRPESVGGLEPPGPSVIAAPPSGGGNILQTLVLPPNKEEQEGGGARVPSAPAPSLAYGAPAAPLSRPAATMVTNVVRPVSSTPVPIASKPFPTSGRAEASPNDTAGARTEMGTGSRVPGGSPLGVSLVYSDKKSAAATSPAPHLVAGPLLGTVGKAPATVTNLLVGTPGYGAPAPPAVQFIAQGAPGGGTTAGSGAGAGSGPNGPVPLGILQPGALGKAGGITQVQYILPTLPQQLQVAPAPAPAPGTKAAAPSGPAPTTSIRFTLPPGTSTNGKVLAATAPTPGIPILQSVPSAPPPKAQSVSPVQAPPPGGSAQLLPGKVLVPLAAPSMSVRGGGAGQPLPLVSPPFSVPVQNGAQPPSKIIQLTPVPVSTPSGLVPPLSPATLPGPTSQPQKVLLPSSTRITYVQSAGGHALPLGTSPASSQAGTVTSYGPTSSVALGFTSLGPSGPAFVQPLLSAGQAPLLAPGQVGVSPVPSPQLPPACAAPGGPVITAFYSGSPAPTSSAPLAQPSQAPPSLVYTVATSTTPPAATILPKGPPAPATATPAPTSPFPSATAGSMTYSLVAPKAQRPSPKAPQKVKAAIASIPVGSFEAGASGRPGPAPRQPLEPGPVREPTAPESELEGQPTPPAPPPLPETWTPTARSSPPLPPPAEERTSAKGPETMASKFPSSSSDWRVPGQGLENRGEPPTPPSPAPAPAVAPGGSSESSSGRAAGDTPERKEAAGTGKKVKVRPPPLKKTFDSVDNRVLSEVDFEERFAELPEFRPEEVLPSPTLQSLATSPRAILGSYRKKRKNSTDLDSAPEDPTSPKRKMRRRSSCSSEPNTPKSAKCEGDIFTFDRTGTEAEDVLGELEYDKVPYSSLRRTLDQRRALVMQLFQDHGFFPSAQATAAFQARYADIFPSKVCLQLKIREVRQKIMQAATPTEQPPGAEAPLPVPPPTGTAAAPAPTPSPAGGPDPTSPSSDSGTAQAAPPLPPPPESGPGQPGWEGAPQPSPPPPGPSTAATGR

SEQ ID NO: 103 - DNA, 5075 bp, synthetic construct
  source 1..5075: mol_type = other DNA
atggactacaaagaccatgacggtgattataaagatcatgacatcgattacaaggatgacgatgacaagtattcggcccacaggcccctgatgcccgcgtccagcgcggcctcccgtggcctcggcatgttcgtgtggacgaatgtggaacctcgctctgtggctgtgttcccttggcactccttagtccccttcctggcacccagccagcctgacccctccgtgcagccgagcgaggcccagcaacctgccagccacccagtggcctccaaccagagcaaagaacctgctgagtcggcagctgttgctcatgaacggccaccaggtgggacagggagtgctgaccctgagcggccccctggagccacatgccctgagagcccaggacccggacccccacaccctttgggggtggtggaatctggtaagggtccgcctcccaccacggaggaggaggcctccggccccccaggagagccccggctggacagtgagacagagagtgaccatgatgatgccttcctctccatcatgtctcctgagatccagttgcctctaccgcccggaaaacgtcggacccagtccctcagtgccctacccaaggaacgggactcatcttctgagaaggatggacgcagccccaacaagcgggagaaggaccacatccggcggcccatgaatgccttcatgatcttcagcaagcggcaccgggccctggtccaccagcgtcatcccaaccaggacaaccggaccgtcagcaagatcctgggcgagtggtggtatgccctggggcccaaggagaagcagaagtaccacgacctggccttccaggtgaaggaggcccacttcaaggcccacccagattggaagtggtgtaacaaggaccgaaagaagtccagctcagaggccaagcccacgagcctggggctggcaggagggcacaaggagacgcgggagcggagcatgtcggagacgggcactgctgctgcccctggggtgtcctctgagctcctgtccgttgcagcccagacactcctgagctcagacaccaaggctccggggagcagctcctgtggggcagaacggctacacacagttgggggacctggctcagcccggccccgagctttctcccacagcggggtacacagcctggacggcggagaagtagacagtcaggcgctacaggaactgacgcagatggtgtctggccctgcatcgtactctggcccaaagccttctacccagtatggagctccaggaccctttgcagcccctggtgagggaggtgccttggcggccactgggcggcccccgctgctgcccacccgagcttctcgttctcagcgtgcggccagtgaggacatgacgagtgatgaggagcgcatggtcatctgtgaggaggaaggggatgatgatgtcattgctgacgatggcttcggcaccactgacattgatctcaagtgcaaggagcgggtgaccgacagcgagagtggggacagctctggggaggacccagagggcaacaagggctttggtcggaaggtgttttcacctgtgatccgttcctcctttacccactgccgccccccactggaccctgagcccccagggcccccggatcctcctgtagcctttggcaaaggctatggttccgccccatcctcctctgcgtcctcgcctgcttcctcctcagcctcggcagccacctccttctcactgggctcaggaaccttcaaggcccaggagtctggtcagggcagcacagcgggccccctacggcccccaccccctggggctgggggtccagcgacaccttccaaggcaacccggttcctcccaatggatcctgccaccttccggcgcaagagacccgaaagtgtgggtggcctggagccaccaggcccctcagtcatcgcggcccctcccagcggaggaggaaacatcctgcagacactggtgctgcccccaaacaaggaggagcaagagggcggcggagccagagtgccctccgcccccgccccatcactggcctatggggccccagcagctcccctgtcccgtcctgccgccaccatggtcaccaatgtggtgcggcctgtcagcagcactcctgtgcccatcgcctctaagcccttccccacctctggccgggctgaggcgtctccaaatgacacagcaggtgccaggactgaaatgggcactgggtctcgggtgcctgggggctccccgctgggtgtcagcttagtgtattcggacaagaagtcggcagcagccacctcaccagccccacacttggtggctggacccctgctgggcactgtggggaaggcgcctgccactgtcactaacctactggtgggcaccccggggtatggggcccctgcgccccctgctgtccagttcattgcccagggggcccctggtggtgggaccactgcgggctcaggagcaggtgctgggagtggccccaatgggccagtacccctgggcatcctgcaaccaggtgccctgggcaaggctgggggaatcacccaggtacagtacatcctgcccacgctgccccagcagcttcaggtggcacctgccccagcaccagcccctgggaccaaggcagcggctcccagcggccctgcacccaccaccagcatccgtttcaccctcccaccgggcacttccaccaacggcaaagtcctggctgccactgcacccactcctggcatccccatcctgcagtctgtaccctccgccccaccccccaaagcccagtcagtttctcccgtgcaggccccgcccccgggtggctcagcccagctgctgcctgggaaggtcctagtgcctctggccgcccctagcatgtcagtgcggggtggaggggccggccagccactgccactggtgagcccgcccttctcagtacctgtgcagaatggtgcccagccccccagcaagatcatccagctgaccccggtgcctgtgagcacacccagcggcctggtgccgcccctgagcccagccacactccctggacccacctctcagcctcagaaggtcctgttgccctcctccaccagaatcacctatgtgcagtcagcgggcgggcacgcgctgcccctgggtaccagccctgcgtccagccaggctggaacagtcacctcgtacgggcccacgagctctgtagctctaggcttcacctcgctggggcccagcggccccgccttcgtgcagcccctgctctcagcaggccaagccccactgctggctcccggtcaggtgggcgtgtcacctgtgcccagtccccagctgccgcctgcctgtgcagcccccggaggtcctgtcataacagcattttactctggcagccctgcacccacctcctcagcacccctggcccagccatcccaggcccccccaagcctggtctacactgtggccaccagcacaaccccacctgcagccaccattctgcccaagggcccgccagcccctgccactgccaccccagccccgactagccctttccccagcgccacagcaggttccatgacctacagcttagtggcccccaaggcccagcggcccagcccgaaggccccccagaaagtgaaggcagccatcgccagcattcccgtggggtcctttgaggcaggtgcctctgggcggcctggccctgcaccccggcagcctctggagcctggcccagtccgagagccaactgccccagagtctgagcttgaggggcagcccacaccaccagcccctccacccctgccagagacctggactcccacggcccggagcagccccccactgcccccacctgctgaggagcggaccagcgccaagggccctgagaccatggccagcaaattccccagctcatcttcagactggcgcgtccctgggcagggcctggagaatcgtggggagcctcccactcctcccagcccggccccagctccagctgtagcccctggtggcagcagcgagagcagcagtgggcgggcagccggggacaccccggagcgcaaggaggcggctggtactggcaagaaggtgaaggtgcggcccccgcccctgaagaagacctttgactctgtggacaacagggtcctgtcagaagtggacttcgaagagcgctttgctgagttgcctgagtttcggcctgaggaggtgctgccctcccccaccctgcagtctctggccacctcaccccgggccatcctgggctcttaccgcaagaagaggaagaactccacggacctggattcagcacccgaggaccccacctcgcccaagcgcaagatgagaagacgctccagctgcagctcggagcccaacacccccaagagtgccaagtgcgagggggacatcttcacctttgaccgtacaggtacagaagccgaggacgtgcttggggagctagagtatgacaaggtgccatactcctccctgcggcgcaccctggaccagcgccgggccctggtcatgcagctctttcaggaccatggcttcttcccgtcagcccaggccacagccgccttccaggcccgctatgcagacatctttccctccaaggtttgtctgcagttgaagatccgtgaggtgcgccagaagatcatgcaggctgccactcccacggagcagccccctggagctgaggctcctctccctgtaccgccccccactggcaccgctgctgcccctgcccccactcccagccccgcagggggccctgaccccacctcacccagctcggactctgggggtggaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagcttta

SEQ ID NO: 104 - protein, 1691 aa, synthetic construct
MDYKDHDGDYKDHDIDYKDDDDKYSAHRPLMPASSAASRGLGMFVWTNVEPRSVAVFPWHSLVPFLAPSQPDPSVQPSEAQQPASHPVASNQSKEPAESAAVAHERPPGGTGSADPERPPGATCPESPGPGPPHPLGVVESGKGPPPTTEEEASGPPGEPRLDSETESDHDDAFLSIMSPEIQLPLPPGKRRTQSLSALPKERDSSSEKDGRSPNKREKDHIRRPMNAFMIFSKRHRALVHQRHPNQDNRTVSKILGEWWYALGPKEKQKYHDLAFQVKEAHFKAHPDWKWCNKDRKKSSSEAKPTSLGLAGGHKETRERSMSETGTAAAPGVSSELLSVAAQTLLSSDTKAPGSSSCGAERLHTVGGPGSARPRAFSHSGVHSLDGGEVDSQALQELTQMVSGPASYSGPKPSTQYGAPGPFAAPGEGGALAATGRPPLLPTRASRSQRAASEDMTSDEERMVICEEEGDDDVIADDGFGTTDIDLKCKERVTDSESGDSSGEDPEGNKGFGRKVFSPVIRSSFTHCRPPLDPEPPGPPDPPVAFGKGYGSAPSSSASSPASSSASAATSFSLGSGTFKAQESGQGSTAGPLRPPPPGAGGPATPSKATRFLPMDPATFRRKRPESVGGLEPPGPSVIAAPPSGGGNILQTLVLPPNKEEQEGGGARVPSAPAPSLAYGAPAAPLSRPAATMVTNVVRPVSSTPVPIASKPFPTSGRAEASPNDTAGARTEMGTGSRVPGGSPLGVSLVYSDKKSAAATSPAPHLVAGPLLGTVGKAPATVTNLLVGTPGYGAPAPPAVQFIAQGAPGGGTTAGSGAGAGSGPNGPVPLGILQPGALGKAGGITQVQYILPTLPQQLQVAPAPAPAPGTKAAAPSGPAPTTSIRFTLPPGTSTNGKVLAATAPTPGIPILQSVPSAPPPKAQSVSPVQAPPPGGSAQLLPGKVLVPLAAPSMSVRGGGAGQPLPLVSPPFSVPVQNGAQPPSKIIQLTPVPVSTPSGLVPPLSPATLPGPTSQPQKVLLPSSTRITYVQSAGGHALPLGTSPASSQAGTVTSYGPTSSVALGFTSLGPSGPAFVQPLLSAGQAPLLAPGQVGVSPVPSPQLPPACAAPGGPVITAFYSGSPAPTSSAPLAQPSQAPPSLVYTVATSTTPPAATILPKGPPAPATATPAPTSPFPSATAGSMTYSLVAPKAQRPSPKAPQKVKAAIASIPVGSFEAGASGRPGPAPRQPLEPGPVREPTAPESELEGQPTPPAPPPLPETWTPTARSSPPLPPPAEERTSAKGPETMASKFPSSSSDWRVPGQGLENRGEPPTPPSPAPAPAVAPGGSSESSSGRAAGDTPERKEAAGTGKKVKVRPPPLKKTFDSVDNRVLSEVDFEERFAELPEFRPEEVLPSPTLQSLATSPRAILGSYRKKRKNSTDLDSAPEDPTSPKRKMRRRSSCSSEPNTPKSAKCEGDIFTFDRTGTEAEDVLGELEYDKVPYSSLRRTLDQRRALVMQLFQDHGFFPSAQATAAFQARYADIFPSKVCLQLKIREVRQKIMQAATPTEQPPGAEAPLPVPPPTGTAAAPAPTPSPAGGPDPTSPSSDSGGGPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 105 - DNA, 1149 bp, synthetic construct
  source 1..1149: mol_type = other DNA
atggactacaaagaccatgacggtgattataaagatcatgacatcgattacaaggatgacgatgacaagccccccaagaggatgaggagggagccaaaaaagaagagaaaggtaaatctggaaggcacgagtggaggaatcctgcaaaaagagttttggagaaatcgaatacagtacaatcaatctcaaaaagatatactccaatcctggttccaacatgatccgttccctgacaaggctgctcgcgagcaactcgcgaaggagattggggttccggagagtaacatccaggtctggtttaaaaactatcgcgtcaaacagcgcaagctggattataaatgcttcagcgagaaggaccaaactcaggggcatgaccaatcccaacacttgacccaggaatatctccctaaagaagcccgccaaaaacaaaccttcataacctggactcagaagaatagacttgtacaagcattcgagcgaaacccatttccagacattgccacgcgcaaaaagctcgccgagcaaaccggactccaagagtccagaatccaaatgtggttccaaaaacaacgctctctctacttgaagaagtccagaatggaacctatgaatctgcttgtagatgacccgaacgagcgccccgatgcgacagtaggatggcaccctataaatctgtttctgccgacggatagtagccactattttagctgttctcactcttcctcagggcacgagactcttcccccagttttgcccagtacacaggctccttgggatccttttagatttcacgtatcccaagggcctaatgtgatgattatgcagccaactcaagcagttcaagaaggcgagaaatcagaccaaccactgattatccccaaccatctgcttacactgcccattctcactaaggatcttgataccccgacgccgttttggcttcaatatcaagaggagcaccagaatcacaaggaacacagtggaagcggcgttccccaagttaagtctcacagccaacccgaacccgagcatagggaacaacagcctcttaacctcggtcagtttgatatctccaacattcttcaacgatgggacgaaatctgtcaagccttgttggcggagtgggaaccgctgaagggcacccattga

SEQ ID NO: 106 - protein, 381 aa, synthetic construct
DYKDHDGDYKDHDIDYKDDDDKPPKRMRREPKKKRKVNLEGTSGGILQKEFWRNRIQYNQSQKDILQSWFQHDPFPDKAAREQLAKEIGVPESNIQVWFKNYRVKQRKLDYKCFSEKDQTQGHDQSQHLTQEYLPKEARQKQTFITWTQKNRLVQAFERNPFPDIATRKKLAEQTGLQESRIQMWFQKQRSLYLKKSRMEPMNLLVDDPNERPDATVGWHPINLFLPTDSSHYFSCSHSSSGHETLPPVLPSTQAPWDPFRFHVSQGPNVMIMQPTQAVQEGEKSDQPLIIPNHLLTLPILTKDLDTPTPFWLQYQEEHQNHKEHSGSGVPQVKSHSQPEPEHREQQPLNLGQFDISNILQRWDEICQALLAEWEPLKGTH